(19) United States
(12) Patent Application Publication
(10) Pub. No.: US 2026/0158129 A1
(43) Pub. Date: Jun. 11, 2026

(54) NUCLEIC ACID INFLUENZA VACCINES AND RESPIRATORY VIRUS COMBINATION VACCINES

(71) Applicant: ModernaTX, Inc., Cambridge, MA (US)

(72) Inventors: Raffael Nachbagauer, Rahway, NJ (US); Farbod Mahmoudinobar, Rahway, NJ (US); Elif Nihal Korkmaz, Brookline, MA (US)

(73) Assignee: ModernaTX, Inc., Cambridge, MA (US)

(21) Appl. No.: 19/398,368

(22) Filed: Nov. 24, 2025

Related U.S. Application Data
(63) Continuation of application No. 19/320,264, filed on Sep. 5, 2025.
Application No. 19/320,264 is a continuation of application No. PCT/US2024/019210, filed on Mar. 8, 2024.
(60) Provisional application No. 63/489,707, filed on Mar. 10, 2023.
(60) Provisional application No. 63/518,923, filed on Aug. 11, 2023.
(60) Provisional application No. 63/582,208, filed on Sep. 12, 2023.

(51) Int. Cl. A61K 39/145; A61K 9/1271; A61K 9/51; A61K 39/00; A61P 31/16; C07K 14/005; C12N 7/00

(52) U.S. Cl. CPC A61K 39/145; A61K 9/1271; A61K 9/5123; A61P 31/16; C07K 14/005; C12N 7/00; A61K 2039/53; A61K 2039/55555; A61K 2039/575; C12N 2760/16222; C12N 2760/16234

(57) ABSTRACT

Some aspects of the disclosure relate to vaccines (e.g., RNA vaccines (e.g., mRNA vaccines)) for seasonal influenza viruses as well as methods of using the vaccines. Also described are combination vaccines (e.g., RNA vaccines (e.g., mRNA vaccines)) for seasonal influenza viruses and other respiratory viruses (e.g., respiratory syncytial viruses and coronaviruses), as well as methods of using the vaccines.

### RELATED APPLICATIONS

This application is a continuation of U.S. application Ser. No. 19/320,264, filed Sep. 5, 2025, which is a continuation of International Patent Application No. PCT/US2024/019210, filed Mar. 8, 2024, which claims the benefit under 35 U.S.C. § 119(e) of U.S. Provisional Application No. 63/489,707, filed Mar. 10, 2023, U.S. Provisional Application No. 63/518,923, filed Aug. 11, 2023, and U.S. Provisional Application No. 63/582,208, filed Sep. 12, 2023, the contents of each of which are incorporated by reference herein in their entirety.

### REFERENCE TO AN ELECTRONIC SEQUENCE LISTING

The contents of the electronic sequence listing (M137870260WO00-SEQ-NTJ.xml; Size: 400,770 bytes; and Date of Creation: Mar. 8, 2024) are incorporated by reference herein in their entirety.

### BACKGROUND

Respiratory diseases, encompassing a range of conditions affecting the gas exchange organs, pose significant health challenges globally. Among these, seasonal influenza, caused by influenza A and B viruses, is a common affliction with substantial health and economic impacts. The sudden onset of symptoms such as fever, cough, and muscle pain can lead to high levels of absenteeism and productivity losses.

In addition to influenza, other respiratory viruses, such as human coronaviruses and the human Respiratory Syncytial Virus (hRSV), contribute to the burden of respiratory illnesses. Certain strains of coronaviruses are commonly associated with mild to moderate upper respiratory tract infections. Some strains, such as Severe Acute Respiratory Syndrome Coronavirus 2 (SARS-CoV-2), have led to global health crises, as seen with the COVID-19 pandemic.

hRSV, a negative-sense, single-stranded RNA virus, presents another significant concern, particularly in children and older adults. In these populations, hRSV infection can progress to more severe conditions like bronchiolitis or pneumonia.

### SUMMARY

Provided are compositions and methods for vaccination against seasonal influenza viruses, and optionally other respiratory viruses including hRSV and SARS-CoV-2. The compositions and methods are based, at least in part, on the finding that certain substitutions in influenza B virus (IBV) hemagglutinin (HA) proteins allows for stabilization of the HA proteins, and consequently improved cell surface expression and immunogenicity. Stabilization was also achieved in influenza A virus (IAV) HA proteins. Such influenza virus HA proteins allow for improved influenza vaccines, such as those containing RNAs (e.g., mRNAs) encoding the influenza virus HA proteins, or other compositions for vaccination (e.g., viral vector or protein-based vaccines).

Accordingly, some aspects relate to an influenza B/Victoria lineage virus hemagglutinin (HA) protein having an amino acid sequence comprising an amino acid substitution relative to a reference influenza B/Victoria lineage virus HA protein amino acid sequence, wherein the influenza B/Victoria lineage virus HA protein comprises one or more of (a)-(s):
(a) a tyrosine at position 381 and a valine at position 288;
(b) a cysteine at position 27 and a cysteine at position 349;
(c) a cysteine at position 295 and a cysteine at position 328;
(d) a cysteine at position 399 and a cysteine at position 473;
(e) a cysteine at position 422 and a cysteine at position 444;
(f) a cysteine at position 118 and a cysteine at position 216;
(g) a cysteine at position 237 and a cysteine at position 261;
(h) a cysteine at position 363 and a cysteine at position 480;
(i) a cysteine at position 364 and a cysteine at position 483;
(j) a cysteine at position 365 and a cysteine at position 476;
(k) a cysteine at position 366 and a cysteine at position 479;
(l) a cysteine at position 367 and a cysteine at position 483;
(m) a cysteine at position 435 and a cysteine at position 428;
(n) a cysteine at position 494 and a cysteine at position 483;
(o) a cysteine at position 494 and a cysteine at position 480;
(p) a proline at position 416, a proline at position 417, a proline at position 434, and a proline at position 433;
(q) a proline at position 434 and a proline at position 433;
® a proline at position 515, and a proline at position 516;
(s) a phenylalanine at position 473;
wherein the positions of (a)-(s) are numbered by alignment of the reference influenza B/Victoria lineage virus HA protein amino acid sequence to SEQ ID NO: 71.

In some embodiments, the influenza B/Victoria lineage virus HA protein comprises one or more of (a)-(s):
(a) H381Y and A288V substitutions;
(b) S27C and Y349C substitutions;
(c) I295C and K328C substitutions;
(d) S399C and H473C substitutions;
(e) L422C and D444C substitutions;
(f) K118C and L216C substitutions;
(g) V237C and D261C substitutions;
(h) G363C and K480C substitutions;
(i) A364C and K483C substitutions;
(j) I365C and A476C substitutions;
(k) A366C and R479C substitutions;
(l) G367C and K483C substitutions;
(m) E435C and A428C substitutions;
(n) N494C and K483C substitutions;
(o) N494C and K480C substitutions;
(p) E416P, L417P, N434P, and H433P substitutions;
(q) N434P and H433P substitutions;
(r) T515P and F516P substitutions; and
(s) an H473F substitution.

In some embodiments, the influenza B/Victoria lineage virus HA protein comprises tyrosine at position 381 and valine at position 288.

In some embodiments, the influenza B/Victoria lineage HA protein comprises H381Y and A288V substitutions.

In some embodiments, the influenza B/Victoria lineage HA protein further comprises one or more of (a)-(r):
(a) S27C and Y349C substitutions;
(b) I295C and K328C substitutions;
(c) S399C and H473C substitutions;
(d) L422C and D444C substitutions;
(e) K118C and L216C substitutions;
(f) V237C and D261C substitutions;
(g) G363C and K480C substitutions;
(h) A364C and K483C substitutions;
(i) I365C and A476C substitutions;
(j) A366C and R479C substitutions;
(k) G367C and K483C substitutions;
(l) E435C and A428C substitutions;
(m) N494C and K483C substitutions;
(n) N494C and K480C substitutions;
(o) E416P, L417P, N434P, and H433P substitutions;
(p) N434P and H433P substitutions;
(q) T515P and F516P substitutions;
(r) an H473F substitution.

In some embodiments, the influenza B/Victoria lineage virus HA protein comprises tyrosine at position 381, valine at position 288, cysteine at position 422, and cysteine at position 444.

In some embodiments, the influenza B/Victoria lineage virus HA protein comprises tyrosine at position 381, valine at position 288, cysteine at position 364, and cysteine at position 483.

In some embodiments, the influenza B/Victoria lineage virus HA protein comprises tyrosine at position 381, valine at position 288, cysteine at position 367, and cysteine at position 483.

In some embodiments, the influenza B/Victoria lineage virus HA protein comprises tyrosine at position 381, valine at position 288, cysteine at position 494, and cysteine at position 483.

In some embodiments, the influenza B/Victoria lineage virus HA protein comprises an amino acid sequence with at least 90% sequence identity to the amino acid sequence of SEQ ID NO: 71.

Some aspects relate to influenza B/Yamagata lineage virus hemagglutinin (HA) protein having an amino acid sequence comprising an amino acid substitution relative to a reference influenza B/Yamagata lineage virus HA protein amino acid sequence, wherein the influenza B/Yamagata lineage virus HA protein comprises one or more of (a)-(i):
(a) a cysteine at position 231 and a cysteine at position 273;
(b) a cysteine at position 295 and a cysteine at position 332;
(c) a cysteine at position 396 and a cysteine at position 510;
(d) a cysteine at position 239 and a cysteine at position 276;
(e) a cysteine at position 367 and a cysteine at position 401;
(f) a cysteine at position 363 and a cysteine at position 404;
(g) a cysteine at position 437 and a cysteine at position 429;
(h) a cysteine at position 451 and a cysteine at position 422; and
(i) a tyrosine at position 381 and a valine at position 290;
wherein the positions of (a)-(i) are numbered by alignment of the reference influenza B/Yamagata lineage virus HA protein amino acid sequence to SEQ ID NO: 70.

In some embodiments, the influenza B/Yamagata lineage virus HA protein comprises one or more of (a)-(i):
(a) A231C and G273C substitutions;
(b) K295C and I332C substitutions;
(c) A396C and L510C substitutions;
(d) V239C and V276C substitutions;
(e) I367C and S401C substitutions;
(f) F363C and E404C substitutions;
(g) E437C and G429C substitutions;
(h) D451C and K422C substitutions; and
(i) H381Y and A290V substitutions.

In some embodiments, the influenza B/Yamagata lineage virus HA protein comprises tyrosine at position 381 and valine substitution at position 290.

In some embodiments, the influenza B/Yamagata lineage virus HA protein comprises cysteine at position 239, cysteine at position 276, cysteine at position 451, and cysteine at position 422.

In some embodiments, the influenza B/Yamagata lineage virus HA protein comprises cysteine at position 367, cysteine at position 401, cysteine at position 451, and cysteine at position 422.

In some embodiments, the influenza B/Yamagata lineage virus HA protein comprises an amino acid sequence with at least 90% sequence identity to the amino acid sequence of SEQ ID NO: 70.

Some aspects relate to an influenza A virus (IAV) H1 hemagglutinin (HA) protein having an amino acid sequence comprising an amino acid substitution relative to a reference IAV H1 HA protein amino acid sequence, wherein the IAV H1 HA protein comprises one or more of (a)-(o):
(a) a cysteine at position 391 and a cysteine at position 37;
(b) a cysteine at position 395 and a cysteine at position 36;
(c) a cysteine at position 461 and a cysteine at position 348;
(d) a proline at position 404 and a proline at position 416;
(e) an isoleucine at position 395 and an isoleucine at position 447;
(f) a glycine at position 456 and an isoleucine at position 402;
(g) a cysteine at position 391, a cysteine at position 37, a proline at position 404, and a proline at position 416;
(h) a cysteine at position 391, a cysteine at position 37, an isoleucine at position 395, and an isoleucine at position 447;
(i) a cysteine at position 391, a cysteine at position 37, a glycine at position 456, and an isoleucine at position 402;
(j) a cysteine at position 395, a cysteine at position 36, a proline at position 404, and a proline at position 416;
(k) a cysteine at position 395, a cysteine at position 36, a glycine at position 456, and an isoleucine at position 402;
(l) a cysteine at position 461, a cysteine at position 348, a proline at position 404, and a proline at position 416;
(m) a cysteine at position 461, a cysteine at position 348, an isoleucine at position 395, and an isoleucine at position 447;
(n) a cysteine at position 461, a cysteine at position 348, a glycine at position 456, and an isoleucine at position 402; and
(o) a cysteine at position 391, a cysteine at position 37, a proline at position 404, a glycine at position 456, and an isoleucine at position 402;
wherein the positions of (a)-(o) are numbered by alignment of the reference IAV H1 HA protein amino acid sequence to SEQ ID NO: 83.

In some embodiments, the IAV H1 HA protein comprises one or more of (a)-(o):
(a) K391C and L37C substitutions;
(b) K395C and V36C substitutions;
(c) N461C and G348C substitutions;
(d) N404P and H416P substitutions;
(e) K395I and E447I substitutions;
(f) D456G and K402I substitutions;
(g) K391C, L37C, N404P, H416P substitutions;
(h) K391C, L37C, K395I, E447I substitutions;
(i) K391C, L37C, D456G, K402I substitutions;
(j) K395C, V36C, N404P, H416P substitutions;
(k) K395C, V36C, D456G, K402I substitutions;
(l) N461C, G348C, N404P, H416P substitutions;
(m) N461C, G348C, K395I, E447I substitutions;
(n) N461C, G348C, D456G, K402I substitutions; and
(o) K391C, L37C, N404P, H416P, D456G, K402I substitutions.

In some embodiments, the IAV H1 HA protein comprises proline at position 404, proline at position 416, cysteine at position 395, and cysteine at position 36.

In some embodiments, the IAV H1 HA protein comprises cysteine at position 456, isoleucine at position 402, cysteine at position 395, and cysteine at position 36.

In some embodiments, the IAV H1 HA protein comprises glycine at position 456, isoleucine at position 402, cysteine at position 395, and cysteine at position 36.

In some embodiments, the IAV H1 HA protein comprises proline at position 404, proline at position 416, cysteine at position 391, and cysteine at position 36.

In some embodiments, the IAV H1 HA protein comprises proline at position 404, proline at position 416, cysteine at position 391, cysteine at position 37, glycine at position 456, and isoleucine at position 402.

In some embodiments, the IAV H1 HA protein comprises an amino acid sequence with at least 90% sequence identity to the amino acid sequence of SEQ ID NO: 83.

Some aspects relate to an influenza A virus (IAV) H1 hemagglutinin (HA) protein having an amino acid sequence comprising an amino acid substitution relative to a reference IAV H1 HA protein amino acid sequence, wherein the IAV H1 HA protein comprises one or more of (a)-(t):
(a) a cysteine at position 410 and a cysteine at position 462;
(b) a cysteine at position 120 and a cysteine at position 419;
(c) a cysteine at position 391 and a cysteine at position 37;
(d) a cysteine at position 395 and a cysteine at position 36;
(e) a cysteine at position 406 and a cysteine at position 430;
(f) a cysteine at position 457 and a cysteine at position 346;
(g) a cysteine at position 461 and a cysteine at position 348;
(h) a proline at position 404 and a proline at position 419;
(i) a proline at position 404 and a proline at position 416;
(j) a proline at position 405 and a proline at position 406;
(k) a proline at position 415 and a proline at position 416;
(l) a tyrosine at position 25 and a glutamate at position 45;
(m) a tyrosine at position 370 and a tryptophan at position 497;
(n) a glycine at position 402, a glycine at position 405, and a glycine at position 407;
(o) an isoleucine at position 395 and an isoleucine at position 447;
(p) a glycine at position 456 and a glycine at position 402;
(q) a cysteine at position 442 and a cysteine at position 423;
(r) a glycine at position 391;
(s) a cysteine at position 410, a cysteine at position 462, a cysteine at position 457, and a cysteine at position 346; and
(t) a cysteine at position 391, a cysteine at position 37, a phenylalanine at position 370, and a phenylalanine at position 455;
wherein the positions of (a)-(t) are numbered by alignment of the reference IAV H1 HA protein amino acid sequence to SEQ ID NO: 95.

In some embodiments, the IAV H1 HA protein comprises one or more of (a)-(t):
(a) V410C and L462C substitutions;
(b) E120C and K419C substitutions;
(c) K391C and L37C substitutions;
(d) K395C and V36C substitutions;
(e) Q406C and D430C substitutions;
(f) S457C and L346C substitutions;
(g) N461C and G348C substitutions;
(h) N404P and K419P substitutions;
(i) N404P and H416P substitutions;
(j) T405P and Q406P substitutions;
(k) N415P and H416P substitutions;
(l) H25Y and H45E substitutions;
(m) H370Y and K497W substitutions;
(n) K402G, T405G, and F407G substitutions;
(o) K395I and E447I substitutions;
(p) D456G and K402I substitutions;
(q) L442C and N423C substitutions;
(r) a K391G substitution;
(s) V410C, L462C, S457C, L346C substitutions; and
(t) K391C, L37C, H370F, H455F substitutions.

In some embodiments, the IAV H1 HA protein comprises cysteine at position 391 and cysteine at position 37.

In some embodiments, the IAV H1 HA protein comprises cysteine at position 395 and cysteine at position 36.

In some embodiments, the IAV H1 HA protein comprises an amino acid sequence with at least 90% sequence identity to the amino acid sequence of SEQ ID NO: 95.

Some aspects relate to an influenza A virus (IAV) H3 hemagglutinin (HA) protein having an amino acid sequence comprising an amino acid substitution relative to a reference IAV H3 HA protein amino acid sequence, wherein the IAV H3 HA protein comprises one or more of (a) (j):

(a) a cysteine at position 40 and a cysteine at position 55;
(b) a cysteine at position 123 and a cysteine at position 421;
(c) a cysteine at position 260 and a cysteine at position 237;
(d) a cysteine at position 392 and a cysteine at position 46;
(e) a cysteine at position 411 and a cysteine at position 428;
(f) a proline at position 402, a proline at position 421, and a proline at position 414;
(g) a glycine at position 403;
(h) a glycine at position 408 and a glycine at position 409;
(i) an isoleucine at position 396; and
(j) an isoleucine at position 219 and a proline at position 504;
wherein the positions of (a)-(j) are numbered by alignment of the reference IAV H3 HA protein amino acid sequence to SEQ ID NO: 82.

In some embodiments, the IAV H3 HA protein comprises one or more of (a)-(j):
(a) T40C and A55C substitutions;
(b) S123C and R421C substitutions;
(c) L260C and P237C substitutions;
(d) Q392C and T46C substitutions;
(e) I411C and Y428C substitutions;
(f) G402P, R421P, and E414P substitutions;
(g) a K403G substitution;
(h) F408G and H409G substitutions;
(i) a K396I substitution; and
(j) T219I and H504P substitutions.

In some embodiments, the IAV H3 HA protein comprises proline at position 402, proline at position 421, and proline at position 414.

In some embodiments, wherein the IAV H3 HA protein comprises an amino acid sequence with at least 90% sequence identity to the amino acid sequence of SEQ ID NO: 82.

In some embodiments, the HA protein is a recombinant protein.

Some aspects relate to a nucleic acid encoding:
(a) the influenza B/Victoria lineage virus HA protein;
(b) the influenza B/Yamagata lineage virus HA protein;
(c) the IAV H1 HA protein; or
(d) the IAV H3 HA protein.

In some embodiments, the nucleic acid is a DNA, a messenger ribonucleic acid (mRNA), a circular ribonucleic acid (RNA), or a self-amplifying ribonucleic acid (saRNA).

Some aspects relate to a viral vector comprising:
(a) the influenza B/Victoria lineage virus HA protein, and/or a nucleic acid encoding the influenza B/Victoria lineage virus HA protein;
(b) the influenza B/Yamagata lineage virus HA protein, and/or a nucleic acid encoding the influenza B/Yamagata lineage virus HA protein;
(c) the IAV H1 HA protein, and/or a nucleic acid encoding the IAV H1 HA protein; and/or
(d) the IAV H3 HA protein, and/or a nucleic acid encoding the IAV H3 HA protein.

Some aspects relate to a vaccine comprising:
(a) the HA protein;
(b) the nucleic acid; or
(c) the viral vector.

In some embodiments, the nucleic acid is mRNA.

Some aspects relate to a messenger ribonucleic acid (mRNA) comprising an open reading frame (ORF) encoding an influenza B/Victoria lineage virus hemagglutinin (HA) protein having an amino acid sequence comprising an amino acid substitution relative to a reference influenza B/Victoria lineage virus HA protein amino acid sequence, wherein the influenza B/Victoria lineage virus HA protein comprises one or more of (a)-(s):
(a) a tyrosine at position 381 and a valine at position 288;
(b) a cysteine at position 27 and a cysteine at position 349;
(c) a cysteine at position 295 and a cysteine at position 328;
(d) a cysteine at position 399 and a cysteine at position 473;
(e) a cysteine at position 422 and a cysteine at position 444;
(f) a cysteine at position 118 and a cysteine at position 216;
(g) a cysteine at position 237 and a cysteine at position 261;
(h) a cysteine at position 363 and a cysteine at position 480;
(i) a cysteine at position 364 and a cysteine at position 483;
(j) a cysteine at position 365 and a cysteine at position 476;
(k) a cysteine at position 366 and a cysteine at position 479;
(l) a cysteine at position 367 and a cysteine at position 483;
(m) a cysteine at position 435 and a cysteine at position 428;
(n) a cysteine at position 494 and a cysteine at position 483;
(o) a cysteine substitution at position 494 and a cysteine at position 480;
(p) a proline at position 416, a proline at position 417, a proline at position 434, and a proline at position 433;
(q) a proline at position 434 and a proline at position 433;
(r) a proline at position 515, and a proline at position 516; and
(s) a phenylalanine at position 473;
wherein the positions of (a)-(s) are numbered by alignment of the reference influenza B/Victoria lineage virus HA protein amino acid sequence to SEQ ID NO: 71.

In some embodiments, the influenza B/Victoria lineage virus HA protein comprises tyrosine at position 381 and valine at position 288.

In some embodiments, the influenza B/Victoria lineage virus HA protein comprises tyrosine at position 381, valine at position 288, cysteine at position 422, and cysteine at position 444.

In some embodiments, the influenza B/Victoria lineage virus HA protein comprises tyrosine at position 381, valine at position 288, cysteine at position 364, and cysteine at position 483.

In some embodiments, the influenza B/Victoria lineage virus HA protein comprises tyrosine at position 381, valine at position 288, cysteine at position 367, and cysteine at position 483.

In some embodiments, the influenza B/Victoria lineage virus HA protein comprises tyrosine at position 381, valine at position 288, cysteine at position 494, and cysteine at position 483.

Some aspects relate to a messenger ribonucleic acid (mRNA) comprising an open reading frame (ORF) encoding an influenza B/Yamagata lineage virus hemagglutinin (HA) protein having an amino acid sequence comprising an amino acid substitution relative to a reference influenza B/Yamagata lineage virus HA protein amino acid sequence, wherein the influenza B/Yamagata lineage virus HA protein comprises one or more of (a)-(i):
(a) a cysteine at position 231 and a cysteine at position 273;
(b) a cysteine at position 295 and a cysteine at position 332;
(c) a cysteine at position 396 and a cysteine at position 510;
(d) a cysteine at position 239 and a cysteine at position 276;
(e) a cysteine at position 367 and a cysteine at position 401;
(f) a cysteine at position 363 and a cysteine at position 404;
(g) a cysteine at position 437 and a cysteine at position 429;
(h) a cysteine at position 451 and a cysteine at position 422; and
(i) a tyrosine at position 381 and a valine at position 290;
wherein the positions of (a)-(i) are numbered by alignment of the reference influenza B/Yamagata lineage virus HA protein amino acid sequence to SEQ ID NO: 70.

In some embodiments, the influenza B/Yamagata lineage virus HA protein comprises tyrosine at position 381 and valine at position 290.

In some embodiments, the influenza B/Yamagata lineage virus HA protein comprises cysteine at position 239, cysteine at position 276, cysteine at position 451, and cysteine at position 422.

In some embodiments, the influenza B/Yamagata lineage virus HA protein comprises cysteine at position 367, cysteine at position 401, cysteine at position 451, and cysteine at position 422.

Some aspects relate to a messenger ribonucleic acid (mRNA) comprising an open reading frame (ORF) encoding an influenza A virus (IAV) H1 hemagglutinin (HA) protein having an amino acid sequence comprising an amino acid substitution relative to a reference IAV H1 HA protein amino acid sequence, wherein the IAV H1 HA protein comprises one or more of (a)-(o):
(a) a cysteine at position 391 and a cysteine at position 37;
(b) a cysteine at position 395 and a cysteine at position 36;
(c) a cysteine at position 461 and a cysteine at position 348;
(d) a proline at position 404 and a proline at position 416;
(e) an isoleucine at position 395 and an isoleucine at position 447;
(f) a glycine at position 456 and an isoleucine at position 402;
(g) a cysteine at position 391, a cysteine at position 37, a proline at position 404, and a proline at position 416;
(h) a cysteine at position 391, a cysteine at position 37, an isoleucine at position 395, and an isoleucine at position 447;
(i) a cysteine at position 391, a cysteine at position 37, a glycine at position 456, and an isoleucine at position 402;
(j) a cysteine at position 395, a cysteine at position 36, a proline at position 404, and a proline at position 416;
(k) a cysteine at position 395, a cysteine at position 36, a glycine at position 456, and an isoleucine at position 402;
(l) a cysteine at position 461, a cysteine at position 348, a proline at position 404, and a proline at position 416;
(m) a cysteine at position 461, a cysteine at position 348, an isoleucine at position 395, and an isoleucine at position 447;
(n) a cysteine at position 461, a cysteine at position 348, a glycine at position 456, and an isoleucine at position 402; and
(o) a cysteine at position 391, a cysteine at position 37, a proline at position 404, a glycine at position 456, and an isoleucine at position 402;
wherein the positions of (a)-(o) are numbered by alignment of the reference IAV H1 HA protein amino acid sequence to SEQ ID NO: 83.

In some embodiments, the IAV H1 HA protein comprises proline at position 404, proline at position 416, cysteine at position 395, and cysteine at position 36.

In some embodiments, the IAV H1 HA protein comprises cysteine at position 456, isoleucine at position 402, cysteine at position 395, and cysteine at position 36.

In some embodiments, the IAV H1 HA protein comprises glycine at position 456, isoleucine at position 402, cysteine at position 395, and cysteine at position 36.

In some embodiments, the IAV H1 HA protein comprises proline at position 404, proline at position 416, cysteine at position 391, and cysteine at position 36.

In some embodiments, the IAV H1 HA protein comprises proline at position 404, proline at position 416, cysteine at position 391, cysteine at position 37, glycine at position 456, and isoleucine at position 402.

Some aspects relate to a messenger ribonucleic acid (mRNA) comprising an open reading frame (ORF) encoding an influenza A virus (IAV) H1 hemagglutinin (HA) protein having an amino acid sequence comprising an amino acid substitution relative to a reference IAV H1 HA protein amino acid sequence, wherein the IAV H1 HA protein comprises one or more of (a)-(t):
(a) a cysteine at position 410 and a cysteine at position 462;
(b) a cysteine at position 120 and a cysteine at position 419;
(c) a cysteine at position 391 and a cysteine at position 37;
(d) a cysteine at position 395 and a cysteine at position 36;
(e) a cysteine at position 406 and a cysteine at position 430;
(f) a cysteine at position 457 and a cysteine at position 346;
(g) a cysteine at position 461 and a cysteine at position 348;
(h) a proline at position 404 and a proline at position 419;
(i) a proline at position 404 and a proline at position 416;
(j) a proline at position 405 and a proline at position 406;
(k) a proline at position 415 and a proline at position 416;
(l) a tyrosine at position 25 and a glutamate at position 45;
(m) a tyrosine at position 370 and a tryptophan at position 497;
(n) a glycine at position 402, a glycine a position 405, and a glycine at position 407;
(o) an isoleucine at position 395 and an isoleucine at position 447;
(p) a glycine at position 456 and a glycine at position 402;
(q) a cysteine at position 442 and a cysteine at position 423;
(r) a glycine at position 391;
(s) a cysteine at position 410, a cysteine at position 462, a cysteine at position 457, and a cysteine at position 346; and
(t) a cysteine at position 391, a cysteine at position 37, a phenylalanine at position 370, and a phenylalanine at position 455;
wherein the positions of (a)-(t) are numbered by alignment of the reference IAV H1 HA protein amino acid sequence to SEQ ID NO: 95.

In some embodiments, the IAV H1 HA protein comprises cysteine at position 391 and cysteine at position 37.

In some embodiments, the IAV H1 HA protein comprises cysteine at position 395 and cysteine at position 36.

Some aspects relate to a messenger ribonucleic acid (mRNA) comprising an open reading frame (ORF) encoding an influenza A virus (IAV) H3 hemagglutinin (HA) protein having an amino acid sequence comprising an amino acid substitution relative to a reference IAV H3 HA protein amino acid sequence, wherein the IAV H3 HA protein comprises one or more of (a)-(j):
(a) a cysteine at position 40 and a cysteine at position 55;
(b) a cysteine at position 123 and a cysteine at position 421;
(c) a cysteine at position 260 and a cysteine at position 237;
(d) a cysteine at position 392 and a cysteine at position 46;
(e) a cysteine at position 411 and a cysteine at position 428;
(f) a proline at position 402, a proline at position 421, and a proline at position 414;
(g) a glycine at position 403;
(h) a glycine at position 408 and a glycine at position 409;
(i) an isoleucine at position 396; and
(j) an isoleucine at position 219 and a proline at position 504;
wherein the positions of (a)-(j) are numbered by alignment of the reference IAV H3 HA protein amino acid sequence to SEQ ID NO: 82.

In some embodiments, the IAV H3 HA protein comprises proline at position 402, proline at position 421, and proline at position 414.

Some aspects relate to an mRNA vaccine comprising:
(a) the mRNA encoding the IAV H1 HA protein;
(b) the mRNA encoding the IAV H3 HA protein; and
(c) the mRNA encoding the influenza B/Victoria lineage virus HA protein.

In some embodiments, the influenza B/Victoria lineage virus HA protein comprises tyrosine at position 381 and valine at position 288.

In some embodiments, the mRNA vaccine further comprises an additional mRNA encoding an additional IAV H3 HA protein.

In some embodiments, the mRNA vaccine further comprises two or more additional mRNAs, each independently encoding an additional IAV H3 HA protein.

In some embodiments, the mRNA vaccine further comprises the mRNA encoding influenza B/Yamagata lineage virus HA protein of any one of claims 45-48.

In some embodiments, the influenza B/Yamagata lineage virus HA protein comprises tyrosine at position 381 and valine at position 290.

In some embodiments, the mRNA vaccine does not comprise an mRNA encoding an influenza B/Yamagata lineage virus HA protein.

In some embodiments, substantially equal masses of different mRNAs encoding different influenza virus HA proteins are present in the mRNA vaccine.

In some embodiments, the mRNA vaccine further comprises:
(i) a first additional mRNA encoding a first IAV neuraminidase (NA) protein of a first IAV NA subtype;
(ii) a second additional mRNA encoding a second IAV NA protein of a different IAV NA subtype than the first IAV NA subtype; and
(iii) a third additional mRNA encoding an influenza B/Victoria lineage virus NA protein.

In some embodiments, the second IAV NA subtype is N2, wherein the mRNA vaccine further comprises an additional mRNA encoding an additional IAV N2 NA protein.

In some embodiments, the second IAV NA subtype is N2, wherein the mRNA vaccine further comprises two or more additional mRNAs each encoding an additional IAV N2 NA protein.

In some embodiments, the mRNA vaccine further comprises an additional mRNA encoding an influenza B/Yamagata lineage virus NA protein.

In some embodiments, the mRNA vaccine does not comprise an mRNA encoding an influenza B/Yamagata lineage virus NA protein.

In some embodiments, substantially equal masses of different mRNAs encoding different influenza virus NA proteins are present in the mRNA vaccine.

In some embodiments, substantially equal masses of (a) mRNAs encoding influenza virus HA proteins, and (b) mRNAs encoding influenza virus NA proteins are present in the mRNA vaccine.

In some embodiments, the mRNA vaccine comprises a 3:1 mass ratio of (a) mRNAs encoding influenza virus HA proteins, to (b) mRNAs encoding influenza virus NA proteins.

In some embodiments, the mRNA vaccine further comprises an mRNA encoding a full-length SARS-CoV-2 Spike (S) glycoprotein comprising one or more proline substitutions relative to SEQ ID NO: 78.

In some embodiments, further comprises an mRNA encoding a protein comprising one or more fragments of a SARS-CoV-2 Spike (S) glycoprotein.

In some embodiments, the one or more fragments comprises a fusion protein comprising (i) an N-terminal domain (NTD) of the SARS-CoV-2 S glycoprotein, (ii) a receptor-binding domain (RBD) of the SARS-CoV-2 S glycoprotein, and (iii) a transmembrane domain.

In some embodiments, the mRNA vaccine further comprises an mRNA encoding a fusion (F) glycoprotein of a human respiratory syncytial virus (hRSV), or a fragment of the hRSV F glycoprotein.

In some embodiments, the mRNA vaccine further comprises a lipid delivery vehicle in contact with the mRNA.

In some embodiments, the lipid delivery vehicle is a lipid nanoparticle comprising 20-60 mol % ionizable lipid, 5-25 mol % non-cationic lipid, 2-4 mol % PEG-modified lipid, and 25-55 mol % sterol.

In some embodiments, the ionizable lipid is a compound of Formula (IL*):

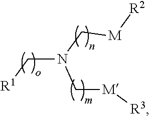

or a salt thereof, wherein:
R¹ is —OH, —NR^{N}—C₄₋₁₀ cycloalkenyl optionally substituted with one or more oxo or —N(R^{N}′R^{N″});
R^{N} is H or C₁₋₆ alkyl;
R^{N′} is H or C₁₋₆ alkyl;
R^{N″} is H or C₁₋₆ alkyl;
is 1, 2, 3, or 4;
n is 4, 5, 6, 7, or 8;
m is 4, 5, 6, 7, or 8;
M is —C(═O)—O—* or —O—C(═O)—*, wherein * indicates attachment to R²;
M′ is —C(═O)—O—* or —O—C(═O)—*, wherein * indicates attachment to R³;
R² is

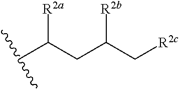

or —(C₁₋₆ alkylene)-(C₃₋₈ cycloalkyl)-C₁₋₆ alkyl;
R^{2a} is —H or C₁₋₁₀ alkyl;
R^{2b} is —H or C₁₋₁₀ alkyl;
R^{2c} is C₁₋₈ alkyl or C₂₋₈ alkenyl;
R³ is

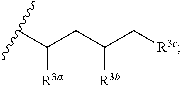

R^{3a} is H or C₁₋₁₀ alkyl;
R^{3b} is H or C₁₋₈ alkyl; and
R^{3c} is C₁₋₁₀ alkyl or C₂₋₈ alkenyl.

In some embodiments, the ionizable lipid is

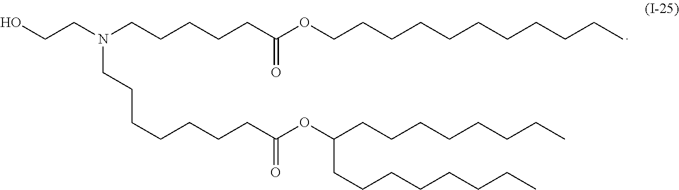 [SMILES: CCCCCCCCCCCOC(=O)CCCCCN(CCO)CCCCCCCC(=O)OC(CCCCCCCC)CCCCCCCC]

In some embodiments, the ionizable lipid is

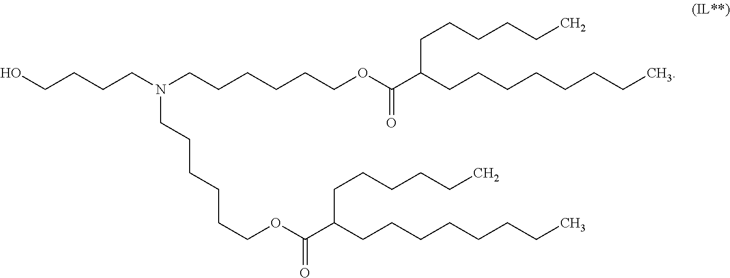 [SMILES: CCCCCCCCC(CCCCCC)C(=O)OCCCCCCN(CCCCO)CCCCCCOC(=O)C(CCCCCC)CCCCCCCC]

In some embodiments, 0.25 mol % to 1.0 mol % of the PEG-modified lipid is present in a core of the lipid nanoparticle.

In some embodiments, 2.0 mol % to 2.75 mol % of the PEG-modified lipid is not in the core of the lipid nanoparticle.

In some embodiments, the PEG-modified lipid is PEG-DMG or 134-hydroxy-3,6,9,12,15,18,21,24,27,30,33,36,39,42,45,48,51,54,57,60,63,66,69,72,75,78,81,84,87,90,93,96,99,102,105,108,111,114,117,120,123,126,129,132-tetratetracontaoxatetratriacontahectyl stearate.

In some embodiments, each mRNA comprises one or more chemically modified nucleotides.

In some embodiments, each mRNA comprises N1-methylpseudouridine.

In some embodiments, substantially all uracil nucleotides of each mRNA are modified to comprise N1-methylpseudouridine.

In some embodiments, each mRNA comprises 5-methylcytidine and 5-methyluridine.

In some embodiments, substantially all cytosine nucleotides of each mRNA are modified to comprise 5-methylcytidine, and substantially all uracil nucleotides of each mRNA are modified to comprise 5-methyluridine.

Some aspects relate to a method comprising administering the vaccine to a subject.

In some embodiments, the subject has not been vaccinated against an influenza virus for at least 6 months prior to administration of the vaccine.

### BRIEF DESCRIPTION OF THE DRAWINGS

FIG. 1A is graph showing polyclonal sera antibody binding in vitro to protein expressed by mRNA encoding mutated influenza B virus hemagglutinin at 72 hours post-transfection into cells with transIT at 250 ng mRNA/1×10⁶ cells. Substitutions in FIG. 1A are numbered according to post-cleavage forms of the HA proteins.

FIG. 1B is graph showing anti-hemagglutinin (CR8059) antibody binding in vitro to protein expressed by mRNA encoding mutated influenza B virus hemagglutinin at 72 hours post-transfection into cells with transIT at 250 ng mRNA/1×10⁶ cells. Substitutions in FIG. 1B are numbered according to post-cleavage forms of the HA proteins.

FIG. 1C is graph showing anti-hemagglutinin (CR8059) antibody binding in vitro to protein expressed by mRNA encoding mutated influenza B virus hemagglutinin at 72 hours post-transfection into cells with transIT at 100 ng mRNA/1×10⁶ cells. Substitutions in FIG. 1C are numbered according to post-cleavage forms of the HA proteins.

FIG. 1D is graph showing polyclonal sera antibody binding in vitro to protein expressed by mRNA encoding mutated influenza B virus hemagglutinin at 72 hours post-transfection into cells with transIT at 100 ng mRNA/1×10⁶ cells. Substitutions in FIG. 1D are numbered according to post-cleavage forms of the HA proteins.

FIG. 2A is graph showing antibody binding titers (fold-rise over wild-type) resulting from injection of mRNA encoding mutated influenza B virus hemagglutinin in mice at 21 days post-injection with a 0.25 μg dose. Substitutions in FIG. 2A are numbered according to post-cleavage forms of the HA proteins.

FIG. 2B is graph showing antibody binding titers (fold-rise over wild-type) result from injection of mRNA encoding mutated influenza B virus hemagglutinin in mice at 36 days post-injection with a 0.25 μg dose. Substitutions in FIG. 2B are numbered according to post-cleavage forms of the HA proteins.

FIG. 3A is a graph showing HAI titers of sera collected from mice injected with 0.25 μg of mRNA encoding a mutated influenza B virus hemagglutinin protein at 21 days post-injection. Substitutions in FIG. 3A are numbered according to post-cleavage forms of the HA proteins.

FIG. 3B is a graph showing HAI titers of sera collected from mice injected with 0.25 μg of mRNA encoding a mutated influenza B virus hemagglutinin protein at 36 days post-injection. Substitutions in FIG. 3B are numbered according to post-cleavage forms of the HA proteins.

FIG. 4A is graph showing antibody binding titers from mice administered mRNA encoding a mutated B virus hemagglutinin protein at 21 days post-injection with a 0.0625 μg dose. Substitutions in FIG. 4A are numbered according to post-cleavage forms of the HA proteins.

FIG. 4B is graph showing antibody binding titers from mice administered mRNA encoding a mutated IBV hemagglutinin protein at 36 days post-injection with a 0.0625 μg dose. Substitutions in FIG. 4B are numbered according to post-cleavage forms of the HA proteins.

FIG. 5A is a graph showing HAI titers of sera collected from mice injected with 0.0625 μg of mRNA encoding a mutated influenza B virus hemagglutinin protein at 21 days post-injection. Substitutions in FIG. 5A are numbered according to post-cleavage forms of the HA proteins.

FIG. 5B is a graph showing an HAI titer of serum collected from mice injected with 0.0625 μg of mRNA encoding a mutated influenza B virus hemagglutinin protein at 36 days post-injection. Substitutions in FIG. 5B are numbered according to post-cleavage forms of the HA proteins.

FIG. 6A is graph showing antibody binding titers from mice administered mRNA encoding a mutated IBV hemagglutinin protein, at 21 days post-injection with a 0.25 μg dose. Substitutions in FIG. 6A are numbered according to post-cleavage forms of the HA proteins.

FIG. 6B is graph showing antibody binding titers from mice administered mRNA encoding a mutated IBV hemagglutinin protein encoded, at 36 days post-injection with a 0.25 μg dose. Substitutions in FIG. 6B are numbered according to post-cleavage forms of the HA proteins.

FIG. 7A is graph showing antibody binding titers from mice administered mRNA encoding a mutated IBV hemagglutinin protein, at 21 days post-injection with a 0.0625 μg dose. Substitutions in FIG. 7A are numbered according to post-cleavage forms of the HA proteins.

FIG. 7B is graph showing antibody binding titers from mice administered mRNA encoding a mutated B hemagglutinin protein, at 36 days post-injection with a 0.0625 μg dose. Substitutions in FIG. 7B are numbered according to post-cleavage forms of the HA proteins.

FIG. 8 shows in vitro expression data 72 hours after cells were transfected with mRNA encoding a mutated influenza B virus hemagglutinin protein (mutations to the protein are shown on the X-axis). Substitutions in FIG. 8 are numbered according to post-cleavage forms of the HA proteins.

FIG. 9 shows IgG antibody binding titers in mice, 36 days after administration of a 0.25 μg dose of mRNA encoding a mutated influenza B virus hemagglutinin protein (mutations to the protein are shown on the X-axis). Substitutions in FIG. 9 are numbered according to post-cleavage forms of the HA proteins.

FIGS. 10A-10C are graphs showing show the effect of IBV HA stabilizing mutations on 4×HA in vitro expression. The graphs show expression levels following staining with CR8059 (a IBV HA-specific antibody; FIG. 10A), 5E04 (an H3 HA-specific antibody; FIG. 10B), and 2B06 (an H1 HA-specific antibody, FIG. 10C).

FIGS. 11A-11B show the HAI titers (FIG. 11A) and neutralizing antibody titers (FIG. 11B) present in samples from mice administered a flu vaccine (4×HA), a SARS-CoV-2 vaccine (NTD-RBD-HAtm), or a combination vaccine including mRNA encoding both influenza virus and coronavirus antigens (NTD-RBD-hAtm).

FIGS. 12A-12C show the effects of stabilizing mutant influenza B/Victoria lineage virus HA proteins comprising the following amino acid mutations relative to wild-type influenza B/Austria/1359417/2021 (B/Victoria lineage) virus HA protein amino acid sequence (SEQ ID NO: 71): (i) H381Y-A288V, (ii) H381Y-A288V-L422C-D444C, (iii) H381Y-A288V-A364C-K483C, (iv) H381Y-A288V-G367C-K483C, and (v) H381Y-288V-N494C-K483C. FIG. 12A shows in vitro surface expression levels of proteins expressed by mRNA encoding either wild-type or a mutant protein following staining with polyclonal sera antibodies or CR8059 (an influenza B virus HA-specific antibody). FIG. 12B shows the melting temperature of soluble proteins in PBS for wild-type and mutant proteins. FIG. 12C summarizes selection criteria for each candidate, with a star indicating the best performing mutant on the basis of polyclonal sera antibody binding, CR8059 antibody binding, melting temperature, and trimer formation.

FIGS. 13A-13C show the effect of stabilizing mutant influenza B/Yamagata lineage virus HA proteins comprising the following amino acid mutations relative to wild-type influenza B/Phuket/3073/2013 (B/Yamagata lineage) virus HA protein amino acid sequence (SEQ ID NO: 70): (i) V239C-V276C-D451C-K422C, and (ii) I367C-S401C-D451C-K422C. FIG. 13A shows in vitro surface expression levels of proteins expressed by high doses of mRNA (0.5 μg mRNA) encoding either wild-type or a candidate protein following staining with polyclonal sera antibodies, CR8059 mAb (an influenza B virus HA-specific antibody), or mAb 042 (an influenza B virus HA head-specific antibody). FIG. 13B shows in vitro surface expression levels of proteins expressed by low doses of mRNA (0.1 μg mRNA) encoding either wild-type or a mutant protein following staining with polyclonal sera antibodies, CR8059 mAb, or mAb 042. FIG. 13C shows the melting temperature of soluble proteins in PBS for wild-type and mutant proteins.

FIGS. 14A-14C show the effect of mutant stabilizing mutant IAV H3 HA proteins comprising the following amino acid mutations relative to wild-type influenza A/Darwin/6/2021(H3N2) virus HA protein amino acid sequence (SEQ ID NO: 82): (i) S123C-R421C, Q392C-T46C, and G402-R421P-E414P. FIG. 14A shows in vitro surface expression levels of proteins expressed by mRNA encoding either wild-type or a candidate protein following staining with polyclonal sera antibodies, 5E04 mAb (an influenza A virus H3 HA-specific antibody). FIG. 14B shows the melting temperature of soluble proteins in PBS for wild-type and mutant proteins. FIG. 14C summarizes selection criteria for each, with a star indicating the best performing mutant on the basis of polyclonal sera antibody binding, 5E04 binding, melting temperature, and trimer formation.

FIG. 15 shows HA-specific IgG titers of sera collected from mice injected with 5 μg/mL of mRNA encoding wild-type or mutant IAV H1 HA protein at 21 days post-injection. Candidate amino acid mutations included (i) N404P-H416P, (ii) K395C-V36C, (iii) D456C-D402I-K395C-V36C, (iv) D456C-D402I-K391C-L37C, (v) N404P-H416P-K391C-L37C, and (vi) N404P-H416-K391C-L37C-D456G-K402I relative to wild-type influenza A/Wisconsin/67/2022(H1N1)pdm09 virus HA protein amino acid sequence (SEQ ID NO: 83).

FIGS. 16A-16C show the effect of stabilizing mutant IAV H1 HA proteins comprising the following amino acid mutations relative to wild-type influenza A/Sydney/5/2021(H1N1)pdm09 virus HA protein amino acid sequence (SEQ ID NO: 95): (i) K391C-L37C, and (ii) K395C-V36C. FIG. 16A shows in vitro surface expression levels of proteins expressed by mRNA encoding either wild-type or a mutant protein following staining with polyclonal sera antibodies, 2B06 mAb (an influenza A virus H1 HA-specific antibody). FIG. 16B shows the melting temperature of soluble proteins in PBS for wild-type and candidate proteins. FIG. 16C summarizes selection criteria for each mutant, with a star indicating the best performing mutant on the basis of polyclonal sera antibody binding, 2B06-binding, melting temperature, and trimer formation.

FIGS. 17A-17F show reactogenicity and immunogenicity data from the Phase 1 clinical trial described in Example 6. FIG. 17A shows local reactogenicity events for all participants (LEFT), young adults (18-49 years old; CENTER), or older adults (50-75 years old; RIGHT) receiving 8 study interventions (FLUBLOK®, 4×HA [50 μg], 4×HA/4×NA at 1:1 HA:NA [50, 100, or 150 μg], or 4×HA/4×NA at 3:1 HA:NA [25, 50 or 100 μg]). FIG. 17B shows systemic reactogenicity events for all participants (LEFT), young adults (18-49 years old; CENTER), or older adults (50-75 years old; RIGHT) receiving 8 study interventions (FLUBLOK®, 4×HA [50 μg], 4×HA/4×NA at 1:1 HA:NA [50, 100, or 150 μg], or 4×HA/4×NA at 3:1 HA:NA [25, 50 or 100 μg]). FIG. 17C shows HAI titers against HIN1, H3N2, B/Victoria and B/Yamagata HA proteins as fold-rise at Day 29 relative to Day 1, for participants treated with each study intervention, by administered dosage of HA components. FIG. 17D shows NAI titers against H1N1, H3N2, B/Victoria and B/Yamagata NA proteins as fold-rise at Day 29 relative to Day 1, for participants treated with each study intervention, by administered dosage of NA components. FIG. 17E shows elicited strain-specific hemagglutination and neuraminidase inhibition titers in all participants receiving FLUBLOK®, 4×HA (50 μg), or 4×HA/4×NA at 1:1 HA:NA (50 μg). FIG. 17F shows elicited strain-specific hemagglutination and neuraminidase inhibition titers for participants aged 50-75 years old and receiving FLUBLOK®, 4×HA (50 μg), or 4×HA/4×NA at 3:1 HA:NA (50 μg).

FIG. 18A shows the geometric mean titer (GMT) ratio against each of the four vaccine-matched strains in adults 18 years of age or older administered the 4×HA [50 μg] mRNA vaccine in which encoded HA proteins contained stabilizing substitutions (as present in SEQ ID NOs: 85, 87, 91, and 94). FIG. 18B shows GMT ratios against each of the four vaccine-matched strains in adults 18 years of age or older administered the 4×HA [50 μg] mRNA vaccine in a previous study, in which encoded HA proteins did not contain substitutions relative to wild-type amino acid sequences.

FIGS. 19A-19K show representative alignments of exemplary influenza virus HA protein sequences within IAV HA subtypes and IBV lineages (FIGS. 19A-19D), pairwise alignments demonstrating alignment to identify residues for substitution in other reference HA protein amino acid sequences (FIGS. 19E-19G), and pairwise alignments showing substitutions as present in modified HA protein amino acid sequences (FIGS. 19H-19K). FIG. 19A shows multiple alignment of influenza A/(H1N1)pdm09 HA proteins. FIG. 19B shows multiple alignment of influenza A/(H3N2) HA proteins. FIG. 19C shows multiple alignment of influenza B/Victoria lineage virus HA proteins. FIG. 19D shows multiple alignment of influenza B/Yamagata lineage virus HA proteins. FIG. 19E shows pairwise alignment of influenza B/Brisbane/60/2008 (B/Victoria lineage) virus and influenza B/Austria/1359417/2021 (B/Victoria lineage) virus HA protein amino acid sequence of SEQ ID NOs: 157 and 71, respectively, with highlighting indicating residues H381 and A288 of SEQ ID NO: 71 and corresponding residues (after alignment) H384 and A291 of SEQ ID NO: 157. FIG. 19F shows pairwise alignment of influenza A/Beijing/262/1995(H1N1) virus and influenza A/Wisconsin/67/2022(H1N1)pdm09 virus HA protein amino acid sequences of SEQ ID NOs: 123 and 83, respectively, with highlighting indicating residues L37 and K391 of SEQ ID NO: 83 and corresponding residues (after alignment) L37 and G390 of SEQ ID NO: 123. FIG. 19G shows pairwise alignment of influenza B/Austria/1359417/2021 virus HA protein with (SEQ ID NO: 71) and without (SEQ ID NO: 174) the signal peptide. FIG. 19H shows alignment of an IAV H1 HA protein comprising K391C and L37C substitutions, as present in SEQ ID NO: 94, relative to the influenza A/Wisconsin/67/2022(H1N1)pdm09 virus HA protein amino acid sequence of SEQ ID NO: 83. FIG. 19I shows alignment of an IAV H3 HA protein comprising G402P, R421P, and E414P substitutions, as present in SEQ ID NO: 91, relative to the influenza A/Darwin/6/2021(H3N2) virus HA protein amino acid sequence of SEQ ID NO: 82. FIG. 19J shows alignment of an influenza B/Victoria lineage HA protein comprising H381Y, A288V, N494C, and K483C substitutions, as present in SEQ ID NO: 87, relative to the influenza B/Austria/1359417/2021 virus HA protein amino acid sequence of SEQ ID NO: 71. FIG. 19K shows alignment of an influenza B/Yamagata lineage HA protein comprising V239C, V276C, D451C, and K422C substitutions, as present in SEQ ID NO: 85, relative to the influenza B/Phuket/1359417/2021 virus HA protein amino acid sequence of SEQ ID NO: 70.

### DETAILED DESCRIPTION

Respiratory viruses are common agents of disease, having a significant impact on morbidity and mortality worldwide. Vaccines to respiratory viruses are designed to stimulate an immune response protective against the viruses. Various types of vaccines exist, including nucleic acid vaccines (e.g., DNA, and RNA such self-amplifying RNA or mRNA vaccines) that use the genetic instructions for antigenic polypeptide production to stimulate the immune response. Protein-based vaccines use an antigenic polypeptide or fragment thereof, either from inactivated viruses or purified subunits. Live attenuated vaccines use weakened live viruses comprising or encoding the antigenic polypeptide(s), while viral vector vaccines employ a virus to deliver the antigenic polypeptide(s) and/or nucleic acid(s) encoding the antigenic polypeptide(s) to cells. Mutations to stabilize the antigenic polypeptide(s) can enhance the effectiveness of these diverse types of vaccines, leading to improved immune responses and protection against the viruses. Some aspects relate to compositions and methods with improved vaccine efficacy, e.g., due to increased stability of the viral antigens. Preferred compositions comprise mRNA vaccines.

### Seasonal Influenza Virus

Seasonal influenza is a contagious respiratory illness caused by influenza viruses, which can lead to annual epidemics with symptoms ranging from mild to severe, and potentially causing complications, particularly in high-risk groups. Without being bound by theory, influenza viruses are believed to spread through respiratory droplets and aerosols, but can also spread through contact with surfaces (fomites). Upon entering the respiratory tract, virions attach to cells via hemagglutinin (HA) proteins, which bind to sialic acid receptors on the cell surface. Virions then enter the cell, where they replicate and produce new virions that bud from the cell membrane. NA proteins on the virion surface cleave sialic acid from the cell surface, leading to release of free virions that may then infect other cells. This process continues until the immune system can clear the infection.

Influenza viruses belong to the Orthomyxoviridae family and are categorized into types A, B, C, and D. Among these, influenza A and B viruses are of significant concern for human health.

Influenza A viruses (IAVs) can be further classified based on two surface proteins, hemagglutinin (sometimes referred to as H or HA) and neuraminidase (sometimes referred to as N or NA). There are 18 known HA subtypes and 11 known N subtypes. However, only H1, H2, and H3, and N1 and N2 subtypes (e.g., A/(H1N1); A(H1N2); A(H2N2); and A(H3N2)) have caused widespread human disease. The genetic diversity of influenza A viruses, due to frequent mutation and reassortment, can result in novel strains with pandemic potential.

Influenza B viruses (IBVs) are not divided into subtypes, but can be broken down into lineages and strains within those lineages. Currently, two lineages circulate in humans: B/Yamagata (e.g., B/Yamagata/16/1988-like) and B/Victoria (e.g., B/Victoria/2/1987-like).

Non-exhaustive lists of A/(H1N1) subtype, A/(H3N2) subtype, B/Victoria lineage, and B/Yamagata lineage isolates are provided in Table IV-1.

A key challenge in influenza control through vaccination is rapid evolution of the viruses, which can alter their antigenic properties and result in evasion of pre-existing immunity (e.g., through infection with, or vaccination with, previously circulating strains). This necessitates regular updates to influenza vaccines to match currently circulating strains. Yet, stability deficiencies associated with vaccine antigens can limit the efficacy of such strain-matched vaccines. Thus, some aspects relate to compositions and methods that improve the stability of influenza virus antigenic polypeptides.

An influenza virus HA protein may comprise one or more mutations relative to a reference influenza virus HA protein. In the context of proteins having one or more mutations (e.g., substitutions), a “reference protein” (e.g., reference HA protein) refers to a protein into which a mutation is introduced to yield a protein having the mutation.

A reference HA protein may be an HA protein of an influenza virus isolate. An “influenza virus isolate” or “isolate of an influenza virus” refers to an influenza virus that has been obtained from an infected host and grown in cell culture. Amino acid sequences of proteins of a virus isolate (e.g., HA) may be determined by sequencing the isolate's genome segments (e.g., segment 4) and/or viral RNA from cells infected with the isolate. As used herein, a reference HA protein amino acid sequence of an influenza virus isolate is the HA protein amino acid sequence encoded by a consensus nucleotide sequence of segment 4 of the isolate. The skilled artisan will appreciate that a replicating virus (e.g., in cell culture) forms a population of virions, that each virion contains a genome that may have one or more mutations relative to a consensus nucleotide sequence (or set of consensus nucleotide sequences, for viruses with segmented genomes like influenza viruses), and that viral genomes may be defined in terms of that consensus nucleotide sequence (or set of consensus nucleotide sequences of genome segments, for segmented viral genomes). See, e.g., Domingo et al., Gene. 1985. 40(1):1-8; and Kuroda et al., PLoS ONE. 2010. 5(4):e10256. A reference HA protein amino acid sequence of an isolate will be understood not to encompass HA protein amino sequences that are not encoded by the consensus nucleotide sequence of genome segment 4 of the isolate, even if such other HA protein amino acid sequences may be encoded by a minority of genomes in a virion population of the isolate. A reference HA protein may be an engineered HA protein that is not encoded by a consensus nucleotide sequence of genome segment 4 of a naturally occurring influenza virus isolate.

The skilled artisan will appreciate that mutations may be applied to any B/Victoria lineage HA protein, B/Yamagata lineage HA protein, H1 HA protein, or H3 HA protein amino acid sequence that is extant at the time this specification is filed. The skilled artisan will also appreciate that the mutations may be applied to B/Victoria lineage HA protein, B/Yamagata lineage HA protein, H1 HA protein, or H3 HA protein amino acid sequences that do not yet exist at the time of filing this specification. Indeed, it is the continued evolution of influenza viruses that leads public health authorities to update the influenza virus isolates recommended for inclusion in seasonal vaccines each year. Thus, when a given influenza virus isolate is recommended for inclusion in a seasonal influenza vaccine, for instance, the skilled artisan could apply a mutation described below to the HA protein of that influenza virus isolate, to produce a mutated form of that recommended isolate's HA protein.

Recommendations for candidate vaccine viruses are typically made annually by the World Health Organization (WHO), following review of surveillance data and discussion of candidate vaccine viruses, which typically occurs early in the calendar year (e.g., February). World Health Organization. (2023 Feb. 24). Recommended composition of influenza virus vaccines for use in the 2023-2024 northern hemisphere influenza season: Questions and answers.

For example, the WHO recommended the following viruses for inclusion in vaccines to be used in the 2023-2024 northern hemisphere influenza season:

### Egg-Based Vaccines:

an A/Victoria/4897/2022(H1N1)pdm09-like virus;
an A/Darwin/9/2021(H3N2)-like virus; and
a B/Austria/1359417/2021 (B/Victoria lineage)-like virus.

### Cell Culture- or Recombinant-Based Vaccines:

an A/Wisconsin/67/2022(H1N1)pdm09-like virus;
an A/Darwin/6/2021(H3N2)-like virus; and
a B/Austria/1359417/2021 (B/Victoria lineage)-like virus. Id.

For quadrivalent vaccines of any (egg, cell culture-, or recombinant-based) type, the WHO recommended inclusion of a B/Phuket/3073/2013 (B/Yamagata lineage)-like virus as the B/Yamagata lineage component. Id. Regarding the term “-like virus”, recommended vaccine viruses are representative of the antigenic group of viruses anticipated to circulate widely in the forthcoming influenza season, and that multiple candidate vaccine viruses may be available that possess HA proteins that are antigenically similar to the recommended vaccine viruses. Id. The term “-like virus” is included in recommendations to allow for use of other candidate vaccine viruses in manufacturing. Id.

Sequence information for influenza viruses recommended for inclusion in seasonal influenza vaccines (e.g., HA and NA protein amino acid sequences and corresponding genome segment nucleotide sequences) is typically available in publicly available databases, such as GenBank and GISAID.

The skilled artisan will appreciate that mutations described in the context of H1 HA proteins may be applied to extant or later-arising H1 HA proteins, mutations described in the context of H3 HA proteins may be applied to extant or later-arising H3 HA proteins, mutations described in the context of B/Victoria lineage HA proteins may be applied to extant or later-arising B/Victoria lineage HA proteins, and mutations described in the context of B/Yamagata lineage HA proteins may be applied to extant or later-arising B/Yamagata lineage HA proteins, as HA proteins within a given subtype or lineage are more similar to each other than to HA proteins of other subtypes or lineages.

At the time of filing of the instant specification, seasonal influenza vaccines are quadrivalent and intended to elicit immunity to an influenza A/(H1N1)pdm09 virus, an influenza A/(H3N2) virus, an influenza B/Victoria lineage virus, and an influenza B/Yamagata lineage virus. However, reassortment between influenza A viruses may result in the formation of novel IAVs that go on to predominate in the population, replacing the predominantly circulating influenza A/(H1N1)pdm09 and A/(H3N2) subtypes. The skilled artisan will appreciate that where mutations are disclosed in the context of H1 HAs of influenza A/(H1N1)pdm09 viruses (e.g., influenza A/Wisconsin/67/2022(H1N1)pdm09 and A/Sydney/5/2021(H1N1)pdm09 viruses), such mutations may also be applied to H1 HA proteins of other IAV subtypes containing H1 HAs (e.g., A/(H1N2). Mutations disclosed in the context of H3 HAs of influenza A/(H3N2) viruses (e.g., influenza A/Darwin/6/2021(H3N2) virus) may similarly be applied to H3 HA proteins of other IAV subtypes containing H3 HAs (e.g., A/(H3N8)).

Influenza virus HA proteins are discussed below in the section entitled “Influenza Virus HA Proteins”. Those skilled in the art will appreciate that influenza virus proteins discussed therein are useful in multiple types of vaccine compositions. In some embodiments, the composition comprises one or more influenza virus proteins. In some embodiments, the composition comprises one or more nucleic acids (e.g., mRNAs) encoding one or more influenza virus proteins. These and other vaccine compositions are discussed below in the section entitled “Vaccine Compositions.”

Thus, discussion of influenza virus proteins can also be applied to nucleic acids encoding said influenza virus proteins and vice versa unless otherwise clear from context. Thus, disclosure related to particular polypeptide mutations is also relevant to nucleic acids encoding those polypeptides with those mutations, unless otherwise clear from context. Likewise, disclosure related to mRNA encoding mutated influenza virus proteins may also be relevant to the mutated influenza virus proteins. Thus, when a composition comprising an mRNA encoding an influenza virus protein having a particular mutation is disclosed, the skilled artisan can infer that the influenza virus protein per se, and compositions comprising the influenza virus protein, are also disclosed. In some embodiments, an influenza virus protein is a recombinant protein. A “recombinant protein” refers to a protein that is produced in a heterologous organism that does not naturally produce the protein or a variant thereof. Non-limiting examples of organisms in which recombinant proteins may be produced include bacteria (e.g., Escherichia coli), yeast (e.g., Saccharomyces cerevisiae), and mammalian cells.

TABLE IV-1
Exemplary IAV and IBV isolates

| | | SEQ ID | SEQ ID |
|---|---|---|---|
| | | NO. | NO. |
| | | of HA | of NA |
| Subtype | | protein | protein |
| (IAV) or | | amino | amino |
| lineage | | acid | acid |
| (IBV) | Isolate | sequence | sequence |
| A/(H1N1) | A/Beijing/262/1995 (H1N1) | 122 | 180 |
| subtype | A/New Caledonia/20/1999 (H1N1) | 123 | 181 |
| | A/Solomon Islands/3/2006 (H1N1) | 124 | 182 |
| | A/Brisbane/59/2007 (H1N1) | 125 | 183 |
| | A/California/7/2009 (H1N1)pdm09 | 127 | 185 |
| | A/Michigan/45/2015 | 128 | 186 |
| | (H1N1)pdm09 | | |
| | A/Brisbane/02/2018 (H1N1)pdm09 | 129 | 187 |
| | A/Guangdong- | 130 | 188 |
| | Maonan/SWL1536/2019 | | |
| | (H1N1)pdm09 | | |
| | A/Hawaii/70/2019 (H1N1)pdm09 | 131 | 189 |
| | A/Victoria/2570/2019 | 132 | 190 |
| | (H1N1)pdm09 | | |
| | A/Wisconsin/588/2019 | 133 | 191 |
| | (H1N1)pdm09 | | |
| | A/Victoria/4897/2022 | 134 | 192 |
| | (H1N1)pdm09 | | |
| | A/Wisconsin/67/2022 | 135 | 193 |
| | (H1N1)pdm09 | | |
| A/(H3N2) | A/Sydney/5/97 (H3N2) | 136 | 194 |
| subtype | A/Moscow/10/1999 (H3N2) | 137 | 195 |
| | A/Fujian/411/2002 (H3N2) | 138 | 196 |
| | A/California/7/2004 (H3N2) | 139 | 197 |
| | A/Wisconsin/67/2005 (H3N2) | 140 | 198 |
| | A/Brisbane/10/2007 (H3N2) | 141 | 199 |
| | A/Perth/16/2009 (H3N2) | 142 | 200 |
| | A/Victoria/361/2011 (H3N2) | 143 | 201 |
| | A/Texas/50/2012 (H3N2) | 144 | 202 |
| | A/Switzerland/9715293/2013 | 145 | 203 |
| | (H3N2) | | |
| | A/Hong Kong/4801/2014 (H3N2) | 146 | 204 |
| | A/Singapore/INFIMH-16- | 147 | 205 |
| | 0019/2016 (H3N2) | | |
| | A/Kansas/14/2017 (H3N2) | 148 | 206 |
| | A/Hong Kong/2671/2019 (H3N2) | 149 | 207 |
| | A/Hong Kong/45/2019 (H3N2) | 150 | 208 |
| | A/Cambodia/e0826360/2020 | 151 | 209 |
| | (H3N2) | | |
| | A/Darwin/9/2021 (H3N2) | 152 | 210 |
| | A/Darwin/6/2021 (H3N2) | 153 | 211 |
| B/Victoria | B/Shangdong/7/1997 | 154 | 212 |
| lineage | B/Hong Kong/330/2001 | 155 | 213 |
| | B/Malaysia/2506/2004 | 156 | 214 |
| | B/Brisbane/60/2008 | 157 | 215 |
| | B/Colorado/06/2017 | 158 | 216 |
| | B/Washington/02/2019 | 159 | 217 |
| | B/Austria/1359417/2021 | 160 | 218 |
| B/Yamagata | B/Beijing/184/1993 | | 219 |
| lineage | B/Sichuan/379/1999 | | 220 |
| | B/Shanghai/361/2002 | | 221 |
| | B/Florida/4/2006 | 164 | 222 |
| | B/Wisconsin/1/2010 | 165 | 223 |
| | B/Massachusetts/2/2012 | 166 | 224 |
| | B/Phuket/3073/2013 | 167 | 225 |

### Influenza Virus HA Proteins

It was surprisingly discovered that influenza virus HA proteins having one or more mutations (such as a substitution) relative to a wild-type HA protein sequence exhibit increased stability, surface expression, and/or immunogenicity as compared to the wild-type influenza virus HA protein. Without being bound by theory, it is believed that such mutations in influenza virus HA proteins can stabilize the conformation of the HA proteins.

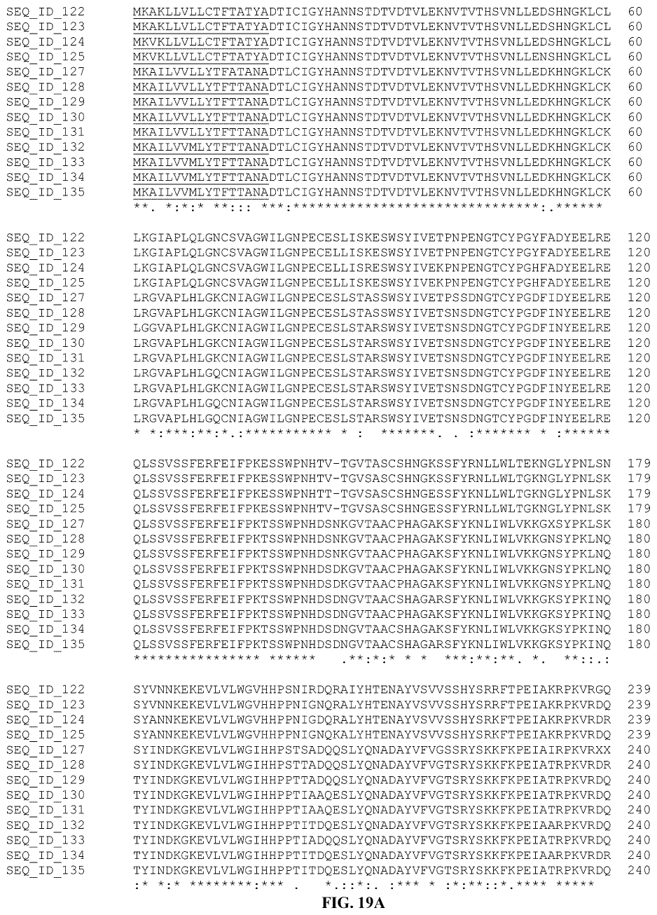

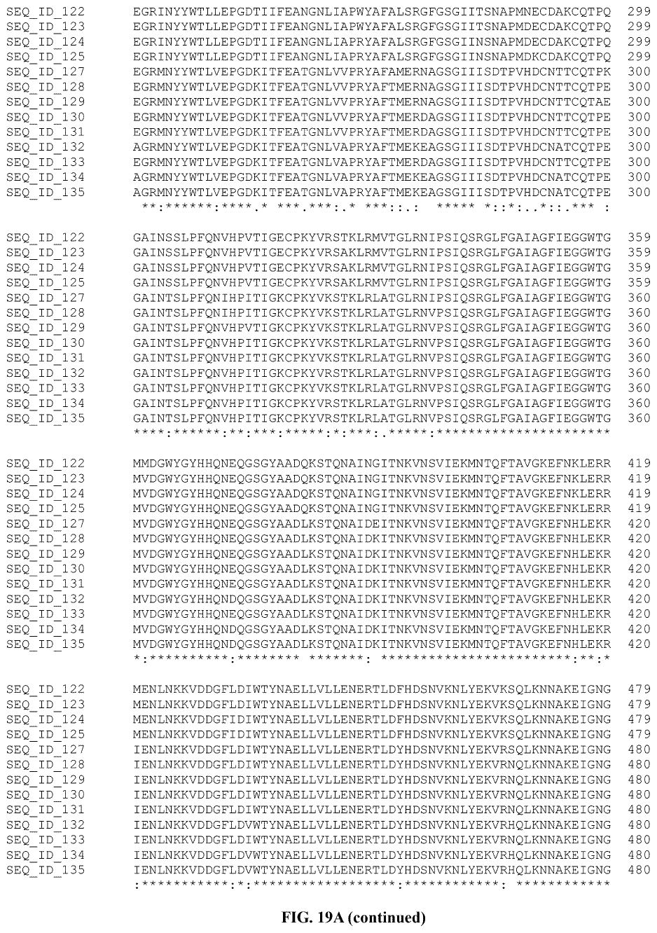

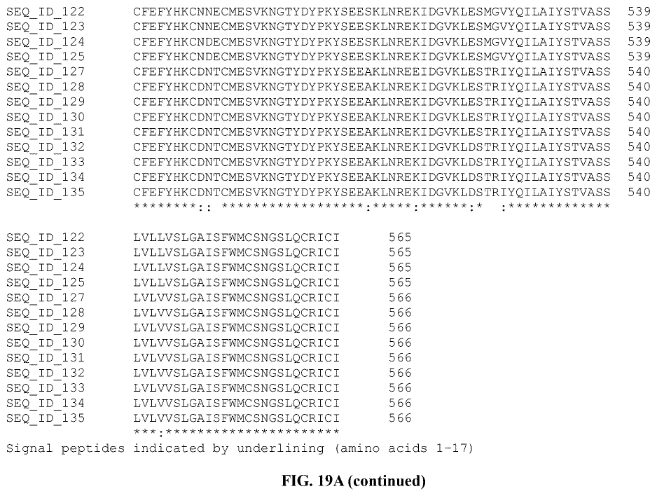

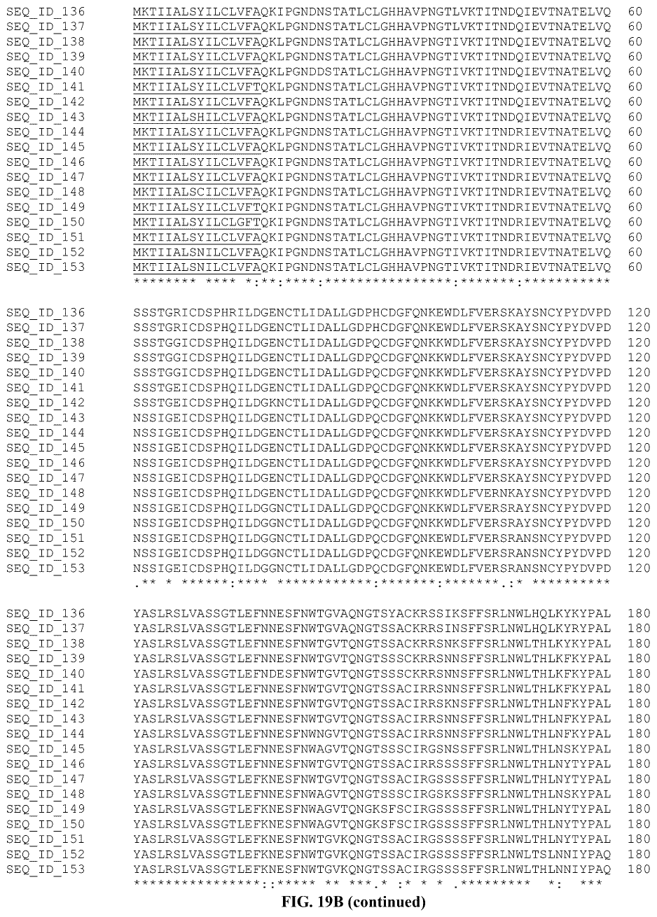

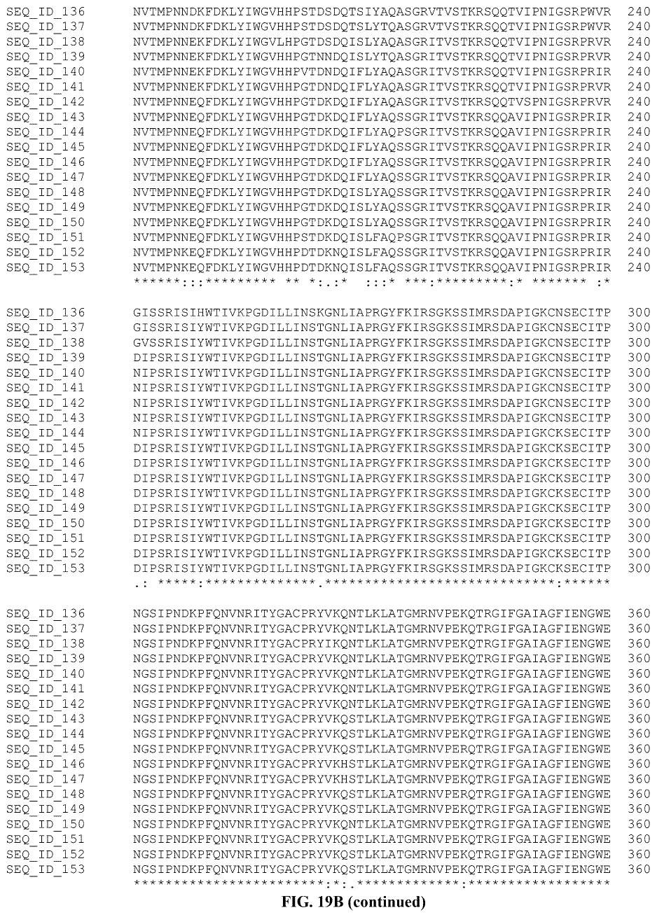

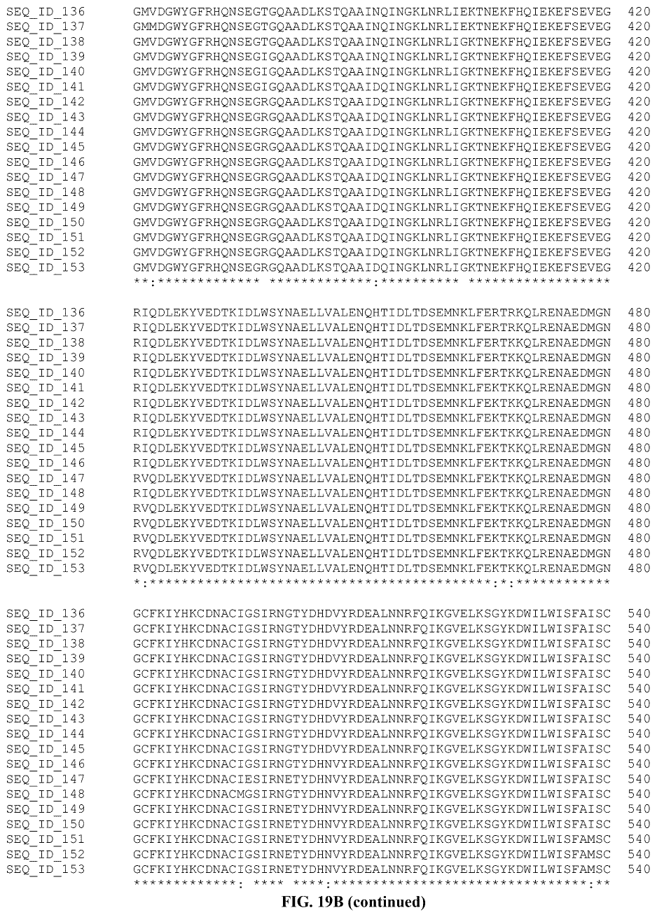

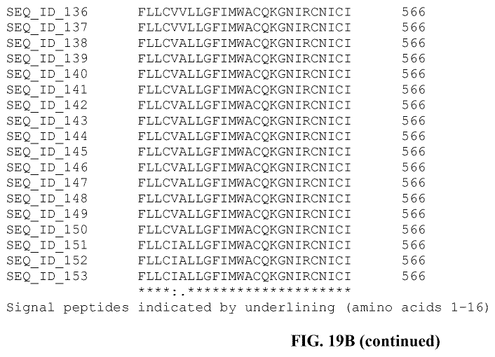

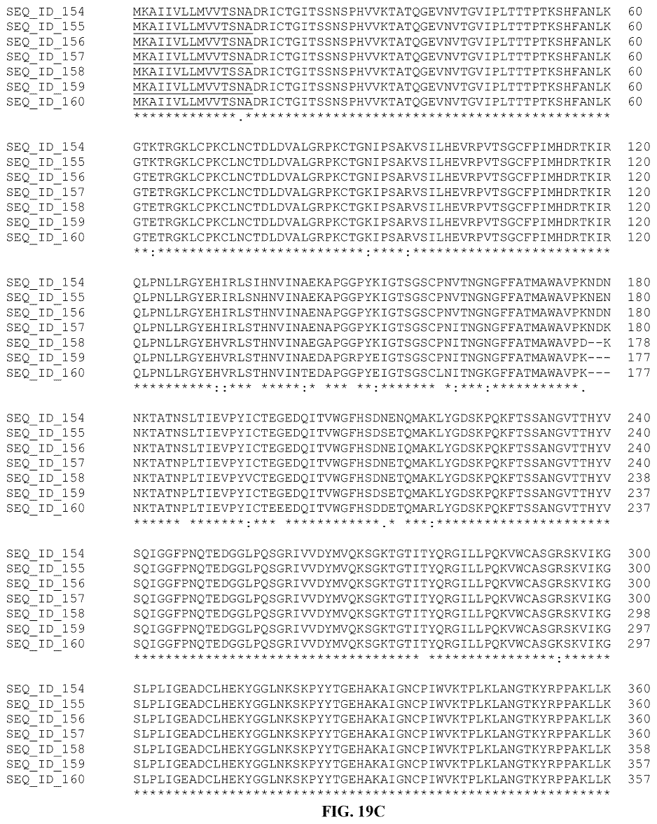

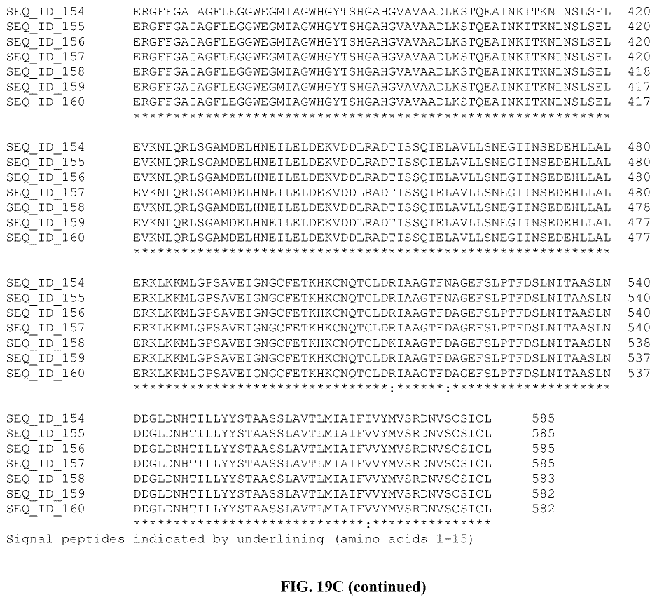

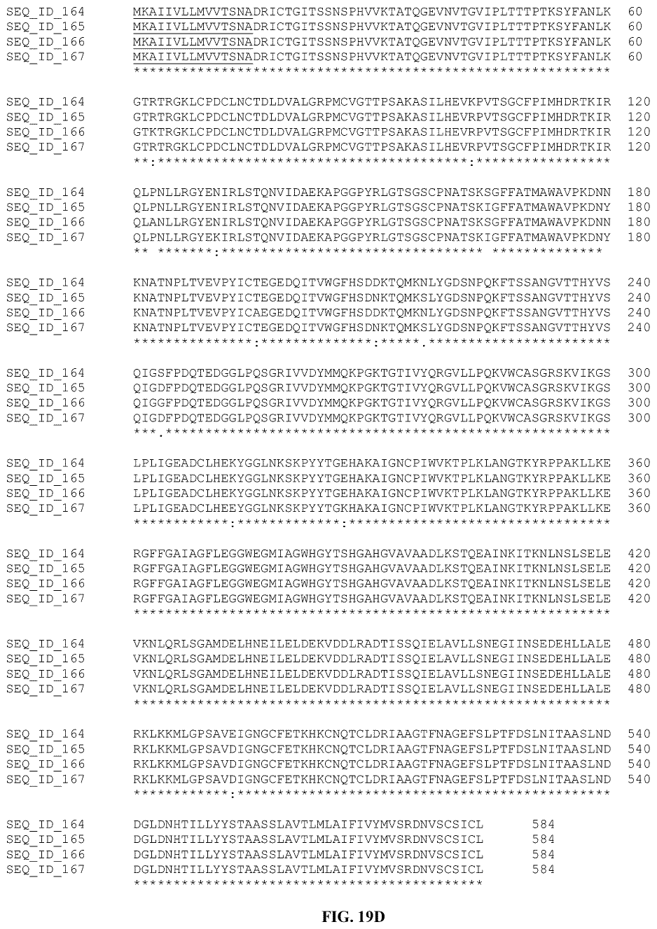

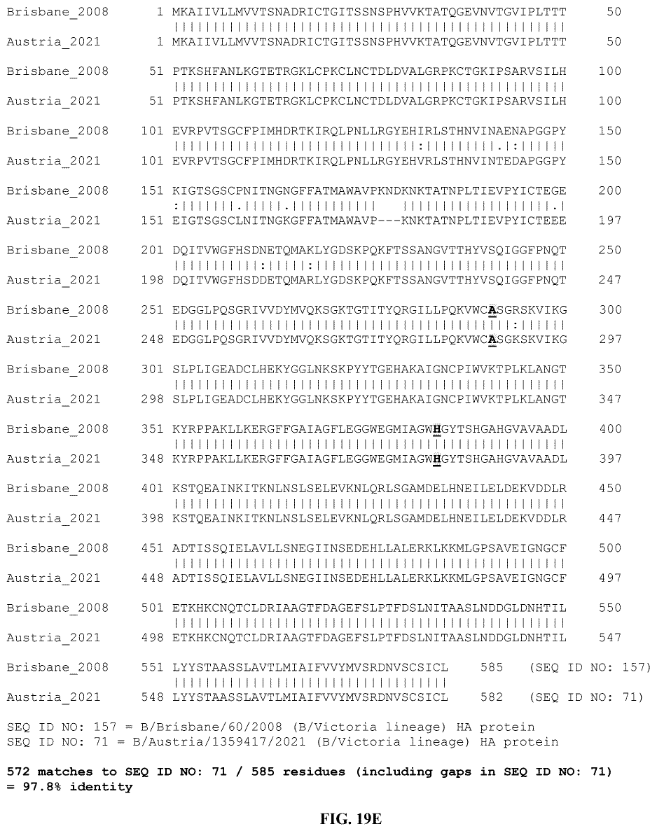

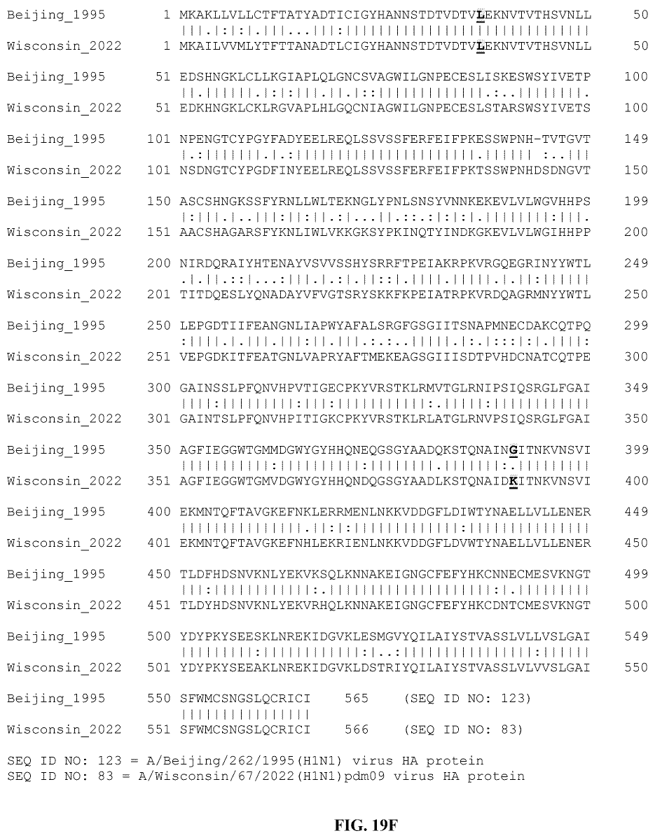

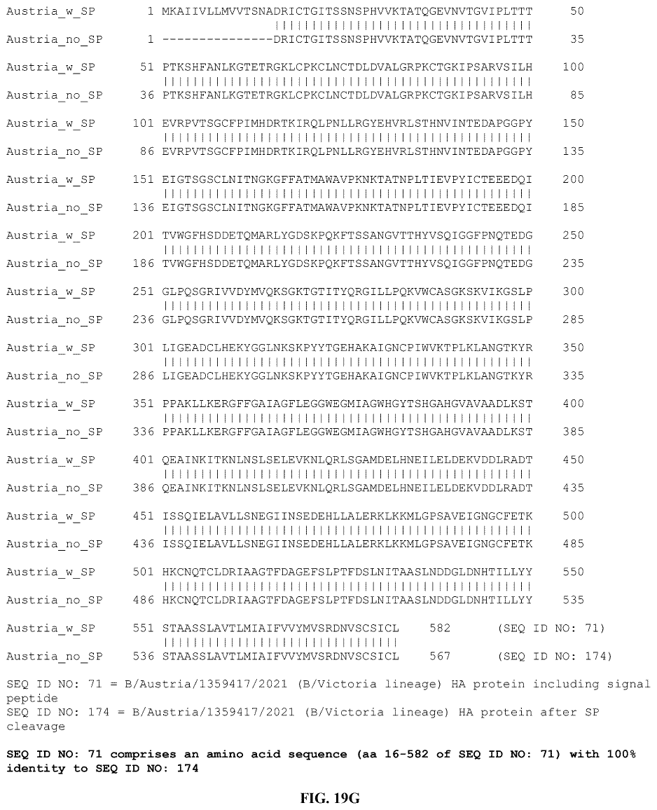

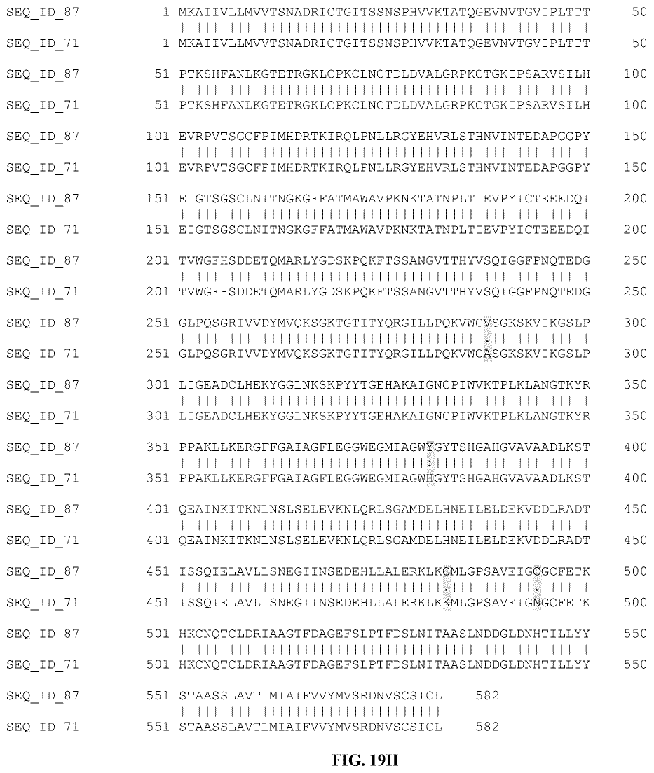

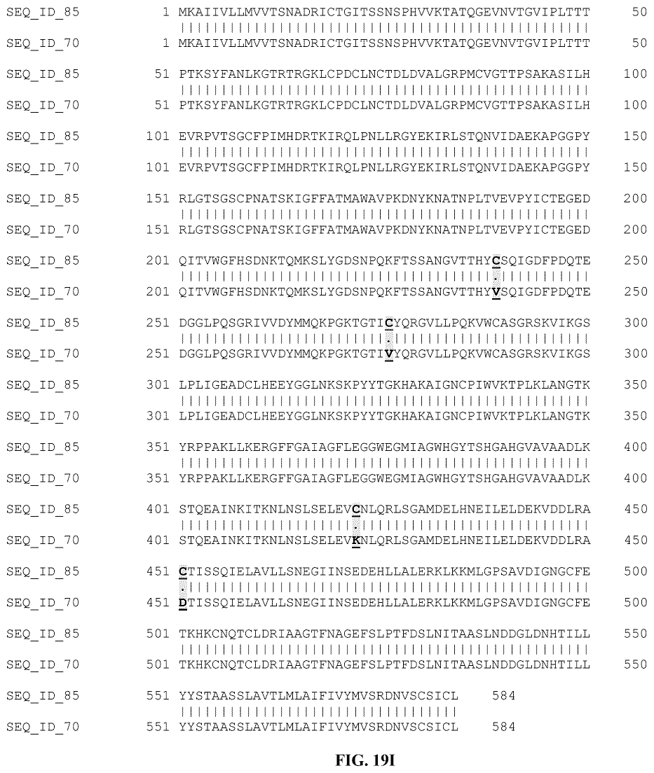

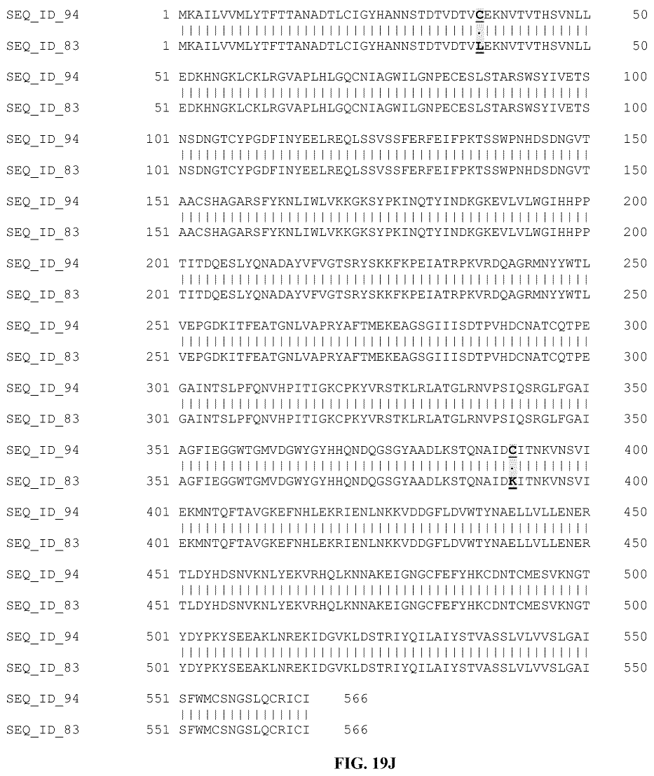

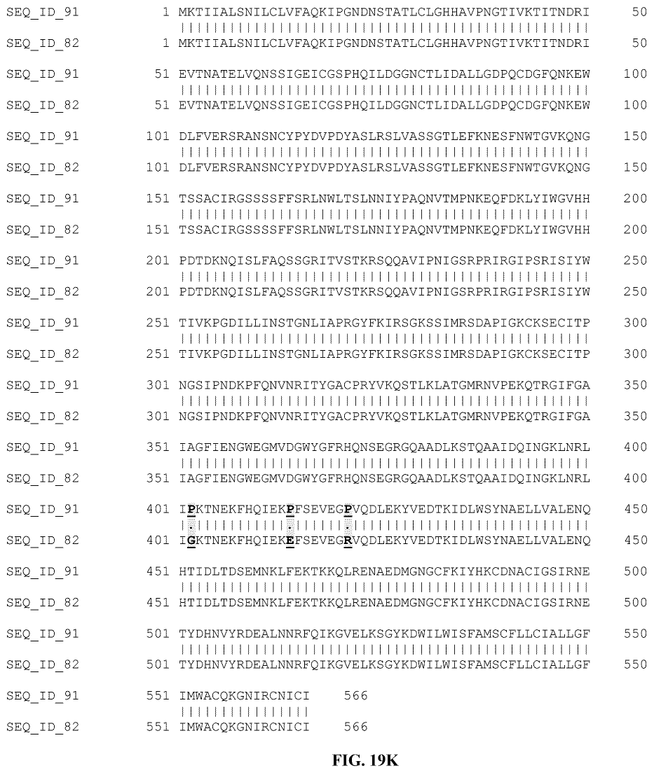

Mutations that may be applied to influenza virus HA proteins are described in Tables HA-1 to HA-5 below. For clarity, mutations are described using amino acid numbering corresponding to specific listed HA amino acid sequences (e.g., of recent vaccine strains A/Wisconsin/67/2022 (H1N1)pdm09 (SEQ ID NO: 83), A/Darwin/6/2021(H3N2) (SEQ ID NO: 82), B/Austria/1359417/2021 (B/Victoria lineage) (SEQ ID NO: 71), and B/Phuket/3073/2013 (B/Yamagata lineage) (SEQ ID NO: 70), and additional strain A/Sydney/5/2021(H1N1)pdm09 (SEQ ID NO: 95)). The person of ordinary skill in the art will appreciate that mutations disclosed in relation to a listed HA amino acid sequence may be applied to other HA amino acid sequences of that IAV HA subtype (e.g., H1, H3), or other HA amino acid sequences of that IBV lineage (i.e., B/Victoria or B/Yamagata lineage). To apply a mutation disclosed with numbering corresponding to a listed amino acid sequence (e.g., SEQ ID NO: 71) to a different reference HA protein, the skilled artisan may align that different reference HA protein's amino acid sequence to the listed amino acid sequence with which the mutation is numbered. For example, to apply an H381Y substitution numbered according to SEQ ID NO: 71 (the listed amino acid sequence), to a reference HA protein, the skilled artisan would align the reference HA protein amino acid sequence to SEQ ID NO: 71, and introduce a tyrosine (Y) at the residue of the reference HA protein that corresponds to H381 of SEQ ID NO: 71. An example of such an alignment is presented in FIG. 19E, the influenza B/Brisbane/60/2008 virus HA protein amino acid sequence of SEQ ID NO: 157, showing that residue H384 of SEQ ID NO: 157 aligns to residue H381 of SEQ ID NO: 71. Accordingly, to apply an H381Y substitution (or tyrosine substitution at position 381 generally) to SEQ ID NO: 157, the skilled artisan would replace residue 384 of SEQ ID NO: 157 with a tyrosine, because residue 384 of SEQ ID NO: 157 aligns to H381 of SEQ ID NO: 71. Another example is presented in FIG. 19F, showing alignment of the influenza A/Beijing/262/1995(H1N1) virus HA protein amino acid sequence of SEQ ID NO: 123 to the influenza A/Wisconsin/62/2022(H1N1)pdm09 virus HA protein amino acid sequence. While both SEQ ID NOs: 123 and 83 contain an L37 residue, residue G390 of SEQ ID NO: 123 aligns to K391 of SEQ ID NO: 83. Thus, to apply a K391C substitution (numbered according to SEQ ID NO: 83) to the amino acid sequence of SEQ ID NO: 123, the skilled artisan would replace G390 of SEQ ID NO: 123 with a cysteine (C), because G390 aligns to K391 of SEQ ID NO: 83.

An Influenza virus HA protein may comprise one or more substitutions relative to a reference HA protein. For example, an influenza B/Victoria lineage virus HA protein having the amino acid sequence of SEQ ID NO: 77 comprises a tyrosine substitution and a valine substitution relative to the reference influenza B/Victoria lineage virus HA protein sequence of SEQ ID NO: 71. The skilled artisan will appreciate the substitutions disclosed in relation to a listed amino acid sequence (e.g., SEQ ID NO: 71) may be referred to in the form X₁[#]X₂, where X₁ is the amino acid at position [#] in a listed amino acid sequence, and X₂ is the amino acid introduced by replacement of the X₁ at position [#]. For example, a tyrosine substitution at position 381 may also be referred to as an H381Y substitution, when using SEQ ID NO: 71 as a listed amino acid sequence, because the histidine (H) at position 381 of SEQ ID NO: 71 is replaced with a tyrosine (Y). Such a substitution may also be referred to as an “X₂ substitution at position [#]”, meaning that a protein comprises an X₂ residue at position [#] (numbered by alignment to a listed sequence), regardless of whether the residue that was present at position [#] in the reference amino acid sequence was X₁, or a different residue other than X₂. The skilled artisan will understand that substitutions disclosed in the form X₁[#]X₂ may also be applied to a reference amino acid sequence in the form of “X₂ substitution at position [#]” that is agnostic to the residue present in the reference amino acid sequence (numbered by alignment to a listed sequence). While the preceding paragraph uses SEQ ID NO: 71 (HA protein of influenza B/Austria/1359417/2021 (B/Victoria lineage) virus) as a reference HA protein amino acid sequence, a reference HA protein may be an HA protein of another influenza virus isolate, or an engineered HA protein that is not encoded by a genome of an isolate.

An influenza virus HA protein may comprise a specified residue at a specified position, with positions being numbered according to a listed amino acid sequence (e.g., SEQ ID NO: 71). As with determining the positions of substitutions numbered according to a listed amino acid sequence, the skilled artisan may align the HA protein sequence to the listed amino acid sequence, to determine whether the HA protein contains the specified residue at that position corresponding to the specified position of the listed amino acid sequence. For example, to determine whether an HA protein contains a tyrosine at position 381 and a valine at position 288, where the positions are numbered by alignment to SEQ ID NO: 71, the skilled artisan would align the amino acid sequence of the HA protein to SEQ ID NO: 71, determine whether the amino acid of the HA protein that aligns to residue 381 of SEQ ID NO: 71 is a tyrosine, and determine whether the amino acid of the HA protein that aligns to residue 288 of SEQ ID NO: 71 is a valine.

Unless otherwise clear from context, mutations in the instant specification are numbered according to full-length amino acid sequences of influenza virus HA proteins (e.g., SEQ ID NOs: 70, 71, 82, 83, and 95), each of which includes the signal peptide of the HA protein (i.e., residue 1 of each sequence is the methionine encoded by the start codon of an ORF encoding the HA protein). The person of ordinary skill will understand that other numbering schemes exist for reference to other forms or subunits of an HA protein, such as a post-signal peptide cleavage form (where residue 1 is the N-terminal amino acid after signal peptide cleavage). For instance, the skilled artisan will understand that reference to A270V+H363Y substitutions in influenza B/Austria/1359417/2021 virus HA protein would correspond to A288V+H381Y of SEQ ID NO: 71, because SEQ ID NO: 71 includes the signal peptide protein at its N-terminus, and after including that signal peptide, the A and H residues separated by 93 amino acids are found at positions 288 and 381 of SEQ ID NO: 71, respectively.

Some embodiments relate to HA proteins comprising one or more mutations selected from: (i) one or more introduced cysteines, where a disulfide bond is formed by at least one of the introduced cysteines; (ii) one or more proline or glycine substitutions in a B loop of the HA protein; (iii) one or more substitutions of a charged or polar cavity-lining residue with a hydrophobic residue; (iv) one or more substitutions of a pH-sensitive histidine in the HA protein; and/or (v) a mutation in, or removal of, a polybasic cleavage site of the HA protein.

In some embodiments, an HA protein comprises one or more substitutions of a pH-sensitive histidine in the HA protein and one or more substitutions of a charged or polar cavity-lining residue with a hydrophobic residue.

In some embodiments, an HA protein comprises an interprotomer disulfide bond formed by at least one introduced cysteine, and one or more proline substitutions in the B loop.

In some embodiments, an HA protein comprises an interprotomer disulfide bond formed by at least one introduced cysteine, and one or more substitutions of a charged or polar cavity-lining residue with a hydrophobic residue.

In some embodiments, an HA protein comprises an interprotomer disulfide bond formed by at least one introduced cysteine, one or more substitutions of a charged or polar cavity-lining residue with a hydrophobic residue, and one or more proline substitutions in the B loop.

In some embodiments, an HA protein comprises an interprotomer disulfide bond formed by at least one introduced cysteine, and an intraprotomer disulfide bond formed by at least one introduced cysteine, where the introduced cysteine(s) of the interpromoter disulfide bond are at different positions than the introduced cysteine(s) of the intraprotomer disulfide bond.

In some embodiments, an HA protein comprises an interprotomer disulfide bond formed by at least one introduced cysteine, and one or more substitutions of a pH-sensitive histidine in the HA protein and one or more substitutions of a charged or polar cavity-lining residue with a hydrophobic residue.

In some embodiments, an HA protein comprises one or more cysteine substitutions, such that a disulfide bond is formed between the introduced cysteines. In some embodiments, the disulfide bond is an interprotomer disulfide bond, which covalently links two protomers of an HA protein containing multiple protomers (e.g., three protomers). In some embodiments, the disulfide bond is an intraprotomer disulfide bond, which covalently links two residues of a single protomer of the HA protein. In some embodiments, the disulfide bond is in the head region (also referred to as the “globular head”, “head”, or HA1 subunit) of the HA protein. In some embodiments, the disulfide bond is in the stalk region (also referred to as the “stem”, “stalk”, or HA2 subunit) of the HA protein. Translation of an open reading frame encoding a full-length HA protein sequence produces a precursor HA0 polypeptide, which is then cleaved (e.g., by a serine protease) to produce an HA1 subunit (N-terminal to the cleavage site) and HA2 subunit (C-terminal to the cleavage site), which are connected by a disulfide bond. See, e.g., Wang et al., J Virol. 89(20):10602-10611. The positions of cleavage sites in IAV H1 HA, IAV H3 HA, influenza B/Victoria lineage virus HA, and influenza B/Yamagata lineage HA proteins are known in the art, allowing the skilled artisan to identify the residues of a given reference full-length HA protein sequence that correspond to HA1 and HA2 subunits of that reference HA protein.

In some embodiments, an HA protein comprises one or more proline substitutions in the B loop of the HA protein. In some embodiments, an HA protein comprises one or more glycine substitutions in the B loop of the HA protein. HA proteins comprise multiple subdomains, including a B loop, which enables the fusion of the viral envelope and endosomal membrane. Proline and glycine are among residues considered helix breakers, such that the introduction of proline or glycine residues can stabilize or destabilize proteins by altering their conformation (Lyu et al. 1990. Science. 250(4981), 669-673). In some embodiments, an HA protein comprises 2, 3, or 4 proline substitutions in the B loop. The location of the B loop in IAV H1 HA, IAV H3 HA, influenza B/Victoria lineage virus HA, and influenza B/Yamagata lineage HA proteins are known in the art, allowing the skilled artisan to identify the residues of a given reference full-length HA protein sequence that correspond to the B loop of that reference HA protein. See, e.g., Mair et al., Biochim Biophys Acta. 2014. 1838(4):1153-1168.

In some embodiments, an HA protein comprises one or more substitutions of a pH-sensitive histidine residue. The protonation state and charge of certain histidine residues of influenza virus HA proteins are sensitive to pH, such that histidine residues that are unprotonated at neutral pH may become protonated, and thus positively charged, as the pH of an endosome drops. Such protonation may result in a conformational change that allows the viral envelope to fuse with the endosomal membrane. For example, H5 HA1 residue His184 is located near positively charged residues in prefusion HA protein structures, and its protonation state acts as molecular switch triggering a conformational change in HA. See, e.g., Kampmann et al., Structure. 2006. 14(10):1481-1487; Mair et al., J Virol. 2014. 88(22):13189-13200. Accordingly, replacement of one or more pH-sensitive histidines can inhibit or prevent a conformational change that occurs at low pH (e.g., transition to postfusion conformation), thereby stabilizing the HA protein.

In some embodiments, an HA protein comprises a substitution in, or removal of, a polybasic cleavage site. Cellular infection by influenza viruses involves cleavage of immature HA monomers at a cleavage site; differences in cleavage motifs of influenza viruses confer distinct tissue- and cell-specificity in which the viruses can replicate, resulting in different pathogenicity (Klenk et al. 1975. Virology. 68:426-439). Highly pathogenic avian influenza (HPAI) viruses typically possess polybasic HA cleavage sites (e.g., RKTR (SEQ ID NO: 120) or RKKR (SEQ ID NO: 121)), a feature which low pathogenic avian influenza (LPAI) viruses lack. In some embodiments, the mutation is a replacement of a polybasic cleavage site of an HPAI virus by a protease cleavage site of a LPAI virus. In some embodiments, the mutation is deletion of a polybasic cleavage site. In some embodiments, the mutation is deletion of an HPAI polybasic cleavage site. In some embodiments, the mutation is substitution of a polybasic cleavage site with an amino acid sequence that does not comprise a polybasic cleavage site. In some embodiments, the mutation is substitution of one or more arginine residues of an HPAI polybasic cleavage site with one or more non-basic residues (e.g. glycine and/or serine).

In some embodiments, an HA protein comprises one or more substitutions of a polar or charged cavity-lining residue with a hydrophobic residue. In some embodiments, a HA protein comprises one or more substitutions of a polar or charged cavity-lining residue with a glycine. Protein interiors may comprise tightly packed side chains which influence the stability of the protein, wherein larger cavities are less stable than narrow cavities (Bueno et al. J Mol Bio. 2006. 358(3):701-712). By replacing small cavity-lining residues with larger, hydrophobic residues, the stability of proteins can be increased by such cavity-filling mutations. Any suitable hydrophobic residue may be used in such a substitution, such as serine, alanine, valine, phenylalanine, histidine, leucine, methionine, valine, or tryptophan.

### Influenza B Virus HA Proteins

Some embodiments relate to influenza B virus HA proteins. Influenza B virus HA proteins may comprise one or more mutations (e.g., substitutions) relative to an amino acid sequence of a reference influenza B virus HA protein. The reference IBV HA protein may be an HA protein of an IBV isolate. The reference IBV HA protein may be an engineered IBV HA protein. Non-limiting examples of mutations include (i) one or more introduced cysteines, where a disulfide bond is formed by at least one of the introduced cysteines; (ii) one or more proline or glycine substitutions in a B loop of the HA protein; (iii) one or more substitutions of a charged or polar cavity-lining residue with a hydrophobic residue; (iv) one or more substitutions of a pH-sensitive histidine in the HA protein; and/or (v) a mutation in, or removal of, a polybasic cleavage site of the HA protein.

### B/Victoria Lineage HA Proteins

Some embodiments relate to influenza B/Victoria lineage virus HA proteins comprising an amino acid substitution relative to a reference influenza B/Victoria lineage virus HA protein. The reference influenza B/Victoria lineage virus HA protein may be an HA protein of an influenza B/Victoria lineage virus isolate. The reference influenza B/Victoria lineage virus HA protein may be an engineered influenza B/Victoria lineage virus HA protein. Non-limiting examples of influenza B/Victoria lineage virus isolates include B/Shangdong/7/1997, B/Hong Kong/330/2001, B/Malaysia/2506/2004, B/Brisbane/60/2008, B/Colorado/06/2017, B/Washington/02/2019, and B/Austria/1359417/2021. Representative amino acid sequences of influenza B/Victoria lineage virus HA proteins of previous seasonal influenza vaccines are provided as SEQ ID NOs: 154-160.

Those of ordinary skill in the art will appreciate that mutations disclosed in relation to a listed sequence, e.g., of influenza B/Austria/1359417/2021 virus HA, may be applied to HA proteins of other influenza B/Victoria lineage viruses or other reference influenza B/Victoria lineage virus HA proteins. For example, in applying an H381Y substitution to an influenza B/Victoria lineage virus HA protein, the skilled artisan would align the amino acid sequence of that reference HA protein to the B/Austria/1359417/2021 virus HA protein sequence of SEQ ID NO: 71, and introduce a Y (tyrosine) at the residue of the reference HA protein amino acid sequence that aligns to H381 (the histidine at position 381) of SEQ ID NO: 71.

Exemplary substitutions that may be present in influenza B/Victoria lineage HA proteins are provided below in Table HA-1. In the event a reference HA protein sequence (e.g. of an isolate) already includes a substitution mentioned in the table, then it will be appreciated that some embodiments will include the residues set forth at positions below without a substitution being made at that particular position. For example, if an isolate already contains a tyrosine at position 381, then embodiments comprising 381Y and 288V can be obtained with only a 288V substitution.

TABLE HA-1
B/Victoria lineage HA substitutions
(B/Austria/1359417/2021 (SEQ ID NO: 71) numbering)

| Substitution(s) | Mutation class |
|---|---|
| H381Y A288V | pH Switch / cavity filling |
| S27C Y349C | Intra-protomer DS (Head) |
| I295C K328C | Intra-protomer DS (Head) |
| S399C H473C | Intra-protomer DS (Stalk) |
| L422C D444C | Intra-protomer DS (Stalk) |
| K118C L216C | Inter-protomer DS (Head) |
| V237C D261C | Inter-protomer DS (Head) |
| G363C K480C | Inter-protomer DS (Stalk) |
| A364C K483C | Inter-protomer DS (Stalk) |
| I365C A476C | Inter-protomer DS (Stalk) |
| A366C R479C | Inter-protomer DS (Stalk) |
| G367C K483C | Inter-protomer DS (Stalk) |
| E435C A428C | Inter-protomer DS (Stalk) |
| N494C K483C | Inter-protomer DS (Stalk) |
| N494C K480C | Inter-protomer DS (Stalk) |
| E416P L417P N434P H433P | Prolines |
| N434P H433P | Prolines |
| T515P F516P | Prolines |
| H473F | pH Switch |

In preferred embodiments, the influenza B/Victoria lineage HA protein comprises a tyrosine substitution at position 381 and a valine substitution at position 288 (e.g., H381Y, A288V). These preferred substitutions can be combined with one or more additional substitutions set forth in the above table.

In some embodiments, the influenza B/Victoria lineage virus HA protein comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises H381Y and A288V substitutions relative to a reference influenza B/Victoria lineage virus HA protein, where the numbering of amino acids replaced in the reference HA protein corresponds to the numbering of SEQ ID NO: 71. An H381Y substitution is a replacement of a pH-sensitive histidine residue, and A290V is a cavity-filling substitution.

In some embodiments, the influenza B/Victoria lineage virus HA protein comprises a cysteine at position 27 and a cysteine at position 349, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 27 and a cysteine substitution at position 349 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises S27C and Y349C substitutions relative to a reference influenza B/Victoria lineage virus HA protein, where the numbering of amino acids replaced in the reference HA protein corresponds to the numbering of SEQ ID NO: 71. S27C and Y349C substitutions result in the formation of an intraprotomer disulfide bond in the head region of the HA protein. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

In some embodiments, the influenza B/Victoria lineage virus HA protein comprises a cysteine at position 295 and a cysteine at position 328, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 295 and a cysteine substitution at position 328 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises I295C and K328C substitutions relative to a reference influenza B/Victoria lineage virus HA protein, where the numbering of amino acids replaced in the reference HA protein corresponds to the numbering of SEQ ID NO: 71. I295C and K328C substitutions result in the formation of an intraprotomer disulfide bond in the head region of the HA protein. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

In some embodiments, the influenza B/Victoria lineage virus HA protein comprises a cysteine at position 399 and a cysteine at position 473, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 399 and a cysteine substitution at position 473 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises S399C and H473C substitutions relative to a reference influenza B/Victoria lineage virus HA protein, where the numbering of amino acids replaced in the reference HA protein corresponds to the numbering of SEQ ID NO: 71. S399C and H473C substitutions result in the formation of an intraprotomer disulfide bond in the stalk region of the HA protein. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

In some embodiments, the influenza B/Victoria lineage virus HA protein comprises a cysteine at position 422 and a cysteine at position 444, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 422 and a cysteine substitution at position 444 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises L422C and D444C substitutions relative to a reference influenza B/Victoria lineage virus HA protein, where the numbering of amino acids replaced in the reference HA protein corresponds to the numbering of SEQ ID NO: 71. L422C and D444C substitutions result in the formation of an intraprotomer disulfide bond in the stalk region of the HA protein. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises a tyrosine at position 381, a valine at position 288, a cysteine at position 422, and a cysteine at position 444, where the positions are numbered by alignment of the amino acid sequence of the influenza B/Victoria lineage virus HA protein to SEQ ID NO: 71

In some embodiments, the influenza B/Victoria lineage virus HA protein comprises a cysteine at position 118 and a cysteine at position 216, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 118 and a cysteine substitution at position 216 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises K118C and L216C substitutions relative to a reference influenza B/Victoria lineage virus HA protein, where the numbering of amino acids replaced in the reference HA protein corresponds to the numbering of SEQ ID NO: 71. K118C and L216C substitutions result in the formation of an interprotomer disulfide bond in the head region of the HA protein. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

In some embodiments, the influenza B/Victoria lineage virus HA protein comprises a cysteine at position 237 and a cysteine at position 261, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 237 and a cysteine substitution at position 261 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises V237C and D261C substitutions relative to a reference influenza B/Victoria lineage virus HA protein, where the numbering of amino acids replaced in the reference HA protein corresponds to the numbering of SEQ ID NO: 71. V237C and D261C substitutions result in the formation of an interprotomer disulfide bond in the head region of the HA protein. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

In some embodiments, the influenza B/Victoria lineage virus HA protein comprises a cysteine at position 363 and a cysteine at position 480, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 363 and a cysteine substitution at position 480 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises G363C and K480C substitutions relative to a reference influenza B/Victoria lineage virus HA protein, where the numbering of amino acids replaced in the reference HA protein corresponds to the numbering of SEQ ID NO: 71. G363C and K480C substitutions result in the formation of an interprotomer disulfide bond in the stalk region of the HA protein. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

In some embodiments, the influenza B/Victoria lineage virus HA protein comprises a cysteine at position 364 and a cysteine at position 483, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 364 and a cysteine substitution at position 483 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises A364C and K483C substitutions relative to a reference influenza B/Victoria lineage virus HA protein, where the numbering of amino acids replaced in the reference HA protein corresponds to the numbering of SEQ ID NO: 71. A364C and K483C substitutions result in the formation of an interprotomer disulfide bond in the stalk region of the HA protein. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71. In some embodiments, the influenza B/Victoria lineage virus HA protein comprises a tyrosine at position 381, a valine at position 288, a cysteine at position 364 and a cysteine at position 483, where the positions are numbered by alignment of the amino acid sequence of the influenza B/Victoria lineage virus HA protein to SEQ ID NO: 71.

In some embodiments, the influenza B/Victoria lineage virus HA protein comprises a cysteine at position 365 and a cysteine at position 476, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 365 and a cysteine substitution at position 476 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises 1365C and A476C substitutions relative to a reference influenza B/Victoria lineage virus HA protein, where the numbering of amino acids replaced in the reference HA protein corresponds to the numbering of SEQ ID NO: 71. 1365C and A476C substitutions result in the formation of an interprotomer disulfide bond in the stalk region of the HA protein. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

In some embodiments, the influenza B/Victoria lineage virus HA protein comprises a cysteine at position 366 and a cysteine at position 479, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 366 and a cysteine substitution at position 479 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises A366C and R479C substitutions relative to a reference influenza B/Victoria lineage virus HA protein, where the numbering of amino acids replaced in the reference HA protein corresponds to the numbering of SEQ ID NO: 71. A366C and R479C substitutions result in the formation of an interprotomer disulfide bond in the stalk region of the HA protein. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

In some embodiments, the influenza B/Victoria lineage virus HA protein comprises a cysteine at position 367 and a cysteine at position 483, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 367 and a cysteine substitution at position 483 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises G367C and K483C substitutions relative to a reference influenza B/Victoria lineage virus HA protein, where the numbering of amino acids replaced in the reference HA protein corresponds to the numbering of SEQ ID NO: 71. G367C and K483C substitutions result in the formation of an interprotomer disulfide bond in the stalk region of the HA protein. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71. In some embodiments, the influenza B/Victoria lineage virus HA protein comprises a tyrosine at position 381, a valine at position 288, a cysteine at position 367 and a cysteine at position 483, where the positions are numbered by alignment of the amino acid sequence of the influenza B/Victoria lineage virus HA protein to SEQ ID NO: 71.

In some embodiments, the influenza B/Victoria lineage virus HA protein comprises a cysteine at position 435 and a cysteine at position 428, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 435 and a cysteine substitution at position 428 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises E435C and A428C substitutions relative to a reference influenza B/Victoria lineage virus HA protein, where the numbering of amino acids replaced in the reference HA protein corresponds to the numbering of SEQ ID NO: 71. E435C and A428C substitutions result in the formation of an interprotomer disulfide bond in the stalk region of the HA protein. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

In some embodiments, the influenza B/Victoria lineage virus HA protein comprises a cysteine at position 494 and a cysteine at position 483, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 494 and a cysteine substitution at position 483 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises N494C and K483C substitutions relative to a reference influenza B/Victoria lineage virus HA protein, where the numbering of amino acids replaced in the reference HA protein corresponds to the numbering of SEQ ID NO: 71. N494C and K483C substitutions result in the formation of an interprotomer disulfide bond in the stalk region of the HA protein. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71. In some embodiments, the influenza B/Victoria lineage virus HA protein comprises a tyrosine at position 381, a valine at position 288, a cysteine at position 494 and a cysteine at position 483, where the positions are numbered by alignment of the amino acid sequence of the influenza B/Victoria lineage virus HA protein to SEQ ID NO: 71.

In some embodiments, the influenza B/Victoria lineage virus HA protein comprises a cysteine at position 494 and a cysteine at position 480, where the positions are numbered by alignment of the amino acid sequence of the influenza B/Victoria lineage virus HA protein to SEQ ID NO: 71. In some embodiments, the influenza B/Victoria lineage virus HA protein comprises a cysteine substitution at position 494 and a cysteine substitution at position 480 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises N494C and K480C substitutions relative to a reference influenza B/Victoria lineage virus HA protein, where the numbering of amino acids replaced in the reference HA protein corresponds to the numbering of SEQ ID NO: 71. N494C and K480C substitutions result in the formation of an interprotomer disulfide bond in the stalk region of the HA protein. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

In some embodiments, the influenza B/Victoria lineage virus HA protein comprises a proline at position 416, a proline at position 417, a proline at position 434, and a at position 433, where the positions are numbered by alignment of the amino acid sequence of the influenza B/Victoria lineage virus HA protein to SEQ ID NO: 71. In some embodiments, the influenza B/Victoria lineage virus HA protein comprises a proline substitution at position 416, a proline substitution at position 417, a proline substitution at position 434, and a substitution at position 433 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises E416P, L417P, N434P, and H433P substitutions relative to a reference influenza B/Victoria lineage virus HA protein, where the numbering of amino acids replaced in the reference HA protein corresponds to the numbering of SEQ ID NO: 71. Proline is a residue considered a helix breaker, and substitution of a residue in the B loop of an HA protein with proline (e.g., E416P, L417P, N434P, and H433P) can the protein by altering its conformation. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

In some embodiments, the influenza B/Victoria lineage virus HA protein comprises a proline at position 434 and a proline at position 433, where the positions are numbered by alignment of the amino acid sequence of the influenza B/Victoria lineage virus HA protein to SEQ ID NO: 71. In some embodiments, the influenza B/Victoria lineage virus HA protein comprises a proline substitution at position 434 and a proline substitution at position 433 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises N434P and H433P substitutions relative to a reference influenza B/Victoria lineage virus HA protein, where the numbering of amino acids replaced in the reference HA protein corresponds to the numbering of SEQ ID NO: 71. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

In some embodiments, the influenza B/Victoria lineage virus HA protein comprises a proline at position 515 and a proline at position 516, where the positions are numbered by alignment of the amino acid sequence of the influenza B/Victoria lineage virus HA protein to SEQ ID NO: 71. In some embodiments, the influenza B/Victoria lineage virus HA protein comprises a proline substitution at position 515 and a proline substitution at position 516 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises T515P and F516P substitutions relative to a reference influenza B/Victoria lineage virus HA protein, where the numbering of amino acids replaced in the reference HA protein corresponds to the numbering of SEQ ID NO: 71. Proline is a residue considered a helix breaker, and substitution of one or more residues in the B loop of an HA protein with proline (e.g., T515P and F516P) can the protein by altering its conformation. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

In some embodiments, the influenza B/Victoria lineage virus HA protein comprises a phenylalanine at position 473, where the positions are numbered by alignment of the amino acid sequence of the influenza B/Victoria lineage virus HA protein to SEQ ID NO: 71. In some embodiments, the influenza B/Victoria lineage virus HA protein comprises a phenylalanine substitution at position 473 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein comprises an H473F substitution relative to a reference influenza B/Victoria lineage virus HA protein, where the numbering of the amino acid replaced in the reference HA protein corresponds to the numbering of SEQ ID NO: 71. Substitution of a pH-sensitive histidine (e.g., H473) can inhibit or prevent the conformational change that occurs at low pH (e.g., during viral fusion), thereby stabilizing the HA protein. In some embodiments, the influenza B/Victoria lineage virus HA protein also comprises a tyrosine at position 381 and a valine at position 288, where the positions are numbered by alignment of the amino acid sequence of the influenza B/Victoria lineage virus HA protein to SEQ ID NO: 71. In some embodiments, the influenza B/Victoria lineage virus HA protein further comprises a tyrosine substitution at position 381 and a valine substitution at position 288 relative to a reference influenza B/Victoria lineage virus HA protein, where the positions are numbered by alignment of the amino acid sequence of the reference influenza B/Victoria lineage virus HA protein to SEQ ID NO: 71. In some embodiments, the influenza B/Victoria lineage virus HA protein further comprises H381Y and A288V substitutions relative to the reference HA protein, where the numbering of the amino acids replaced in the reference HA protein corresponds to the numbering of SEQ ID NO: 71.

Substitutions described in this subsection relating to B/Victoria lineage HA proteins may be combined. For example, any of the pairs of cysteine substitutions may be combined with one or more proline substitutions in the B loop and/or substitutions of pH-sensitive histidines. Pairs of cysteine substitutions that form different disulfide bonds (e.g., interprotomer versus intraprotomer bonds, head versus stalk region bonds) may also be combined. In some embodiments, the influenza B/Victoria lineage HA protein comprises one or more substitutions of a pH-sensitive histidine in the HA protein and one or more substitutions of a charged or polar cavity-lining residue with a hydrophobic residue.

In some embodiments, the influenza B/Victoria lineage HA protein comprises an amino acid sequence with at least 90%, at least 91%, at least 92%, at least 93%, at least 94%, at least 95%, at least 96%, at least 97%, at least 98%, or at least 99% sequence identity to the amino acid sequence of SEQ ID NO: 71. In some embodiments, the influenza B/Victoria lineage HA protein comprises an amino acid sequence with at least 90% sequence identity to the amino acid sequence of SEQ ID NO: 71.

### B/Yamagata Lineage HA Proteins

Some embodiments relate to influenza B/Yamagata lineage virus HA proteins comprising an amino acid substitution relative to a reference influenza B/Yamagata lineage virus HA protein. The reference influenza B/Yamagata lineage virus HA protein may be an HA protein of an influenza B/Yamagata lineage virus isolate. The reference influenza B/Yamagata lineage virus HA protein may be an engineered influenza B/Yamagata lineage virus HA protein. Non-limiting examples of influenza B/Yamagata lineage virus isolates include B/Beijing/184/1993, B/Sichuan/379/1999, B/Shanghai/361/2002, B/Florida/4/2006, B/Wisconsin/1/2010, B/Massachusetts/2/2012, and B/Phuket/3073/2013. Representative amino acid sequences of influenza B/Yamagata lineage virus HA proteins of previous seasonal influenza vaccines are provided as SEQ ID NOs: 164-167.

Those of ordinary skill in the art will appreciate that mutations disclosed in relation to a listed sequence, e.g., of influenza B/Phuket/3073/2013 virus HA, may be applied to HA proteins of other influenza B/Yamagata lineage viruses or other reference influenza B/Victoria lineage virus HA proteins. For example, in applying an A231C substitution to another influenza B/Yamagata lineage virus HA protein, the skilled artisan would align the amino acid sequence of that reference HA protein to the influenza B/Phuket/3073/2013 virus HA protein sequence of SEQ ID NO: 70, and introduce a C at the residue of the reference HA protein amino acid sequence that aligns to A231 of SEQ ID NO: 70.

Exemplary substitutions that may be present in influenza B/Yamgata lineage HA proteins are provided below in Table HA-2. In the event a reference HA protein sequence (e.g. of an isolate) already includes a substitution mentioned in the table, then it will be appreciated that some embodiments will include the residues set forth at positions below without a substitution being made at that particular position. For example, if an isolate already contains a tyrosine at position 381, then embodiments comprising 381Y and 290V can be obtained with only a 290V substitution.

TABLE 2
B/Yamagata lineage HA substitutions
(B/Phuket/3073/2013 (SEQ ID NO: 70) numbering)

| Substitution(s) | Mutation class |
|---|---|
| A231C G273C | Intra-protomer DS (Head) |
| K295C I332C | Intra-protomer DS (Head) |
| A396C L510C | Intra-protomer DS (Stalk) |
| V239C V276C | Inter-protomer DS (Head) |
| I367C S401C | Inter-protomer DS (Stalk) |
| F363C E404C | Inter-protomer DS (Stalk) |
| E437C G429C | Inter-protomer DS (Stalk) |
| D451C K422C | Inter-protomer DS (Stalk) |
| H381Y A290V | pH Switch (H381Y) / Cavity Filling (A290V) |

In some embodiments, the influenza B/Yamagata lineage virus HA protein comprises a cysteine at position 231 and a cysteine at position 273, where the positions are numbered by alignment of the amino acid sequence of the influenza B/Yamagata lineage virus HA protein to SEQ ID NO: 70. In some embodiments, the influenza B/Yamagata lineage virus HA protein comprises a cysteine substitution at position 231 and a cysteine substitution at position 273 relative to a reference influenza B/Yamagata lineage virus HA protein, where the positions are numbered by alignment of the amino acid sequence of the reference influenza B/Yamagata lineage virus HA protein to SEQ ID NO: 70. In some embodiments, the influenza B/Yamagata lineage virus HA protein comprises A231C and G273C substitutions relative to a reference influenza B/Yamagata lineage virus HA protein, where the numbering of amino acids replaced in the reference HA protein corresponds to the numbering of SEQ ID NO: 70. A231C and G273C substitutions result in the formation of an intraprotomer disulfide bond in the head region of the HA protein.

In some embodiments, the influenza B/Yamagata lineage virus HA protein comprises a cysteine at position 295 and a cysteine at position 332, where the positions are numbered by alignment of the amino acid sequence of the influenza B/Yamagata lineage virus HA protein to SEQ ID NO: 70. In some embodiments, the influenza B/Yamagata lineage virus HA protein comprises a cysteine substitution at position 295 and a cysteine substitution at position 332 relative to a reference influenza B/Yamagata lineage virus HA protein, where the positions are numbered by alignment of the amino acid sequence of the reference influenza B/Yamagata lineage virus HA protein to SEQ ID NO: 70. In some embodiments, the influenza B/Yamagata lineage virus HA protein comprises K295C and I332C substitutions relative to a reference influenza B/Yamagata lineage virus HA protein, where the numbering of amino acids replaced in the reference HA protein corresponds to the numbering of SEQ ID NO: 70. K295C and I332C substitutions result in the formation of an intraprotomer disulfide bond in the head region of the HA protein.

In some embodiments, the influenza B/Yamagata lineage virus HA protein comprises a cysteine at position 396 and a cysteine at position 510, where the positions are numbered by alignment of the amino acid sequence of the influenza B/Yamagata lineage virus HA protein to SEQ ID NO: 70. In some embodiments, the influenza B/Yamagata lineage virus HA protein comprises a cysteine substitution at position 396 and a cysteine substitution at position 510 relative to a reference influenza B/Yamagata lineage virus HA protein, where the positions are numbered by alignment of the amino acid sequence of the reference influenza B/Yamagata lineage virus HA protein to SEQ ID NO: 70. In some embodiments, the influenza B/Yamagata lineage virus HA protein comprises A396C and L510C substitutions relative to a reference influenza B/Yamagata lineage virus HA protein, where the numbering of amino acids replaced in the reference HA protein corresponds to the numbering of SEQ ID NO: 70. A396C and L510C substitutions result in the formation of an intraprotomer disulfide bond in the stalk region of the HA protein.

In some embodiments, the influenza B/Yamagata lineage virus HA protein comprises a cysteine at position 239 and a cysteine at position 276, where the positions are numbered by alignment of the amino acid sequence of the influenza B/Yamagata lineage virus HA protein to SEQ ID NO: 70. In some embodiments, the influenza B/Yamagata lineage virus HA protein comprises a cysteine substitution at position 239 and a cysteine substitution at position 276 relative to a reference influenza B/Yamagata lineage virus HA protein, where the positions are numbered by alignment of the amino acid sequence of the reference influenza B/Yamagata lineage virus HA protein to SEQ ID NO: 70. In some embodiments, the influenza B/Yamagata lineage virus HA protein comprises V239C and V276C substitutions relative to a reference influenza B/Yamagata lineage virus HA protein, where the numbering of amino acids replaced in the reference HA protein corresponds to the numbering of SEQ ID NO: 70. V239C and V276C substitutions result in the formation of an interprotomer disulfide bond in the head region of the HA protein. In some embodiments, the influenza B/Yamagata lineage virus HA protein comprises a cysteine at position 239, a cysteine at position 276, a cysteine at position 451, and a cysteine at position 422, where the positions are numbered by alignment of the amino acid sequence of the influenza B/Yamagata lineage virus HA protein to SEQ ID NO: 70.

In some embodiments, the influenza B/Yamagata lineage virus HA protein comprises a cysteine at position 367 and a cysteine at position 401, where the positions are numbered by alignment of the amino acid sequence of the influenza B/Yamagata lineage virus HA protein to SEQ ID NO: 70. In some embodiments, the influenza B/Yamagata lineage virus HA protein comprises a cysteine substitution at position 367 and a cysteine substitution at position 401 relative to a reference influenza B/Yamagata lineage virus HA protein, where the positions are numbered by alignment of the amino acid sequence of the reference influenza B/Yamagata lineage virus HA protein to SEQ ID NO: 70. In some embodiments, the influenza B/Yamagata lineage virus HA protein comprises I367C and S401C substitutions relative to a reference influenza B/Yamagata lineage virus HA protein, where the numbering of amino acids replaced in the reference HA protein corresponds to the numbering of SEQ ID NO: 70. I367C and S401C substitutions result in the formation of an interprotomer disulfide bond in the stalk region of the HA protein. In some embodiments, the influenza B/Yamagata lineage virus HA protein comprises a cysteine at position 367, a cysteine at position 401, a cysteine at position 451, and a cysteine at position 422, where the positions are numbered by alignment of the amino acid sequence of the influenza B/Yamagata lineage virus HA protein to SEQ ID NO: 70.

In some embodiments, the influenza B/Yamagata lineage virus HA protein comprises a cysteine at position 363 and a cysteine at position 404, where the positions are numbered by alignment of the amino acid sequence of the influenza B/Yamagata lineage virus HA protein to SEQ ID NO: 70. In some embodiments, the influenza B/Yamagata lineage virus HA protein comprises a cysteine substitution at position 363 and a cysteine substitution at position 404 relative to a reference influenza B/Yamagata lineage virus HA protein, where the positions are numbered by alignment of the amino acid sequence of the reference influenza B/Yamagata lineage virus HA protein to SEQ ID NO: 70. In some embodiments, the influenza B/Yamagata lineage virus HA protein comprises F363C and E404C substitutions relative to a reference influenza B/Yamagata lineage virus HA protein, where the numbering of amino acids replaced in the reference HA protein corresponds to the numbering of SEQ ID NO: 70. F363C and E404C substitutions result in the formation of an interprotomer disulfide bond in the stalk region of the HA protein.

In some embodiments, the influenza B/Yamagata lineage virus HA protein comprises a cysteine at position 437 and a cysteine at position 429, where the positions are numbered by alignment of the amino acid sequence of the influenza B/Yamagata lineage virus HA protein to SEQ ID NO: 70. In some embodiments, the influenza B/Yamagata lineage virus HA protein comprises a cysteine substitution at position 437 and a cysteine substitution at position 429 relative to a reference influenza B/Yamagata lineage virus HA protein, where the positions are numbered by alignment of the amino acid sequence of the reference influenza B/Yamagata lineage virus HA protein to SEQ ID NO: 70. In some embodiments, the influenza B/Yamagata lineage virus HA protein comprises E437C and G429C substitutions relative to a reference influenza B/Yamagata lineage virus HA protein, where the numbering of amino acids replaced in the reference HA protein corresponds to the numbering of SEQ ID NO: 70. E437C and G429C substitutions result in the formation of an interprotomer disulfide bond in the stalk region of the HA protein.

In some embodiments, the influenza B/Yamagata lineage virus HA protein comprises a cysteine at position 451 and a cysteine at position 422, where the positions are numbered by alignment of the amino acid sequence of the influenza B/Yamagata lineage virus HA protein to SEQ ID NO: 70. In some embodiments, the influenza B/Yamagata lineage virus HA protein comprises a cysteine substitution at position 451 and a cysteine substitution at position 422 relative to a reference influenza B/Yamagata lineage virus HA protein, where the positions are numbered by alignment of the amino acid sequence of the reference influenza B/Yamagata lineage virus HA protein to SEQ ID NO: 70. In some embodiments, the influenza B/Yamagata lineage virus HA protein comprises D451C and K422C substitutions relative to a reference influenza B/Yamagata lineage virus HA protein, where the numbering of amino acids replaced in the reference HA protein corresponds to the numbering of SEQ ID NO: 70. D451C and K422C substitutions result in the formation of an interprotomer disulfide bond in the stalk region of the HA protein.

In some embodiments, the influenza B/Yamagata lineage virus HA protein comprises a tyrosine at position 381 and a valine at position 290, where the positions are numbered by alignment of the amino acid sequence of the influenza B/Yamagata lineage virus HA protein to SEQ ID NO: 70. In some embodiments, the influenza B/Yamagata lineage virus HA protein comprises a tyrosine substitution at position 381 and a valine substitution at position 290 relative to a reference influenza B/Yamagata lineage virus HA protein, where the positions are numbered by alignment of the amino acid sequence of the reference influenza B/Yamagata lineage virus HA protein to SEQ ID NO: 70. In some embodiments, the influenza B/Yamagata lineage virus HA protein comprises H381Y and A290V substitutions relative to a reference influenza B/Yamagata lineage virus HA protein, where the numbering of amino acids replaced in the reference HA protein corresponds to the numbering of SEQ ID NO: 70. H381Y is a pH-sensitive histidine residue replacement substitution, and A290V is a cavity-filling substitution.

Substitutions described in this subsection relating to B/Yamagata lineage HA proteins may be combined. For example, any of the pairs of cysteine substitutions may be combined with one or more substitutions of pH-sensitive histidines and/or cavity-filling substitutions. Pairs of cysteine substitutions that form different disulfide bonds (e.g., interprotomer v. intraprotomer bonds, head v. stalk region bonds) may also be combined. In some embodiments, the influenza B/Yamagata lineage HA protein comprises one or more substitutions of a pH-sensitive histidine in the HA protein and one or more substitutions of a charged or polar cavity-lining residue with a hydrophobic residue.

In some embodiments, the influenza B/Yamagata lineage virus HA protein comprises H381Y and A290V substitutions relative to a reference influenza B/Yamagata lineage virus HA protein, where the numbering of amino acids replaced in the reference HA protein corresponds to the numbering of SEQ ID NO: 70 in addition to one or more other substitutions described in this section.

In some embodiments, the influenza B/Yamagata lineage HA protein comprises an amino acid sequence with at least 90%, at least 91%, at least 92%, at least 93%, at least 94%, at least 95%, at least 96%, at least 97%, at least 98%, or at least 99% sequence identity to the amino acid sequence of SEQ ID NO: 70. In some embodiments, the influenza B/Yamagata lineage HA protein comprises an amino acid sequence with at least 90% sequence identity to the amino acid sequence of SEQ ID NO: 70.

### Influenza a Virus HA Proteins

Some embodiments relate to influenza A virus HA proteins. Influenza A virus HA proteins may comprise one or more mutations (e.g., substitutions) relative to an amino acid sequence of an HA protein of an influenza A virus isolate.

### H1 HA Proteins

Some embodiments relate to IAV H1 HA proteins comprising an amino acid substitution relative to a reference IAV H1 HA protein. The reference IAV H1 HA protein may be an HA protein of an isolate of an influenza A virus of an IAV subtype containing an H1 HA protein, such as an influenza A/(H1N1)pdm09 virus. H1 HA proteins are present, e.g., on influenza A viruses of the A/(H1N1)pdm09 subtype, but other IAV subtypes expressing H1 HA proteins have been identified (such as the H1N2 subtype endemic in pigs and occasionally observed in humans). Non-limiting examples of influenza A/(H1N1)pdm09 virus isolates (and related influenza A/(H1N1) virus isolates preceding the 2009 influenza A/(H1N1) virus pandemic) include A/Beijing/262/1995 (H1N1), A/New Caledonia/20/1999(H1N1), A/Solomon Islands/3/2006(H1N1), A/Brisbane/59/2007(H1N1), A/California/7/2009(H1N1)pdm09, A/Michigan/45/2015 (H1N1)pdm09, A/Brisbane/02/2018(H1N1)pdm09, A/Guangdong-Maonan/SWL1536/2019 (H1N1)pdm09, A/Hawaii/70/2019(H1N1)pdm09, (A/Victoria/2570/2019(H1N1)pdm09, A/Wisconsin/588/2019(H1N1)pdm09, A/Victoria/4897/2022(H1N1)pdm09, and A/Wisconsin/67/2022(H1N1)pdm09. Representative amino acid sequences of influenza A/(H1N1)pdm09 virus HA proteins of previous seasonal influenza vaccines are provided as SEQ ID NOs: 122-135.

Those of ordinary skill in the art will appreciate that mutations disclosed in relation to a listed sequence, e.g., of influenza A/Wisconsin/67/2022(H1N1)pdm09 virus HA, may be applied to other IAV H1 HA proteins. For example, in applying a K391C substitution to another IAV H1 HA protein, the skilled artisan would align the amino acid sequence of that reference HA protein to the influenza A/Wisconsin/67/2022(H1N1)pdm09 virus HA protein sequence of SEQ ID NO: 83, and introduce a C at the residue of the reference HA protein amino acid sequence that aligns to K391 of SEQ ID NO: 83. Similarly, mutations disclosed in relation to a reference sequence of influenza A/Sydney/5/2021(H1N1)pdm09 virus HA may be applied to a different reference HA protein by alignment of the reference HA protein amino acid sequence to the amino acid sequence of SEQ ID NO: 95. Such alignment allows the skilled artisan to identify the residue in the reference HA protein amino acid sequence that corresponds to the mutated residue of the influenza A/Sydney/5/2021(H1N1)pdm09 virus HA protein (e.g., to identify the residue of the reference HA protein amino acid sequence that corresponds to V410 of the A/Sydney/5/2021(H1N1)pdm09 virus HA protein amino acid sequence, to determine where to introduce a C, in applying a V410C substitution).

Exemplary substitutions that may be present in IAV H1 HA proteins are provided below in Tables HA-3 and HA-4. In the event a reference HA protein sequence (e.g. of an isolate) already includes a substitution mentioned in the table, then it will be appreciated that some embodiments will include the residues set forth at positions below without a substitution being made at that particular position. For example, if an isolate already contains an isoleucine at position 395, then embodiments comprising 395I and 447I can be obtained with only a 447I substitution.

TABLE HA-3
A/(H1N1) subtype HA substitutions
(A/Wisconsin/67/2022 (H1N1)pdm09 (SEQ ID NO: 83) numbering)

| Substitution(s) | Mutation class |
|---|---|
| K391C L37C | Inter-protomer DS (Stalk) |
| K395C V36C | Inter-protomer DS (Stalk) |
| N461C G348C | Inter-protomer DS (Stalk) |
| N404P H416P | Proline (Pro) |
| K395I E447I | Hydrophobic Interaction (Hyd) |
| D456G K402I | Hydrophobic Interaction (Hyd) |
| K391C L37C N404P H416P | InterDS + Pro |
| K391C L37C K395I E447I | InterDS + Hyd |
| K391C L37C D456G K402I | InterDS + Hyd |
| K395C V36C N404P H416P | InterDS + Pro |
| K395C V36C D456G K402I | InterDS + Hyd |
| N461C G348C N404P H416P | InterDS + Pro |
| N461C G348C K395I E447I | InterDS + Hyd |
| N461C G348C D456G K402I | InterDS + Hyd |
| K391C L37C N404P H416P | InterDS + Pro + Hyd |
| D456G K402I | |

TABLE HA-4
A/(H1N1) subtype HA substitutions
(A/Sydney/5/2021(H1N1)pdm09 (SEQ ID NO: 95) numbering)

| Substitution(s) | Mutation class |
|---|---|
| V410C L462C | Intra-protomer DS |
| E120C K419C | Inter-protomer DS |
| K391C L37C | Inter-protomer DS |
| K395C V36C | Inter-protomer DS |
| Q406C D430C | Inter-protomer DS |
| S457C L346C | Inter-protomer DS |
| N461C G348C | Inter-protomer DS |
| N404P K419P | Proline |
| N404P H416P | Proline |
| T405P Q406P | Proline |
| N415P H416P | Proline |
| H25Y H45E | pH Switch |
| H370Y K497W | pH Switch |
| K402G T405G F407G | Glycine |
| K395I E447I | Cavity Filling |
| D456G K402I | Glycine(s) (D456G) / Cavity |
| | Filling (K402I) |
| L442C N423C | Inter-protomer DS |
| K391G | Glycine(s) |
| V410C L462C S457C L346C | Intra- and Inter-protomer DS |
| K391C L37C H370F H455F | Inter-protomer DS + pH Switch |

Some embodiments relate to IAV H1 HA proteins having one or more substitutions relative to a reference H1 HA protein, wherein the numbering of amino acids replaced in the reference IAV H1 HA protein corresponds to the numbering of SEQ ID NO: 83, which represents the HA protein of the influenza A/Wisconsin/67/2022(H1N1)pdm09 virus isolate.

In some embodiments, the IAV H1 HA protein comprises a cysteine at position 391 and a cysteine at position 37, where the positions are numbered by alignment of the amino acid sequence of the IAV H1 HA protein to SEQ ID NO: 83. In some embodiments, the IAV H1 HA protein comprises a cysteine substitution at position 391 and a cysteine substitution at position 37 relative to a reference IAV H1 HA protein, where the positions are numbered by alignment of the amino acid sequence of the reference IAV H1 HA protein to SEQ ID NO: 83. In some embodiments, the IAV H1 HA protein comprises K391C and L37C substitutions relative to a reference IAV H1 HA protein, where the numbering of amino acids replaced in the reference IAV H1 HA protein corresponds to the numbering of SEQ ID NO: 83. K391C and L37C substitutions result in the formation of an interprotomer disulfide bond in the stalk region of the HA protein.

In some embodiments, the IAV H1 HA protein comprises a cysteine at position 395 and a cysteine at position 36, where the positions are numbered by alignment of the amino acid sequence of the IAV H1 HA protein to SEQ ID NO: 83. In some embodiments, the IAV H1 HA protein comprises a cysteine substitution at position 395 and a cysteine substitution at position 36 relative to a reference IAV H1 HA protein, where the positions are numbered by alignment of the amino acid sequence of the reference IAV H1 HA protein to SEQ ID NO: 83. In some embodiments, the IAV H1 HA protein comprises K395C and V36C substitutions relative to a reference IAV H1 HA protein, where the numbering of amino acids replaced in the reference IAV H1 HA protein corresponds to the numbering of SEQ ID NO: 83. K395C and V36C substitutions result in the formation of an interprotomer disulfide bond in the stalk region of the HA protein.

In some embodiments, the IAV H1 HA protein comprises a cysteine at position 461 and a cysteine at position 348, where the positions are numbered by alignment of the amino acid sequence of the IAV H1 HA protein to SEQ ID NO: 83. In some embodiments, the IAV H1 HA protein comprises a cysteine substitution at position 461 and a cysteine substitution at position 348 relative to a reference IAV H1 HA protein, where the positions are numbered by alignment of the amino acid sequence of the reference IAV H1 HA protein to SEQ ID NO: 83. In some embodiments, the IAV H1 HA protein comprises N461C and G348C substitutions relative to a reference IAV H1 HA protein, where the numbering of amino acids replaced in the reference IAV H1 HA protein corresponds to the numbering of SEQ ID NO: 83. N461C and G348C substitutions result in the formation of an interprotomer disulfide bond in the stalk region of the HA protein.

In some embodiments, the IAV H1 HA protein comprises a proline at position 404 and a proline at position 416, where the positions are numbered by alignment of the amino acid sequence of the IAV H1 HA protein to SEQ ID NO: 83. In some embodiments, the IAV H1 HA protein comprises a proline substitution at position 404 and a proline substitution at position 416 relative to a reference IAV H1 HA protein, where the positions are numbered by alignment of the amino acid sequence of the reference IAV H1 HA protein to SEQ ID NO: 83. In some embodiments, the IAV H1 HA protein comprises N404P and H416P substitutions relative to a reference IAV H1 HA protein, where the numbering of amino acids replaced in the reference IAV H1 HA protein corresponds to the numbering of SEQ ID NO: 83. N404P and H416P are proline substitutions in the B loop of the HA protein, stabilizing the HA protein by changing its conformation. In some embodiments, the IAV H1 HA protein comprises a proline at position 404, a proline at position 416, a cysteine at position 395, and a cysteine at position 36, where the positions are numbered by alignment of the amino acid sequence of the IAV H1 HA protein to SEQ ID NO: 83. In some embodiments, the IAV H1 HA protein comprises a proline at position 404, a proline at position 416, a cysteine at position 391, and a cysteine at position 36, where the positions are numbered by alignment of the amino acid sequence of the IAV H1 HA protein to SEQ ID NO: 83. In some embodiments, the IAV H1 HA protein comprises a proline at position 404, a proline at position 416, a cysteine at position 391, a cysteine at position 37, a glycine at position 456, and an isoleucine at position 402, where the positions are numbered by alignment of the amino acid sequence of the IAV H1 HA protein to SEQ ID NO: 83.

In some embodiments, the IAV H1 HA protein comprises a isoleucine at position 395 and a isoleucine at position 447, where the positions are numbered by alignment of the amino acid sequence of the IAV H1 HA protein to SEQ ID NO: 83. In some embodiments, the IAV H1 HA protein comprises a isoleucine substitution at position 395 and a isoleucine substitution at position 447 relative to a reference IAV H1 HA protein, where the positions are numbered by alignment of the amino acid sequence of the reference IAV H1 HA protein to SEQ ID NO: 83. In some embodiments, the IAV H1 HA protein comprises K395I and E447I substitutions relative to a reference IAV H1 HA protein, where the numbering of amino acids replaced in the reference IAV H1 HA protein corresponds to the numbering of SEQ ID NO: 83. K395I and E447I substitutions are replacements of charged lysine and glutamate residues with hydrophobic isoleucine residues.

In some embodiments, the IAV H1 HA protein comprises a glycine at position 456 and a isoleucine at position 402, where the positions are numbered by alignment of the amino acid sequence of the IAV H1 HA protein to SEQ ID NO: 83. In some embodiments, the IAV H1 HA protein comprises a glycine substitution at position 456 and a isoleucine substitution at position 402 relative to a reference IAV H1 HA protein, where the positions are numbered by alignment of the amino acid sequence of the reference IAV H1 HA protein to SEQ ID NO: 83. In some embodiments, the IAV H1 HA protein comprises D456G and K402I substitutions relative to a reference IAV H1 HA protein, where the numbering of amino acids replaced in the reference IAV H1 HA protein corresponds to the numbering of SEQ ID NO: 83. D456G and K402I substitutions are replacements of charged aspartate and lysine residues with uncharged glycine and uncharged, hydrophobic isoleucine residues. In some embodiments, the IAV H1 HA protein comprises a glycine at position 456, an isoleucine at position 402, a cysteine at position 395, and a cysteine at position 36, where the positions are numbered by alignment of the amino acid sequence of the IAV H1 HA protein to SEQ ID NO: 83.

In some embodiments, the amino acid substitution comprises a cysteine substitution at position 456 and a isoleucine substitution at position 402, where the positions are numbered by alignment of the amino acid sequence of the influenza B/Victoria lineage virus HA protein to SEQ ID NO: 83. In some embodiments, the IAV H1 HA protein comprises a cysteine substitution at position 456 and a isoleucine substitution at position 402, where the positions are numbered by alignment of the amino acid sequence of the IAV H1 HA protein to SEQ ID NO: 83. In some embodiments, the IAV H1 HA protein comprises D456C and K402I substitutions relative to a reference IAV H1 HA protein, where the numbering of amino acids replaced in the reference IAV H1 HA protein corresponds to the numbering of SEQ ID NO: 83. In some embodiments, the IAV H1 HA protein comprises a cysteine at position 456, an isoleucine at position 402, a cysteine at position 395, and a cysteine at position 36, where the positions are numbered by alignment of the amino acid sequence of the IAV H1 HA protein to SEQ ID NO: 83.

In some embodiments, the IAV H1 HA protein comprises an amino acid sequence with at least 90%, at least 91%, at least 92%, at least 93%, at least 94%, at least 95%, at least 96%, at least 97%, at least 98%, or at least 99% sequence identity to the amino acid sequence of SEQ ID NO: 83. In some embodiments, the IAV H1 HA protein comprises an amino acid sequence with at least 90% sequence identity to the amino acid sequence of SEQ ID NO: 83.

Some embodiments relate to IAV H1 HA proteins having one or more substitutions relative to a reference H1 HA protein, wherein the numbering of amino acids replaced in the reference IAV H1 HA protein corresponds to the numbering of SEQ ID NO: 95, which represents the HA protein of the influenza A/Sydney/5/2021(H1N1)pdm09 virus isolate.

In some embodiments, the IAV H1 HA protein comprises a cysteine at position 410 and a cysteine at position 462, where the positions are numbered by alignment of the amino acid sequence of the IAV H1 HA protein to SEQ ID NO: 95. In some embodiments, the IAV H1 HA protein comprises a cysteine substitution at position 410 and a cysteine substitution at position 462 relative to a reference IAV H1 HA protein, where the positions are numbered by alignment of the amino acid sequence of the reference IAV H1 HA protein to SEQ ID NO: 95. In some embodiments, the IAV H1 HA protein comprises V410C and L462C substitutions relative to a reference IAV H1 HA protein, where the numbering of amino acids replaced in the reference IAV H1 HA protein corresponds to the numbering of SEQ ID NO: 95. V410C and L462C substitutions result in the formation of an intraprotomer disulfide bond in the HA protein.

In some embodiments, the IAV H1 HA protein comprises a cysteine at position 120 and a cysteine at position 419, where the positions are numbered by alignment of the amino acid sequence of the IAV H1 HA protein to SEQ ID NO: 95. In some embodiments, the IAV H1 HA protein comprises a cysteine substitution at position 120 and a cysteine substitution at position 419 relative to a reference IAV H1 HA protein, where the positions are numbered by alignment of the amino acid sequence of the reference IAV H1 HA protein to SEQ ID NO: 95. In some embodiments, the IAV H1 HA protein comprises E120C and K419C substitutions relative to a reference IAV H1 HA protein, where the numbering of amino acids replaced in the reference IAV H1 HA protein corresponds to the numbering of SEQ ID NO: 95. E120C and K419C substitutions result in the formation of an interprotomer disulfide bond in the HA protein.

In some embodiments, the IAV H1 HA protein comprises a cysteine at position 391 and a cysteine at position 37, where the positions are numbered by alignment of the amino acid sequence of the IAV H1 HA protein to SEQ ID NO: 95. In some embodiments, the IAV H1 HA protein comprises a cysteine substitution at position 391 and a cysteine substitution at position 37 relative to a reference IAV H1 HA protein, where the positions are numbered by alignment of the amino acid sequence of the reference IAV H1 HA protein to SEQ ID NO: 95. In some embodiments, the IAV H1 HA protein comprises K391C and L37C substitutions relative to a reference IAV H1 HA protein, where the numbering of amino acids replaced in the reference IAV H1 HA protein corresponds to the numbering of SEQ ID NO: 95. K391C and L37C substitutions result in the formation of an interprotomer disulfide bond in the HA protein.

In some embodiments, the IAV H1 HA protein comprises a cysteine at position 395 and a cysteine at position 36, where the positions are numbered by alignment of the amino acid sequence of the IAV H1 HA protein to SEQ ID NO: 95. In some embodiments, the IAV H1 HA protein comprises a cysteine substitution at position 395 and a cysteine substitution at position 36 relative to a reference IAV H1 HA protein, where the positions are numbered by alignment of the amino acid sequence of the reference IAV H1 HA protein to SEQ ID NO: 95. In some embodiments, the IAV H1 HA protein comprises K395C and V36C substitutions relative to a reference IAV H1 HA protein, where the numbering of amino acids replaced in the reference IAV H1 HA protein corresponds to the numbering of SEQ ID NO: 95. K395C and V36C substitutions result in the formation of an interprotomer disulfide bond in the HA protein.

In some embodiments, the IAV H1 HA protein comprises a cysteine at position 406 and a cysteine at position 430, where the positions are numbered by alignment of the amino acid sequence of the IAV H1 HA protein to SEQ ID NO: 95. In some embodiments, the IAV H1 HA protein comprises a cysteine substitution at position 406 and a cysteine substitution at position 430 relative to a reference IAV H1 HA protein, where the positions are numbered by alignment of the amino acid sequence of the reference IAV H1 HA protein to SEQ ID NO: 95. In some embodiments, the IAV H1 HA protein comprises Q406C and D430C substitutions relative to a reference IAV H1 HA protein, where the numbering of amino acids replaced in the reference IAV H1 HA protein corresponds to the numbering of SEQ ID NO: 95. Q406C and D430C substitutions result in the formation of an interprotomer disulfide bond in the HA protein.

In some embodiments, the IAV H1 HA protein comprises a cysteine at position 457 and a cysteine at position 346, where the positions are numbered by alignment of the amino acid sequence of the IAV H1 HA protein to SEQ ID NO: 95. In some embodiments, the IAV H1 HA protein comprises a cysteine substitution at position 457 and a cysteine substitution at position 346 relative to a reference IAV H1 HA protein, where the positions are numbered by alignment of the amino acid sequence of the reference IAV H1 HA protein to SEQ ID NO: 95. In some embodiments, the IAV H1 HA protein comprises S457C and L346C substitutions relative to a reference IAV H1 HA protein, where the numbering of amino acids replaced in the reference IAV H1 HA protein corresponds to the numbering of SEQ ID NO: 95. S457C and L346C substitutions result in the formation of an interprotomer disulfide bond in the HA protein.

In some embodiments, the IAV H1 HA protein comprises a cysteine at position 461 and a cysteine at position 348, where the positions are numbered by alignment of the amino acid sequence of the IAV H1 HA protein to SEQ ID NO: 95. In some embodiments, the IAV H1 HA protein comprises a cysteine substitution at position 461 and a cysteine substitution at position 348 relative to a reference IAV H1 HA protein, where the positions are numbered by alignment of the amino acid sequence of the reference IAV H1 HA protein to SEQ ID NO: 95. In some embodiments, the IAV H1 HA protein comprises N461C and G348C substitutions relative to a reference IAV H1 HA protein, where the numbering of amino acids replaced in the reference IAV H1 HA protein corresponds to the numbering of SEQ ID NO: 95. N461C and G348C substitutions result in the formation of an interprotomer disulfide bond in the HA protein.

In some embodiments, the IAV H1 HA protein comprises a proline at position 404 and a proline at position 419, where the positions are numbered by alignment of the amino acid sequence of the IAV H1 HA protein to SEQ ID NO: 95. In some embodiments, the IAV H1 HA protein comprises a proline substitution at position 404 and a proline substitution at position 419 relative to a reference IAV H1 HA protein, where the positions are numbered by alignment of the amino acid sequence of the reference IAV H1 HA protein to SEQ ID NO: 95. In some embodiments, the IAV H1 HA protein comprises N404P and K419P substitutions relative to a reference IAV H1 HA protein, where the numbering of amino acids replaced in the reference IAV H1 HA protein corresponds to the numbering of SEQ ID NO: 95. N404P and K419P substitutions are proline substitutions in the B loop of the HA protein.

In some embodiments, the IAV H1 HA protein comprises a proline at position 404 and a proline at position 416, where the positions are numbered by alignment of the amino acid sequence of the IAV H1 HA protein to SEQ ID NO: 95. In some embodiments, the IAV H1 HA protein comprises a proline substitution at position 404 and a proline substitution at position 416 relative to a reference IAV H1 HA protein, where the positions are numbered by alignment of the amino acid sequence of the reference IAV H1 HA protein to SEQ ID NO: 95. In some embodiments, the IAV H1 HA protein comprises N404P and H416P substitutions relative to a reference IAV H1 HA protein, where the numbering of amino acids replaced in the reference IAV H1 HA protein corresponds to the numbering of SEQ ID NO: 95. N404P and H416P substitutions are proline substitutions in the B loop of the HA protein.

In some embodiments, the IAV H1 HA protein comprises a proline at position 405 and a proline at position 406, where the positions are numbered by alignment of the amino acid sequence of the IAV H1 HA protein to SEQ ID NO: 95. In some embodiments, the IAV H1 HA protein comprises a proline substitution at position 405 and a proline substitution at position 406 relative to a reference IAV H1 HA protein, where the positions are numbered by alignment of the amino acid sequence of the reference IAV H1 HA protein to SEQ ID NO: 95. In some embodiments, the IAV H1 HA protein comprises T405P and Q406P substitutions relative to a reference IAV H1 HA protein, where the numbering of amino acids replaced in the reference IAV H1 HA protein corresponds to the numbering of SEQ ID NO: 95. T405P and Q406P substitutions are proline substitutions in the B loop of the HA protein.

In some embodiments, the IAV H1 HA protein comprises a proline at position 415 and a proline at position 416, where the positions are numbered by alignment of the amino acid sequence of the IAV H1 HA protein to SEQ ID NO: 95. In some embodiments, the IAV H1 HA protein comprises a proline substitution at position 415 and a proline substitution at position 416 relative to a reference IAV H1 HA protein, where the positions are numbered by alignment of the amino acid sequence of the reference IAV H1 HA protein to SEQ ID NO: 95. In some embodiments, the IAV H1 HA protein comprises N415P and H416P substitutions relative to a reference IAV H1 HA protein, where the numbering of amino acids replaced in the reference IAV H1 HA protein corresponds to the numbering of SEQ ID NO: 95. N415P and H416P substitutions are proline substitutions in the B loop of the HA protein.

In some embodiments, the IAV H1 HA protein comprises a tyrosine at position 25 and a glutamic acid at position 45, where the positions are numbered by alignment of the amino acid sequence of the IAV H1 HA protein to SEQ ID NO: 95. In some embodiments, the IAV H1 HA protein comprises a tyrosine substitution at position 25 and a glutamic acid substitution at position 45 relative to a reference IAV H1 HA protein, where the positions are numbered by alignment of the amino acid sequence of the reference IAV H1 HA protein to SEQ ID NO: 95. In some embodiments, the IAV H1 HA protein comprises H25Y and H45E substitutions relative to a reference IAV H1 HA protein, where the numbering of amino acids replaced in the reference IAV H1 HA protein corresponds to the numbering of SEQ ID NO: 95. H25Y and H45E substitutions are replacements of pH-sensitive histidines.

In some embodiments, the IAV H1 HA protein comprises a tyrosine at position 370 and a tryptophan at position 497, where the positions are numbered by alignment of the amino acid sequence of the IAV H1 HA protein to SEQ ID NO: 95. In some embodiments, the IAV H1 HA protein comprises a tyrosine substitution at position 370 and a tryptophan substitution at position 497 relative to a reference IAV H1 HA protein, where the positions are numbered by alignment of the amino acid sequence of the reference IAV H1 HA protein to SEQ ID NO: 95. In some embodiments, the IAV H1 HA protein comprises H370Y and K497W substitutions relative to a reference IAV H1 HA protein, where the numbering of amino acids replaced in the reference IAV H1 HA protein corresponds to the numbering of SEQ ID NO: 95. An H370Y substitution is a replacement of a pH-sensitive histidine, and a K497W substitution is a cavity-filling substitution.

In some embodiments, the IAV H1 HA protein comprises a glycine at position 402, a glycine at position 405, and a glycine at position 407, where the positions are numbered by alignment of the amino acid sequence of the IAV H1 HA protein to SEQ ID NO: 95. In some embodiments, the IAV H1 HA protein comprises a glycine substitution at position 402, a glycine substitution at position 405, and a glycine substitution at position 407 relative to a reference IAV H1 HA protein, where the positions are numbered by alignment of the amino acid sequence of the reference IAV H1 HA protein to SEQ ID NO: 95. In some embodiments, the IAV H1 HA protein comprises K402G, T405G, and F407G substitutions relative to a reference IAV H1 HA protein, where the numbering of amino acids replaced in the reference IAV H1 HA protein corresponds to the numbering of SEQ ID NO: 95. K402G, T405G, and F407G substitutions are glycine substitutions in the B loop of the HA protein.

In some embodiments, the IAV H1 HA protein comprises a isoleucine at position 395 and a isoleucine at position 447, where the positions are numbered by alignment of the amino acid sequence of the IAV H1 HA protein to SEQ ID NO: 95. In some embodiments, the IAV H1 HA protein comprises a isoleucine substitution at position 395 and a isoleucine substitution at position 447 relative to a reference IAV H1 HA protein, where the positions are numbered by alignment of the amino acid sequence of the reference IAV H1 HA protein to SEQ ID NO: 95. In some embodiments, the IAV H1 HA protein comprises K395I and E447I substitutions relative to a reference IAV H1 HA protein, where the numbering of amino acids replaced in the reference IAV H1 HA protein corresponds to the numbering of SEQ ID NO: 95. K395I and E447I substitutions are cavity-filling substitutions

In some embodiments, the IAV H1 HA protein comprises a glycine at position 456 and a isoleucine at position 402, where the positions are numbered by alignment of the amino acid sequence of the IAV H1 HA protein to SEQ ID NO: 95. In some embodiments, the IAV H1 HA protein comprises a glycine substitution at position 456 and a isoleucine substitution at position 402 relative to a reference IAV H1 HA protein, where the positions are numbered by alignment of the amino acid sequence of the reference IAV H1 HA protein to SEQ ID NO: 95. In some embodiments, the IAV H1 HA protein comprises D456G and K402I substitutions relative to a reference IAV H1 HA protein, where the numbering of amino acids replaced in the reference IAV H1 HA protein corresponds to the numbering of SEQ ID NO: 95. AD456G substitution is a glycine substitution in the B loop, and a K402I substitution is a cavity-filling substitution.

In some embodiments, the IAV H1 HA protein comprises a cysteine at position 442 and a cysteine at position 423, where the positions are numbered by alignment of the amino acid sequence of the IAV H1 HA protein to SEQ ID NO: 95. In some embodiments, the IAV H1 HA protein comprises a cysteine substitution at position 442 and a cysteine substitution at position 423 relative to a reference IAV H1 HA protein, where the positions are numbered by alignment of the amino acid sequence of the reference IAV H1 HA protein to SEQ ID NO: 95. In some embodiments, the IAV H1 HA protein comprises L442C and N423C substitutions relative to a reference IAV H1 HA protein, where the numbering of amino acids replaced in the reference IAV H1 HA protein corresponds to the numbering of SEQ ID NO: 95. L442C and N423C substitutions result in the formation of an interprotomer disulfide bond in the HA protein.

In some embodiments, the IAV H1 HA protein comprises a glycine at position 391, where the positions are numbered by alignment of the amino acid sequence of the IAV H1 HA protein to SEQ ID NO: 95. In some embodiments, the IAV H1 HA protein comprises a glycine substitution at position 391 relative to a reference IAV H1 HA protein, where the positions are numbered by alignment of the amino acid sequence of the reference IAV H1 HA protein to SEQ ID NO: 95. In some embodiments, the IAV H1 HA protein comprises a K391G substitution relative to a reference IAV H1 HA protein, where the numbering of amino acids replaced in the reference IAV H1 HA protein corresponds to the numbering of SEQ ID NO: 95. A K391G substitution is a glycine substitution in the B loop of the HA protein.

In some embodiments, the IAV H1 HA protein comprises an amino acid sequence with at least 90%, at least 91%, at least 92%, at least 93%, at least 94%, at least 95%, at least 96%, at least 97%, at least 98%, or at least 99% sequence identity to the amino acid sequence of SEQ ID NO: 95. In some embodiments, the IAV H1 HA protein comprises an amino acid sequence with at least 90% sequence identity to the amino acid sequence of SEQ ID NO: 95.

Substitutions described in this subsection relating to IAV H1 HA proteins may be combined. For example, any of the pairs of cysteine substitutions may be combined with one or more proline substitutions in the B loop, glycine substitutions in the B loop, substitutions of pH-sensitive histidines, and/or cavity-filling substitutions. Pairs of cysteine substitutions that form different disulfide bonds (e.g., interprotomer versus intraprotomer bonds, head versus stalk region bonds) may also be combined. Preferred combinations of substitutions are discussed below.

In some embodiments, the IAV H1 HA protein comprises an interprotomer disulfide bond formed by at least one introduced cysteine, and one or more proline substitutions in the B loop. In some embodiments, the IAV H1 HA protein comprises an interprotomer disulfide bond formed by at least one introduced cysteine, and one or more substitutions of a charged or polar cavity-lining residue with a hydrophobic residue. In some embodiments, the IAV H1 HA protein comprises an interprotomer disulfide bond formed by at least one introduced cysteine, one or more substitutions of a charged or polar cavity-lining residue with a hydrophobic residue, and one or more proline substitutions in the B loop.

In some embodiments, the IAV H1 HA protein comprises an interprotomer disulfide bond formed by at least one introduced cysteine, and an intraprotomer disulfide bond formed by at least one introduced cysteine, where the introduced cysteine(s) of the interpromoter disulfide bond are at different positions than the introduced cysteine(s) of the intraprotomer disulfide bond.

In some embodiments, the IAV H1 HA protein comprises an interprotomer disulfide bond formed by at least one introduced cysteine, and one or more substitutions of a pH-sensitive histidine in the HA protein and one or more substitutions of a charged or polar cavity-lining residue with a hydrophobic residue.

In some embodiments, the IAV H1 HA protein comprises a cysteine at position 391, a cysteine at position 37, a proline at position 404, and a proline at position 416, where the positions are numbered by alignment of the amino acid sequence of the IAV H1 HA protein to SEQ ID NO: 83. In some embodiments, the IAV H1 HA protein comprises a cysteine substitution at position 391, a cysteine substitution at position 37, a proline substitution at position 404, and a proline substitution at position 416 relative to a reference IAV H1 HA protein, where the positions are numbered by alignment of the amino acid sequence of the reference IAV H1 HA protein to SEQ ID NO: 83. In some embodiments, the IAV H1 HA protein comprises K391C, L37C, N404P, H416P substitutions relative to a reference IAV H1 HA protein, where the numbering of amino acids replaced in the reference IAV H1 HA protein corresponds to the numbering of SEQ ID NO: 83.

In some embodiments, the IAV H1 HA protein comprises a cysteine at position 391, a cysteine at position 37, a isoleucine at position 395, and a isoleucine at position 447, where the positions are numbered by alignment of the amino acid sequence of the IAV H1 HA protein to SEQ ID NO: 83. In some embodiments, the IAV H1 HA protein comprises a cysteine substitution at position 391, a cysteine substitution at position 37, a isoleucine substitution at position 395, and a isoleucine substitution at position 447 relative to a reference IAV H1 HA protein, where the positions are numbered by alignment of the amino acid sequence of the reference IAV H1 HA protein to SEQ ID NO: 83. In some embodiments, the IAV H1 HA protein comprises K391C, L37C, K395I, E447I substitutions relative to a reference IAV H1 HA protein, where the numbering of amino acids replaced in the reference IAV H1 HA protein corresponds to the numbering of SEQ ID NO: 83.

In some embodiments, the IAV H1 HA protein comprises a cysteine at position 391, a cysteine at position 37, a glycine at position 456, and a isoleucine at position 402, where the positions are numbered by alignment of the amino acid sequence of the IAV H1 HA protein to SEQ ID NO: 83. In some embodiments, the IAV H1 HA protein comprises a cysteine substitution at position 391, a cysteine substitution at position 37, a glycine substitution at position 456, and a isoleucine substitution at position 402 relative to a reference IAV H1 HA protein, where the positions are numbered by alignment of the amino acid sequence of the reference IAV H1 HA protein to SEQ ID NO: 83. In some embodiments, the IAV H1 HA protein comprises K391C, L37C, D456G, K402I substitutions relative to a reference IAV H1 HA protein, where the numbering of amino acids replaced in the reference IAV H1 HA protein corresponds to the numbering of SEQ ID NO: 83.

In some embodiments, the IAV H1 HA protein comprises a cysteine at position 395, a cysteine at position 36, a proline at position 404, and a proline at position 416, where the positions are numbered by alignment of the amino acid sequence of the IAV H1 HA protein to SEQ ID NO: 83. In some embodiments, the IAV H1 HA protein comprises a cysteine substitution at position 395, a cysteine substitution at position 36, a proline substitution at position 404, and a proline substitution at position 416 relative to a reference IAV H1 HA protein, where the positions are numbered by alignment of the amino acid sequence of the reference IAV H1 HA protein to SEQ ID NO: 83. In some embodiments, the IAV H1 HA protein comprises K395C, V36C, N404P, H416P substitutions relative to a reference IAV H1 HA protein, where the numbering of amino acids replaced in the reference IAV H1 HA protein corresponds to the numbering of SEQ ID NO: 83.

In some embodiments, the IAV H1 HA protein comprises a cysteine at position 395, a cysteine at position 36, a glycine at position 456, and a isoleucine at position 402, where the positions are numbered by alignment of the amino acid sequence of the IAV H1 HA protein to SEQ ID NO: 83. In some embodiments, the IAV H1 HA protein comprises a cysteine substitution at position 395, a cysteine substitution at position 36, a glycine substitution at position 456, and a isoleucine substitution at position 402 relative to a reference IAV H1 HA protein, where the positions are numbered by alignment of the amino acid sequence of the reference IAV H1 HA protein to SEQ ID NO: 83. In some embodiments, the IAV H1 HA protein comprises K395C, V36C, D456G, K402I substitutions relative to a reference IAV H1 HA protein, where the numbering of amino acids replaced in the reference IAV H1 HA protein corresponds to the numbering of SEQ ID NO: 83.

In some embodiments, the IAV H1 HA protein comprises a cysteine at position 461, a cysteine at position 348, a proline at position 404, and a proline at position 416, where the positions are numbered by alignment of the amino acid sequence of the IAV H1 HA protein to SEQ ID NO: 83. In some embodiments, the IAV H1 HA protein comprises a cysteine substitution at position 461, a cysteine substitution at position 348, a proline substitution at position 404, and a proline substitution at position 416 relative to a reference IAV H1 HA protein, where the positions are numbered by alignment of the amino acid sequence of the reference IAV H1 HA protein to SEQ ID NO: 83. In some embodiments, the IAV H1 HA protein comprises N461C, G348C, N404P, H416P substitutions relative to a reference IAV H1 HA protein, where the numbering of amino acids replaced in the reference IAV H1 HA protein corresponds to the numbering of SEQ ID NO: 83.

In some embodiments, the IAV H1 HA protein comprises a cysteine at position 461, a cysteine at position 348, a isoleucine at position 395, and a isoleucine at position 447, where the positions are numbered by alignment of the amino acid sequence of the IAV H1 HA protein to SEQ ID NO: 83. In some embodiments, the IAV H1 HA protein comprises a cysteine substitution at position 461, a cysteine substitution at position 348, a isoleucine substitution at position 395, and a isoleucine substitution at position 447 relative to a reference IAV H1 HA protein, where the positions are numbered by alignment of the amino acid sequence of the reference IAV H1 HA protein to SEQ ID NO: 83. In some embodiments, the IAV H1 HA protein comprises N461C, G348C, K395I, E447I substitutions relative to a reference IAV H1 HA protein, where the numbering of amino acids replaced in the reference IAV H1 HA protein corresponds to the numbering of SEQ ID NO: 83.

In some embodiments, the IAV H1 HA protein comprises a cysteine at position 461, a cysteine at position 348, a glycine at position 456, and a isoleucine at position 402, where the positions are numbered by alignment of the amino acid sequence of the IAV H1 HA protein to SEQ ID NO: 83. In some embodiments, the IAV H1 HA protein comprises a cysteine substitution at position 461, a cysteine substitution at position 348, a glycine substitution at position 456, and a isoleucine substitution at position 402 relative to a reference IAV H1 HA protein, where the positions are numbered by alignment of the amino acid sequence of the reference IAV H1 HA protein to SEQ ID NO: 83. In some embodiments, the IAV H1 HA protein comprises N461C, G348C, D456G, K402I substitutions relative to a reference IAV H1 HA protein, where the numbering of amino acids replaced in the reference IAV H1 HA protein corresponds to the numbering of SEQ ID NO: 83.

In some embodiments, the IAV H1 HA protein comprises a cysteine at position 391, a cysteine at position 37, a proline at position 404, a proline at position 416, a glycine at position 456, and a isoleucine at position 402, where the positions are numbered by alignment of the amino acid sequence of the IAV H1 HA protein to SEQ ID NO: 83. In some embodiments, the IAV H1 HA protein comprises a cysteine substitution at position 391, a cysteine substitution at position 37, a proline substitution at position 404, a proline substitution at position 416, a glycine substitution at position 456, and a isoleucine substitution at position 402 relative to a reference IAV H1 HA protein, where the positions are numbered by alignment of the amino acid sequence of the reference IAV H1 HA protein to SEQ ID NO: 83. In some embodiments, the IAV H1 HA protein comprises K391C, L37C, N404P, H416P, D456G, K402I substitutions relative to a reference IAV H1 HA protein, where the numbering of amino acids replaced in the reference IAV H1 HA protein corresponds to the numbering of SEQ ID NO: 83.

In some embodiments, the IAV H1 HA protein comprises a cysteine at position 410, a cysteine at position 462, a cysteine at position 457, and a cysteine at position 346, where the positions are numbered by alignment of the amino acid sequence of the IAV H1 HA protein to SEQ ID NO: 95. In some embodiments, the IAV H1 HA protein comprises a cysteine substitution at position 410, a cysteine substitution at position 462, a cysteine substitution at position 457, and a cysteine substitution at position 346 relative to a reference IAV H1 HA protein, where the positions are numbered by alignment of the amino acid sequence of the reference IAV H1 HA protein to SEQ ID NO: 95. In some embodiments, the IAV H1 HA protein comprises V410C, L462C, S457C, L346C substitutions relative to a reference IAV H1 HA protein, where the numbering of amino acids replaced in the reference IAV H1 HA protein corresponds to the numbering of SEQ ID NO: 95.

In some embodiments, the IAV H1 HA protein comprises a cysteine at position 391, a cysteine at position 37, a phenylalanine at position 370, and a phenylalanine at position 455, where the positions are numbered by alignment of the amino acid sequence of the IAV H1 HA protein to SEQ ID NO: 95. In some embodiments, the IAV H1 HA protein comprises a cysteine substitution at position 391, a cysteine substitution at position 37, a phenylalanine substitution at position 370, and a phenylalanine substitution at position 455 relative to a reference IAV H1 HA protein, where the positions are numbered by alignment of the amino acid sequence of the reference IAV H1 HA protein to SEQ ID NO: 95. In some embodiments, the IAV H1 HA protein comprises K391C, L37C, H370F, H455F substitutions relative to a reference IAV H1 HA protein, where the numbering of amino acids replaced in the reference IAV H1 HA protein corresponds to the numbering of SEQ ID NO: 95.

### H3 HA Proteins

Some embodiments relate to IAV H3 HA proteins comprising an amino acid substitution relative to a reference IAV H3 HA protein. The reference IAV H3 HA protein may be an HA protein of an influenza A virus of an IAV subtype containing an H3 HA protein, such as an influenza A/(H3N2) virus. H3 HA proteins are present, e.g., on influenza A viruses of the H3N2 subtype, but other IAV subtypes expressing H3 HA proteins have been identified (such as the H3N8 subtype endemic in birds, horses, and dogs). Non-limiting examples of influenza A/(H3N2) virus isolates include A/Sydney/5/97(H3N2), A/Moscow/10/1999(H3N2), A/Fujian/411/2002(H3N2), A/California/7/2004(H3N2), A/Wisconsin/67/2005(H3N2), A/Brisbane/10/2007(H3N2), A/Perth/16/2009(H3N2), A/Victoria/361/2011(H3N2), A/Texas/50/2012(H3N2), A/Switzerland/9715293/2013(H3N2), A/Hong Kong/4801/2014(H3N2), A/Singapore/INFIMH-16-0019/2016(H3N2), A/Kansas/14/2017(H3N2), A/Hong Kong/2671/2019(H3N2), A/Hong Kong/45/2019(H3N2), A/Cambodia/e0826360/2020(H3N2), A/Darwin/9/2021(H3N2), and A/Darwin/6/2021(H3N2). Representative amino acid sequences of influenza A/(H3N2) virus HA proteins of previous seasonal influenza vaccines are provided as SEQ ID NOs: 136-153.

Those of ordinary skill in the art will appreciate that mutations disclosed in relation to a listed sequence, e.g., of influenza A/Darwin/6/2021(H3N2) virus HA, may be applied to HA proteins of other influenza A/(H3N2) viruses or other reference IAV H3 HA proteins. For example, in applying a T40C substitution to another IAV H3 HA protein, the skilled artisan would align the amino acid sequence of that reference HA protein to the influenza A/Darwin/6/2021(H3N2) virus HA protein sequence of SEQ ID NO: 82 and introduce a C at the residue of the reference HA protein amino acid sequence that aligns to T40 of SEQ ID NO: 82.

Exemplary substitutions that may be present in IAV H1 HA proteins are provided below in Table HA-5. In the event a reference HA protein sequence (e.g. of an isolate) already includes a substitution mentioned in the table, then it will be appreciated that some embodiments will include the residues set forth at positions below without a substitution being made at that particular position. For example, if an isolate already contains an isoleucine at position 219, then embodiments comprising 219I and H504P can be obtained with only a 504P substitution.

TABLE HA-5
A/(H3N2) subtype HA substitutions
(A/Darwin/6/2021(H3N2) (SEQ ID NO: 82) numbering)

| Substitution(s) | Mutation class |
|---|---|
| T40C A55C | Intra-protomer DS (Head) |
| S123C R421C | Inter-protomer DS (Head) |
| L260C P237C | Inter-protomer DS (Head) |
| Q392C T46C | Inter-protomer DS (Stalk) |
| I411C Y428C | Inter-protomer DS (Stalk) |
| G402P R421P E414P | Proline(s) |
| K403G | Glycine(s) |
| F408G H409G | Glycine(s) |
| K396I | Cavity Filling |
| T219I H504P | Cavity Filling (T219I) / Proline (H504P) |

In some embodiments, the IAV H3 HA protein comprises a cysteine at position 40 and a cysteine at position 55, where the positions are numbered by alignment of the amino acid sequence of the IAV H3 HA protein to SEQ ID NO: 82. In some embodiments, the IAV H3 HA protein comprises a cysteine substitution at position 40 and a cysteine substitution at position 55 relative to a reference IAV H3 HA protein, where the positions are numbered by alignment of the amino acid sequence of the reference IAV H3 HA protein to SEQ ID NO: 82. In some embodiments, the IAV H3 HA protein comprises T40C and A55C substitutions relative to a reference IAV H3 HA protein, where the numbering of amino acids replaced in the reference IAV H3 HA protein corresponds to the numbering of SEQ ID NO: 82. T40C and A55C substitutions result in the formation of an intraprotomer disulfide bond in the head region of the HA protein.

In some embodiments, the IAV H3 HA protein comprises a cysteine at position 123 and a cysteine at position 421, where the positions are numbered by alignment of the amino acid sequence of the IAV H3 HA protein to SEQ ID NO: 82. In some embodiments, the IAV H3 HA protein comprises a cysteine substitution at position 123 and a cysteine substitution at position 421 relative to a reference IAV H3 HA protein, where the positions are numbered by alignment of the amino acid sequence of the reference IAV H3 HA protein to SEQ ID NO: 82. In some embodiments, the IAV H3 HA protein comprises S123C and R421C substitutions relative to a reference IAV H3 HA protein, where the numbering of amino acids replaced in the reference IAV H3 HA protein corresponds to the numbering of SEQ ID NO: 82. S123C and R421C substitutions result in the formation of an interprotomer disulfide bond in the head region of the HA protein.

In some embodiments, the IAV H3 HA protein comprises a cysteine at position 260 and a cysteine at position 237, where the positions are numbered by alignment of the amino acid sequence of the IAV H3 HA protein to SEQ ID NO: 82. In some embodiments, the IAV H3 HA protein comprises a cysteine substitution at position 260 and a cysteine substitution at position 237 relative to a reference IAV H3 HA protein, where the positions are numbered by alignment of the amino acid sequence of the reference IAV H3 HA protein to SEQ ID NO: 82. In some embodiments, the IAV H3 HA protein comprises L260C and P237C substitutions relative to a reference IAV H3 HA protein, where the numbering of amino acids replaced in the reference IAV H3 HA protein corresponds to the numbering of SEQ ID NO: 82. L260C and P237C substitutions result in the formation of an interprotomer disulfide bond in the head region of the HA protein.

In some embodiments, the IAV H3 HA protein comprises a cysteine at position 392 and a cysteine at position 46, where the positions are numbered by alignment of the amino acid sequence of the IAV H3 HA protein to SEQ ID NO: 82. In some embodiments, the IAV H3 HA protein comprises a cysteine substitution at position 392 and a cysteine substitution at position 46 relative to a reference IAV H3 HA protein, where the positions are numbered by alignment of the amino acid sequence of the reference IAV H3 HA protein to SEQ ID NO: 82. In some embodiments, the IAV H3 HA protein comprises Q392C and T46C substitutions relative to a reference IAV H3 HA protein, where the numbering of amino acids replaced in the reference IAV H3 HA protein corresponds to the numbering of SEQ ID NO: 82. Q392C and T46C substitutions result in the formation of an interprotomer disulfide bond in the stalk region of the HA protein.

In some embodiments, the IAV H3 HA protein comprises a cysteine at position 411 and a cysteine at position 428, where the positions are numbered by alignment of the amino acid sequence of the IAV H3 HA protein to SEQ ID NO: 82. In some embodiments, the IAV H3 HA protein comprises a cysteine substitution at position 411 and a cysteine substitution at position 428 relative to a reference IAV H3 HA protein, where the positions are numbered by alignment of the amino acid sequence of the reference IAV H3 HA protein to SEQ ID NO: 82. In some embodiments, the IAV H3 HA protein comprises I411C and Y428C substitutions relative to a reference IAV H3 HA protein, where the numbering of amino acids replaced in the reference IAV H3 HA protein corresponds to the numbering of SEQ ID NO: 82. I411C and Y428C substitutions result in the formation of an interprotomer disulfide bond in the stalk region of the HA protein.

In some embodiments, the IAV H3 HA protein comprises a proline at position 402, a proline at position 421, and a proline at position 414, where the positions are numbered by alignment of the amino acid sequence of the IAV H3 HA protein to SEQ ID NO: 82. In some embodiments, the IAV H3 HA protein comprises a proline substitution at position 402, a proline substitution at position 421, and a proline substitution at position 414 relative to a reference IAV H3 HA protein, where the positions are numbered by alignment of the amino acid sequence of the reference IAV H3 HA protein to SEQ ID NO: 82. In some embodiments, the IAV H3 HA protein comprises G402P, R421P, and E414P substitutions relative to a reference IAV H3 HA protein, where the numbering of amino acids replaced in the reference IAV H3 HA protein corresponds to the numbering of SEQ ID NO: 82. G402P, R421P, and E414P substitutions are proline substitutions in the B loop of the HA protein.

In some embodiments, the IAV H3 HA protein comprises a glycine at position 403, where the positions are numbered by alignment of the amino acid sequence of the IAV H3 HA protein to SEQ ID NO: 82. In some embodiments, the IAV H3 HA protein comprises a glycine substitution at position 403 relative to a reference IAV H3 HA protein, where the positions are numbered by alignment of the amino acid sequence of the reference IAV H3 HA protein to SEQ ID NO: 82. In some embodiments, the IAV H3 HA protein comprises a K403G substitution relative to a reference IAV H3 HA protein, where the numbering of amino acids replaced in the reference IAV H3 HA protein corresponds to the numbering of SEQ ID NO: 82. A K403G substitution is a glycine substitution in the B loop of the HA protein.

In some embodiments, the IAV H3 HA protein comprises a glycine at position 408 and a glycine at position 409, where the positions are numbered by alignment of the amino acid sequence of the IAV H3 HA protein to SEQ ID NO: 82. In some embodiments, the IAV H3 HA protein comprises a glycine substitution at position 408 and a glycine substitution at position 409 relative to a reference IAV H3 HA protein, where the positions are numbered by alignment of the amino acid sequence of the reference IAV H3 HA protein to SEQ ID NO: 82. In some embodiments, the IAV H3 HA protein comprises F408G and H409G substitutions relative to a reference IAV H3 HA protein, where the numbering of amino acids replaced in the reference IAV H3 HA protein corresponds to the numbering of SEQ ID NO: 82. F408G and H409G substitutions are glycine substitutions in the B loop of the HA protein.

In some embodiments, the IAV H3 HA protein comprises a isoleucine at position 396, where the positions are numbered by alignment of the amino acid sequence of the IAV H3 HA protein to SEQ ID NO: 82. In some embodiments, the IAV H3 HA protein comprises a isoleucine substitution at position 396 relative to a reference IAV H3 HA protein, where the positions are numbered by alignment of the amino acid sequence of the reference IAV H3 HA protein to SEQ ID NO: 82. In some embodiments, the IAV H3 HA protein comprises a K396I substitution relative to a reference IAV H3 HA protein, where the numbering of amino acids replaced in the reference IAV H3 HA protein corresponds to the numbering of SEQ ID NO: 82. A K396I substitution is a cavity-filling substitution.

In some embodiments, the IAV H3 HA protein comprises a isoleucine at position 219 and a proline at position 504, where the positions are numbered by alignment of the amino acid sequence of the IAV H3 HA protein to SEQ ID NO: 82. In some embodiments, the IAV H3 HA protein comprises a isoleucine substitution at position 219 and a proline substitution at position 504 relative to a reference IAV H3 HA protein, where the positions are numbered by alignment of the amino acid sequence of the reference IAV H3 HA protein to SEQ ID NO: 82. In some embodiments, the IAV H3 HA protein comprises T219I and H504P substitutions relative to a reference IAV H3 HA protein, where the numbering of amino acids replaced in the reference IAV H3 HA protein corresponds to the numbering of SEQ ID NO: 82. A T219I substitution is a cavity-filling substitution, and an H504P substitution is a proline substitution in the B loop of the HA protein.

Substitutions described in this subsection relating to IAV H3 HA proteins may be combined. For example, any of the pairs of cysteine substitutions may be combined with one or more proline substitutions in the B loop, glycine substitutions in the B loop, substitutions of pH-sensitive histidines, and/or cavity-filling substitutions. Pairs of cysteine substitutions that form different disulfide bonds (e.g., interprotomer v. intraprotomer bonds, head v. stalk region bonds) may also be combined. In some embodiments, the IAV H3 HA protein comprises one or more substitutions of a pH-sensitive histidine in the HA protein and one or more substitutions of a charged or polar cavity-lining residue with a hydrophobic residue.

In some embodiments, the IAV H3 HA protein comprises an amino acid sequence with at least 90%, at least 91%, at least 92%, at least 93%, at least 94%, at least 95%, at least 96%, at least 97%, at least 98%, or at least 99% sequence identity to the amino acid sequence of SEQ ID NO: 82. In some embodiments, the IAV H3 HA protein comprises an amino acid sequence with at least 90% sequence identity to the amino acid sequence of SEQ ID NO: 82.

### Virus and Antigen Characterization

Influenza viruses and protein sequences thereof may be classified as belonging to a given IAV subtype or IBV lineage using any suitable classification method. Non-limiting examples of classification methods include sequence-based analyses (e.g., sequence comparison and phylogenetic tree building) and antigenic characterization (e.g., HAI assays, microneutralization assays, and immunofluorescence).

### Sequence Analyses

Amino acid sequences of influenza virus antigens may be analyzed to classify them as belonging to a given IAV subtype or IBV lineage. For example, an HA amino acid sequence is classified into the H1 subtype, H3 subtype, B/Victoria lineage, B/Yamagata lineage on the basis of similarity to extant H1 HA, H3 HA, B/Victoria lineage HA, or B/Yamagata lineage HA amino sequences. As another example, an HA amino acid sequence may be fit into a phylogenetic tree of extant HA amino acid sequences, and classified as belonging to a given IAV subtype or IBV lineage according to its most likely place in the phylogenetic tree.

Nucleotide sequences of influenza virus genome segments may similarly be compared to extant genome segments to classify the influenza virus as belonging to a given IAV subtype or IBV lineage, on the basis of percentage identity to extant sequences of a given subtype or lineage. As another example, an influenza virus genome segment nucleotide sequence may be fit into a phylogenetic tree of extant genome segment nucleotide sequences, and classified as belonging to a given IAV subtype or IBV lineage according to its most likely place in the phylogenetic tree.

### Antigenic Characterization

Serological methods such as the HAI test are useful for many epidemiological and immunological studies and for evaluation of the antibody response following vaccination. Serological methods are also useful in situations where identification of the virus is not feasible (e.g. after viral shedding has stopped). The HAI test is used to identify circulating influenza viruses that are antigenically similar to influenza viruses from previous seasons.

The hemagglutination inhibition (HAI) test is a classical laboratory procedure for the classification or subtyping of hemagglutinating viruses and further determining the antigenic characteristics of influenza viral isolates provided that the reference antisera used contain antibodies to currently circulating viruses (see, e.g., Pedersen J C Methods Mol Biol. 2014; 1161:11-25). The antisera used are based on antigen preparations derived from either the wild-type strain or a high-growth reassortant made using the wild-type strain or an antigenically equivalent strain. Effective hemagglutinin inhibition by sera or antibodies specific to H1 HA but not sera or antibodies specific to H3 HA, for example, indicates that an influenza virus expresses an HA belonging to the H1 subtype.

The microneutralization assay is a highly sensitive and specific assay for detecting virus-specific neutralizing antibodies to influenza viruses in human and animal sera, and in some embodiments, includes the detection of human antibodies to avian subtypes. Testing can be carried out quickly once a novel virus is identified and often before purified viral proteins become available for use in other assays. Neutralization of an influenza virus cells by sera or antibodies specific to H1 HA but not sera or antibodies specific to H3 HA, for example, indicates that an influenza virus expresses an HA belonging to the H1 subtype.

Immunofluorescence antibody (IFA) staining of virus-infected cells in original clinical specimens and field isolates is a rapid and sensitive method for diagnosing respiratory and other viral infections. In some embodiments, IFA staining is performed on isolates rather than original clinical specimens, as this allows any virus that is present to first be amplified, and if required used in other studies. Staining of influenza virus-infected cells by antibodies specific to H1 HA but not antibodies specific to H3 HA, for example, indicates that an influenza virus expresses an HA belonging to the H1 subtype.

### Vaccine Compositions

Some aspects relate to compositions for use as vaccines against seasonal influenza viruses, and optionally other respiratory viruses (e.g., coronaviruses and/or respiratory syncytial viruses).

Some embodiments relate to compositions comprising nucleic acids (e.g., RNAs, (e.g., mRNAs)) encoding respiratory virus (e.g., influenza virus, RSV, SARS-CoV-2) antigens, where the nucleic acids encoding different antigens are present at certain ratios. A “ratio” of two nucleic acids (e.g., encoding proteins A and B) may refer to a molar ratio (the number of nucleic acid molecules encoding protein A, relative to the number of nucleic acid molecules encoding protein B), or a mass ratio (the mass of nucleic acids encoding protein A, relative to the mass of nucleic acids encoding protein B). Unless indicated otherwise or otherwise clear from context, reference to nucleic acids (e.g., RNAs (e.g., mRNAs)) being present at a “ratio” refers to a mass ratio of the nucleic acids.

### Multivalent Vaccines

Some aspects relate to multivalent vaccines that comprise components to protect a subject against more than one influenza virus. Multivalent influenza vaccines can include three (trivalent), four (quadrivalent), five (pentavalent), or more (such as octavalent) components that each independently are designed to protect against one of a variety of influenza virus strains. For instance, a trivalent vaccine can include RNA(s) encoding an influenza A/(H1N1) virus protein, an influenza A/(H3N2) virus protein, and an influenza B/Victoria lineage virus protein. Some trivalent compositions comprise RNA(s) encoding two influenza A virus HA proteins and one influenza B virus HA proteins. Quadrivalent vaccines can include RNA(s) encoding three influenza A virus proteins (e.g., HA proteins) and one influenza B virus protein (e.g., HA protein). Some quadrivalent vaccines include mRNA encoding two influenza A virus proteins (e.g., HA proteins) and two influenza B virus proteins (e.g., HA proteins).

In some aspects, a multivalent vaccine comprises three mRNAs, a first encoding an influenza A/(H1N1) virus HA protein, a second encoding an influenza A/(H3N2) virus HA protein, and a third encoding an influenza B/Victoria lineage virus HA protein. In some embodiments, the mRNAs are present at a 1:1:1:1 mass ratio.

In some aspects, a multivalent vaccine comprises four mRNAs, a first encoding an influenza A/(H1N1) virus HA protein, a second encoding an influenza A/(H3N2) virus HA protein, a third encoding an influenza B/Victoria lineage virus HA protein, and a fourth encoding influenza B/Yamagata lineage virus HA protein. In some embodiments, the mRNAs are present at a 1:1:1:1 mass ratio.

In some aspects, a multivalent vaccine comprises eight mRNAs, a first encoding an IAV H1 HA protein, a second encoding an IAV H3 HA protein, a third encoding an influenza B/Victoria lineage virus HA protein, an influenza B/Yamagata lineage virus HA protein, a fifth encoding an IAV N1 NA protein, a sixth encoding an IAV N2 NA protein, a seventh encoding an influenza B/Victoria lineage virus NA protein, and an eighth encoding an influenza B/Yamagata lineage virus NA protein. In some embodiments, the mRNAs are present at a 1:1:1:1:1:1:1:1 mass ratio. In some embodiments, the mRNAs are present at a 3:3:3:3:1:1:1:1 mass ratio (i.e., the mRNAs encoding the HA proteins are present at 3 times the amount (by mass) of mRNAs encoding the NA proteins).

In some aspects, a multivalent vaccine comprises five mRNAs, a first encoding an IAV H1 HA protein, a second encoding a first IAV H3 HA protein, a third encoding a second IAV H3 HA protein, a fourth encoding an influenza B/Victoria lineage virus HA protein, and a fifth encoding an influenza B/Yamagata lineage HA protein. In some embodiments, the mRNAs are present at a 1:1:1:1:1 mass ratio.

In some aspects, a multivalent vaccine comprises four mRNAs, a first encoding an IAV H1 HA protein, a second encoding a first IAV H3 HA protein, a third encoding a second IAV H3 HA protein, and a fourth encoding an influenza B/Victoria lineage virus HA protein, where the vaccine does not comprise an mRNA encoding an influenza B/Yamagata lineage virus HA protein. In some embodiments, the mRNAs are present at a 1:1:1:1 mass ratio.

In some aspects, a multivalent vaccine comprises six mRNAs, a first encoding an IAV H1 HA protein, a second encoding a first IAV H3 HA protein, a third encoding a second IAV H3 HA protein, a fourth encoding a third IAV H3 HA protein, a fifth encoding an influenza B/Victoria lineage virus HA protein, and a sixth encoding an influenza B/Yamagata lineage HA protein. In some embodiments, the mRNAs are present at a 1:1:1:1:1:1 mass ratio.

In some aspects, a multivalent vaccine comprises five mRNAs, a first encoding an IAV H1 HA protein, a second encoding a first IAV H3 HA protein, a third encoding a second IAV H3 HA protein, a fourth encoding a third IAV H3 HA protein, and a fifth encoding an influenza B/Victoria lineage virus HA protein, where the vaccine does not comprise an mRNA encoding an influenza B/Yamagata lineage HA protein. In some embodiments, the mRNAs are present at a 1:1:1:1:1 mass ratio.

Some embodiments relate to multivalent vaccines comprising mRNAs encoding multiple H3 HA proteins derived from distinct influenza A/(H3N2) viruses. Separate H3 HA proteins may belong to different clades of the A/(H3N2) subtype. In some embodiments, each H3 HA protein encoded by an mRNA of a vaccine is derived from an influenza virus of a distinct clade within the A/(H3N2) subtype. In some embodiments, each H3 HA protein encoded by an mRNA of a vaccine differs from each other H3 HA protein encoded by other mRNAs of the vaccine by at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 substitutions. Where two H3 HA proteins vary in length, the number of substitutions present between their amino acid sequences is calculated after aligning the amino acid sequences as discussed in the section entitled “Protein variants and alignment.”

In some embodiments of multivalent vaccines, the influenza B/Victoria lineage virus HA protein is an influenza B/Victoria lineage virus HA protein described in the section entitled “B/Victoria lineage HA proteins.” In some embodiments, the influenza B/Victoria lineage HA protein comprises an amino acid sequence with at least 90% sequence identity to the amino acid sequence of SEQ ID NO: 71.

In some embodiments of multivalent vaccines, the influenza B/Yamagata lineage virus HA protein is an influenza B/Yamagata lineage virus HA protein described in the section entitled “B/Yamagata lineage HA proteins.” In some embodiments, the influenza B/Yamagata lineage HA protein comprises an amino acid sequence with at least 90% sequence identity to the amino acid sequence of SEQ ID NO: 70.

In some embodiments of multivalent vaccines, the IAV H1 HA protein is an IAV H1 HA protein described in the section entitled “H1 HA proteins.” In some embodiments, the IAV H1 HA protein comprises an amino acid sequence with at least 90% sequence identity to the amino acid sequence of SEQ ID NO: 83. In some embodiments, the IAV H1 HA protein comprises an amino acid sequence with at least 90% sequence identity to the amino acid sequence of SEQ ID NO: 95.

In some embodiments of multivalent vaccines, the IAV H3 HA protein is an IAV H3 HA protein described in the section entitled “H3 HA proteins.” Where a multivalent vaccine includes multiple H3 HA proteins (e.g., comprises multiple mRNAs encoding different H3 HA proteins), the different H3 HA proteins may each comprise the same substitutions, or different substitutions described in the section entitled “H3 HA proteins.” In some embodiments, one or more H3 HA proteins does not comprise a substitution described in the section entitled “H3 HA proteins.” In some embodiments, the IAV H3 HA protein comprises an amino acid sequence with at least 90% sequence identity to the amino acid sequence of SEQ ID NO: 82. In some embodiments, each IAV H3 HA protein comprises an amino acid sequence with at least 90% sequence identity to the amino acid sequence of SEQ ID NO: 82.

### Combination Vaccines

Some embodiments of vaccines include combination vaccines. A “combination vaccine”, as used herein, refers to a vaccine comprising one or more components for eliciting an immune response (i) to one or more influenza viruses, and (ii) to one or more viruses other than an influenza virus. As noted previously, discussion of combination vaccines comprising one or more RNAs (e.g., mRNAs) encoding proteins of different viruses may also be applied, inter alia, to combination vaccines comprising the same proteins (e.g., as isolated proteins or proteins present in viral vectors). The skilled artisan will appreciate that for combination vaccines comprising one or more RNAs (e.g., mRNAs) encoding two or more proteins, the two or more proteins may be encoded a single RNA, or different RNAs of the combination vaccine.

For example, a combination vaccine may include one or more RNAs, each encoding an antigen of a virus of a different family (e.g., a first antigen of an influenza virus (Orthomyxoviridae), and a second antigen of a coronavirus (Coronaviriade) or respiratory syncytial virus (Pneumoviridae)). In some embodiments, a composition includes one or more RNAs (e.g., mRNAs) encoding at least one influenza virus antigen, and at least one antigen of a different virus (e.g., a coronavirus or a virus from the Pneumoviridae family (e.g., respiratory syncytial virus (RSV)). In some embodiments, the different virus is SARS-CoV-2; that is, in some embodiments, a composition comprises one or more RNAs collectively encoding at least one influenza virus antigen, and at least one SARS-CoV-2 antigen. In some embodiments the different virus is human respiratory syncytial virus (hRSV); that is, in some embodiments, a composition comprises one or more RNAs collectively encoding at least one influenza virus antigen and at least one hRSV antigen. In some embodiments, a composition includes one or more RNAs collectively encoding at least one influenza virus antigen and at least one antigen of each of two different viruses (e.g. a coronavirus and an hRSV). In some embodiments, the different viruses are SARS-CoV-2 and hRSV; that is, in some embodiments, a composition comprises one or more RNAs collectively encoding at least one influenza virus antigen, at least one SARS-CoV-2 antigen, and at least one hRSV antigen.

With respect to the SARS-CoV-2 antigens of the combination vaccines, in some embodiments, the combination vaccine comprises 1, 2, 3, 4, 5, or 6 RNAs (e.g., mRNAs) encoding different coronavirus antigens, wherein each antigen comprises at least one mutation and/or at least one deletion relative to the amino acid sequence of SEQ ID NO: 78. In some embodiments, the combination vaccine comprises an RNA encoding a wild-type SARS-CoV-2 S protein antigen or the antigenic fragment thereof. In some embodiments, a combination vaccine includes an RNA encoding a fusion protein comprising at least two domains of a SARS-CoV-2 Spike (S) protein, and less than the full-length spike protein. In some embodiments, a combination vaccine comprises a first RNA encoding a first fusion protein comprising at least two domains of a SARS-CoV-2 Spike (S) protein and less than the full-length S protein, and a second RNA encoding a second fusion protein comprising at least two domains of a SARS-CoV-2 Spike (S) protein and less than the full-length S protein. In some embodiments, the two RNAs are present in the combination vaccine in a 1:1 ratio. In some embodiments, the two RNAs are present in the combination vaccine in a 1:2, 1:3, 1:4, 1:5, 1:6, 1:7, 1:8, 1:9, or 1:10 ratio.

With respect to the antigens of viruses from the Pneumoviridae family, in some embodiments, the combination vaccine comprises 1, 2, 3, 4, 5, or 6 RNAs (e.g., mRNAs) encoding different antigens of viruses from the Pneumoviridae family (e.g., RSV) wherein each antigen comprises at least one mutation and/or at least one deletion relative to a reference Pneumoviridae family virus antigen. In some embodiments, the combination vaccine comprises an RNA encoding a wild-type hRSV F glycoprotein antigen or antigenic fragment thereof. In some embodiments, a combination vaccine includes an RNA encoding a hRSV F glycoprotein variant lacking a cytoplasmic tail. In some embodiments, a combination vaccine includes an RNA encoding a hRSV F glycoprotein variant lacking a cytoplasmic tail and further comprising one or more modifications relative to a wild-type hRSV F glycoprotein. In some embodiments, a combination vaccine comprises a first RNA encoding a first hRSV F glycoprotein variant lacking a cytoplasmic tail, and a second RNA encoding a second hRSV F glycoprotein variant lacking a cytoplasmic tail and further comprising one or more modifications relative to the wild-type hRSV F glycoprotein. In some embodiments, the two RNAs are present in the combination vaccine in a 1:1 ratio. In some embodiments, the two RNAs are present in the combination vaccine in a 1:2, 1:3, 1:4, 1:5, 1:6, 1:7, 1:8, 1:9, or 1:10 ratio.

In some embodiments, the combination vaccine comprises one or more RNAs (e.g., mRNAs) encoding influenza virus antigens (e.g., mRNA encoding HA antigens) and one or more RNAs (e.g., mRNAs) encoding antigens of at least one different respiratory virus (e.g., mRNA encoding a SARS-CoV-2 fusion protein or an hRSV F glycoprotein). In some embodiments, the ratio of RNAs encoding influenza virus antigens to RNAs encoding the at least one different respiratory virus antigen in the combination vaccine is 5:1, 4:1, 3:1, 2:1, or 1:1. In some embodiments, the ratio of RNAs encoding influenza virus antigens to RNAs encoding the at least one different respiratory virus antigen in the combination vaccine is 10:1. In some embodiments, the ratio of RNAs encoding influenza virus antigens to RNAs encoding the at least one different respiratory virus antigen in the combination vaccine is 15:1. In some embodiments, the ratio of RNAs encoding influenza virus antigens to RNAs encoding the at least one different respiratory virus antigen in the combination vaccine is 20:1. In some embodiments, the ratio of RNAs encoding influenza virus antigens to RNAs encoding the at least one different respiratory virus antigen in the combination vaccine is 25:1. In some embodiments, the ratio of RNAs encoding influenza virus antigens to RNAs encoding the at least one different respiratory virus antigen in the combination vaccine is 30:1.

In some embodiments, the combination vaccine comprises (i) one or more RNAs (e.g., mRNAs) encoding one or more influenza virus proteins, (ii) one or more RNAs (e.g., mRNAs) encoding one or more SARS-CoV-2 proteins, and (ii) one or more RNAs (e.g., mRNAs) encoding one or more hRSV proteins. In some embodiments, each of (i) RNA(s) encoding influenza virus protein(s), (ii) RNA(s) encoding SARS-CoV-2 protein(s), and (iii) RNA(s) encoding hRSV protein(s) are present in the combination vaccine in substantially equal masses. In some embodiments, the RNA(s) encoding influenza virus protein(s) are present in the combination vaccine at 3 times the mass of RNA(s) encoding SARS-CoV-2 protein(s), and 3 times the mass of RNA(s) encoding hRSV protein(s). In some embodiments, the RNA(s) encoding influenza virus protein(s) are present in the combination vaccine at 4 times the mass of RNA(s) encoding SARS-CoV-2 protein(s), and 4 times the mass of RNA(s) encoding hRSV protein(s). In some embodiments, the RNA(s) encoding influenza virus protein(s) are present in the combination vaccine at 5 times the mass of RNA(s) encoding SARS-CoV-2 protein(s), and 5 times the mass of RNA(s) encoding hRSV protein(s). In some embodiments, the RNA(s) encoding influenza virus protein(s) are present in the combination vaccine at 2 times the mass of RNA(s) encoding SARS-CoV-2 protein(s), and 4 times the mass of RNA(s) encoding hRSV protein(s). In some embodiments, the RNA(s) encoding influenza virus protein(s) are present in the combination vaccine at 4 times the mass of RNA(s) encoding SARS-CoV-2 protein(s), and 2 times the mass of RNA(s) encoding hRSV protein(s). In some embodiments, the RNA(s) encoding influenza virus protein(s) are present in the combination vaccine at 0.5 the mass of RNA(s) encoding SARS-CoV-2 protein(s), and 0.5 times the mass of RNA(s) encoding hRSV protein(s). In some embodiments, the mass ratio of (i) RNA(s) encoding influenza virus protein(s), to (ii) RNA(s) encoding SARS-CoV-2 protein(s), to (iii) RNA(s) encoding hRSV protein(s) in the combination vaccine is 4:1:1, 4:2:1, 4:3:2, 4:3:3, or 2:1:1.

In some embodiments, the combination vaccine comprises mRNA polynucleotide wherein each polynucleotide encodes a different respiratory virus antigenic polypeptide. In some embodiments, the first, second and third mRNA polynucleotides are present in the combination vaccine in a ratio of 1:1:1. In some embodiments, the combination vaccine comprises a ratio of mRNA polynucleotides encoding respiratory virus antigenic polypeptides of 4:1:1 from the first virus (e.g., influenza virus) to the second virus to the third virus. In some embodiments, the combination vaccine comprises a ratio of mRNA polynucleotides encoding respiratory virus antigenic polypeptides of 3:1:1 from the first virus (e.g., influenza virus) to the second virus to the third virus. In some embodiments, the combination vaccine comprises a ratio of mRNA polynucleotides encoding respiratory virus antigenic polypeptides of 5:1:1 from the first virus (e.g., influenza virus) to the second virus to the third virus. In some embodiments, the combination vaccine comprises a ratio of mRNA polynucleotides encoding respiratory virus antigenic polypeptides of 4:2:1 from the first virus (e.g., influenza virus) to the second virus to the third virus. In some embodiments, the combination vaccine comprises a ratio of mRNA polynucleotides encoding respiratory virus antigenic polypeptides of 1:2:1 from the first virus (e.g., influenza virus) to the second virus to the third virus. In some embodiments, the combination vaccine comprises a ratio of mRNA polynucleotides encoding respiratory virus antigenic polypeptides of 1:2:2 from the first virus (e.g., influenza virus) to the second virus to the third virus. In some embodiments, the combination vaccine comprises a ratio of mRNA polynucleotides encoding respiratory virus antigenic polypeptides of 8:2:2, 4:1:1, 4:2:2, 4:2:1, 4:3:2, 4:3:3, 4:3:2, or 4:2:2 from the first virus to the second virus to the third virus.

In some embodiments, each of the mRNA polynucleotides in the combination vaccine is complementary with and does not interfere with each other mRNA polynucleotide in the combination vaccine. That is, an antigen produced from administration of the combination vaccine do not significantly interfere with the immune response to any other of the antigens produced in response to the vaccine in such a way that would diminish the ability of the antigens to provoke a protective immune response in a subject. In some embodiments, the combination vaccine is additive with respect to neutralizing antibodies relative to each individual antigen in a vaccine.

Thus, compositions (e.g., RNA vaccines (e.g., mRNA vaccines)) may target one or more antigen(s) of the same strain/species, or one or more antigen(s) of different strains/species, e.g., antigens which induce immunity to organisms which are found in the same geographic areas where the risk of respiratory virus (e.g. influenza virus and/or coronavirus and/or respiratory syncytial virus) infection is high.

Combination vaccines comprising RNA (e.g., mRNA) polynucleotides encoding at least two respiratory virus antigenic polypeptides from at least two different respiratory virus families (e.g., influenza viruses of Orthomyxoviridae, respiratory syncytial virus of Pneumoviridae, and coronavirus of Coronaviridae) may be used for treating and/or preventing respiratory virus infections. In some embodiments, a combination vaccine comprises mRNA polynucleotides encoding antigens from the Orthomyxoviridae family (e.g., influenza virus antigens) and the Coronaviridae family (e.g., SARS-CoV-2). In some embodiments, a combination vaccine comprises mRNA polynucleotides encoding antigens from the Orthomyxoviridae family (e.g., influenza virus) and the Pneumoviridae family (e.g., human respiratory syncytial virus). In some embodiments, a composition comprises RNA (e.g., mRNA) polynucleotides encoding at least three respiratory antigenic polypeptides from at least three different respiratory viruses. In some embodiments, the three different viruses are from the Orthomyxoviridae (e.g., influenza virus), Coronaviridae (e.g., SARS-CoV-2) and Pneumoviridae families (e.g., human respiratory syncytial virus).

In some embodiments, one or more RNAs (e.g., mRNAs) encoding polypeptides from at least two different respiratory virus families are encapsulated in a single lipid nanoparticle. Some embodiments comprise one or more RNAs (e.g., mRNAs) encoding polypeptides from at least two different respiratory virus families, wherein the composition comprises lipid nanoparticles encapsulating one or more RNAs (e.g., mRNAs) encoding polypeptides from a single respiratory virus family.

In some embodiments, a combination vaccine comprises a combination of proteins or nucleic acids (e.g., RNAs (e.g., mRNAs)) collectively encoding the combination, the combination comprising: (i) an IAV H1 HA protein, (ii) an IAV H3 HA protein, (iii) an influenza B/Victoria lineage virus HA protein, and (iv) a SARS-CoV-2 S protein or fragment thereof.

In some embodiments, a combination vaccine comprises a combination of proteins or nucleic acids (e.g., RNAs (e.g., mRNAs) collectively encoding the combination, the combination comprising: (i) an IAV H1 HA protein, (ii) an IAV H3 HA protein, (iii) an influenza B/Victoria lineage virus HA protein, and (iv) an hRSV F protein or fragment thereof.

In some embodiments, a combination vaccine comprises a combination of proteins or nucleic acids (e.g., RNAs (e.g., mRNAs) collectively encoding the combination, the combination comprising: (i) an IAV H1 HA protein, (ii) an IAV H3 HA protein, (iii) an influenza B/Victoria lineage virus HA protein, (iv) a SARS-CoV-2 S protein or fragment thereof, and (v) an hRSV protein or fragment thereof.

In some embodiments, the combination comprises 2 IAV H3 HAs. In some embodiments, the combination comprises 3 IAV H3 HAs. In some embodiments, the combination comprises 4, 5, 6, 7, 8, 9, or 10 IAV H3 HAs.

In some embodiments, the combination further comprises an influenza B/Yamagata lineage HA protein. In some embodiments, the combination does not comprise an influenza B/Yamagata lineage HA protein.

In some embodiments, the combination further comprises: (i) an IAV N1 NA protein, (ii) an IAV N2 NA protein, and (iii) an influenza B/Victoria lineage NA protein. In some embodiments, the combination further comprises an influenza B/Yamagata lineage NA protein. In some embodiments, the combination does not comprise an influenza B/Yamagata lineage NA protein.

In some embodiments of combination vaccines, the influenza B/Victoria lineage virus HA protein is an influenza B/Victoria lineage virus HA protein described in the section entitled “B/Victoria lineage HA proteins.” In some embodiments, the influenza B/Victoria lineage HA protein comprises an amino acid sequence with at least 90% sequence identity to the amino acid sequence of SEQ ID NO: 71.

In some embodiments of combination vaccines, the influenza B/Yamagata lineage virus HA protein is an influenza B/Yamagata lineage virus HA protein described in the section entitled “B/Yamagata lineage HA proteins.” In some embodiments, the influenza B/Yamagata lineage HA protein comprises an amino acid sequence with at least 90% sequence identity to the amino acid sequence of SEQ ID NO: 70.

In some embodiments of combination vaccines, the IAV H1 HA protein is an IAV H1 HA protein described in the section entitled “H1 HA proteins.” In some embodiments, the IAV H1 HA protein comprises an amino acid sequence with at least 90% sequence identity to the amino acid sequence of SEQ ID NO: 83. In some embodiments, the IAV H1 HA protein comprises an amino acid sequence with at least 90% sequence identity to the amino acid sequence of SEQ ID NO: 95.

In some embodiments of combination vaccines, the IAV H3 HA protein is an IAV H3 HA protein described in the section entitled “H3 HA proteins.” Where a combination vaccine includes multiple H3 HA proteins (e.g., comprises multiple mRNAs encoding different H3 HA proteins), the different H3 HA proteins may each comprise the same substitutions, or different substitutions described in the section entitled “H3 HA proteins.” In some embodiments, one or more H3 HA proteins does not comprise a substitution described in the section entitled “H3 HA proteins.” In some embodiments, the IAV H3 HA protein comprises an amino acid sequence with at least 90% sequence identity to the amino acid sequence of SEQ ID NO: 82. In some embodiments, each IAV H3 HA protein comprises an amino acid sequence with at least 90% sequence identity to the amino acid sequence of SEQ ID NO: 82.

In some embodiments, the SARS-CoV-2 S protein or fragment thereof is a full-length S glycoprotein described in the “Coronaviruses” section below. In some embodiments, the SARS-CoV-2 S protein or fragment thereof is a protein comprising one or more fragments of the SARS-CoV-2 S glycoprotein, described in the “Coronaviruses” section below.

In some embodiments, the hRSV F protein or fragment thereof is an hRSV protein or fragment thereof described in the “Respiratory Syncytial Virus (hRSV)” section below.

### Coronaviruses

Some embodiments of combination vaccines include coronavirus antigens or nucleic acids encoding coronavirus antigens. Coronaviruses are a family of enveloped, positive-sense, single-stranded RNA viruses that infect mammals and birds. Notable human coronaviruses that cause respiratory illnesses include 229E, NL63, OC43, HKU1, Middle East Respiratory Syndrome Coronavirus (MERS-CoV), and Severe Acute Respiratory Syndrome Coronavirus (SARS-CoV). Coronaviruses derive their name from the crown-like spikes on their surface, which are formed by the viral spike glycoprotein (S protein). The S protein binds to host receptors and mediates viral entry into cells. Other viral structural proteins include the envelope (E), membrane (M), and nucleocapsid (N) proteins. SARS-CoV-2 belongs to the betacoronavirus genus and is the causative agent of COVID-19. It has a size of 29.8-30 kb (see, e.g., Chan et al. 2000, supra; Kim et al. 2020 Cell, May 14; 181(4):914-921.e10.). The SARS-CoV-2 genome and is organized into specific genes encoding structural proteins and nonstructural proteins (Nsps). The order of the structural proteins in the genome is 5′-replicase (open reading frame (ORF)1/ab)-structural proteins [Spike (S)-Envelope I-Membrane (M)-Nucleocapsid (N)]-3′. The genome of coronaviruses includes a variable number of open reading frames that encode accessory proteins, nonstructural proteins, and structural proteins (Song et al. 2019 Viruses; 11(1): p. 59). Most of the antigenic epitopes are located in the structural proteins (Cui et al. 2019 Nat. Rev. Microbiol.; 17(3):181-192). Spike surface glycoprotein(S), a small envelope protein (E), matrix protein (M), and nucleocapsid protein (N) are four main structural proteins. Since S-protein contributes to cell tropism and virus entry and also it is capable to induce neutralizing antibodies (NAb) and protective immunity, it can be considered one of the most important targets in coronavirus vaccine development among all other structural proteins.

Variant viral strains of SARS-CoV-2 may emerge at times. These strains may emerge, for instance, seasonally. Thus, in exemplary aspects, the vaccines may be designed to combat seasonal coronavirus strains, and as such are vaccines for use in an upcoming or forthcoming Northern hemisphere season or Southern hemisphere season. Based on an understanding of circulating coronaviruses at a given point in time, the vaccines can be designed to combat such viruses as they are predicted to be those that will be circulating or prevalent in the upcoming or forthcoming virus season.

A preferred protein is the Spike (S) protein, which is on the surface of coronaviruses, including SARS-CoV-2. An example of a wild-type SARS-CoV-2 Spike protein is provided by the amino acid sequence of SEQ ID NO: 78. In some embodiments, the SARS-CoV-2 antigen is a full-length S protein. In some embodiments, the SARS-CoV-2 antigen comprises a full-length S protein with two or more proline substitutions. In some embodiments, the S protein comprises two proline substitutions at residues corresponding to K986V and V987P of SEQ ID NO: 78. In some embodiments, the S protein does not comprise a proline substitution.

In some embodiments, the SARS-CoV-2 antigen does not comprise a full-length S protein. For example, in some embodiments, the SARS-CoV-2 antigen comprises a receptor binding domain (RBD) of the S protein. In some embodiments, the SARS-CoV-2 antigen comprises a fusion protein comprising an RBD and an N-terminal domain (NTD) of the S protein. In some embodiments, the fusion protein comprises an RBD and an NTD of the S protein, and a transmembrane domain (TD). In some embodiments, the fusion protein comprises, in N-to-C-terminal order, the NTD-RBD-TD. In some embodiments, the RBD and NTD are linked through a linker. In some embodiments, the linker is a cleavable linker. In some embodiments, the linker is a non-cleavable linker. In some embodiments, the linker is a glycine-serine (GS) linker.

In some embodiments, the TD is a betacoronavirus TD. In some embodiments, the TD is a heterologous TD. In some embodiments the TD is a non-betacoronavirus TD. In some embodiments, the TD is an influenza virus hemagglutinin (HA) TD. In some embodiments, the TD is an influenza A virus H1 HA TD.

In some embodiments, the SARS-CoV-2 antigen comprises at least one mutation present in an S protein of a circulating SARS-CoV-2 isolate, as compared to the S protein amino acid sequence of SEQ ID NO: 78. In some embodiments, the SARS-CoV-2 antigen comprises at least 1, 2, 3, 4, 5, 6, 7, 8, 9, 10, 11, 12, 13, 14, 15, 16, 17, 18, 19, or 20 mutations present in a circulating SARS-CoV-2 S protein, as compared to SEQ ID NO: 78. In some embodiments, the SARS-CoV-2 antigen comprises an amino acid sequence with at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identity to SEQ ID NO: 78.

In some embodiments, the SARS-CoV-2 antigen comprises at least one mutation present in an RBD of an S protein of a circulating SARS-CoV-2 isolate, as compared to the S protein of SEQ ID NO: 78. In some embodiments, the SARS-CoV-2 Spike protein antigen comprises at least 1, 2, 3, 4, 5, 6, 7, 8, 9, 10, 11, 12, 13, 14, 15, 16, 17, 18, 19, or 20 RBD mutation(s) compared to SEQ ID NO: 78. In SEQ ID NO: 78, the RBD corresponds to amino acids 330-528, and so the skilled artisan will appreciate that “a mutation present in an RBD of an S protein” as compared to the S protein of SEQ ID NO: 78 refers to (i) a substitution at a position corresponding to any one of amino acids 330-528 of SEQ ID NO: 78, (ii) a deletion of any one or more of amino acids 330-528 of SEQ ID NO: 78, and/or (iii) an insertion of one or more amino acids at a position from amino acids 330-528 of SEQ ID NO: 78. In some embodiments, the SARS-CoV-2 antigen comprises an amino acid sequence with at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identity to SEQ ID NO: 60.

Mutations present in a SARS-CoV-2 antigen of a combination vaccine may be present on any circulating SARS-CoV-2 isolate. For example, at the time of filing the instant specification, isolates of the JN.1 variant are circulating. The SARS-CoV-2 sub-variant JN.1 (also known as BA.2.86.1.1), is a subvariant (sublineage) of the SARS-CoV-2 “Omicron” variant, and was first observed in August 2023; this variant is closely related to BA.2.86. The mutations observed in this variant are believed to provide high potential for immune evasion, particularly the L455F “FLip” mutation—a mutation also observed in XBB lineage variants (e.g., HK.3 and EG.5.1). Mutations observed in JN.1 include A31D, V238L, K1155R, N1708S, A1892T, V24F, R252K, T35I, ins16MPLF, R21T, S50L, Δ69-70, V127F, F157S, R158G, N211del, L212I, V213G, L216F, H245N, A264D, I332V, K356T, R403K, V445H, N450D, L452W, L455S, N481K, V483del, E484K, E554K, A570V, P621S, P681H, S939F, and P1143L.

Several sub-variants of the XBB variant have also been identified recently: HV.1, JD.1.1, HK.3, and EG.5. These sub-variants share several mutations, including T19I, L24S, del25/27, V83A, G142D, del144/144, H146Q, Q183E, V213E, G252V, G339H, R346T, L368I, S371F, S373P, S375F, T376A, D405N, R408S, K417N, N440K, V445P, G446S, F456L, N460K, S477N, T478K, E484A, F486P, F490S, Q498R, N501Y, Y505H, D614G, H655Y, N679K, P681H, N764K, D796Y, Q954H, and N969K; some variants include additional mutations, such as: Q52H, F157L, L452R, L455F, and A475V.

The skilled artisan will appreciate that SARS-CoV-2 antigens of combination vaccines may include mutations present in any SARS-CoV-2 S protein that is extant at the time the instant specification. The skilled artisan will also appreciate that SARS-CoV-2 S protein amino acid sequences that do not exist at the time of filing the instant specification may be analyzed for mutations relative to SEQ ID NO: 78, and those mutations may be applied to SARS-CoV-2 antigens of the combination vaccines.

In some embodiments, a combination vaccine comprises two or more coronavirus antigens or nucleic acids encoding two or more coronavirus antigens, such as SARS-CoV-2 antigens from different SARS-CoV-2 variant strains. In some embodiments, the composition comprises one or more RNAs collectively encoding two or more SARS-CoV-2 variant antigens.

### Respiratory Syncytial Virus (RSV)

Some embodiments of combination vaccines include human respiratory syncytial virus antigens or nucleic acids encoding human respiratory syncytial virus antigens. Human respiratory syncytial virus (hRSV; also known as human orthopneumovirus) is a negative-sense, single-stranded ribonucleic acid (RNA) virus of the Pneumoviridae family. The virus is present in at least two antigenic subgroups, known as Group A and Group B.

The envelope of hRSV contains three surface glycoproteins: F, G, and SH. The G and F proteins are protective antigens and targets of neutralizing antibodies. The F protein, however, is more conserved across hRSV strains and types (A and B). hRSV F protein is a type I fusion glycoprotein that is well conserved between clinical isolates, including between the hRSV-A and hRSV-B antigenic subgroups. The F protein transitions between prefusion and more stable postfusion states, thereby facilitating entry into target cells. hRSV F glycoprotein is initially synthesized as an F0 precursor protein. hRSV F0 folds into a trimer, which is activated by furin cleavage into the mature prefusion protein comprising F1 and F2 subunits (Bolt, et al., Virus Res., 68:25, 2000). Although targets for neutralizing monoclonal antibodies exist on the postfusion conformation of F protein, the neutralizing Ab response primarily targets the F protein prefusion conformation in people naturally infected with hRSV (Magro M et al., Proc Natl Acad Sci USA 2012; 109(8):3089-94; Ngwuta J O et al., Sci Transl Med 2015; 7(309):309ra162). Consistent with this, hRSV F protein stabilized in the prefusion conformation produces a greater neutralizing immune response in animal models than that observed with hRSV F protein stabilized in the post fusion conformation (McLellan et al., Science, 342:592-598, 2013). Thus, stabilized prefusion hRSV F proteins are good candidates for inclusion in an hRSV vaccine. Other RSV proteins include the small hydrophobic (SH), matrix (M), nucleocapsid (N), phosphoprotein (P), polymerase (L) and nonstructural NS1/NS2 proteins. A preferred protein is the F glycoprotein, which present on the surface of hRSV, including wild-type hRSV and mutant strains, such as the hRSV F protein having an amino acid sequence of SEQ ID NO: 98.

hRSV commonly causes bronchiolitis. Most infected adults develop mild cold-like symptoms such as congestion, low-grade fever, and wheezing. Infants and small children may suffer more severe symptoms such as bronchiolitis and pneumonia. The disease may be transmitted among humans via contact with respiratory secretions.

Some embodiments relate to stabilized prefusion RSV F proteins that comprise mutations to prevent the transition of the protein into its post-fusion conformation. For example, in some embodiments, the stabilized prefusion RSV F protein comprises proline residue (e.g., an S215P substitution) and/or isoleucine (e.g., N67I substitution) substitutions. As an example, the DS-Cav1 variant, a stabilized prefusion RSV F protein, contains an additional disulfide bond (S155C/S290C) as well as two cavity-filling mutations (S190F/V207L). Another stabilized prefusion RSV F protein is PR-DM, which comprises one proline substitution (S215P) and one mutation in the F2 subunit (N67I).

In some embodiments, a stabilized prefusion hRSV F glycoprotein variant lacks a cytoplasmic tail. In some embodiments, the cytoplasmic tail comprises the C-terminal 20-30, 20-25, 15-30, 15-25, 15-20, 10-30, 10-25, 10-20, 10-15, 5-30, 5-25, 5-20, or 5-15 amino acids of the of the hRSV F glycoprotein variant. In some embodiments, the cytoplasmic tail comprises the C-terminal 25 amino acids (e.g., CKARSTPVTLSKDQLSGINNIAFSN(SEQ ID NO: 101)) of the hRSV F glycoprotein. In some embodiments, the cytoplasmic tail comprises the C-terminal 20 amino acids (e.g., TPVTLSKDQLSGINNIAFSN(SEQ ID NO: 102)) of the hRSV F glycoprotein. In some embodiments, the cytoplasmic tail comprises the C-terminal 15 amino acids (e.g., SKDQLSGINNIAFSN(SEQ ID NO: 103)) of the hRSV F glycoprotein. In some embodiments, the cytoplasmic tail comprises the C-terminal 10 amino acids (e.g., SGINNIAFSN(SEQ ID NO: 104)) of the hRSV F glycoprotein.

In some embodiments, a stabilized prefusion hRSV F glycoprotein variant lacks a cytoplasmic tail, wherein the RSV F glycoprotein variant has at least 80%, at least 85%, at least 90%, at least 95% identity to a wild-type hRSV F glycoprotein (e.g., a wild-type hRSV F glycoprotein comprising the sequence of SEQ ID NO: 98), and lacks a cytoplasmic tail. In some embodiments, stabilized prefusion hRSV F glycoprotein variant lacks a cytoplasmic tail, wherein the RSV F glycoprotein variant has at least 80%, at least 85%, at least 90%, at least 95% identity to the sequence of SEQ ID NO: 99. In some embodiments, stabilized prefusion hRSV F glycoprotein variant comprises the sequence of SEQ ID NO: 99.

In some embodiments, an hRSV F glycoprotein variant that lacks a cytoplasmic tail further comprises a modification, relative to the wild-type hRSV F glycoprotein (e.g., SEQ ID NO: 98), selected from the group consisting of: a P102X substitution, a substitution of amino acids 104-144 with a linker molecule, an A149X substitution, an S155X substitution, an S190X substitution, a V207X substitution, an S290X substitution, a L373X substitution, an I379X substitution, an M447X substitution, and a Y458X substitution, wherein X is any amino acid (e.g., A, R, N, D, C, E, Q, G, H, I, L, K, M, F, P, S, T, W, Y, or V) other than the amino acid replaced in the wild-the hRSV F glycoprotein amino acid sequence. In some embodiments, an hRSV F glycoprotein variant that lacks a cytoplasmic tail further comprises a modification, relative to the wild-type hRSV F glycoprotein, (SEQ ID NO: 98), selected from the group consisting of: a P102A substitution, a substitution of amino acids 104-144 with a linker molecule, an A149C substitution, an S155C substitution, an S190F substitution, a V207L substitution, an S290C substitution, a L373R substitution, an I379V substitution, an M447V substitution, and a Y458C substitution. In some embodiments, an hRSV F glycoprotein variant that lacks a cytoplasmic tail further comprises a P102A substitution. In some embodiments, an hRSV F glycoprotein variant that lacks a cytoplasmic tail further comprises a substitution of amino acids 104-144 with a linker molecule. In some embodiments, an hRSV F glycoprotein variant that lacks a cytoplasmic tail further comprises an A149C substitution. In some embodiments, an hRSV F glycoprotein variant that lacks a cytoplasmic tail further comprises an S155C substitution. In some embodiments, an hRSV F glycoprotein variant that lacks a cytoplasmic tail further comprises an S190F substitution. In some embodiments, an hRSV F glycoprotein variant that lacks a cytoplasmic tail further comprises a V207L substitution. In some embodiments, an hRSV F glycoprotein variant that lacks a cytoplasmic tail further comprises an S290C substitution. In some embodiments, an hRSV F glycoprotein variant that lacks a cytoplasmic tail further comprises an L373R substitution. In some embodiments, an hRSV F glycoprotein variant that lacks a cytoplasmic tail further comprises an I379V substitution. In some embodiments, an hRSV F glycoprotein variant that lacks a cytoplasmic tail further comprises an M447V substitution. In some embodiments, an hRSV F glycoprotein variant that lacks a cytoplasmic tail further comprises a Y458C substitution.

In some embodiments, an hRSV F glycoprotein variant that lacks a cytoplasmic tail further comprises the following modifications, relative to the wild-type hRSV F glycoprotein: a P102A substitution, a substitution of amino acids 104-144 with a linker molecule, an A149C substitution, an S155C substitution, an S190F substitution, a V207L substitution, an S290C substitution, a L373R substitution, an I379V substitution, an M447V substitution, and a Y458C substitution.

In some embodiments, an hRSV F glycoprotein comprises an amino acid sequence with at least 80%, at least 85%, at least 90%, at least 95%, at least 96%, at least 97%, at least 98%, or at least 99% identity to the amino acid sequence of SEQ ID NO: 99. In some embodiments, the hRSV F glycoprotein comprises the amino acid sequence of SEQ ID NO: 99. In some embodiments, the hRSV F glycoprotein consists of the amino acid sequence of SEQ ID NO: 99.

In some embodiments, the composition comprises an RNA having an ORF encoding an hRSV F glycoprotein, where the ORF comprises a nucleotide sequence with at least 70%, at least 75%, least 80%, at least 85%, at least 90%, at least 95%, at least 96%, at least 97%, at least 98%, or at least 99% identity to the nucleotide sequence of SEQ ID NO: 113. In some embodiments, the ORF comprises the nucleotide sequence of SEQ ID NO: 113.

In some embodiments, an hRSV F glycoprotein comprises an F1 subunit and an F2 subunit, the F1 subunit comprising at least 80%, at least 85%, at least 90%, at least 95%, at least 96%, at least 97%, at least 98%, or at least 99% identity to the amino acid sequence of SEQ ID NO: 106, and the F2 subunit comprising at least 80%, at least 85%, at least 90%, at least 95%, at least 96%, at least 97%, at least 98%, or at least 99% identity to the amino acid sequence of SEQ ID NO: 107. In some embodiments, the F1 subunit comprises the amino acid sequence of SEQ ID NO: 106, and the F2 subunit comprises the amino acid sequence of SEQ ID NO: 107. In some embodiments, the F1 subunit consists of the amino acid sequence of SEQ ID NO: 106, and the F2 subunit consists of the amino acid sequence of SEQ ID NO: 107. In some embodiments, the hRSV F glycoprotein comprises an amino acid sequence with at least 80%, at least 85%, at least 90%, at least 95%, at least 96%, at least 97%, at least 98%, or at least 99% identity to the amino acid sequence of SEQ ID NO: 108. In some embodiments, the hRSV F glycoprotein comprises the amino acid sequence of SEQ ID NO: 108. In some embodiments, the hRSV F glycoprotein consists of the amino acid sequence of SEQ ID NO: 108.

In some embodiments, an hRSV F glycoprotein comprises an F1 subunit and an F2 subunit, the F1 subunit comprising at least 80%, at least 85%, at least 90%, at least 95%, at least 96%, at least 97%, at least 98%, or at least 99% identity to the amino acid sequence of SEQ ID NO: 109, and the F2 subunit comprising at least 80%, at least 85%, at least 90%, at least 95%, at least 96%, at least 97%, at least 98%, or at least 99% identity to the amino acid sequence of SEQ ID NO: 110. In some embodiments, the F1 subunit comprises the amino acid sequence of SEQ ID NO: 109, and the F2 subunit comprises the amino acid sequence of SEQ ID NO: 110. In some embodiments, the F1 subunit comprises the amino acid sequence of SEQ ID NO: 109, and the F2 subunit comprises the amino acid sequence of SEQ ID NO: 110. In some embodiments, the F1 subunit consists of the amino acid sequence of SEQ ID NO: 109, and the F2 subunit consists of the amino acid sequence of SEQ ID NO: 110. In some embodiments, the hRSV F glycoprotein comprises an amino acid sequence with at least 80%, at least 85%, at least 90%, at least 95%, at least 96%, at least 97%, at least 98%, or at least 99% identity to the amino acid sequence of SEQ ID NO: 111. In some embodiments, the hRSV F glycoprotein comprises the amino acid sequence of SEQ ID NO: 111. In some embodiments, the hRSV F glycoprotein consists of the amino acid sequence of SEQ ID NO: 111.

In some embodiments, an hRSV F glycoprotein comprises an amino acid sequence with at least 80%, at least 85%, at least 90%, at least 95%, at least 96%, at least 97%, at least 98%, or at least 99% identity to the amino acid sequence of SEQ ID NO: 112.

### Protein Variants and Alignment

Some embodiments relate to proteins having one or more mutations (e.g., substitutions) relative to a reference amino acid sequence and/or numbered according to a listed amino acid sequence.

Some embodiments relate to amino acid or nucleotide sequences having a specified percentage sequence identity to a comparator amino acid or nucleotide sequence, respectively. The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. “Percent (%) identity” or “percent (%) sequence identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity.

The percent sequence identity that a candidate sequence (e.g., as present in a claimed protein or nucleic acid) has to a comparator sequence (e.g., having a SEQ ID NO: specified herein) is calculated by (i) aligning the candidate sequence to the comparator sequence, (ii) determining the number of matching residues (amino acids or nucleotides) between the aligned candidate and comparator sequences, and (iii) dividing the number of matching residues by the length of the comparator sequence, including any gaps introduced into the comparator sequence when the two sequences are aligned.

For example, alignment of a candidate amino acid sequence of the influenza B/Brisbane/60/2008 virus HA protein amino acid sequence of SEQ ID NO: 157 to a comparator amino acid sequence of the influenza B/Austria/1359417/2021 virus HA protein amino acid sequence of SEQ ID NO: 71 reveals 572 matching residues (FIG. 19E). While SEQ ID NO: 71 is only 582 amino acids long, an internal gap three amino acids in length is introduced into SEQ ID NO: 71 in the alignment (FIG. 19E), making the denominator 585. Thus, candidate sequence SEQ ID NO: 157 has 572/585=97.8% sequence identity to comparator sequence SEQ ID NO: 71.

The skilled artisan will appreciate that to determine whether a candidate protein or nucleic acid comprises an amino acid sequence or nucleotide sequence with a given percentage sequence identity to a comparator sequence, the denominator (length of comparator sequence plus internal gaps) in calculating sequence identity need not include gaps shown at the ends of the comparator sequence in an alignment, as such gaps are added where a candidate sequence contains additional amino acids or nucleotides that extend beyond the portions that align to the N-terminal end and/or C-terminal end (amino acid sequences), or 5′ end or 3′ end (nucleotide sequences) of the comparator sequence. For example, alignment of the influenza B/Austria/1359417/2021 virus HA protein amino acid sequence of SEQ ID NO: 71, including the signal peptide, to its post-signal peptide cleavage form of SEQ ID NO: 174, results in a gap at the N-terminus of the comparator sequence, where the signal peptide is not present (FIG. 19F). A protein having the full-length amino acid sequence of SEQ ID NO: 71 would still comprise an amino acid sequence with 100% identity to SEQ ID NO: 174, because candidate sequence SEQ ID NO: 71 as aligned to comparator sequence SEQ ID NO: 174 contains 567 matches, and the number of amino acids in the comparator sequence of SEQ ID NO: 174 is 567 (567/567=100%).

Where an alignment between two sequences is contemplated, the first sequence (e.g., candidate sequence) is aligned to the second sequence (e.g., comparator sequence) using the Needleman-Wunsch algorithm for global alignment of the two sequences. Needleman & Wunsch, J Mol Biol. 1970. 48:443-453. Where two protein sequences are aligned, the Needleman-Wunsch algorithm uses a BLOSUM62 substitution scoring matrix, a Gap Open penalty of 10, a Gap Extend penalty of 0.5, and no End Gap penalties. Where two nucleotide sequences are aligned, the alignment uses an DNAFULL substitution scoring matrix, a Gap Open penalty of 10, a Gap Extend penalty of 0.5, and no End Gap penalties. The skilled artisan will appreciate that at the time of filing the instant specification, these parameters are the default parameters of the EMBOSS Needle pairwise comparison tool provided by European Bioinformatics Institute (see ebi.ac.uk). Other suitable alignment programs may be used to obtain a global alignment using these parameters, such as BLAST, or the Needleman-Wunsch algorithm may be implemented in a scripting language (e.g., Python).

### Linkers and Cleavable Peptides

Some embodiments of proteins include a linker between at least one pair of portions of the protein. The linker may be, for example, a cleavable linker or protease-sensitive linker. In some embodiments, the linker is selected from the group consisting of F2A linker, P2A linker, T2A linker, E2A linker, and combinations thereof (see, e.g., WO 2017/127750). This family of self-cleaving peptide linkers, referred to as 2A peptides, has been described in the art (see, e.g., Kim, J. H. et al., PLoS ONE 2011; 6: e18556).

In some embodiments, the linker is an F2A linker. In some embodiments, the linker is a GS linker. GS linkers are polypeptide linkers that include glycine and serine amino acids repeats. They comprise flexible and hydrophilic residues and can be used to perform fusion of protein subunits without interfering in the folding and function of the protein domains, and without formation of secondary structures. In some embodiments, a protein comprises a GS linker that is 3 to 20 amino acids long. For example, the GS linker may have a length of (or have a length of at least) 3, 4, 5, 6, 7, 8, 9, 10, 11, 12, 13, 14, 15, 16, 17, 18, 19, or 20 amino acids. In some embodiments, a GS linker is (or is at least) 15 amino acids long (e.g., GGSGGSGGSGGSGGG (SEQ ID NO: 114)). In some embodiments, a GS linker is (or is at least) 8 amino acids long (e.g., GGGSGGGS (SEQ ID NO: 115)). In some embodiments, a GS linker is (or is at least) 7 amino acids long (e.g., GGGSGGG (SEQ ID NO: 116)). In some embodiments, a GS linker comprises the amino acid sequence GGGSGG (SEQ ID NO: 117). In some embodiments, a GS linker is (or is at least) 4 amino acid long (e.g., GGGS (SEQ ID NO: 118)). In some embodiments, the GS linker comprises (GGGS)n (SEQ ID NO: 118), where n is any integer from 1-5. In some embodiments, a GS linker is (or is at least) 4 amino acid long (e.g., GSGG (SEQ ID NO: 119)). In some embodiments, the GS linker comprises (GSGG)n (SEQ ID NO: 119), where n is any integer from 1-5. In some embodiments, a linker is a glycine linker, for example having a length of (or a length of at least) 3 amino acids (e.g., GGG). In some embodiments, a protein encoded by an RNA (e.g., mRNA) includes two or more linkers, which may be the same or different from each other. The skilled artisan will appreciate that other linkers may be suitable for use in proteins.

### Signal Peptides

In some embodiments, a protein comprises a signal peptide. Signal peptides comprise the N-terminal 15-60 amino acids of proteins. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (e.g., other than influenza virus HA, influenza virus NA, hRSV F, and SARS-CoV-2 S glycoproteins) may also be used in a protein.

The native signal peptide of a protein may be determined using any suitable method, such as a signal peptide prediction tool. Signal peptide prediction tools use bioinformatic algorithms, such as neural network, machine learning, and/or language model-based approaches, in combination with annotated protein databases (e.g., UniProt) to predict the signal peptide sequence within a given amino acid sequence. See, e.g., Teufel et al., Nat Biotechnol. 2022. 40(7):1023-1025 (SignalP 6.0)

Non-exhaustive examples of signal peptides of HA and NA proteins of influenza A/(H1N1) subtype, A/(H3N2) subtype, B/Victoria lineage, and B/Yamagata lineage isolates are provided in Table SP-1.

TABLE SP-1
Exemplary IAV and IBV HA and NA protein signal peptides

| Subtype | | HA protein | |
|---|---|---|---|
| (IAV) or | | signal | NA protein signal |
| lineage (IBV) | Isolate | peptide | peptide |
| A/(H1N1) | A/Wisconsin/ | MKAILVVMLYTFTTA | MNPNQKIITIGSVCMTI |
| subtype | 67/2022 | NA (SEQ ID NO: 168) | GTANLILQIGNI |
| | (H1N1)pdm09 | | (SEQ ID NO: 226) |
| A/(H3N2) | A/Darwin/6/2021 | MKTIIALSNILCLVFA | MNPNQKIITIGSVSLTIS |
| subtype | (H3N2) | (SEQ ID NO: 169) | TICFFMQIAIL |
| | | | (SEQ ID NO: 227) |
| B/Victoria | B/Austria/ | MKAIIVLLMVVTSNA | MLPSTIQTLTLFLTSGG |
| lineage | 1359417/2021 | (SEQ ID NO: 170) | VLLSLYVSASLSYL |
| | | | (SEQ ID NO: 228) |
| B/Yamagata | B/Phuket/ | MKAIIVLLMVVTSNA | MLPSTIQTLTLFLTSGG |
| lineage | 3073/2013 | (SEQ ID NO: 171) | VLLSLYVSASLSYL |
| | | | (SEQ ID NO: 229) |

### Nucleic Acids

Provided are compositions comprising nucleic acids. In some embodiments, the nucleic acids comprise DNA. In some embodiments, the nucleic acids comprise RNA, such as self-amplifying RNA, circular RNA, or mRNA. Preferably, the nucleic acid comprises mRNA.

Except where otherwise noted, nucleic acid sequences set forth in the instant application may recite “T”s in a representative DNA sequence but where the sequence represents RNA, the “T”'s would be substituted for “U”s. Thus, any of the DNAs disclosed and identified by a particular sequence herein also discloses the corresponding RNA sequence where each “T” of the DNA sequence is substituted with “U.”.

Messenger RNA (mRNA)

Messenger RNA (mRNA) is RNA that encodes a (at least one) protein or a fragment thereof and can be translated to produce the encoded protein or fragment in vitro, in vivo, in situ, or ex vivo. mRNA comprises an open reading frame (ORF) encoding the protein or fragment thereof. In some embodiments, the mRNA further comprises a 5′ untranslated region (UTR), 3′ UTR, a polyA tail, and/or a 5′ cap analog.

The disclosed mRNA may encode a single protein or fragment or they may be polycistronic constructs, which encode more than one protein or fragment separately within the same mRNA molecule. Additionally or alternatively, the disclosed mRNA may encode a fusion protein or fragment thereof.

i. Open Reading Frame (ORF)

An open reading frame (ORF) is a continuous stretch of DNA or RNA beginning with a start codon (e.g., methionine (ATG or AUG)) and ending with a stop codon or codons (e.g., TAA, TAG, TGA, UAA, UAG, UGA, UGAUGA or UGAUAAUAG). For clarity: the stop codon itself is not considered a part of the ORF. An ORF typically encodes a protein or fragment thereof.

ii. Untranslated Regions (UTRs)

In some embodiments, mRNA comprises one or more regions or parts which act or function as an untranslated region. A 5′ untranslated region” (5′ UTR) is a region of an mRNA that is upstream (i.e., 5′) from the start codon and does not encode a polypeptide. A 3′ untranslated region” (3′UTR) is a region of an mRNA that is downstream (i.e., 3′) from the stop codon and also does not encode a polypeptide

The 5′ UTR may start at the transcription start site and continues to the start codon but does not include the start codon. The 3′ UTR may start immediately following the stop codon and continue until a transcriptional termination signal. A variety of 5′ UTR and 3′ UTR sequences are known. Exemplary UTR sequences include SEQ ID NOs: 1-35 (5′ UTRs) and 36-44 (3′ UTRs), which are shown in Tables S-1 (5′ UTRs) and S-2 (3′ UTRs) of the section “Exemplary Sequences”. In some embodiments, the 5′ UTR comprises a sequence provided in Table S-1 or a sequence with at least 80%, 85%, 90%, 95%, 96%, 97%, 98%, 99% or 100% identity to a 5′ UTR sequence provided in Table S-1, or a variant or a fragment thereof. In some embodiments, the 3′ UTR comprises a sequence provided in Table S-2 or a sequence with at least 80%, 85%, 90%, 95%, 96%, 97%, 98%, 99% or 100% identity to a 3′ UTR sequence provided in Table S-2, or a variant or a fragment thereof.

Each RNA species in a multivalent RNA composition may comprise an IDR sequence that is not a sequence isomer of an IDR sequence of another RNA species in a multivalent RNA composition (e.g., the IDR sequence does not have the same number of adenosine nucleotides, the same number of cytosine nucleotides, the same number of guanine nucleotides, and the same number of uracil nucleotides (and consequently the same mass) as another IDR sequence in the composition, even if those sequences have different sequences).

Each RNA species in a multivalent RNA composition may comprise an IDR sequence having a mass that differs from the mass of IDR sequences of each other RNA species in a multivalent RNA composition. For example, the mass of each IDR sequence may differ from the mass of other IDR sequences by at least 9 Da, at least 25 Da, at least 25 Da, or at least 50 Da. Use of IDR sequences with distinct masses allows RNA fragments comprising different IDR sequences to be distinguished using mass-based analysis methods (e.g., mass spectrometry), which do not require reverse transcription, amplification, or sequencing of RNAs.

Each RNA species in an RNA composition may comprises an IDR sequence with a different length. For example, each IDR sequence may have a length independently selected from 0 to 25 nucleotides. The length of a nucleic acid influences the rate at which the nucleic acid traverses a chromatography column, and so the use of IDR sequences of different lengths on different RNA species allows RNA fragments having different IDR sequences to be distinguished using chromatography-based methods (e.g., LC-UV).

Combinations of features may be included in flanking regions and may be contained within other features. For example, the ORF may be flanked by a 5′ UTR which may contain a strong Kozak translational initiation signal and/or a 3′ UTR which may include an oligo (dT) sequence for templated addition of a poly-A tail. 5′ UTR may comprise a first polynucleotide fragment and a second polynucleotide fragment from the same and/or different genes such as the 5′ UTRs described in US 2010/0293625 and WO 2015/085318.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US 2010/0129877.

For the purposes of the present disclosure, a UTR may also include one or more translation enhancer elements (TEE). As a non-limiting example, the TEE may include those described in US 2009/0226470, herein incorporated by reference, and those known in the art.

iii. PolyA Tail

In some embodiments, the mRNA contains a 3′-polyA tail. A polyA tail may contain 10 to 300 adenosine monophosphates. It can, in some instances, comprise up to about 400 adenine nucleotides. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine nucleotides. In some embodiments, a polyA tail contains 50 to 250 adenosine nucleotides. In some embodiments, a polyA tail has a length of about 50, about 100, about 150, about 200, about 250, about 300, about 350, or about 400 nucleotides. In some embodiments, a polyA tail has a length of 100 nucleotides.

In some embodiments, an mRNA may comprise two polyA sequences separated by an intervening nucleotide sequence. In some embodiments, the intervening nucleotide sequence comprises no more than 3, no more than two, no more than 1, or no adenosine nucleotides. In some embodiments, the intervening sequence comprises 3 adenosine nucleotides. In some embodiments, the intervening sequence is no more than 30, no more than 25, no more than 20, no more than 15, or no more than 10 nucleotides long. In some embodiments, the intervening sequence consists of 10 nucleotides. In some embodiments, the intervening sequence comprises the sequence of GCAUAUGACU. In some embodiments, the intervening sequence does not begin with an adenosine nucleotide, and does not end with an adenosine nucleotide. In some embodiments, the first polyA sequences comprises at least 15, at least 20, at least 25, or at least 30 consecutive adenosine nucleotides. In some embodiments, the second polyA sequences comprises at least 55, at least 60, at least 65, or at least 70 consecutive adenosine nucleotides. In some embodiments, the first polyA sequence comprises 30 consecutive adenosine nucleotides. In some embodiments, the second polyA sequence comprises 70 adenosine nucleotides.

### iv. 5′ Cap

In some embodiments, mRNA comprises a 5′ end cap or a “5′ terminal cap.” A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap.

5′-capping of polynucleotides may be completed concomitantly during an in vitro transcription reaction using, for example, the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, MA). 5′-capping of modified mRNA may be completed post-transcriptionally using, for example, a Vaccinia Virus Capping Enzyme to generate the “Cap 0” structure: m7G(5′)ppp(5′)G (New England BioLabs, Ipswich, MA). A Cap 1 structure may be generated using both Vaccinia Virus Capping Enzyme and a 2′-O methyl-transferase to generate: m7G(5′)ppp(5′)G-2′-O-methyl. A Cap 2 structure may be generated from the Cap 1 structure followed by the 2′-O-methylation of the 5′-antepenultimate nucleotide using a 2′-0 methyl-transferase. A Cap 3 structure may be generated from the Cap 2 structure followed by the 2′-O-methylation of the 5′-preantepenultimate nucleotide using a 2′-O methyl-transferase. Enzymes may be derived from a recombinant source. Other cap analogs, such as a 7 mG(5′)ppp(5′)NlmpNp cap, may be used.

### Chemical Modifications

An mRNA may include nucleotides that are not chemically modified (i.e., unmodified nucleotides), nucleotides that are chemically modified, or both. Nucleotides that are not chemically modified are the standard ribonucleotides consisting of adenosine, guanosine, cytidine, and uridine.

Some embodiments of mRNAs comprise modified nucleosides and/or nucleotides. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside in combination with a phosphate group. Modifications to nucleotides or nucleosides can be at the sugar or nucleobase. Modified nucleotides may by synthesized by any useful method, such as, for example, chemically, enzymatically, or recombinantly.

In some embodiments, modified nucleobases in mRNA comprise N1-methyl-pseudouridine (m1ψ), N1-ethyl-pseudouridine (e1ψ), 5-methoxy-uridine (mo5U), 5-methyl-uridine (m5U), 5-methyl-cytidine (m5C), and/or pseudouridine (ψ). In some embodiments, modified nucleobases in mRNAs comprise 5-methoxymethyl uridine, 5-methylthio uridine, 1-methoxymethyl pseudouridine, 5-methyl cytidine, and/or 5-methoxy cytidine. In some embodiments, the mRNA includes a combination of at least two (e.g., 2, 3, 4 or more) of any of the aforementioned modified nucleobases, including but not limited to chemical modifications.

In some embodiments, a mRNA comprises 1-methyl-pseudouridine (mlv) substitutions at one or more or all uridine positions of the mRNA.

In some embodiments, a mRNA comprises 1-methyl-pseudouridine (m1ψ) substitutions at one or more or all uridine positions of the mRNA and 5-methyl cytidine substitutions at one or more or all cytidine positions of the mRNA.

In some embodiments, a mRNA comprises pseudouridine (ψ) substitutions at one or more or all uridine positions of the mRNA.

In some embodiments, a mRNA comprises pseudouridine (ψ) substitutions at one or more or all uridine positions of the mRNA and 5-methylcytidine substitutions at one or more or all cytidine positions of the mRNA.

In some embodiments, a mRNA comprises uridine at one or more or all uridine positions of the mRNA.

In some embodiments, a mRNA comprises 5-methyl-uridine and 5-methyl cytidine at one or more or all uridine and cytidine positions, respectively, of the mRNA.

In some embodiments, mRNAs are uniformly modified (e.g., fully modified, modified throughout the entire sequence) for a particular modification. For example, a mRNA can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above. In some embodiments, the ORF is uniformly modified for a particular modification, such as 1-methyl-pseudouridine. In some embodiments, the uniform modification does not include the mRNA cap. For instance, a cap with different modifications from the remainder of the mRNA can be added co-transcriptionally or post-transcriptionally to the mRNA.

### Codon Optimization

In some embodiments, an ORF encoding a protein or fragment thereof is codon optimized. Codon optimization methods are known in the art. For example, an ORF of any one or more of the sequences listed below may be codon optimized. Codon optimization, in some embodiments, may be used to match codon frequencies in target and host organisms to ensure proper folding; bias GC content to increase RNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and RNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95%, less than 90%, less than 85%, less than 80%, or less than 75% sequence identity to a naturally-occurring or wild-type sequence open reading frame (e.g., a naturally-occurring or wild-type mRNA sequence encoding a protein or fragment thereof). In some embodiments, a codon optimized sequence shares between 65% and 85% sequence identity to a naturally-occurring or wild-type sequence (e.g., a naturally-occurring or wild-type RNA or DNA sequence encoding a protein or fragment thereof).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a protein or fragment thereof encoded by a non-codon-optimized sequence.

### Self-amplifying RNA

In some embodiments, an RNA is a self-amplifying RNA. A self-amplifying RNA is an RNA encoding one or more proteins that, individually or in conjunction, are capable of replicating the self-amplifying RNA. In some embodiments, the proteins encoded by the self-amplifying RNA are non-structural proteins nsP1, nsP2, nsP3, and nsP4, which form an RNA-dependent RNA polymerase (RdRp), or replicase, that is capable of replicating the self-amplifying RNA. By encoding proteins that are capable of replicating the RNA, a self-amplifying RNA is capable of self-amplification in a cell, provided that the cell can translate the RNA and produce the encoded protein(s). A self-amplifying RNA may be referred to as an RNA replicon.

When a self-amplifying RNA is translated, the one or more encoded viral non-structural proteins are translated. A “viral non-structural protein” is a protein encoded by a virus but that is not part of the virus particle. The viral non-structural proteins, in the context of self-amplifying RNA, replicate the nucleotide sequences encoding the desired protein from the self-amplifying RNA via the sub-genomic viral promoters. Such replication driven by the viral sub-genomic promoter using the viral non-structural proteins enhances the expression level of the encoded protein. In some embodiments, the viral non-structural proteins are from a single-strand positive-sense RNA viruses. In some embodiments, the viral non-structural proteins are from an Alphavirus, belonging to the Togaviridae family. In some embodiments, the alphavirus is Sindbis or Venezuelan equine encephalitis virus. In some embodiments, the viral non-structural protein is an RNA-dependent RNA polymerase (RdRp) polyprotein P1234 (also termed NSP1-4).

Upon translation, P1234 is rapidly cleaved into P123 and nsP4 by autoproteolytic activity originating from the nsP2 (proteinase) portion of the polyprotein. Alphaviral RNA synthesis occurs at the plasma membrane of a cell, where the nsPs, together with alphaviral RNA, form membrane invaginations (or “spherules”). These spherules contain dsRNA created by replication of “+” strand viral genomic RNA into “−” strand anti-genomic RNA. The “−” strand serves as a template from which additional “+” strand genomic RNA (synthesized from the 5′ UTR) or a shorter subsequence of the genomic RNA (termed subgenomic RNA) is synthesized from the subgenomic viral promoter region located near the end of the nonstructural protein ORF. The “+” strand genomic RNA and the subgenomic RNA are exported out of the spherules into the cytoplasm where they are translated by endogenous ribosomes. The exported “+” strand genomic RNA can associate with nsPs and form additional spherules, thus resulting in exponential increase of replicon RNA.

The viral non-structural proteins facilitate the replication of the nucleotide sequences encoding the desired protein via the subgenomic viral promoters (also referred to as “subgenomic promoters” herein). A “subgenomic viral promoter” refers to a promoter the drives the transcription of subgenomic mRNAs. Typically, an mRNA is transcribed from genomic DNAs and episomal DNAs (e.g., plasmids). Some viruses may transcribe subgenomic mRNAs from a RNA replicon that is produced from its genomic RNA. Many positive-sense RNA viruses produce subgenomic mRNAs as one of the common infection techniques used by these viruses and generally transcribe late viral genes. Subgenomic viral promoters range from 20 nucleotide (Sindbis virus) to over 100 nucleotides (Beet necrotic yellow vein virus) and are usually found upstream of the transcription start. In some embodiments, the subgenomic viral promoter is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides long, or longer. Subgenomic viral promoters have been described in the art, e.g., in PCT Publication No. WO 2016/040359, and Wagner et al., Nature Chemical Biology, DOI: 10.1038/s41589-018-0146-9 (2018).

### Circular RNA

In some embodiments, an RNA is a circular RNA. A circular RNA is an RNA with no 5′ terminal nucleotide or 3′ terminal nucleotide. Every nucleotide in a circular RNA is covalently bonded to both (1) a 5′ adjacent nucleotide; and (2) a 3′ adjacent nucleotide. In a circular RNA with a nucleotide sequence comprising every nucleotide of the circular RNA in 5′-to-3′ order, the last nucleotide of the nucleotide sequence is covalently bonded to the first nucleotide of the nucleotide sequence.

A circular RNA may be a circular mRNA, comprising one or more 5′ UTRs, an open reading frame, and one or more 3′ UTRs. A circular RNA may comprise a polyA region, as described in the section entitled “PolyA Tails”. The skilled artisan will appreciate that a polyA tail, when incorporated in a circular RNA, is referred to as a polyA region because a circular RNA does not have an end as a linear mRNA does.

A circular RNA may comprise an internal ribosome entry site (IRES). Inclusion of an IRES permits the translation of one or more open reading frames from a circular RNA, as circular RNAs do not comprise a 5′ from which a ribosome may initiate translation as with capped linear mRNAs. The IRES element attracts a eukaryotic ribosomal translation initiation complex and promotes translation initiation. See, e.g., Kaufman et al., Nucleic Acid Res. 1991 19:4485-4490; Gurtu et al., Biochem Biophys Res Commun. 1996. 229:295-298; Rees et al., BioTechniques. 1996. 20:102-110; Kobayashi et al., BioTechniques. 1996. 21:399-402; and Mosser et al., BioTechniques. 1997. 22:150-161. A multitude of IRES sequences are available and include sequences derived from a wide variety of viruses, such as from leader sequences of picornaviruses such as the encephalomyocarditis virus (EMCV) UTR (Jang et al., J Virol. 1989. 63:1651-1660), the polio leader sequence, the hepatitis A virus leader, the hepatitis C virus IRES, human rhinovirus type 2 IRES (Dobrikova et al., Proc Natl Acad Sci USA. 2003. 100(25):15125-15130), an IRES element from the foot and mouth disease virus (Ramesh et al., Nucleic Acid Res. 1996. 24:2697-2700), a giardiavirus IRES (Garlapati et al., J Biol Chem. 2004. 279(5):3389-3397). Additionally or alternatively, a circular RNA may comprise any of a variety of nonviral IRES sequences, such as IRES sequences from yeast, as well as the human angiotensin II type 1 receptor IRES (Martin et al., Mol Cell Endocrinol. (2003) 212:51-61), fibroblast growth factor IRESs (FGF-1 IRES and FGF-2 IRES, Martineau et al., Mol Cell Biol. 2004. 24(17):7622-7635), vascular endothelial growth factor (VEGF) IRES (Baranick et al., Proc Natl Acad Sci USA. 2008. 105(12):4733-4738, Stein et al., Mol Cell Biol. 1998. 18(6):3112-3119, Bert et al., RNA. 2006. 12(6):1074-1083), and insulin-like growth factor II (IGF-II) IRES (Pedersen et al., Biochem J. 2002. 363 (Pt 1):37-44). These elements are commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures. In some embodiments, a circular RNA comprises a coxsackievirus B3 (CVB3) IRES. See Gharbi et al., PLoS One. 2022. 17(10):e0274162. In some embodiments, a circular RNA comprises an EMCV IRES. In some embodiments, a circular RNA comprises a salivirus IRES. See Sweeney et al., J Virol. 2012. 86(3):1468-1486. In some embodiments, the salivirus IRES is present in or derived from Salivirus FHB (SaliFHB). See GenBank Accession No. KM023140.1.

### Viral Vectors

Some aspects relate to viral vectors comprising or encoding influenza virus proteins. In some embodiments, the protein is comprised in a viral vector. In some embodiments, a viral vector comprises a nucleic acid encoding the protein.

Any suitable virus may be used as a viral vector. Non-limiting examples of viruses that may be used as viral vectors include retrovirus (e.g., lentivirus), adenovirus, adeno-associated virus (AAV), vesicular stomatitis virus (VSV), herpesvirus, Rous sarcoma virus, measles virus, poxvirus, gammavirus, alphavirus, murine stem cell virus, Moloney murine leukemia virus, and bovine leukemia virus. In some embodiments, the viral vector is a VSV vector. In some embodiments, the viral vector is a measles virus vector. In some embodiments, the viral vector is an adenovirus vector. These and other viral vectors suitable for expression of heterologous proteins (i.e., proteins not naturally expressed by a virus from which the viral vector is derived) are known in the art.

In some embodiments, a viral vector comprises an influenza B/Victoria lineage HA protein, or a nucleic acid encoding the influenza B/Victoria lineage HA protein. In some embodiments, the influenza B/Victoria lineage virus HA protein is an influenza B/Victoria lineage virus HA protein described in the section entitled “B/Victoria lineage HA proteins.” In some embodiments, the influenza B/Victoria lineage HA protein comprises an amino acid sequence with at least 90% sequence identity to the amino acid sequence of SEQ ID NO: 71.

In some embodiments, a viral vector comprises an influenza B/Yamagata lineage HA protein, or a nucleic acid encoding the influenza B/Yamagata lineage HA protein. In some embodiments, the influenza B/Yamagata lineage virus HA protein is an influenza B/Yamagata lineage virus HA protein described in the section entitled “B/Yamagata lineage HA proteins.” In some embodiments, the influenza B/Yamagata lineage HA protein comprises an amino acid sequence with at least 90% sequence identity to the amino acid sequence of SEQ ID NO: 70.

In some embodiments of viral vectors, a viral vector comprises an IAV H1 HA protein, or a nucleic acid encoding the IAV H1 HA protein. In some embodiments, the IAV H1 HA protein is an IAV H1 HA protein described in the section entitled “H1 HA proteins.” In some embodiments, the IAV H1 HA protein comprises an amino acid sequence with at least 90% sequence identity to the amino acid sequence of SEQ ID NO: 83. In some embodiments, the IAV H1 HA protein comprises an amino acid sequence with at least 90% sequence identity to the amino acid sequence of SEQ ID NO: 95.

In some embodiments, a viral vector comprises an IAV H3 HA protein, or a nucleic acid encoding the IAV H3 HA protein. In some embodiments, IAV H3 HA protein is an IAV H3 HA protein described in the section entitled “H3 HA proteins.” Where a combination or multivalent vaccine includes multiple H3 HA proteins (e.g., comprises multiple viral vectors comprising or encoding different H3 HA proteins), the different H3 HA proteins may each comprise the same substitutions, or different substitutions described in the section entitled “H3 HA proteins.” In some embodiments, one or more H3 HA proteins does not comprise a substitution described in the section entitled “H3 HA proteins.” In some embodiments, the IAV H3 HA protein comprises an amino acid sequence with at least 90% sequence identity to the amino acid sequence of SEQ ID NO: 82. In some embodiments, each IAV H3 HA protein comprises an amino acid sequence with at least 90% sequence identity to the amino acid sequence of SEQ ID NO: 82.

### Nucleic Acid Production

### In Vitro Transcription (IVT) of RNA

cDNA encoding RNA polynucleotides may be transcribed using an in vitro transcription (IVT) system. In vitro transcription of RNA is known in the art and is described in International Publication WO 2014/152027, which is incorporated by reference herein to the extent it discloses IVT methods. In some embodiments, the RNA is prepared in accordance with any one or more of the methods described in WO 2018/053209 and WO 2019/036682, each of which is incorporated by reference herein to the extent it discloses RNA production methods.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of an RNA polynucleotide, for example, but not limited to influenza virus mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

An in vitro transcription system typically comprises a transcription buffer, nucleotide triphosphates (NTPs), an RNase inhibitor, and a polymerase.

The NTPs may be manufactured in house, may be selected from a supplier, or may be synthesized. The NTPs may be selected from natural and unnatural NTPs, and may be selected from unmodified (e.g., ATP, GTP, UTP, CTP) or modified NTPs.

Any number of RNA polymerases or variants may be used to transcribe RNA. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7 mG(5′)ppp(5′)NlmpNp.

In some embodiments the RNA polymerase is a wild-type RNA polymerase. In some embodiments, the RNA polymerase is an RNA polymerase variant, such as those described in WO 2020/172239, incorporated herein by reference to the extent it describes RNA polymerase variants. RNA polymerase variants may include at least one amino acid substitution, relative to the wild-type (WT) RNA polymerase. A WT T7 RNA polymerase is represented by SEQ ID NO: 81. In some embodiments, the RNA polymerase is a variant RNA polymerase comprising the amino acid sequence of any one of SEQ ID NOs: 176-179.

### Purification

Purification of the nucleic acids may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNATM oligo-T capture probes (EXIQON® Inc, Vedbaek, Denmark); HPLC based purification methods such as, but not limited to, strong anion exchange HPLC, weak anion exchange HPLC, reverse phase HPLC (RP-HPLC), and hydrophobic interaction HPLC (HIC-HPLC); and/or tangential flow filtration. The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

### Lipid Compositions

In some embodiments, the nucleic acids are formulated as a lipid composition, such as a composition comprising a lipid nanoparticle, a liposome, and/or a lipoplex. In some embodiments, the lipid composition (e.g., lipid nanoparticle, liposome, and/or lipoplex) does not comprise protamine. In some embodiments, the lipid composition does comprise protamine. In some embodiments, nucleic acids are formulated as lipid nanoparticle (LNP) compositions. Lipid nanoparticles typically comprise ionizable lipid (e.g., ionizable amino lipid), non-cationic lipid (e.g., phospholipid), structural lipid (e.g., sterol), and PEG-modified lipid components along with the nucleic acid cargo of interest. The lipid nanoparticles can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises a molar ratio of 20-60% ionizable lipid, 5-25% non-cationic lipid, 25-55% structural lipid, and 0.5-15% PEG-modified lipid.

In some embodiments, the lipid nanoparticle comprises a molar ratio of 20-60% ionizable lipid, 5-30% non-cationic lipid, 10-55% structural lipid, and 0.5-15% PEG-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable lipid. In some embodiments, the lipid nanoparticle comprises 20 mol %, 30 mol %, 40 mol %, 50 mol %, or 60 mol % ionizable lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable lipid.

In some embodiments, the lipid nanoparticle comprises 45-55 mole percent (mol %) ionizable lipid. For example, lipid nanoparticle may comprise 45, 46, 47, 48, 49, 50, 51, 52, 53, 54, or 55 mol % ionizable lipid.

### Ionizable Lipids

In some embodiments, the ionizable lipid is a compound of Formula (IL*)

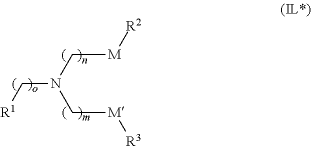

or a salt thereof, wherein:
R¹ is —OH, —NR^{N}—C₄₋₁₀ cycloalkenyl optionally substituted with one or more oxo or —N(R^{N}′R^{N″});
R^{N} is H or C₁₋₆ alkyl;
R^{N′} is H or C₁₋₆ alkyl;
R^{N″} is H or C₁₋₆ alkyl;
is 1, 2, 3, or 4;
n is 4, 5, 6, 7, or 8;
m is 4, 5, 6, 7, or 8;
M is-C(═O)—O—* or —O—C(═O)—*, wherein * indicates attachment to R²;
M′ is —C(═O)—O—* or —O—C(═O)—*, wherein * indicates attachment to R³;
R² is

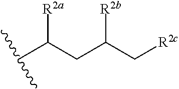

or —(C₁₋₆ alkylene)-(C₃₋₈ cycloalkyl)-C₁₋₆ alkyl;
R^{2a} is —H or C₁₋₁₀ alkyl;
R^{2b} is —H or C₁₋₁₀ alkyl;
R^{2c} is C₁₋₈ alkyl or C₂₋₈ alkenyl;
R³ is

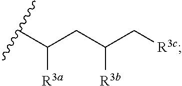

R^{3a} is H or C₁₋₁₀ alkyl;
R^{3b} is H or C₁₋₈ alkyl; and
R^{3c} is C₁₋₁₀ alkyl or C₂₋₈ alkenyl.

In some embodiments, the ionizable lipid is of Formula (IL**—I):

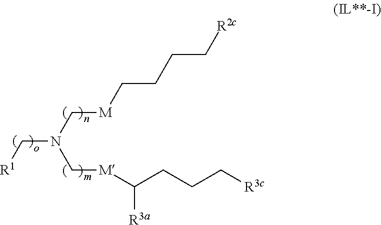

or a salt thereof, wherein:
R¹ is —OH;
is 2, 3, or 4;
n is 4, 5, 6, 7, or 8;
M is —C(═O)—O—*, wherein * indicates attachment to R²;
m is 6, 7, or 8;
M′ is —C(═O)—O—*, wherein * indicates attachment to R³;
R^{2c} is C₄₋₈ alkyl;
R^{3a} is C₇₋₁₀ alkyl; and
R^{3c} is C₃₋₅ alkyl.

In some embodiments, the ionizable lipid is of Formula (IL**—III):

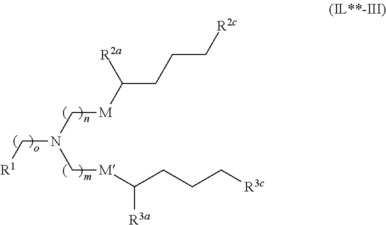

or a salt thereof, wherein:
R¹ is NR^{N}—C₄₋₁₀ cycloalkenyl optionally substituted with one or more oxo or —N(R^{N′}R^{N″});
   R^{N} is H;
   R^{N′} is C₁₋₂ alkyl;
   R^{N″} is H;
is 2, 3, or 4;
n is 6, 7, or 8;
M is —C(═O)—O—*, wherein * indicates attachment to R²;
m is 6, 7, or 8;
M′ is —C(═O)—O—*, wherein * indicates attachment to R³;
R^{2a} is C₇₋₁₀ alkyl;
R^{2c} is C₄₋₆ alkyl;
R^{3a} is C₁₋₃ alkyl; and
R^{3c} is C₄₋₆ alkyl.

In some embodiments, the ionizable lipid is of Formula (IL**—IV):

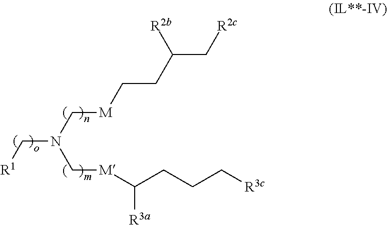

or a salt thereof, wherein:
R¹ is OH;
is 2, 3, or 4;
n is 6, 7, or 8;
M is —C(—O)—O—*, wherein * indicates attachment to R²;
m is 6, 7, or 8;
M′ is —C(═O)—O—*, wherein * indicates attachment to R³;
R^{2b} is C₃₋₅ alkyl;
R^{2c} is C₂₋₄ alkyl;
R^{3a} is C₇₋₁₀ alkyl; and
R^{3c} is C₄₋₆ alkyl.

In some embodiments, the ionizable lipid is of Formula (IL*—I):

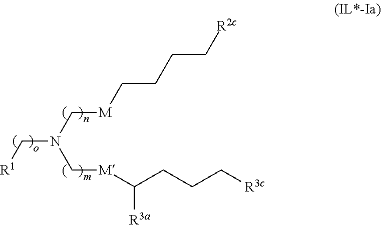

or a salt thereof, wherein:
R¹, o, m, n, M, M′, R^{2c}, and R^{3c} are as defined for variable IL*; and
R^{3a} is C₁₋₈ alkyl.

In some embodiments, ionizable lipid is of Formula (IL*-Ia):

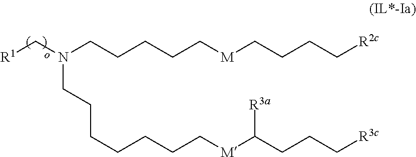

or a salt thereof, wherein:
R¹, o, m, n, M, M′, R^{2c}, and R^{3c} are as defined for Formula IL*; and
R^{3a} is C₁₋₈ alkyl.

In some embodiments, the ionizable lipid is of Formula (IL*-Ia′):

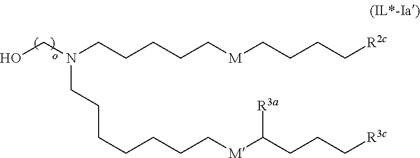

or a salt thereof, wherein:
o, M, M′, R^{2c} and R^{3c} are as defined for variable IL*; and
R^{3a} is C₁₋₈ alkyl.

In some embodiments, the ionizable lipid is of Formula (IL*-Iia):

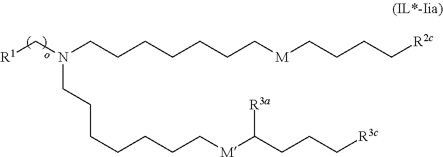

or a salt thereof, wherein:
R¹, o, m, n, M, M′, R^{2c}, and R^{3c} are as defined for Formula IL*; and
R^{3a} is C₁₋₈ alkyl.

In some embodiments, the ionizable lipid is of Formula (IL*-II′):

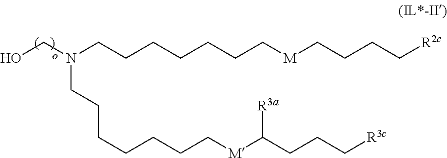

or a salt thereof, wherein:
o, M, M′, R^{2c} and R^{3c} are as defined for variable IL*; and
R^{3a} is C₁₋₈ alkyl.

In some embodiments, the ionizable lipid is of Formula (IL*—III):

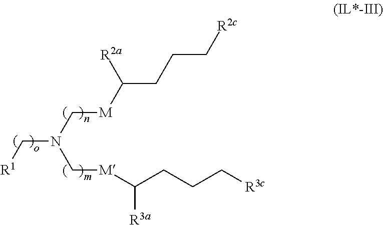

or a salt thereof, wherein:
R¹, o, m, n, M, M′, R^{2c}, and R^{3c} are as defined for variable IL*;
R^{2a} is a C₁₋₈ alkyl; and
R^{3a} is C₁₋₈ alkyl.

In some embodiments, the ionizable lipid is of Formula (IL*-IIIa):

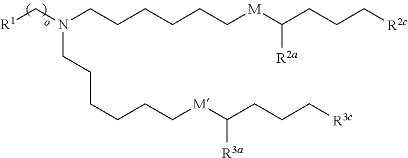

or a salt thereof, wherein:
R¹, o, m, n, M, M′, R^{2c}, and R^{3e} are as defined for variable IL*;
R^{2b} is a C₁₋₈ alkyl; and
R^{3a} is C₁₋₈ alkyl.

In some embodiments, the ionizable lipid is of Formula (IL*-IIIa):

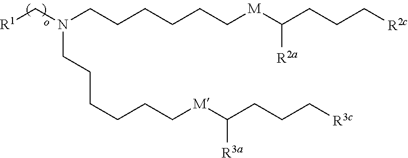

or a salt thereof, wherein:
R¹, o, M, M′, R^{2c}, and R^{3c} are as defined for variable IL*;
R^{2a} is a C₁₋₈ alkyl; and
R^{3a} is C₁₋₈ alkyl.

In some embodiments, the ionizable lipid is of Formula (IL*-IIIa′):

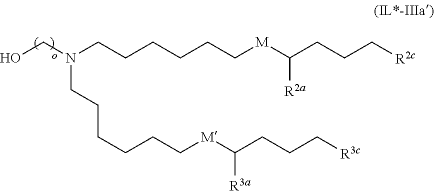

or a salt thereof, wherein:
R¹, o, M, M′, R^{2c}, and R^{3c} are as defined for variable IL*;
R^{2a} is a C₁₋₈ alkyl; and
R^{3a} is C₁₋₈ alkyl.

In some embodiments, the ionizable lipid is of Formula (IL*-IIIb):

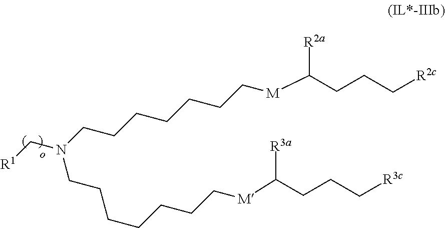

or a salt thereof, wherein:
R¹, o, M, M′, R^{2c}, and R^{3c} are as defined for variable IL*;
R^{2a} is a C₁₋₈ alkyl; and
R^{3a} is C₁₋₈ alkyl.

In some embodiments, the ionizable lipid is of Formula (IL*-IIIb′):

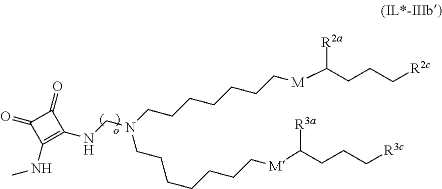

or a salt thereof, wherein:
R¹, o, M, M′, R^{2c}, and R^{3c} are as defined for variable IL*;
R^{2a} is a C₁₋₈ alkyl; and
R^{3a} is C₁₋₈ alkyl.

In some embodiments, the ionizable lipid is of Formula (IL*—IV):

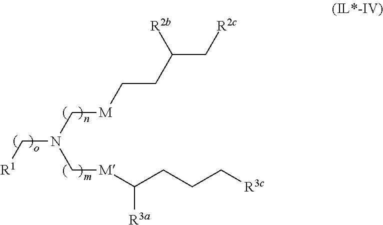

or a salt thereof, wherein:
R¹, o, m, n, M, M′, R^{2c}, and R^{3c} are as defined for variable IL*;
R^{2b} is a C₁₋₈ alkyl; and
R^{3a} is C₁₋₈ alkyl.

In some embodiments, the ionizable lipid is of Formula (IL*-Iva):

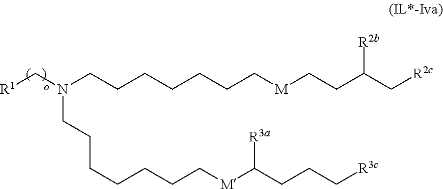

or a salt thereof, wherein:
R¹, o, m, n, M, M′, R^{2c}, and R^{3c} are as defined for variable IL*;
R^{2b} is a C₁₋₈ alkyl; and
R^{3a} is C₁₋₈ alkyl.

In some embodiments, the ionizable lipid is of Formula (IL*-Iva′):

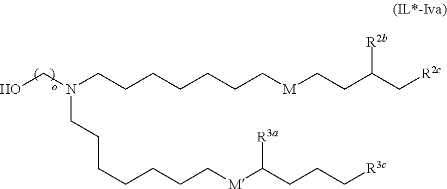

or a salt thereof, wherein:
o, M, M′, R^{2c}, and R^{3c} are as defined for variable IL*;
R^{2a} is a C₁₋₈ alkyl; and
R^{3a} is C₁₋₈ alkyl.
   Variables o, R¹, R^{N}, R^{N′}, R^{N″} of Ionizable Lipid

In some embodiments of the ionizable lipid, o is 1.

In some embodiments of the ionizable lipid, o is 2.

In some embodiments of the ionizable lipid, o is 3.

In some embodiments of the ionizable lipid, o is 4.

In some embodiments of the ionizable lipid, R¹ is —OH.

In some embodiments of the ionizable lipid, R^{N} is H.

In some embodiments of the ionizable lipid, R^{N} is methyl.

In some embodiments of the ionizable lipid, R^{N} is ethyl.

In some embodiments of the ionizable lipid, R¹ is —NR^{N}-cyclobutenyl, wherein the cyclobutenyl is optionally substituted with one or more oxo or —N(R^{N′}R^{N″}).

In some embodiments of the ionizable lipid, R^{N′} is H.

In some embodiments of the ionizable lipid, R^{N′} is methyl.

In some embodiments of the ionizable lipid, R^{N′} is ethyl.

In some embodiments of the ionizable lipid, R^{N″} is H.

In some embodiments of the ionizable lipid, R^{N″} is methyl.

In some embodiments of the ionizable lipid, R^{N″} is ethyl.

In some embodiments of the ionizable lipid, R^{N′} is H and R^{N″} is methyl.

In some embodiments of the ionizable lipid, R¹ is

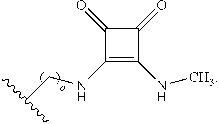

In some embodiments of the ionizable lipid, R¹ is

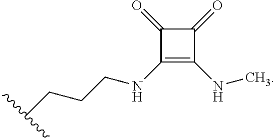 [SMILES: *CCCNc1c(NC)c(=O)c1=O]

### Variables m and n of the Ionizable Lipid

In some embodiments of the ionizable lipid, m is 4.

In some embodiments of the ionizable lipid, m is 5.

In some embodiments of the ionizable lipid, m is 6.

In some embodiments of the ionizable lipid, m is 7.

In some embodiments of the ionizable lipid, m is 8.

In some embodiments of the ionizable lipid, m is 4.

In some embodiments of the ionizable lipid, n is 5.

In some embodiments of the ionizable lipid, n is 6.

In some embodiments of the ionizable lipid, n is 7.

In some embodiments of the ionizable lipid, n is 8.

In some embodiments of the ionizable lipid, n is 5 and m is 7.

In some embodiments of the ionizable lipid, n is 7 and m is 7.

In some embodiments of the ionizable lipid, m is 6 and n is 6.

### Variables M and M′ of Ionizable Lipid

In some embodiments of the ionizable lipid, M is —O—C(═O)—*, wherein * indicates attachment to R².

In some embodiments of the ionizable lipid, M is —C(—O)—O—* wherein * indicates attachment to R².

In some embodiments of the ionizable lipid, M′ is —O—C(═O)—*, wherein * indicates attachment to R³.

In some embodiments of the ionizable lipid, M′ is —C(═O)—O—* wherein * indicates attachment to R³.

In some embodiments of the ionizable lipid, M is —O—C(═O)—*, wherein * indicates attachment to R², and M′ is —C(—O)—O—* wherein * indicates attachment to R³

Variables R², R^{2a}, R^{2b}, R^{2c} of Ionizable Lipid

In some embodiments of the ionizable lipid, R² is

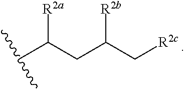

In some embodiments of the ionizable lipid, R^{2a} is hydrogen.

In some embodiments of the ionizable lipid, R^{2a} is methyl.

In some embodiments of the ionizable lipid, R^{2a} is ethyl.

In some embodiments of the ionizable lipid, R^{2a} is propyl.

In some embodiments of the ionizable lipid, R^{2a} is butyl.

In some embodiments of the ionizable lipid, R^{2a} is pentyl.

In some embodiments of the ionizable lipid, R^{2a} is hexyl.

In some embodiments of the ionizable lipid, R^{2a} is heptyl.

In some embodiments of the ionizable lipid, R^{2a} is octyl.

In some embodiments of the ionizable lipid, R^{2b} is hydrogen.

In some embodiments of the ionizable lipid, R^{2b} is methyl.

In some embodiments of the ionizable lipid, R^{2b} is ethyl.

In some embodiments of the ionizable lipid, R^{2b} is propyl.

In some embodiments of the ionizable lipid, R^{2b} is butyl.

In some embodiments of the ionizable lipid, R^{2b} is pentyl.

In some embodiments of the ionizable lipid, R^{2b} is hexyl.

In some embodiments of the ionizable lipid, R^{2b} is heptyl.

In some embodiments of the ionizable lipid, R^{2b} is octyl.

In some embodiments of the ionizable lipid, R^{2a} is hydrogen and R^{2b} is hydrogen.

In some embodiments of the ionizable lipid, R^{2a} is hexyl and R^{2b} is hydrogen.

In some embodiments of the ionizable lipid, R^{2a} is octyl and R^{2b} is hydrogen.

In some embodiments of the ionizable lipid, R^{2a} is hydrogen and R^{2b} is butyl.

In some embodiments of the ionizable lipid, R^{2c} is methyl.

In some embodiments of the ionizable lipid, R^{2c} is ethyl.

In some embodiments of the ionizable lipid, R^{2c} is propyl.

In some embodiments of the ionizable lipid, R^{2c} is butyl.

In some embodiments of the ionizable lipid, R^{2c} is pentyl.

In some embodiments of the ionizable lipid, R^{2c} is hexyl.

In some embodiments of the ionizable lipid, R^{2c} is heptyl.

In some embodiments of the ionizable lipid, R^{2c} is octyl.

In some embodiments of the ionizable lipid, R² is —(C₁₋₆ alkylene)-(C₃₋₈ cycloalkyl)-C₁₋₆ alkyl.

In some embodiments of the ionizable lipid, R² is —(C₁₋₆ alkylene)-(cyclohexyl)-C₁₋₆ alkyl.

In some embodiments of the ionizable lipid, R² is —(C₁₋₆ alkylene)-(cyclopentyl)-C₁₋₆ alkyl.

Variables R³, R^{3a}, R^{3b}, and R^{3c} of Ionizable Lipid

In some embodiments of the ionizable lipid, R³ is

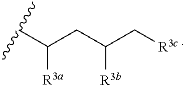

In some embodiments of the ionizable lipid, R^{3a} is hydrogen.

In some embodiments of the ionizable lipid, R^{3a} is methyl.

In some embodiments of the ionizable lipid, R^{3a} is ethyl.

In some embodiments of the ionizable lipid, R^{3a} is propyl.

In some embodiments of the ionizable lipid, R^{3a} is butyl.

In some embodiments of the ionizable lipid, R^{3a} is pentyl.

In some embodiments of the ionizable lipid, R^{3a} is hexyl.

In some embodiments of the ionizable lipid, R^{3a} is heptyl.

In some embodiments of the ionizable lipid, R^{3a} is octyl.

In some embodiments of the ionizable lipid, R^{3b} is hydrogen.

In some embodiments of the ionizable lipid, R^{3b} is methyl.

In some embodiments of the ionizable lipid, R^{3b} is ethyl.

In some embodiments of the ionizable lipid, R^{3b} is propyl.

In some embodiments of the ionizable lipid, R^{3b} is butyl.

In some embodiments of the ionizable lipid, R^{3b} is pentyl.

In some embodiments of the ionizable lipid, R^{3b} is hexyl.

In some embodiments of the ionizable lipid, R^{3b} is heptyl.

In some embodiments of the ionizable lipid, R^{3b} is octyl.

In some embodiments of the ionizable lipid, R^{3a} is octyl and R^{3b} is hydrogen.

In some embodiments of the ionizable lipid, R^{3a} is ethyl and R^{3b} is hydrogen.

In some embodiments of the ionizable lipid, R^{3a} is hexyl and R^{3b} is hydrogen.

In some embodiments of the ionizable lipid, R^{3c} is methyl.

In some embodiments of the ionizable lipid, R^{3c} is ethyl.

In some embodiments of the ionizable lipid, R^{3c} is propyl.

In some embodiments of the ionizable lipid, R^{3c} is butyl.

In some embodiments of the ionizable lipid, R^{3c} is pentyl.

In some embodiments of the ionizable lipid, R^{3c} is hexyl.

In some embodiments of the ionizable lipid, R^{3c} is heptyl.

In some embodiments of the ionizable lipid, R^{3c} is octyl.

It is understood that, for an ionizable lipid, variables o, R¹, R^{N}, R^{N′}, R^{N′}, m, n, M, M′, R², R^{2a}, R^{2b}, R^{2c}, R³, R^{3a}, R^{3b}, and R^{3c} can each be, where applicable, selected from the groups described herein, and any group described herein for any of variables o, R¹, R^{N}, R^{N′}, R^{N′}, m, n, M, M′, R², R^{2a}, R^{2b}, R^{2c}, R³, R^{3a}, R^{3b}, and R^{3c} can be combined, where applicable, with any group described herein for one or more of the remainder of variables o, R¹, R^{N}, R^{N′}, R^{N′}, m, n, M, M′, R², R^{2a}, R^{2b}, R^{2c}, R³, R^{3a}, R^{3b}, and R^{3c}.

In some embodiments, the ionizable lipid is a compound selected from:

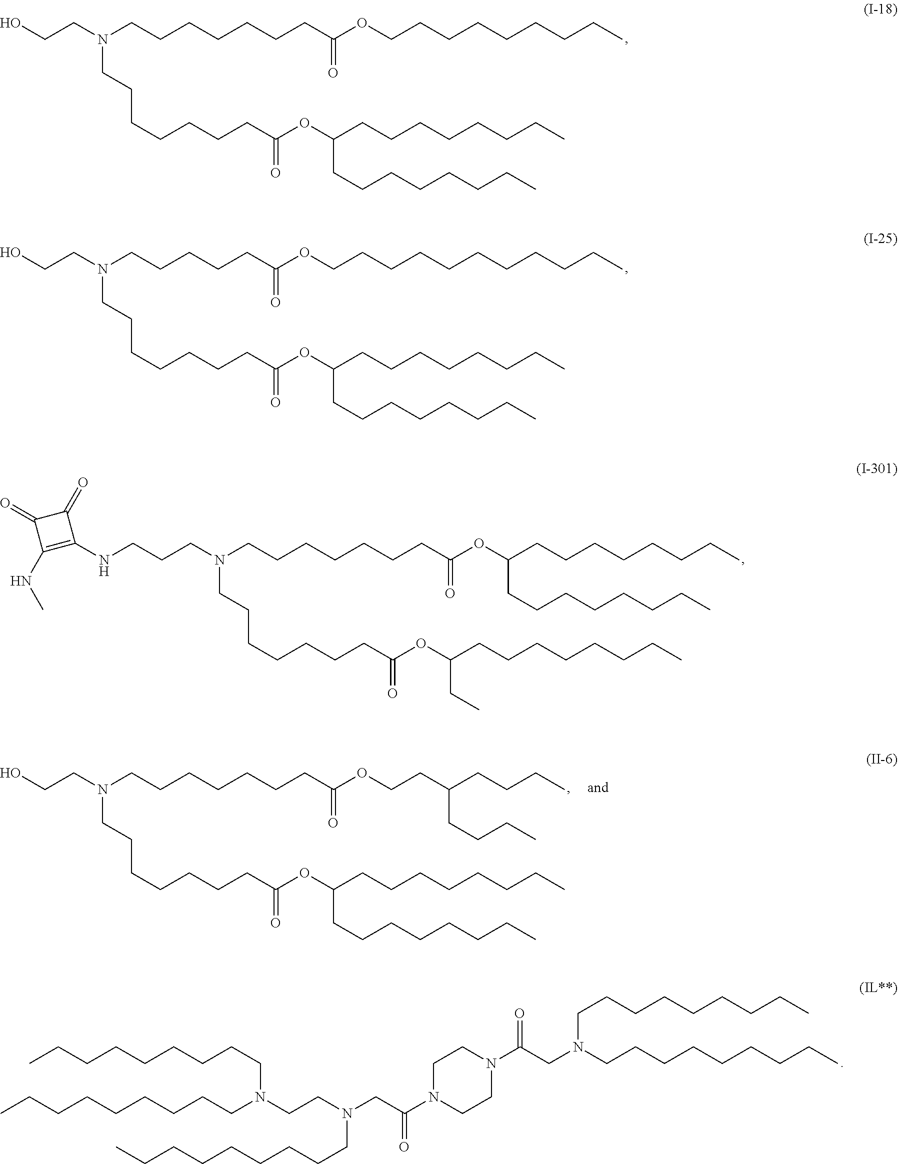

In some embodiments, the ionizable lipid is

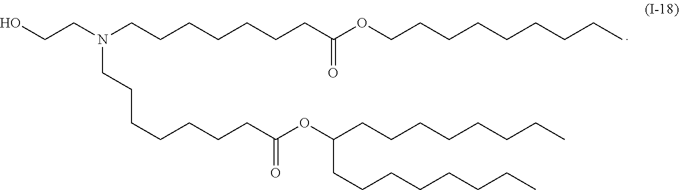 [SMILES: CCCCCCCCCOC(=O)CCCCCCCN(CCO)CCCCCCCC(=O)OC(CCCCCCCC)CCCCCCCC]

In some embodiments, the ionizable lipid is

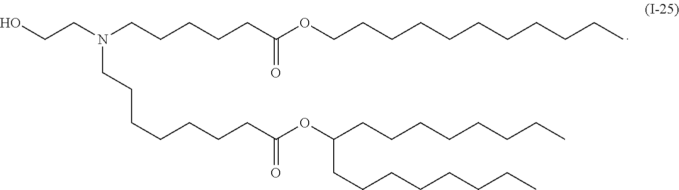 [SMILES: CCCCCCCCCCCOC(=O)CCCCCN(CCO)CCCCCCCC(=O)OC(CCCCCCCC)CCCCCCCC]

In some embodiments, the ionizable lipid is

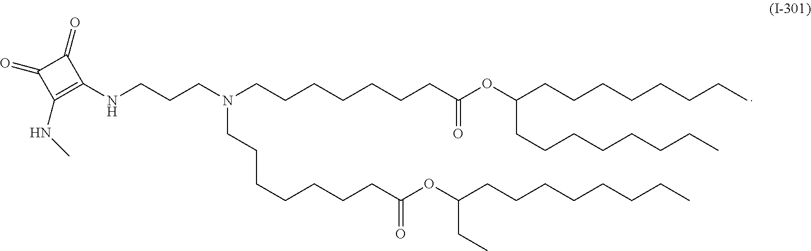 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the ionizable lipid is

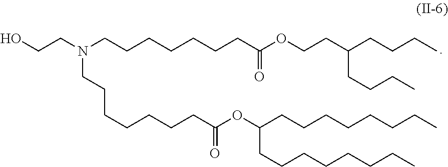 [SMILES: CCCCCCCCC(CCCCCCCC)OC(=O)CCCCCCCN(CCO)CCCCCCCC(=O)OCCC(CCCC)CCCC]

Without wishing to be bound by theory, it is understood that an ionizable lipid may have a positive or partial positive charge at physiological pH. Such lipids may be referred to as cationic or ionizable (amino) lipids. Lipids may also be zwitterionic, i.e., neutral molecules having both a positive and a negative charge.

### Formula (AI)

In some embodiments, the ionizable amino lipid of a lipid nanoparticle is a compound of Formula (AI):

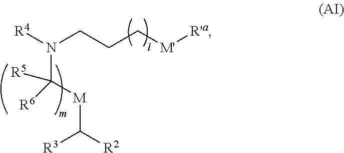

or its N-oxide, or a salt or isomer thereof,
wherein R′^{a} is R′^{branched}; wherein
R′^{branched} is:

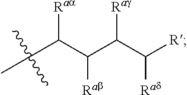

wherein

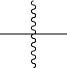

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

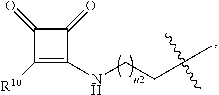

wherein

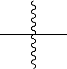

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (AI), R′^{a} is R′^{branched}; R′^{branched} is

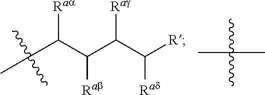

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments of the compounds of Formula (AI), R′^{a} is R′^{branched}; R′^{branched} is

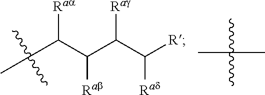

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 3; and m is 7.

In some embodiments of the compounds of Formula (AI), R′^{a} is R′^{branched}; R′^{branched} is

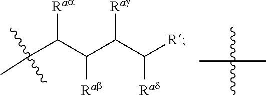

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

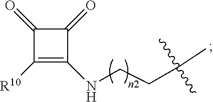

R¹⁰NH(C₁₋₆ alkyl); n2 is 2; R⁵ is H; each R⁶ is H; M and M′ are each —C(O)O—; R′ is a C₁₋₁₂ alkyl; l is 5; and m is 7.

In some embodiments of the compounds of Formula (AI), R′^{a} is R′^{branched}; R′^{branched} is

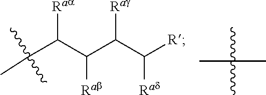

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments, the compound of Formula (AI) is selected from:

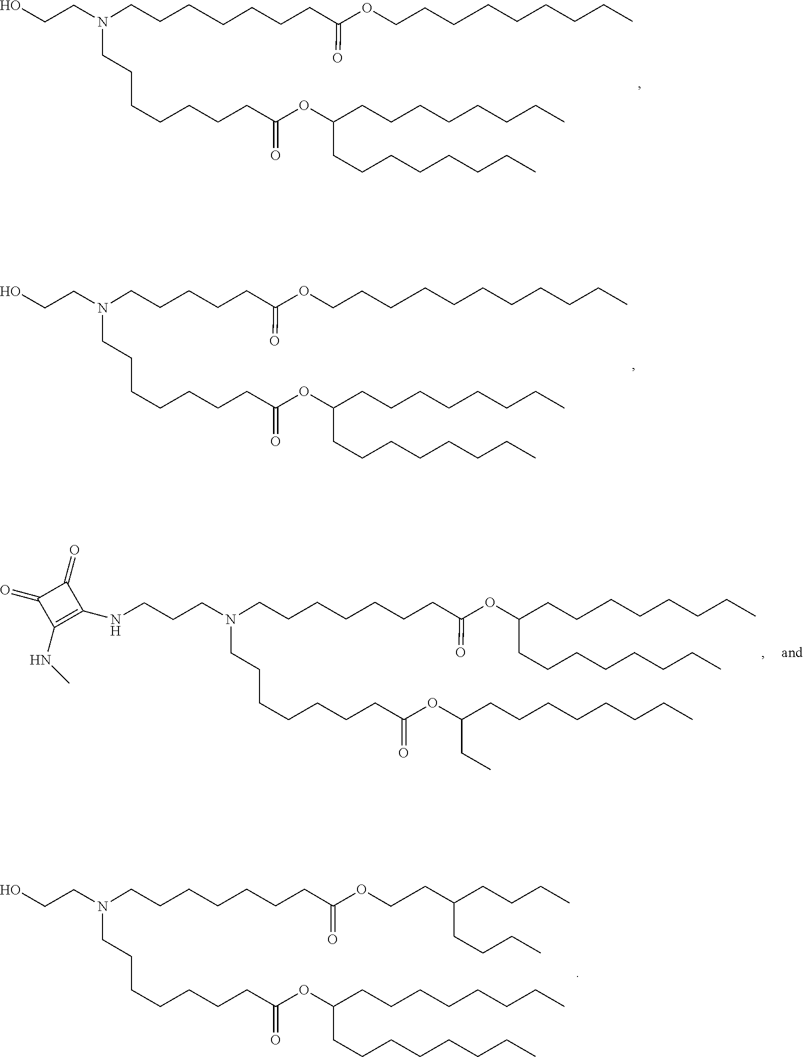 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the ionizable amino lipid of Formula (AI) is a compound of Formula (AIa):

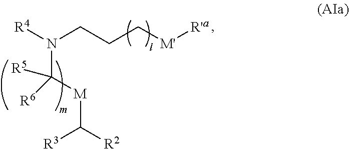

or its N-oxide, or a salt or isomer thereof,
wherein R′^{a} is R′^{branched}, wherein
R′^{branched} is:

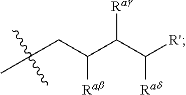

wherein

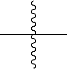

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

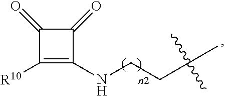

wherein

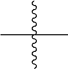

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid of Formula (AI) is a compound of Formula (AIb):

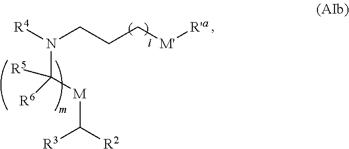

or its N-oxide, or a salt or isomer thereof,
wherein R′^{a} is R′^{branched}, wherein
R′^{branched} is:

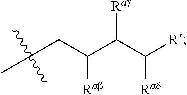

wherein

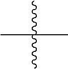

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (AI) or (AIb), R′^{a} is R′^{branched}; R′^{branched} is

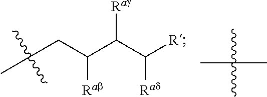

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments of Formula (AI) or (AIb), R′^{a} is R′^{branched}; R′^{branched} is

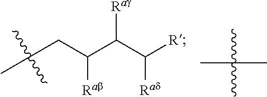

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 3; and m is 7.

In some embodiments of Formula (AI) or (AIb), R′^{a} is R′^{branched}; R′^{branched} is

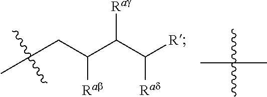

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments, the ionizable amino lipid of Formula (AI) is a compound of Formula (AIc):

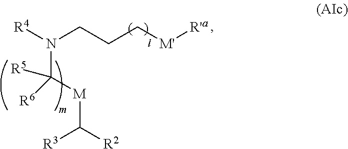

or its N-oxide, or a salt or isomer thereof,
wherein R′^{a} is R′^{branched}, wherein
R′^{branched} is:

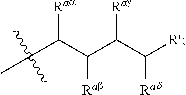

wherein

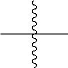

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

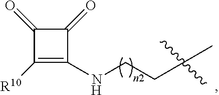

wherein

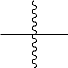

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}, R′^{branched} is

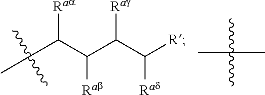

denotes a point of attachment; R^{aβ}, R^{aγ}, and R^{aδ} are each H; R^{aδ} is C₂₋₁₂ alkyl; R² and R³ are each C₁₋₁₄ alkyl; R⁴ is

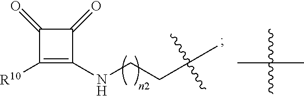

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; l is 5; and m is 7.

In some embodiments, the compound of Formula (AIc) is:

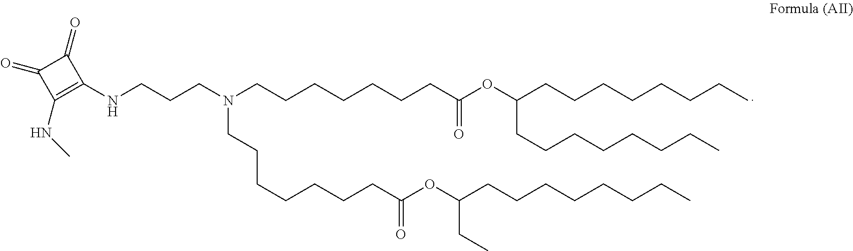 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the ionizable amino lipid is a compound of Formula (AII):

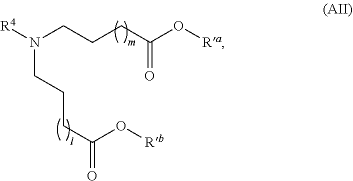

or its N-oxide, or a salt or isomer thereof,
wherein R′^{a} is R′^{branched} or R′ cyclic, wherein
R′^{branched} is:

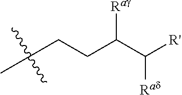

and R′ cyclic is:

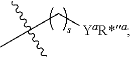

and
′^{b} is:

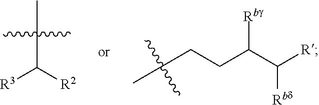

wherein

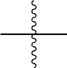

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

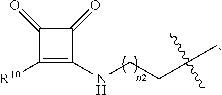

wherein

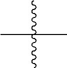

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-a):

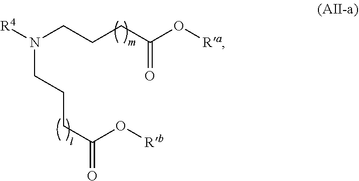

or its N-oxide, or a salt or isomer thereof,
wherein R′^{a} is R′^{branched} or R′ cyclic, wherein
R′^{branched} is:

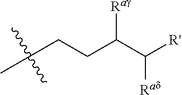

### and R′b is:

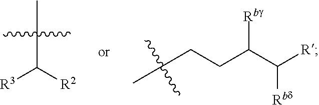

wherein

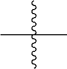

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

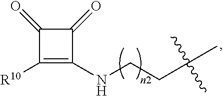

wherein

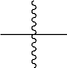

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-b):

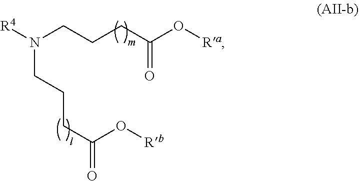

or its N-oxide, or a salt or isomer thereof,
wherein R′^{a} is R′^{branched} or R′^{cyclic}; wherein
R′^{branched} is:

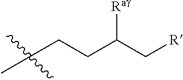

### and R′b is:

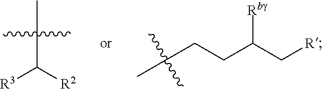

wherein

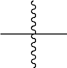

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

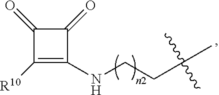

wherein

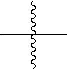

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-c):

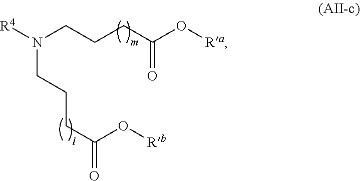

or its N-oxide, or a salt or isomer thereof,
wherein R′^{a} is R′^{branched} or R′^{cyclic}; wherein
R′^{branched} is:

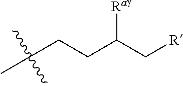

### and R′b is:

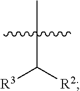

wherein

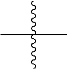

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

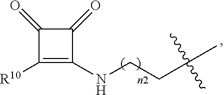

wherein

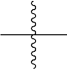

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-d):

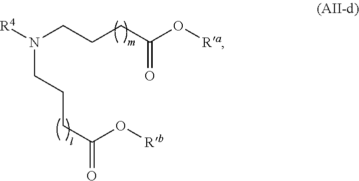

or its N-oxide, or a salt or isomer thereof, wherein R′^{a} is R′^{branched} or R′^{cyclic}; wherein
R′^{branched} is:

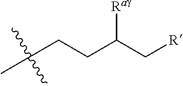

### and R′b is:

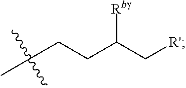

wherein

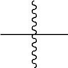

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

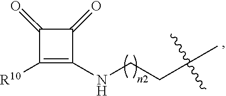

wherein

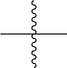

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid of Formula (AII) is a compound of Formula (AII-e):

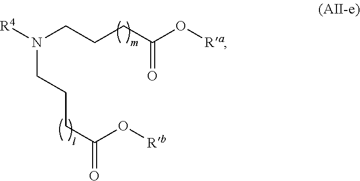

or its N-oxide, or a salt or isomer thereof,
wherein R′^{a} is R′^{branched} or R′^{cyclic}; wherein
R′^{branched} is:

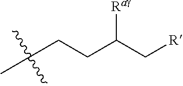

### and R′b is:

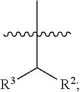

wherein

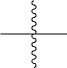

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (AII), (AII-a), (AII-b), (AII-c), (AII-d), or (AII-e), m and l are each independently selected from 4, 5, and 6. In some embodiments of the compound of Formula (AII), (AII-a), (AII-b), (AII-c), (AII-d), or (AII-e), m and l are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C₂₋₅ alkyl.

In some embodiments of the compound of Formula (AII), (AII-a), (AII-b), (AII-c), (AII-d), or (AII-e), R′b is:

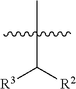

and R² and R³ are each independently a C₁₋₁₄ alkyl. In some embodiments of the compound of Formula (AII), (AII-a), (AII-b), (AII-c), (AII-d), or (AII-e), R′^{b} is:

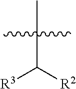

and R² and R³ are each independently a C₆₋₁₀ alkyl. In some embodiments of the compound of Formula (AII), (AII-a), (AII-b), (AII-c), (AII-d), or (AII-e), R′^{b} is:

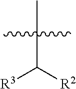

and R² and R³ are each a C₈ alkyl.

In some embodiments of the compound of Formula (AII), (AII-a), (AII-b), (AII-c), (AII-d), or (AII-e), R′^{branched} is:

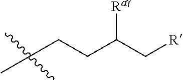

### and R′^{b} is:

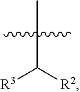

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

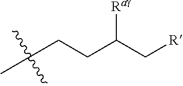

### and R′^{b} is:

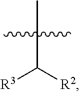

R^{aγ} is a C₂₋₆ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

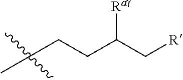

### and R′^{b} is:

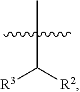

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

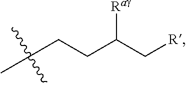

### R′^{b} is:

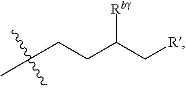

and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

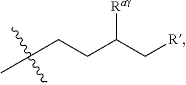

### R′^{b} is:

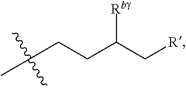

and R^{aγ} and R^{bγ} are each a C₂₋₆ alkyl.

In some embodiments of the compound of Formula (AII), (AII-a), (AII-b), (AII-c), (AII-d), or (AII-e), m and l are each independently selected from 4, 5, and 6 and each R′ independently is a C₁₋₁₂ alkyl. In some embodiments of the compound of Formula (AII), (AII-a), (AII-b), (AII-c), (AII-d), or (AII-e), m and l are each 5 and each R′ independently is a C₂₋₅ alkyl.

In some embodiments of the compound of (AII), (AII-a), (AII-b), (AII-c), (AII-d), or (AII-e), R′^{branched} is:

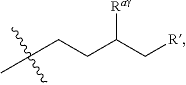

### R′^{b} is:

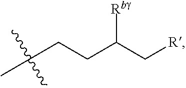

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

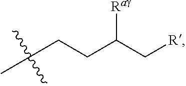

### R′^{b} is:

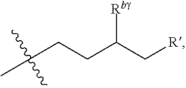

m and l are each 5, each R′ independently is a C₂₋₅ alkyl, and R^{aγ} and R^{bγ} are each a C₂₋₆ alkyl.

In some embodiments of the compound of Formula (AII), (AII-a), (AII-b), (AII-c), (AII-d), or (AII-e), R′^{branched} is:

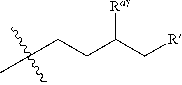

### and R′^{b} is:

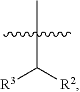

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

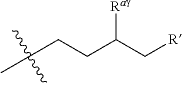

### and R′^{b} is:

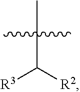

m and l are each 5, R′ is a C₂₋₅ alkyl, R^{aγ} is a C₂₋₆ alkyl, and R² and R³ are each a C₈ alkyl.

In some embodiments of the compound of (AII), (AII-a), (AII-b), (AII-c), (AII-d), or (AII-e), R⁴ is

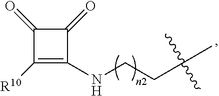

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

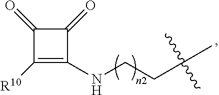

wherein R¹⁰ is NH(CH₃) and n2 is 2.

In some embodiments of the compound of Formula (AII), (AII-a), (AII-b), (AII-c), (AII-d), or (AII-e), R′^{branched} is:

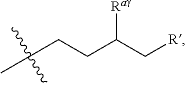

### R′^{b} is:

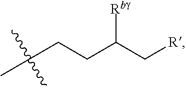

m and l are each independently selected from 4, 5, and 6, each R′ independently is a C₁₋₁₂ alkyl, R^{aγ} and R^{bγ} are each a C₁₋₁₂ alkyl, and R⁴ is

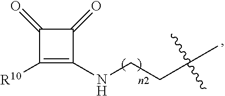

wherein R¹⁰ is NH(C₁₋₆ alkyl), and n2 is 2. In some embodiments of the compound of Formula (AII), (AII-a), (AII-b), (AII-c), (AII-d), or (AII-e), R′^{branched} is:

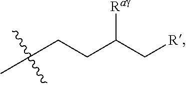

### R′^{b} is:

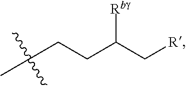

m and l are each 5, each R′ independently is a C₂₋₅ alkyl, R^{aγ} and R^{bγ} are each a C₂₋₆ alkyl, and R⁴ is

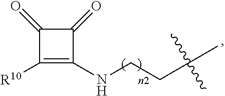

wherein R¹⁰ is NH(CH₃) and n2 is 2.

In some embodiments of the compound of Formula (AII), (AII-a), (AII-b), (AII-c), (AII-d), or (AII-e), R′^{branched} is:

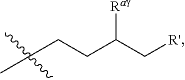

### and R′^{b} is:

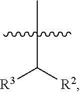

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

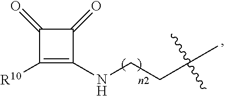

wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is:

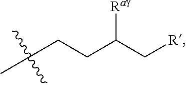

### and R′^{b} is:

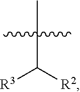

m and l are each 5, R′ is a C₂₋₅ alkyl, R^{aγ} is a C₂₋₆ alkyl, R² and R³ are each a C₈ alkyl, and R⁴ is

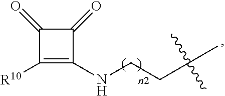

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂)ₙOH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

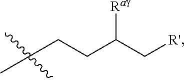

### R′^{b} is:

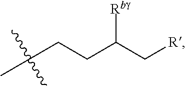

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AII), (AII-a), (AII-b), (AII-c), (AII-d), or (AII-e), R′^{branched} is:

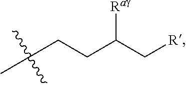

### R′^{b} is:

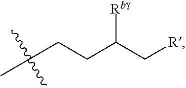

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid of Formula (AII) is a compound of Formula (AII-f):

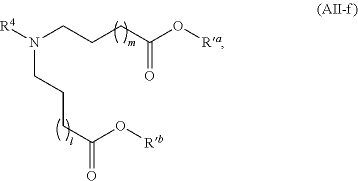

or its N-oxide, or a salt or isomer thereof,
wherein R′^{a} is R′^{branched} or R′^{cyclic}; wherein
R′^{branched} is:

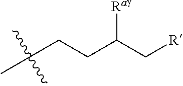

### and R′^{b} is:

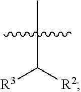

wherein

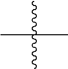

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.
In some embodiments of the compound of Formula (AII-f), m and l are each 5, and n is 2, 3, or 4.

In some embodiments of the compound of Formula (AII-f) R′ is a C₂₋₅ alkyl, R^{aγ} is a C₂₋₆ alkyl, and R² and R³ are each a C₆₋₁₀ alkyl.

In some embodiments of the compound of Formula (AII-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the ionizable amino lipid of Formula (AII) is a compound of Formula (AII-g):

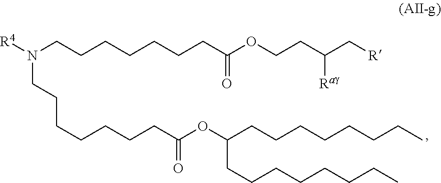

or its N-oxide, or a salt or isomer thereof; wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

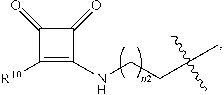

wherein

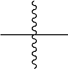

denotes a point of attachment, R¹⁰ is NH(C₁₋₆ alkyl), and n2 is selected from the group consisting of 1, 2, and 3.

In some embodiments, the ionizable amino lipid of Formula (AII) is a compound of Formula (AII-h):

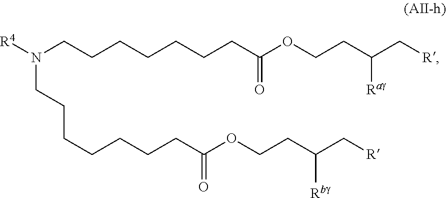

or its N-oxide, or a salt or isomer thereof; wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

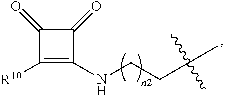

wherein

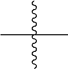

denotes a point of attachment, R¹⁰ is NH(C₁₋₆ alkyl), and n2 is selected from the group consisting of 1, 2, and 3.

In some embodiments of the compound of Formula (AII-g) or (AII-h), R⁴ is

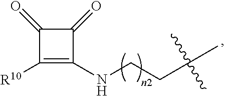

wherein
R¹⁰ is NH(CH₃) and n2 is 2.

In some embodiments of the compound of Formula (AII-g) or (AII-h), R⁴ is —(CH₂)₂OH.

### Formula (AIII)

In some embodiments, the ionizable amino lipids of a lipid nanoparticle may be one or more of compounds of Formula (AIII):

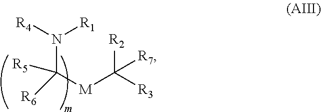

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected
from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which:

R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N (R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(—CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR,
—CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (AIII) includes those in which

R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In certain embodiments, a subset of compounds of Formula (AIII) includes those of Formula (AIII-A):

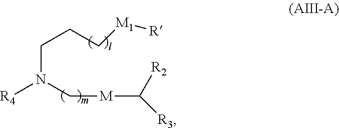

or its N-oxide, or a salt or isomer thereof, wherein l is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is
—OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl;
M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and
R₂ and R₃ are independently selected from the group consisting of H, C₁₋₁₄ alkyl, and C₂₋₁₄ alkenyl.

For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (AIII) includes those of Formula (AIII-B):

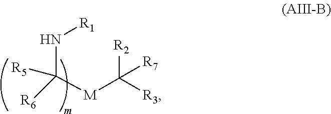

or its N-oxide, or a salt or isomer thereof in which all variables are as defined herein. For example, m is selected from 5, 6, 7, 8, and 9; R⁴ is hydrogen, unsubstituted C₁₋₃ alkyl, or —(CH₂)ₙQ, in which Q is
H, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl;
M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and
R₂ and R₃ are independently selected from the group consisting of H, C₁₋₁₄ alkyl, and C₂₋₁₄ alkenyl.

For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (AIII) includes those of Formula (AIII-C):

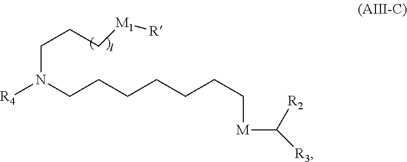

or its N-oxide, or a salt or isomer thereof, wherein l is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl;
M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and
R₂ and R₃ are independently selected from the group consisting of H, C₁₋₁₄ alkyl, and
C₂₋₁₄ alkenyl.

In some embodiments, the compounds of Formula (AIII) are of Formula (AIII-D),

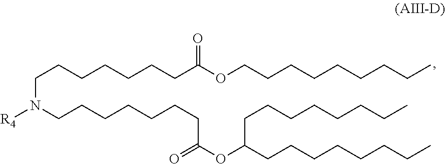

or their N-oxides, or salts or isomers thereof, wherein R₄ is as described in this Lipid Compositions section.

In another embodiment, the compounds of Formula (AIII) are of Formula (AIII-E),

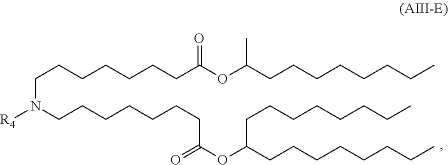

or their N-oxides, or salts or isomers thereof, wherein R₄ is as described in this in this Lipid Compositions section.

In another embodiment, the compounds of Formula (AIII) are of Formula (AIII-F) or (AIII-G):

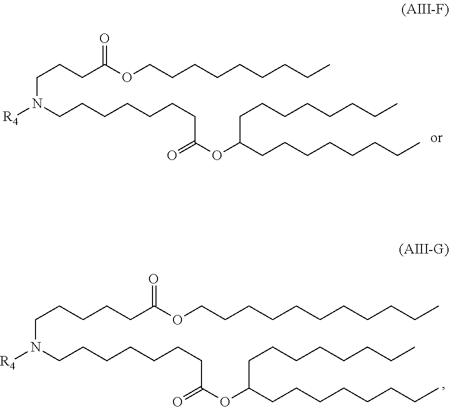

or their N-oxides, or salts or isomers thereof, wherein R₄ is as described in this in this Lipid Compositions section.

In another embodiment, the compounds of Formula (AIII) are of Formula (AIII-H):

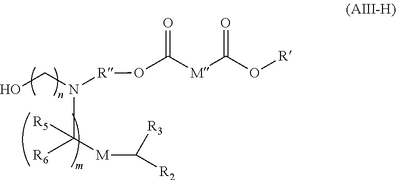

or their N-oxides, or salts or isomers thereof,
wherein M is —C(O)O— or —OC(O)—, M″ is C₁₋₆ alkyl or C₂₋₆ alkenyl, R² and R³ are independently selected from the group consisting of C₅₋₁₄ alkyl and C₅₋₁₄ alkenyl, and n is selected from 2, 3, and 4.

In a further embodiment, the compounds of Formula (AIII) are of Formula (AIII-I):

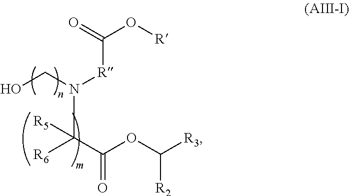

or their N-oxides, or salts or isomers thereof, wherein n is 2, 3, or 4; and m, R′, R″, and R₂ through R₆ are as described in this in this Lipid Compositions section. For example, each of R₂ and R₃ may be independently selected from the group consisting of C₅₋₁₄ alkyl and C₅₋₁₄ alkenyl.

In some embodiments, an ionizable amino lipid of comprises a compound having structure:

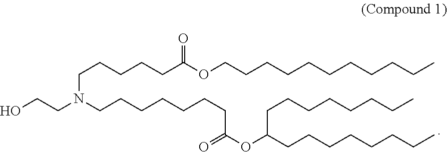 [SMILES: CCCCCCCCCCCOC(=O)CCCCCN(CCO)CCCCCCCC(=O)OC(CCCCCCCC)CCCCCCCC]

In some embodiments, an ionizable amino lipid comprises a compound having structure:

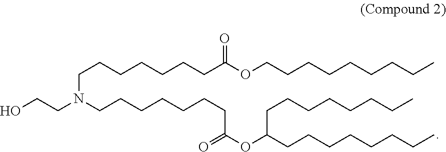 [SMILES: CCCCCCCCCOC(=O)CCCCCCCN(CCO)CCCCCCCC(=O)OC(CCCCCCCC)CCCCCCCC]

In a further embodiment, the compounds of Formula (AIII) are of Formula (AIII-J),

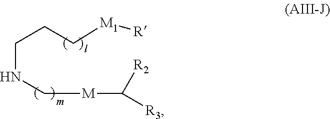

or their N-oxides, or salts or isomers thereof,
wherein l is selected from 1, 2, 3, 4, and 5; m is selected from 5, 6, 7, 8, and 9; M₁ is a bond or M′; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

The central amine moiety of a lipid according to Formula (AIII), (AIII-A), (AIII-B), (AIII-C), (AIII-D), (AIII-E), (AIII-F), (AIII-G), (AIII-H), (AIII-I), or (AIII-J) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

### Formula (AIV)

In some embodiments, the ionizable amino lipids of a lipid nanoparticle may be one or more of compounds of formula (AIV),

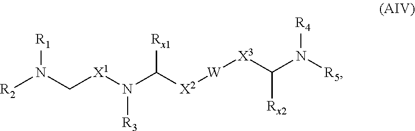

or salts or isomers thereof, wherein
W is

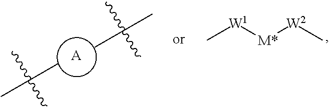

ring A is

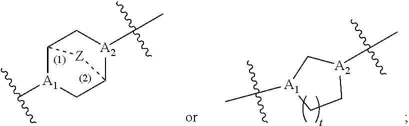

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

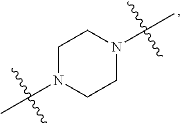 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (AIVa)-(AIVh):

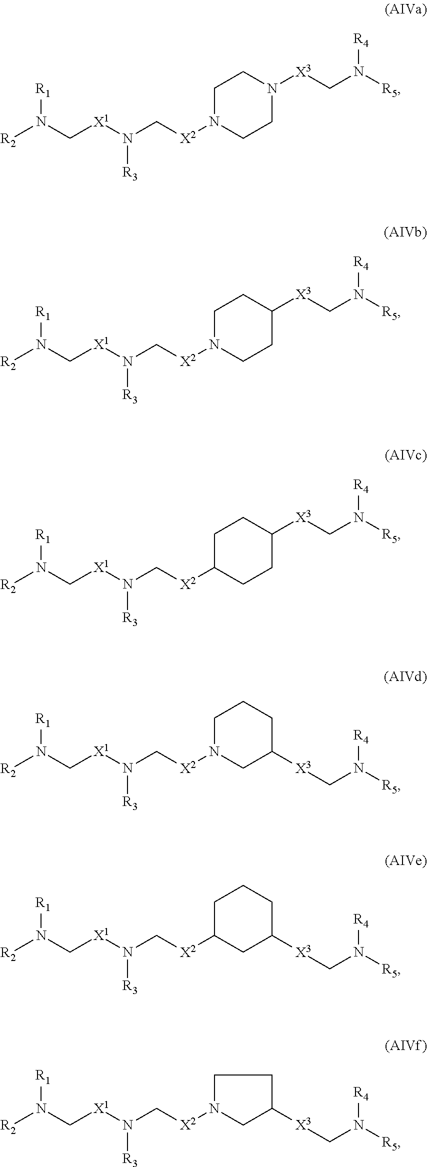

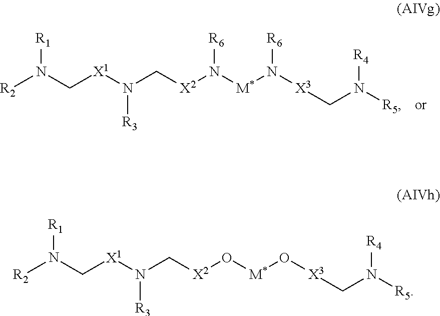

In some embodiments, the ionizable amino lipid is

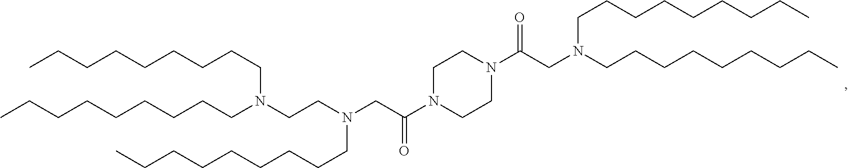 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (AIV), (AIVa), (AIVb), (AIVc), (AIVd), (AIVe), (AIVf), (AIVg), or (AIVh) may be protonated at a physiological pH. Thus, a lipid may have a positive or partial positive charge at physiological pH.

### Formula (AV)

In some embodiments, the lipid nanoparticle comprises a lipid having the structure:

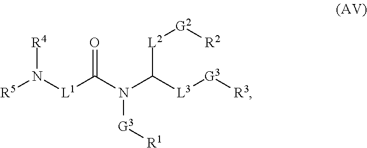

or a pharmaceutically acceptable salt, tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C₁-C₁₈ alkylene;
G¹ is a direct bond, —(CH₂)ₙO(C═O)—, —(CH₂)ₙ (C═O)O—, or —(C═O)—;
G² and G³ are each independently —(C═O)O— or -0(C═O)—; and n is an integer greater than 0.

### Formula (AVI)

In some embodiments, the lipid nanoparticle comprises a lipid having the structure:

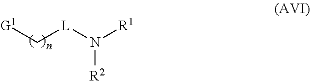

or a pharmaceutically acceptable salt, tautomer, or stereoisomer thereof, wherein:
G¹ is —N(R³)R⁴ or —OR⁵;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when Lis C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and
n is an integer from 1 to 12.

### Formula (AVII)

In some embodiments, the lipid nanoparticle comprises a lipid having the structure:

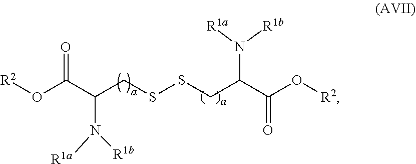

or a pharmaceutically acceptable salt thereof, wherein:
each R^{1a} is independently hydrogen, R^{1c}, or R^{1d},
each R^{1b} is independently R^{1c} or R^{1d},
each R^{1c} is independently —[CH₂]₂C(O)X¹R³,
each R^{1d} Is independently —C(O)R⁴;
each R² is independently —[C(R^{2a})₂]_{c}R^{2b},
each R^{2a} is independently hydrogen or C₁-C₆ alkyl;
R^{2b} is —N(L₁-B)₂; —(OCH₂CH₂)₆OH; or —(OCH₂CH₂)_{b}OCH₃;
each R³ and R⁴ is independently C₆-C₃₀ aliphatic;
each I.₃ is independently C₁-C₁₀ alkylene;
each B is independently hydrogen or an ionizable nitrogen-containing group;
each X¹ is independently a covalent bond or O;
each a is independently an integer of 1-10;
each b is independently an integer of 1-10; and
each c is independently an integer of 1-10.

### Formula (AVIII)

In some embodiments, the lipid nanoparticle comprises a lipid having the structure:

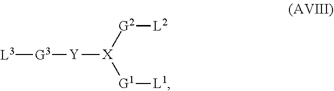

or a pharmaceutically acceptable salt, prodrug or stereoisomer thereof, wherein:
X is N, and Y is absent; or X is CR, and Y is NR;
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
L³ is —O(C═O)R³ or —(C═O)OR³;
G¹ and G² are each independently C₂-C₁₂ alkylene or C₂-C₁₂ alkenylene;
G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
each R is independently H or C₁-C₁₂ alkyl;

R¹, R² and R³ are each independently C₁-C₂₄ alkyl or C₂-C₂₄ alkenyl; and x is 0, 1 or 2, and
wherein each alkyl, alkenyl, alkylene, alkenylene, heteroalkylene and heteroalkenylene is independently substituted or unsubstituted unless otherwise specified.

### Formula (AIX)

In some embodiments, the lipid nanoparticle comprises a lipid having the structure:

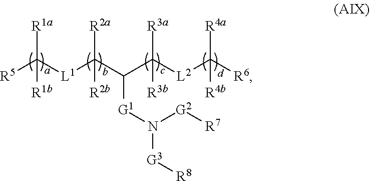

or a pharmaceutically acceptable salt, tautomer, prodrug or stereoisomer thereof, wherein:
L¹ and L² are each independently -0(C=0)0-, —(C=0)0-, —C(=0)-, -0-, —S(0)x-ₛ-S—S—, —C(=0)S—, —SC(=0)-, —NR^{a}C(=0)-, —C(=0)NR^{a}—, —NR^{a}C(=0)NR^{a}—, —OC(=0)NR^{a}—, —NR^{a}C(=0)0- or a direct bond;
G¹ is C, —C₂ alkylene, —(C=0)-, -0(C=0)-, —SC(=0)-, —NR^{a}C(=0)- or a direct bond;
G² is —C(0)-, —(CO)O—, —C(=0)S—, —C(=0)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{Ib} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4A} and R^{4B} are, at each occurrence, independently either: (a) H or C₁-C₁₂ alkyl; or (b) R^{4A} is H or C₁-C₁₂ alkyl, and R^{4B} together with the carbon atom to which it is bound is taken together with an adjacent R^{4B} and the carbon atom to which it is bound to form a carbon-carbon double bond;
R⁵ and R⁶ are each independently H or methyl;
R⁷ is H or C, —C₂₀ alkyl;
R⁸ is OH, —N(R⁹)(C=0)R¹⁰, —(C=0)NR⁹R¹⁰, —NR⁹R¹⁰, —(C=0)OR″¹ or -0(C=0)R″, provided that G³ is C₄-C₆ alkylene when R⁸ is —NR⁹R¹⁰,
R⁹ and R¹⁰ are each independently H or C₁-C₁₂ alkyl;
R″ is aralkyl;
a, b, c and d are each independently an integer from 1 to 24; and x is 0, 1 or 2,
wherein each alkyl, alkylene and aralkyl is optionally substituted.

### Formula (AX)

In some embodiments, the lipid nanoparticle comprises a lipid having the structure:

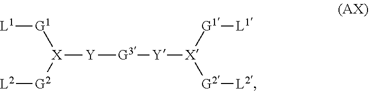

or a pharmaceutically acceptable salt, prodrug or stereoisomer thereof, wherein:
X and X′ are each independently N or CR;
Y and Y′ are each independently absent, —O(C═O)—, —(C═O)O— or NR, provided that:
a) Y is absent when X is N;
b) Y′ is absent when X′ is N;
c) Y is —O(C═O)—, —(C═O)O— or NR when X is CR; and
d) Y′ is —O(C═O)—, —(C═O)O— or NR when X′ is CR,
L¹ and L^{1′} are each independently —O(C═O)R′, —(C═O)OR′, —C(═O)R′, —OR¹, —S(O)_{z}R′, —S—SR¹, —C(═O)SR′, —SC(═O)R′, —NR^{a}C(═O)R′, —C(═O)NR^{b}R^{c}, —NR^{a}C(═O)NR^{b}R^{c}, —OC(═O)NR^{b}R^{c} or —NR^{a}C(═O)OR′;
L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹. G^{1′}, G² and G^{2′} are each independently C₂-C₁₂ alkylene or C₂-C₁₂ alkenylene;
G is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{c} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and wherein each alkyl, alkenyl, alkylene, alkenylene, heteroalkylene and heteroalkenylene is independently substituted or unsubstituted unless otherwise specified.

### Formula (AXI)

In some embodiments, the lipid nanoparticle comprises a lipid having the structure:

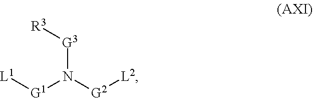

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl; and
x is 0, 1 or 2, and
wherein each alkyl, alkenyl, alkylene, alkenylene, cycloalkylene, cycloalkenylene, aryl and aralkyl is independently substituted or unsubstituted unless otherwise specified.

In some embodiments, the lipid nanoparticle comprises a lipid having the structure:

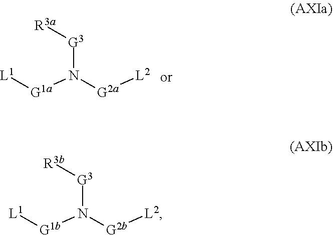

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f},
—OC(═O)NR^{e}R^{f}; —NR^{d}C(═O)OR² or a direct bond to R²;
G^{1a} and G^{2b} are each independently C₂-C₁₂ alkylene or C₂-C₁₂ alkenylene;
G^{1b} and G^{2b} are each independently C₁-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R^{3a} is —C(═O)N(R^{4a})R^{5a} or —C(═O)OR⁶;
R^{3b} is —NR^{4b}C(=O)R^{5b};
R^{4a} is C₁-C₁₂ alkyl;
R^{4b} is H, C₁-C₁₂ alkyl or C₂-C₁₂ alkenyl;
R^{5a} is H, C₁-C₈ alkyl or C₂-C₈ alkenyl;
R^{5b} is C₂-C₁₂ alkyl or C₂-C₁₂ alkenyl when R^{4b} is H; or R^{5b} is C₁-C₁₂ alkyl or C₂-C₁₂ alkenyl when R^{4b} is C₁-C₁₂ alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

### Formula (AXII)

In some embodiments, the lipid nanoparticle comprises a lipid having the structure:

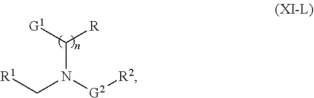

or a pharmaceutically acceptable salt, prodrug or stereoisomer thereof, wherein:
G¹ is —OH, —R³R⁴, —(C=0)R⁵ or —R³(C=0)R⁵;
G² is —CH₂— or —(C=0)-;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated Ci-C₆ alkyl;
R⁵ is optionally substituted straight or branched, saturated or unsaturated Ci-C₆ alkyl; and
n is an integer from 2 to 6.

### Formula (AXIII)

In some embodiments, the lipid nanoparticle comprises a lipid having the structure:

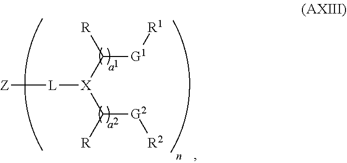

or a pharmaceutically acceptable salt, prodrug or stereoisomer thereof, wherein:
one of G¹ or G² is, at each occurrence, —O(C═O)—, —(C═O)O—, —C(═O)—, —O—, —S(O), —S—S—, —C(═O)S—, SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;
L is, at each occurrence, ˜O(C═O)—, wherein ˜ represents a covalent bond to X;
X is CR^{a};
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

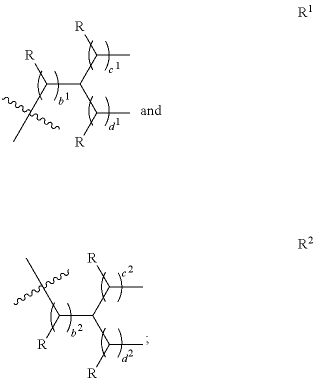

a¹ and a² are, at each occurrence, independently an integer from 3 to 12; b¹ and b² are, at each occurrence, independently 0 or 1;
c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

### Formula (AXIV)

In some embodiments, the lipid nanoparticle comprises a lipid having the structure:

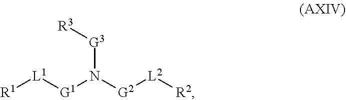

or a pharmaceutically acceptable salt, prodrug or stereoisomer thereof, wherein:
one of L¹ or L² is —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, R^{a}C(═O)R^{a}—, —OC(═O)R^{a}— or —R^{a}C(═O)O—, and the other of L¹ or L² is —O(C═O)—, —(C═O)O—, —C(═O)—, —O—, —S(O)ₓ—, —S—S—, —C(═O)S—, SC(═O)—, —R^{a}C(═O)—, —C(═O)R^{a}—, R^{a}C(═O)R^{a}—, —OC(═O)R^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —R⁵C(═O)R⁴;
R⁴ is C₁-C₁₂ alkyl;
R⁵ is H or C₁-C₆ alkyl; and
x is 0, 1 or 2.

### Formula (AXV)

In some embodiments, the lipid nanoparticle comprises a lipid having the structure:

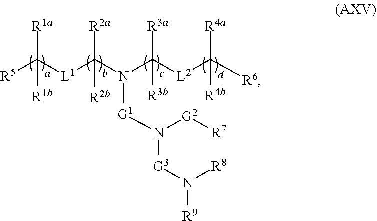

or a pharmaceutically acceptable salt, tautomer, prodrug or stereoisomer thereof, wherein:
L¹ and L² are each independently -0(C=0)-, —(C=0)0-, —C(=0)-, -0-, —S(0)ₓ—, —S—S—, —C(=0)S—, —SC(=0)-, —R^{a}C(=0)-, —C(=0)R^{a}—, —R^{a}C(=0)R^{a}—, —OC(=0)R^{a}—, —R^{a}C(=0)0- or a direct bond;
G¹ is C₁-C₂ alkylene, —(C=0)-, -0(C=0)-, —SC(=0)-, —R^{a}C(=0)- or a direct bond:
G² is —C(=0)-, —(C=0)0-, —C(=0)S—, —C(=0)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

### Formula (AXVI)

In some embodiments, the lipid nanoparticle comprises a lipid having the structure:

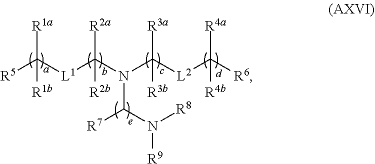

or a pharmaceutically acceptable salt, tautomer, prodrug or stereoisomer thereof, wherein:
L¹ and L² are each independently -0(C=0)-, —(C=0)0- or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{Ib} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24; b and c are each independently an integer from 1 to 24; and e is 1 or 2,
provided that:
at least one of R^{1a}, R^{2a}, R^{3a} or R^{4a} is C₁-C₁₂ alkyl, or at least one of L¹ or L² is -0(C=0)- or —(C=0)0-; and
R^{1a} and R^{1b} are not isopropyl when a is 6 or n-butyl when a is 8.

### Formula (AXVII)

In some embodiments, the lipid nanoparticle comprises a lipid having the structure:

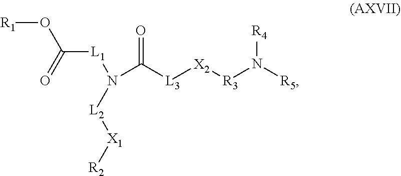

or a pharmaceutically acceptable salt thereof, wherein
R₁ and R₂ are the same or different, each a linear or branched alkyl with 1-9 carbons, or as alkenyl or alkynyl with 2 to 11 carbon atoms,
L₁ and L₂ are the same or different, each a linear alkyl having 5 to 18 carbon atoms, or form a heterocycle with N,
X₁ is a bond, or is —CG-G-whereby L2-CO—O—R₂ is formed,
X₂ is S or O,
L₃ is a bond or a lower alkyl, or form a heterocycle with N,
R₃ is a lower alkyl, and
R₄ and R₅ are the same or different, each a lower alkyl.
   Compounds (A₁)-(A₁₁)

In some embodiments, the lipid nanoparticle comprises an ionizable lipid having the structure:

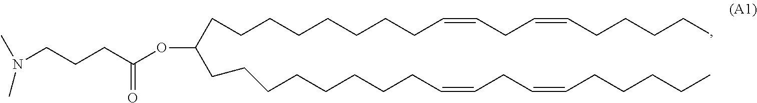

or a pharmaceutically acceptable salt thereof.

In some embodiments, the lipid nanoparticle comprises a lipid having the structure:

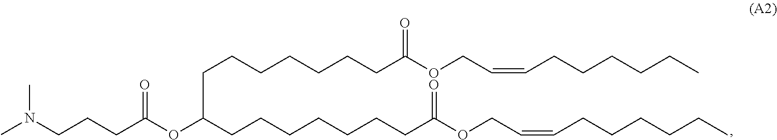 [SMILES: CCCCCC/C=C\COC(=O)CCCCCCCC(CCCCCCCC(=O)OC/C=C\CCCCCC)OC(=O)CCCN(C)C]

or a pharmaceutically acceptable salt thereof.

In some embodiments, the lipid nanoparticle comprises a lipid having the structure:

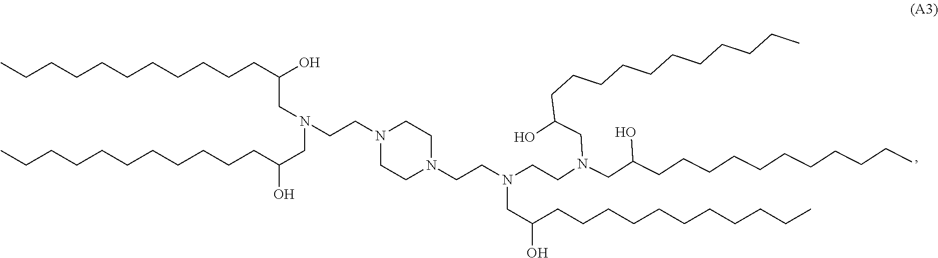 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

or a pharmaceutically acceptable salt thereof.

In some embodiments, the lipid nanoparticle comprises a lipid having the structure:

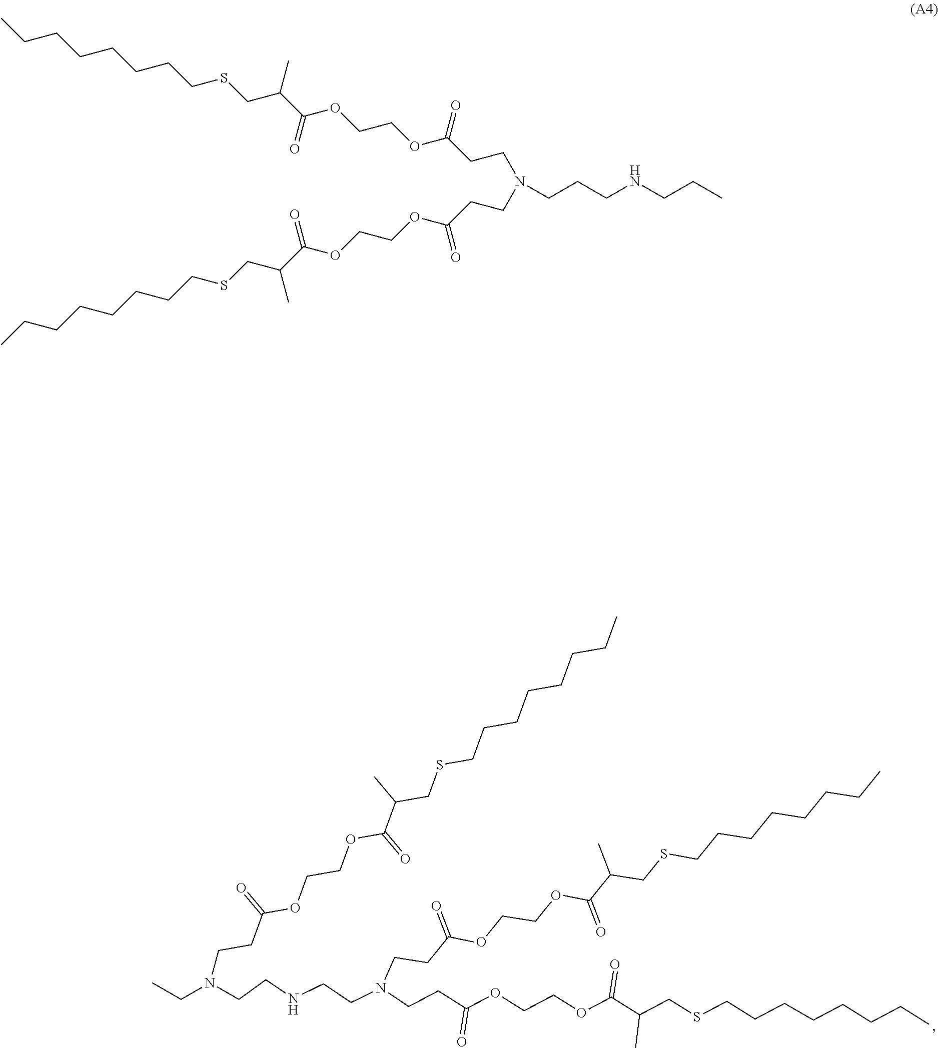

or a pharmaceutically acceptable salt thereof.

In some embodiments, the lipid nanoparticle comprises a lipid having the structure:

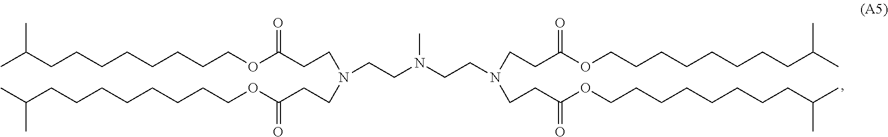 [SMILES: CC(C)CCCCCCCCOC(=O)CCN(CCC(=O)OCCCCCCCCC(C)C)CCN(C)CCN(CCC(=O)OCCCCCCCCC(C)C)CCC(=O)OCCCCCCCCC(C)C]

or a pharmaceutically acceptable salt thereof.

In some embodiments, the lipid nanoparticle comprises a lipid having the structure:

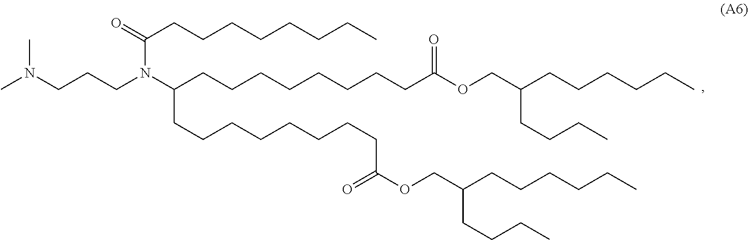 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

or a pharmaceutically acceptable salt thereof.

In some embodiments, the lipid nanoparticle comprises a lipid having the structure:

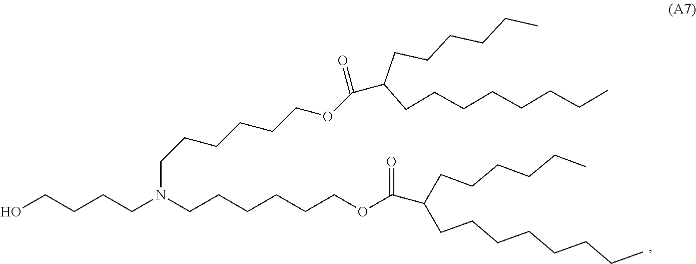 [SMILES: CCCCCCCCC(CCCCCC)C(=O)OCCCCCCN(CCCCO)CCCCCCOC(=O)C(CCCCCC)CCCCCCCC]

or a pharmaceutically acceptable salt thereof.

In some embodiments, the lipid nanoparticle comprises a lipid having the structure:

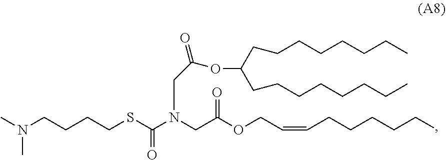

or a pharmaceutically acceptable salt thereof.

In some embodiments, the lipid nanoparticle comprises a lipid having the structure:

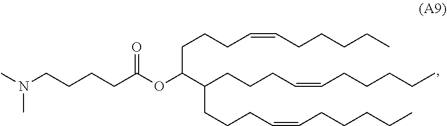 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments, the lipid nanoparticle comprises a lipid having the structure:

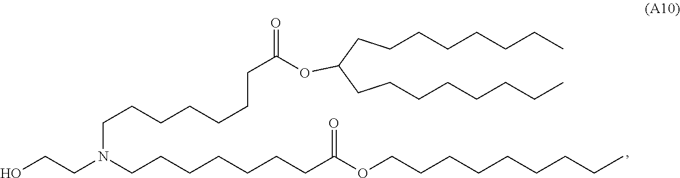 [SMILES: CCCCCCCCCOC(=O)CCCCCCCN(CCO)CCCCCCCC(=O)OC(CCCCCCCC)CCCCCCCC]

or a pharmaceutically acceptable salt thereof.

In some embodiments, the lipid nanoparticle comprises a lipid having the structure:

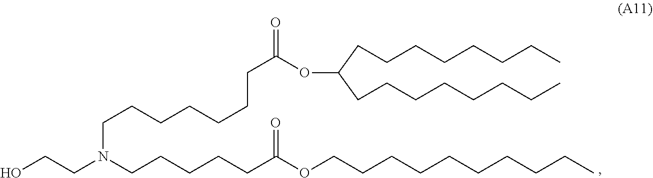 [SMILES: CCCCCCCCCCOC(=O)CCCCCN(CCO)CCCCCCCC(=O)OC(CCCCCCCC)CCCCCCCC]

or a pharmaceutically acceptable salt thereof.

### Non-Cationic Lipids

In certain embodiments, lipid nanoparticles comprise one or more non-cationic lipids. Non-cationic lipids may be phospholipids.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, a non-cationic lipid comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPc), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In certain embodiments, the lipid composition of the lipid nanoparticle compositions can comprise one or more phospholipids, for example, one or more saturated or (poly) unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine 1,2-dilinoleoyl-sn-glycero-3-(DOPE), phosphocholine (DLPc), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

### Formula (HI)

In certain embodiments, a phospholipid is an analog or variant of DSPC. In certain embodiments, a phospholipid is a compound of Formula (HI):

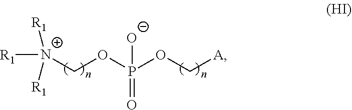

or a salt thereof, wherein:
each R1 is independently optionally substituted alkyl; or optionally two R1 are joined together with the intervening atoms to form optionally substituted monocyclic carbocyclyl or optionally substituted monocyclic heterocyclyl; or optionally three R1 are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

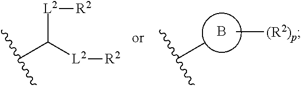

each instance of L2 is independently a bond or optionally substituted C1-6 alkylene, wherein one methylene unit of the optionally substituted C1-6 alkylene is optionally replaced with O, N(RN), S, C(O), C(O)N(RN), NRNC(O), C(O)O, OC(O), OC(O)O, OC(O)N(RN), —NRNC(O)O, or NRNC(O)N(RN);
each instance of R2 is independently optionally substituted C1-30 alkyl, optionally substituted C1-30 alkenyl, or optionally substituted C1-30 alkynyl; optionally wherein one or more methylene units of R² are independently replaced with optionally substituted carbocyclylene, optionally substituted heterocyclylene, optionally substituted arylene, optionally substituted heteroarylene, N(RN), O, S, C(O), C(O)N(RN), NRNC(O), NRNC(O)N(RN), C(O)O, OC(O), OC(O)O, OC(O)N(RN), NRNC(O)O, C(O)S, SC(O), C(═NRN), C(═NRN)N(RN), NRNC(═NRN), NRNC(═NRN)N(RN), C(S), C(S)N(RN), NRNC(S), NRNC(S)N(RN), S(O), OS(O), S(O)O, OS(O)O, OS(O)2, S(O)2O, OS(O)2O, N(RN)S(O), S(O)N(RN), N(RN)S(O)N(RN), OS(O)N(RN), N(RN)S(O)O, S(O)2, N(RN)S(O)2, S(O)2N(RN), N(RN)S(O)2N(RN), OS(O)2N(RN), or N(RN)S(O)2O;
each instance of RN is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound is not of the formula:

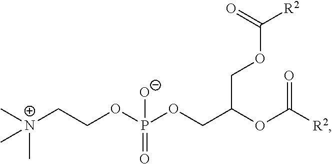

wherein each instance of R2 is independently unsubstituted alkyl, unsubstituted alkenyl, or unsubstituted alkynyl.

In some embodiments, the phospholipids may be one or more of the phospholipids described in PCT Application No. PCT/US2018/037922.

In some embodiments, the lipid nanoparticle comprises a molar ratio of 5-25% non-cationic lipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% non-cationic lipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% phospholipid lipid.

### Structural Lipids

The lipid composition of a pharmaceutical composition can comprise one or more structural lipids. As used herein, the term “structural lipid” includes sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises 30-45 mol % sterol, optionally 35-40 mol %, for example, 30-31 mol %, 31-32 mol %, 32-33 mol %, 33-34 mol %, 35-35 mol %, 35-36 mol %, 36-37 mol %, 38-38 mol %, 38-39 mol %, or 39-40 mol %. In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or 40 mol % cholesterol.

### Polyethylene Glycol (PEG)-Lipids

The lipid composition of a pharmaceutical composition can comprise one or more polyethylene glycol (PEG)-modified lipids.

As used herein, the term “PEG-lipid” or “PEG-modified lipid” refers to polyethylene glycol (PEG)-modified lipids. Non-limiting examples of PEG-modified lipids include PEG-modified phosphatidylethanolamine and phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG-modified lipid can be PEG-c-DOMG, PEG-DMG, PEG-DLPE, PEG-DMPE, PEG-DPPC, or a PEG-DSPE lipid.

In some embodiments, the PEG-modified lipid includes, but not limited to 1,2-dimyristoyl-sn-glycerol methoxypolyethylene glycol (PEG-DMG), 1,2-distearoyl-sn-glycero-3-phosphoethanolamine-N-[amino (polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-modified lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG, and/or PEG-DPG.

In some embodiments, the lipid moiety of the PEG-modified lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-modified lipid is PEG₂ₖ-DMG.

In some embodiments, lipid nanoparticles can comprise a PEG-modified lipid which is a non-diffusible PEG. Non-limiting examples of non-diffusible PEGs include PEG-DSG and PEG-DSPE.

PEG-modified lipids are known in the art, such as those described in U.S. Pat. No. 8,158,601 and International Publ. No. WO 2015/130584 A2, which are incorporated herein by reference to the extent they describe PEG-modified lipids.

In general, some of the other lipid components (e.g., PEG-modified lipids) of various formulae may be synthesized as described International Patent Application No. PCT/US2016/000129, filed Dec. 10, 2016, entitled “Compositions and Methods for Delivery of Therapeutic Agents,” which is incorporated by reference to the extent they disclose lipid components and production.

The lipid component of a lipid nanoparticle composition may include one or more molecules comprising polyethylene glycol, such as PEG or PEG-modified lipids. Such species may be alternately referred to as PEGylated lipids. A PEG-modified lipid is a lipid modified with polyethylene glycol. A PEG-modified lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG-modified lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

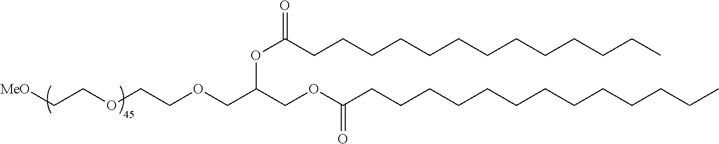

In some embodiments, PEG-modified lipids can be PEGylated lipids described in International Publication No. WO 2012/099755, which is herein incorporated by reference to the extent it discloses PEG-modified lipids. Any of these exemplary PEG-modified lipids may be modified to comprise a hydroxyl group on the PEG chain. In certain embodiments, the PEG-modified lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

### Formula (PI)

In certain embodiments, a PEG-modified lipid is a compound of Formula (PI):

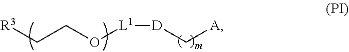

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

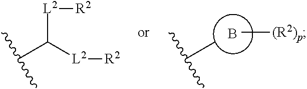

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (PI) is a PEG-OH lipid (i.e., R³ is —OR^{O}, and R^{O} is hydrogen). In certain embodiments, the compound of Formula (PI) is of Formula (PI—OH):

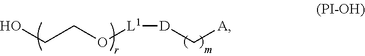

or a salt thereof.

### Formula (PII)

In certain embodiments, a PEG-modified lipid is a PEGylated fatty acid. In certain embodiments, a PEG-modified lipid is a compound of Formula (PII). In some embodiments, compounds of Formula (PII) have the following formula:

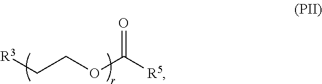

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, s (O)₂, n (R^{N})S(O)₂, S(O)₂N(R^{N}), N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (PII) is of Formula (PII—OH):

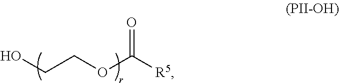

or a salt thereof. In some embodiments, r is 40-50.

In yet other embodiments the compound of Formula (PII) is:

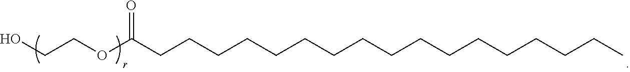

or a salt thereof.

In some embodiments, the compound of Formula (PII) is

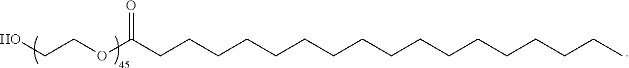

In some embodiments, the lipid composition of the pharmaceutical composition does not comprise a PEG-modified lipid.

In some embodiments, the PEG-modified lipids may be one or more of the PEG-modified lipids described in U.S. application Ser. No. 15/674,872.

In some embodiments, the lipid nanoparticle comprises a molar ratio of 0.5-15% PEG-modified lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG-modified lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises 1-5% PEG-modified lipid, optionally 1-3 mol %, for example 1.5 to 2.5 mol %, 1-2 mol %, 2-3 mol %, 3-4 mol %, or 4-5 mol %. In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

Some embodiments comprise adding PEG to a composition comprising an LNP encapsulating a nucleic acid (e.g., which already includes PEG in the amounts listed above). In embodiments comprise adding about 0.5 mo % or more PEG to an LNP composition, such as about 1 mol %, about 1.5 mol %, about 2 mol %, about 2.5 mol %, about 3 mol %, about 3.5 mol %, about 4 mol %, about 5 mol %, or more after formation of an LNP composition (e.g., which already contains PEG in amount listed elsewhere herein).

In some embodiments, a lipid nanoparticle comprises a first PEG-modified lipid in a core of the LNP, and a second PEG-modified lipid outside of the core of the LNP. The first and second PEG-modified lipids of the core and outside the core may the same PEG-modified lipids (i.e., have the same structure), or be different PEG-modified lipids (i.e., have different structures). In some embodiments, both PEG-modified lipids are 134-hydroxy-3,6,9,12,15,18,21,24,27,30,33,36,39,42,45,48,51,54,57,60,63,66,69,72,75,78,81,84,87,90,93,96,99,102,105,108,111,114,117,120,123,126,129,132-tetratetracontaoxatetratriacontahectyl stearate. In some embodiments, both PEG-modified lipids are PEG-DMG. In some embodiments, the first PEG-modified lipid is PEG-DMG and the second PEG-modified lipid is 134-hydroxy-3,6,9,12,15,18,21,24,27,30,33,36,39,42,45,48,51,54,57,60,63,66,69,72,75,78,81,84,87,90,93,96,99,102,105,108,111,114,117,120,123,126,129,132-tetratetracontaoxatetratriacontahectyl stearate. In some embodiments, the first PEG-modified lipid is 134-hydroxy-3,6,9,12,15,18,21,24,27,30,33,36,39,42,45,48,51,54,57,60,63,66,69,72,75,78,81,84,87,90,93,96,99,102,105,108,111,114,117,120,123,126,129,132-tetratetracontaoxatetratriacontahectyl stearate and the second PEG-modified lipid is PEG-DMG. In some embodiments, 0.25 mol % to 1.0 mol % (as a percentage of lipids in the LNP) of the first PEG-modified lipid is in the core of the lipid nanoparticle. In some embodiments, 0.25 mol % to 0.50 mol % of the first PEG-modified lipid is in the core of the lipid nanoparticle. In some embodiments, 0.25 mol %, 0.50 mol %, 0.75 mol %, or 1.0 mol % of the first PEG-modified lipid is in the core of the LNP. In some embodiments, 2.0 mol % to 2.75 mol % of the second PEG-modified lipid is outside the core of the LNP. In some embodiments, 2.0 mol %, 2.25 mol %, 2.5 mol %, or 2.75 mol % of the second PEG-modified lipid is outside the core of the LNP. In some embodiments, the LNP comprises 3.0 mol % PEG-modified lipids.

LNPs having certain amounts of a PEG-modified lipid in the core and certain amounts of a PEG-modified lipid outside of the core, and methods of producing the same, are disclosed in PCT Publication No. WO 2023/018773, which is incorporated by reference herein to the extent it discloses lipid nanoparticles and methods of producing lipid nanoparticles.

In some embodiments, the lipid nanoparticle comprises 20-60 mol % ionizable lipid, 5-25 mol % non-cationic lipid, 25-55 mol % sterol, and 0.5-15 mol % PEG-modified lipid.

In some embodiments, a LNP comprises an ionizable lipid of Compound (I-18), wherein the non-cationic lipid is DSPC, the structural lipid that is cholesterol, and the PEG-modified lipid is DMG-PEG. In some embodiments, a LNP comprises 20-60 mol % ionizable lipid of Compound (I-18), 5-25 mol % DSPC, 25-55 mol % cholesterol, and 2-4 mol % DMG-PEG.

In some embodiments, a LNP comprises an ionizable lipid of Compound (I-25), wherein the non-cationic lipid is DSPC, the structural lipid that is cholesterol, and the PEG-modified lipid is DMG-PEG. In some embodiments, a LNP comprises 20-60 mol % ionizable lipid of Compound (I-25), 5-25 mol % DSPC, 25-55 mol % cholesterol, and 2-4 mol % DMG-PEG.

In some embodiments, a LNP comprises an ionizable lipid of Compound (I-301), wherein the non-cationic lipid is DSPC, the structural lipid that is cholesterol, and the PEG-modified lipid is DMG-PEG. In some embodiments, a LNP comprises 20-60 mol % ionizable lipid of Compound (I-301), 5-25 mol % DSPC, 25-55 mol % cholesterol, and 2-4 mol % DMG-PEG.

In some embodiments, a LNP comprises an ionizable lipid of Compound (II-6), wherein the non-cationic lipid is DSPC, the structural lipid that is cholesterol, and the PEG-modified lipid is DMG-PEG. In some embodiments, a LNP comprises 20-60 mol % ionizable lipid of Compound (II-6), 5-25 mol % DSPC, 25-55 mol % cholesterol, and 2-4 mol % DMG-PEG.

In some embodiments, a LNP comprises an ionizable lipid of Compound (IL**), wherein the non-cationic lipid is DSPC, the structural lipid that is cholesterol, and the PEG-modified lipid is DMG-PEG. In some embodiments, a LNP comprises 20-60 mol % ionizable lipid of Compound (IL**), 5-25 mol % DSPC, 25-55 mol % cholesterol, and 2-4 mol % DMG-PEG.

In some embodiments, a LNP comprises an ionizable lipid of any of Formula (IL*), a phospholipid comprising DSPC, a structural lipid, and a PEG-modified lipid comprising PEG-DMG. In some embodiments, a LNP comprises 20-60 mol % ionizable lipid of Formula (IL*), 5-25 mol % phospholipid comprising DSPC, 25-55 mol % structural lipid, and 2-4 mol % PEG-modified lipid comprising DMG-PEG.

In some embodiments, a LNP comprises an ionizable lipid of any of Formula (IL*), a phospholipid comprising DSPC, a structural lipid, and a PEG-modified lipid comprising a compound having Formula (PII). In some embodiments, a LNP comprises 20-60 mol % ionizable lipid of Formula (IL*), 5-25 mol % phospholipid comprising DSPC, 25-55 mol % structural lipid, and 2-4 mol % PEG-modified lipid comprising a compound having Formula (PII).

In some embodiments, a LNP comprises an ionizable lipid of Formula (IL*), a phospholipid comprising a compound having Formula (HI), a structural lipid, and the PEG-modified lipid comprising a compound having Formula (PI) or (PII). In some embodiments, a LNP comprises 20-60 mol % ionizable lipid of Formula (IL*), 5-25 mol % phospholipid comprising a compound having Formula (HI), 25-55 mol % structural lipid, and 2-4 mol % PEG-modified lipid comprising a compound having Formula (PI) or (PII).

In some embodiments, a LNP comprises an ionizable lipid of Formula (IL*), a phospholipid comprising a compound having Formula (HI), a structural lipid, and the PEG-modified lipid comprising a compound having Formula (PI) or (PII). In some embodiments, a LNP comprises 20-60 mol % ionizable lipid of Formula (IL*), 5-25 mol % phospholipid comprising a compound having Formula (HI), 25-55 mol % structural lipid, and 2-4 mol % PEG-modified lipid modified lipid comprising a compound having Formula (PI) or (PII).

In some embodiments, a LNP comprises an ionizable lipid of Formula (IL*), a phospholipid having Formula (HI), a structural lipid, and a PEG-modified lipid comprising a compound having Formula (PII). In some embodiments, a LNP comprises 20-60 mol % ionizable lipid of Formula (IL*), 5-25 mol % phospholipid having Formula (HI), 25-55 mol % structural lipid, and 2-4 mol % PEG-modified lipid comprising a compound having Formula (PII).

In some embodiments, a LNP comprises an ionizable amino lipid of Compound 1, wherein the non-cationic lipid is DSPC, the structural lipid that is cholesterol, and the PEG-modified lipid is DMG-PEG. In some embodiments, a LNP comprises 20-60 mol % ionizable lipid of Compound 1, 5-25 mol % DSPC, 25-55 mol % cholesterol, and 2-4 mol % PEG-modified lipid DMG-PEG.

In some embodiments, a LNP comprises an ionizable amino lipid of Compound 2, wherein the non-cationic lipid is DSPC, the structural lipid that is cholesterol, and the PEG-modified lipid is DMG-PEG. In some embodiments, a LNP comprises 20-60 mol % ionizable lipid of Compound 2, 5-25 mol % DSPC, 25-55 mol % cholesterol, and 2-4 mol % PEG-modified lipid DMG-PEG.

In some embodiments, a LNP comprises an ionizable amino lipid of any of Formula (AIII), (AIV), or (AV), a phospholipid comprising DSPC, a structural lipid, and a PEG-modified lipid comprising PEG-DMG. In some embodiments, a LNP comprises 20-60 mol % ionizable lipid of any of Formula (AIII), (AIV), or (AV), 5-25 mol % DSPC, 25-55 mol % structural lipid, and 2-4 mol % PEG-modified lipid comprising DMG-PEG.

In some embodiments, a LNP comprises an ionizable amino lipid of any of Formula (AIII), (AIV), or (AV), a phospholipid comprising DSPC, a structural lipid, and a PEG-modified lipid comprising a compound having Formula (PII). In some embodiments, a LNP comprises 20-60 mol % ionizable lipid of any of Formula (AIII), (AIV), or (AV), 5-25 mol % DSPC, 25-55 mol % structural lipid, and 2-4 mol % PEG-modified lipid comprising a compound having Formula (PII).

In some embodiments, a LNP comprises an ionizable amino lipid of Formula (AIII), (AIV), or (AV), a phospholipid comprising a compound having Formula (HI), a structural lipid, and the PEG-modified lipid comprising a compound having Formula (PI) or (PII). In some embodiments, a LNP comprises 20-60 mol % ionizable lipid of Formula (AIII), (AIV), or (AV), 5-25 mol % phospholipid comprising a compound having Formula (HI), 25-55 mol % structural lipid, and 2-4 mol % PEG-modified lipid comprising a compound having Formula (PI) or (PII).

In some embodiments, a LNP comprises an ionizable amino lipid of Formula (AIII), (AIV), or (AV), a phospholipid comprising a compound having Formula (HI), a structural lipid, and the PEG-modified lipid comprising a compound having Formula (PI) or (PII). In some embodiments, a LNP comprises 20-60 mol % ionizable lipid of Formula (AIII), (AIV), or (AV), 5-25 mol % phospholipid comprising a compound having Formula (HI), 25-55 mol % structural lipid, and 2-4 mol % PEG-modified lipid comprising a compound having Formula (PI) or (PII).

In some embodiments, a LNP comprises an ionizable amino lipid of Formula (AIII), (AIV), or (AV), a phospholipid having Formula (HI), a structural lipid, and a PEG-modified lipid comprising a compound having Formula (PII). In some embodiments, a LNP comprises 20-60 mol % ionizable lipid of Formula (AIII), (AIV), or (AV), 5-25 mol % phospholipid having Formula (HI), 25-55 mol % structural lipid, and 2-4 mol % PEG-modified lipid comprising a compound having Formula (PII).

In some embodiments, the lipid nanoparticle comprises 49 mol % ionizable lipid, 10 mol % DSPC, 38.5 mol % cholesterol, and 2.5 mol % DMG-PEG.

In some embodiments, the lipid nanoparticle comprises 49 mol % ionizable lipid, 11 mol % DSPC, 38.5 mol % cholesterol, and 1.5 mol % DMG-PEG.

In some embodiments, the lipid nanoparticle comprises 48 mol % ionizable lipid, 11 mol % DSPC, 38.5 mol % cholesterol, and 2.5 mol % DMG-PEG.

In some embodiments, a LNP comprises an N:P ratio of from about 2:1 to about 30:1.

In some embodiments, a LNP comprises an N:P ratio of about 6:1.

In some embodiments, a LNP comprises an N:P ratio of about 3:1, 4:1, or 5:1.

In some embodiments, a LNP comprises a wt/wt ratio of the ionizable lipid component to the RNA of from about 10:1 to about 100:1.

In some embodiments, a LNP comprises a wt/wt ratio of the ionizable lipid component to the RNA of about 20:1.

In some embodiments, a LNP comprises a wt/wt ratio of the ionizable lipid component to the RNA of about 10:1.

Some embodiments comprise a composition having one or more LNPs having a diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. Some embodiments comprise a composition having a mean LNP diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. In some embodiments, the composition has a mean LNP diameter from about 30 nm to about 150 nm, or a mean diameter from about 60 nm to about 120 nm.

In some embodiments, an LNP further comprises one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-modified lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

In some embodiments, a LNP comprises one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be “knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way. In some embodiments, the composition comprises a liposome. A liposome is a lipid particle comprising lipids arranged into one or more concentric lipid bilayers around a central region. The central region of a liposome may comprise an aqueous solution, suspension, or other aqueous composition.

In some embodiments, the composition comprises a lipoplex. A lipoplex is a lipid particle comprising a cationic liposome and a nucleic acid (e.g., mRNA). Lipoplexes may be formed by contacting a liposome comprising a cationic lipid with a nucleic acid. A lipoplex may comprise multiple concentric lipid bilayers, each concentric bilayer separated by one or more nucleic acids. The central region of the lipoplex may comprise an aqueous solution, suspension, or other aqueous composition.

In some embodiments, the composition comprises a lipopolyplex. A lipopolyplex is a lipid particle comprising a lipid bilayer surrounding a complex of a cationic polymer and a nucleic acid (e.g., mRNA). See Midoux & Pichon, Expert Rev Vaccines. 2015. 14(2):221-234. A lipopolyplex may be formed by contacting a cationic liposome (e.g., liposome comprising a cationic lipid) with the complex of nucleic acid and cationic polymer. The central region of the lipopolyplex may comprise an aqueous solution, suspension, or other aqueous composition.

In some embodiments, the composition comprises a cationic nanoemulsion. A cationic nanoemulsion comprises a cationic lipid, hydrophilic surfactant, and hydrophobic surfactant.

A liposome, lipoplex, lipopolyplex, or cationic nanoemulsion may comprise a sterol. A liposome, lipoplex, lipopolyplex, or cationic nanoemulsion may comprise a neutral lipid. A liposome, lipoplex, lipopolyplex, or cationic nanoemulsion may comprise a PEG-modified lipid.

### Stabilizing Compounds

Some embodiments of compositions are stabilized pharmaceutical compositions. Various non-viral delivery systems, including nanoparticle formulations, present attractive opportunities to overcome many challenges associated with RNA delivery. Lipid nanoparticles (LNPs) have drawn particular attention in recent years as various LNP formulations have shown promise in a variety of pharmaceutical applications. However, lipids have been shown to degrade nucleic acids, including mRNA, and lipid nanoparticle formulations undergo rapid loss of purity when stored as refrigerated liquids. Moreover, the storage stability of mRNA encapsulated within LNPs is lower than that of unencapsulated mRNA.

A class of compounds has been found to stabilize nucleic acids within a lipid carrier such as an LNP, an unexpected and unprecedented discovery which enables applications including extended refrigerated liquid shelf-life, extended in-use periods at room temperature, and extended in-use stability at physiological temperatures up to higher temperatures such as 40° C. Such stabilizing compounds solve a critical problem, as current manufacturing processes and formulations experience a 5-10% purity loss during LNP formation and processing that is typical with current large-scale LNP production.

In some embodiments, the stabilized pharmaceutical composition comprises a nucleic acid formulation comprising a nucleic acid and a stabilizing compound (e.g., a compound of Formula (I), of Formula (II), or a tautomer or solvate thereof). In some embodiments, the stabilized pharmaceutical composition comprises a nucleic acid formulation comprising a nucleic acid and a lipid, and a compound of Formula (I):

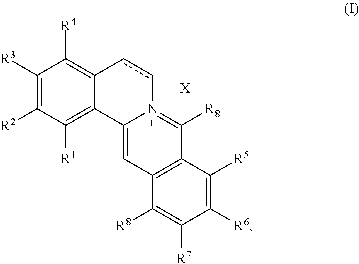

or a tautomer or solvate thereof, wherein:
[symbol] is a single bond or a double bond;
R¹ is H; R² is OCH₃, or together with R³ is OCH₂O; R³ is OCH₃, or together with R² is OCH₂O; R⁴ is H; R⁵ is H or OCH₃; R⁶ is OCH₃; R⁷ is H or OCH₃; R⁸ is H; R⁹ is H or CH₃; and X is a pharmaceutically acceptable anion, e.g., a halide such as chloride.

In some embodiments, the compound of Formula (I) has the structure of:

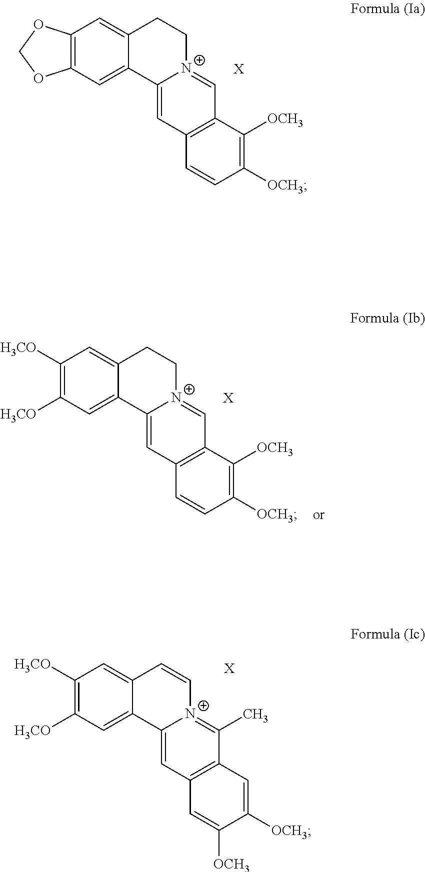

or a tautomer or solvate thereof.

In some embodiments, the stabilized pharmaceutical composition comprises a nucleic acid formulation comprising a nucleic acid and a lipid, and a compound of Formula (II):

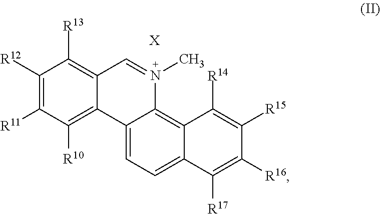

or a tautomer or solvate thereof, wherein:

R¹⁰ is H; R¹¹ is H; R¹² together with R¹³ is OCH₂O; R¹⁴ is H; R¹⁵ together with R¹⁶ is OCH₂O; R¹⁷ is H; and X is a pharmaceutically acceptable anion, e.g., a halide such as chloride.

In some embodiments, the compound of Formula (II) has the structure of:

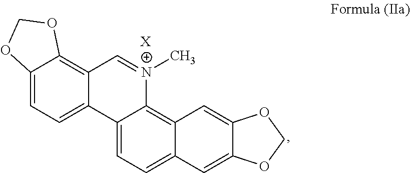

or a tautomer or solvate thereof.

Stabilizing compounds of Formulas (I), (Ia), (Ib), (Ic), (II), and (Iia) are described in International Application No. PCT/US2022/025967, which is incorporated by reference herein in its entirety.

In some embodiments, the nucleic acid formulation comprises lipid nanoparticles. In some embodiments, the nucleic acid is mRNA.

In some embodiments, the stabilizing compound (“the compound”) has a purity of at least 70%, 80%, 90%, 95%, or 99%. In some embodiments, the compound contains fewer than 100 ppm of elemental metals. In some embodiments, the stabilized pharmaceutical composition (“the composition”) comprises a pharmaceutically acceptable metal chelator, e.g., EDTA (ethylenediaminetetraacetic acid) or DTPA (diethylenetriaminepentaacetic acid).

In some embodiments, the composition is an aqueous solution. In some embodiments, the compound is present at a concentration between about 0.1 mM and about 10 mM in the aqueous solution. In some embodiments, the aqueous solution has a pH of or about 5 to 8, including pH of about 5, 5.5, 6, 6.5, 7, 7.5, or 8. In some embodiments, the aqueous solution does not comprise NaCl. In some embodiments, the aqueous solution comprises NaCl in a concentration of or about 150 mM. In some embodiments, the aqueous solution comprises a phosphate buffer, a tris buffer, an acetate buffer, a histidine buffer, or a citrate buffer.

In some embodiments, microbial growth in the composition is inhibited by the compound.

In some embodiments, the composition is characterized as having a mRNA purity level of greater than 60%, greater than 70%, greater than 80%, or greater than 90% main peak mRNA purity after at least thirty days of storage. In some embodiments, the composition comprises a mRNA purity level of greater than 50% main peak mRNA purity after at least six months of storage. In some embodiments, the storage is at room temperature.

In some embodiments, the composition comprises a lipid nanoparticle encapsulating a mRNA, and the composition comprises less than 50%, less than 60%, less than 70%, less than 80%, less than 90%, or less than 95% RNA fragments after at least thirty days of storage. In some embodiments, the storage temperature is greater than room temperature. In some embodiments, the storage temperature is about 4° C.

In some embodiments, the compound interacts with the nucleic acid comprised within a lipid nanostructure (e.g., a lipid nanoparticle, liposome, or lipoplex), e.g., via pi-pi stacking and/or by changing backbone helicity of the nucleic acid. In some embodiments, the compound intercalates with a nucleic acid. In some embodiments, the compound binds with a nucleic acid, e.g., reversible binding, and/or binding to the stranded regions of the nucleic acid. In some embodiments, the compound self-associates, binds to nucleic acid ribose contacts, and/or binds to nucleic acid base contacts. In some embodiments, the compound does not substantially bind to nucleic acid phosphate contacts. In some embodiments, the positive charge of the compound contributes to nucleic acid binding. In some embodiments, the interacts with the nucleic acid with a binding affinity defined by an equilibrium dissociation constant of less than 10-3 M (e.g., less than 10-4 M, less than 10-5 M, less than 10-5 M, less than 10-7 M, less than 10-8 M, or less than 10-9 M).

In some embodiments, the compound interacts with a nucleic acid and provides shielding from solvent, e.g., water. In some embodiments, the compound shields ribose from solvent more than the compound shields the phosphate groups of the nucleic acid. In some embodiments, the solvent exposure is measured by the solvent accessible surface area (SASA). In some embodiments, a stabilizing compound decreases the solvent accessible area of ribose to about 5-10 nm². In some embodiments, a stabilizing compound decreases the solvent accessible area of ribose to about 6-8 nm². In some embodiments, a stabilizing compound decreases the solvent accessible area of phosphate to about 9-12 nm². In some embodiments, a stabilizing compound decreases the solvent accessible area of phosphate to about 10-11 nm².

In some embodiments, a nucleic acid that is conformationally stabilized by the compound exhibits thermal unfolding temperatures (measured by circular dichroism or DSC, for example) that are higher than in the absence of the compound. In some embodiments, the compound confers increased stability, e.g., thermal stability, to the nucleic acid in a folded structure, e.g., relative to its unfolded or less folded or more linear form. In some embodiments, the compound causes compaction of the nucleic acid upon interaction with the nucleic acid. In some embodiments, the compound causes a decrease in the hydrodynamic radius of the nucleic acid molecule upon interaction with the nucleic acid. In some embodiments, a stabilizing compound causes compaction or a decrease in the hydrodynamic radius of a nucleic acid molecule by 5%, 10%, 15%, 20%, 25%, 30%, 35%, 40%, 45%, 50%, 55%, 60%, or more. In some embodiments, a stabilizing compound causes compaction or a decrease in the hydrodynamic radius of a nucleic acid molecule when the compound is in a concentration of 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 60 μM, 70 μM, 80 μM, 90 μM, or 100 μM.

### Pharmaceutical Compositions

Provided are compositions comprising proteins or nucleic acids encoding proteins of interest. In some embodiments, the nucleic acids comprise DNA. In some embodiments, the nucleic acids comprise RNA, such as self-amplifying RNA, circular RNA, or mRNA. Preferably, the nucleic acid comprises mRNA. In some embodiments, the proteins are comprised in one or more viral vectors. In some embodiments, the proteins are encoded by one or more nucleic acids comprised in one or more viral vectors.

Compositions may not comprise antigens per se, but rather comprise nucleic acids, in particular mRNA(s), that encode antigens or antigenic sequences that, once delivered to a cell, tissue or subject, can be translated and ultimately produce an immune response. Delivery of nucleic acids, in particular mRNA(s), can be achieved by inclusion of nucleic acids in appropriate carriers or delivery vehicles (e.g., lipid nanoparticles) such that upon administration to cells, tissues or subjects, nucleic acid is taken up by cells which, in turn, express protein(s) encoded by the nucleic acids, e.g., mRNAs. Upon delivery and uptake by cells of the body, the mRNAs are translated in the cytosol and protein antigens are generated by the host cell machinery. The encoded protein antigen(s) are presented and elicit an adaptive humoral and cellular immune response.

Some embodiments of compositions (e.g., pharmaceutical compositions), methods, kits and reagents comprising mRNAs encoding influenza virus antigens and optionally other respiratory virus antigens are suitable for prevention or treatment of influenza virus in humans and other mammals, for example. Compositions (e.g., pharmaceutical compositions) can be used as therapeutic or prophylactic agents. They may be used in medicine to prevent and/or treat an influenza virus infection. In some embodiments, the compositions are used to treat and/or prevent influenza and also one or more additional respiratory infectious diseases, such as coronavirus and/or respiratory syncytial virus infection. Preferably, the composition is a vaccine, and more preferably the composition is an mRNA vaccine.

In some embodiments, a composition containing RNA can be administered to a subject (e.g., a mammalian subject, such as a human subject), and the RNA polynucleotides are translated in vivo to produce an antigenic polypeptide (antigen).

The term “pharmaceutical composition” refers to the combination of an active agent with a carrier, inert or active, making the composition especially suitable for therapeutic use in vivo or ex vivo. A “pharmaceutically acceptable carrier”, after being administered to or upon a subject, does not cause undesirable physiological effects.

The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carriers include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences. In some embodiments, the pharmaceutical composition does not comprise protamine.

Compositions may be immunizing compositions (e.g., RNA vaccines (e.g., mRNA vaccines)), and used in methods, kits and reagents for prevention and/or treatment of respiratory virus (e.g., influenza virus) infection in humans and other mammals. Immunizing compositions can be used as therapeutic or prophylactic agents. In some embodiments, immunizing compositions are used to provide prophylactic protection from influenza virus infection. In some embodiments, immunizing compositions are used to treat an influenza virus infection. In some embodiments, embodiments, immunizing compositions are used in the priming of immune effector cells, for example, to activate peripheral blood mononuclear cells (PBMCs) ex vivo, which are then infused (re-infused) into a subject.

Pharmaceutical compositions may be in a form for administration intramuscularly, intranasally, subcutaneously, or intradermally. Preferably, the pharmaceutical compositions are in a form for administration intramuscularly.

### Uses, Dosing, Administration

Provided are methods of administering a composition to a subject (e.g., a mammalian subject, preferably a human subject) in an effective amount to induce an antigen-specific immune response.

An “effective amount,” “pharmaceutically effective amount,” or “immunogenically effective amount” of a composition refers to a nontoxic but sufficient amount of an agent to provide the desired biological result. That result may be induction of an antibody response and/or T cell response to an antigen. An appropriate effective amount in any individual case may be determined by one of ordinary skill in the art using routine experimentation. Typically, an effective amount of a composition provides an induced or boosted immune response as a function of antigen production in the cells of the subject.

The effective amount (e.g., effective dose) of RNA in a composition may be as low as 5 μg, administered for example as a single dose. In some embodiments, the effective amount is a total dose of 5 μg-300 μg, for example, 5 μg-30 μg, 5 μg-25 μg, 5 μg-20 μg, 5 μg-15 μg, 5 μg −10 μg, 10 μg-30 μg, 10 μg-25 μg, 10 μg-20 μg, 10 μg-15 μg, 15 μg-30 μg, 15 μg-25 μg, 15 μg-20 μg, 20 μg-30 μg, 25 μg-30 μg, 25 μg-50 μg, 25 μg-150 μg, 25 μg-200 μg, 25 μg-250 μg, or 25 μg-300 μg. In some embodiments, the effective amount is 50 μg RNA. In some embodiments, the effective dose (e.g., effective amount) is at least 10 μg and less than 25 μg of RNA. In some embodiments, the effective dose (e.g., effective amount) is at least 5 μg and less than 25 μg of RNA. For example, the effective amount may be a total dose of 2.5 μg, 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. Any of the doses described above may be an effective amount for a booster dose; for example, in some embodiments, the booster dose is a total dose of 15 μg, 27.5 μg, 30 μg, 50 μg, 52.5 μg, 55 μg, or 60 μg RNA. In some embodiments, the booster dose is 50 μg RNA.

Any suitable method may be used to determine the ability of a composition to elicit an immune response (e.g., antigen-specific antibody production, antigen-specific CD8+ T cell activation or proliferation, and/or antigen-specific CD4+ T cell activation or proliferation). Antibody responses to an antigen may be measured, for example, by ELISA, HAI assay, or microneutralization assay of sera. T cell responses to an antigen may be measured, for example, by flow cytometry for analysis of cell subsets. In some embodiments, the effectiveness of a composition is determined using an animal model. Suitable animal models for influenza include mice, guinea pigs, ferrets, and swine. For example, a composition may be administered to an animal, after which blood, sera, peripheral blood mononuclear cells, lymph nodes, the spleen, and/or other organs of the animal may be analyzed for antibody and/or cellular responses. Viral challenge following administration of a composition may also be used to measure the efficacy of a composition. For example, a composition may be administered to an animal, after which the animal is inoculated with a virus, and the animal is monitored for signs of infection-related pathology (e.g., weight loss, respiratory defects, death) or viral replication (e.g., viral shedding, viremia). In some embodiments, the animal model is a mouse model. In some embodiments, the animal model is a ferret model. In some embodiments, the animal model is a guinea pig model. In some embodiments, a composition induces sterilizing immunity to a virus. A host with sterilizing immunity to a virus does not become infected (experience signs or symptoms of viral infection) following exposure to the virus.

In some embodiments, a composition is administered a single time. In some embodiments, a first composition is administered, and then a second composition is administered later. In some embodiments the two or more compositions are administered at least 1 week, at least 2 weeks, at least 3 weeks, at least 4 weeks, at least 5 weeks, at least 6 weeks, at least 7 week, and least 8 weeks, at least 3 months, at least 4 months, at least 5 months, at least 6 months, at least 7 months, at least 8 months, at least 9 months, at least 10 months, at least 11 months, or at least 12 months apart.

In some embodiments, a subject has not been vaccinated against an influenza virus for at least 2, at least 4, at least 6, at least 8, at least 10, or at least 12 months prior to administration of a composition. In some embodiments, a subject has not previously been vaccinated against an influenza virus comprising a reference influenza virus HA protein. In some embodiments, a subject has not previously been administered a composition comprising a reference HA protein or a nucleic acid encoding the reference HA protein.

Pharmaceutical compositions may be administered intramuscularly, intranasally, subcutaneously, or intradermally. Preferably, the pharmaceutical compositions are administered intramuscularly.

### EXAMPLES

### Example 1. Stabilizing Mutations: Influenza B Virus Hemagglutinin

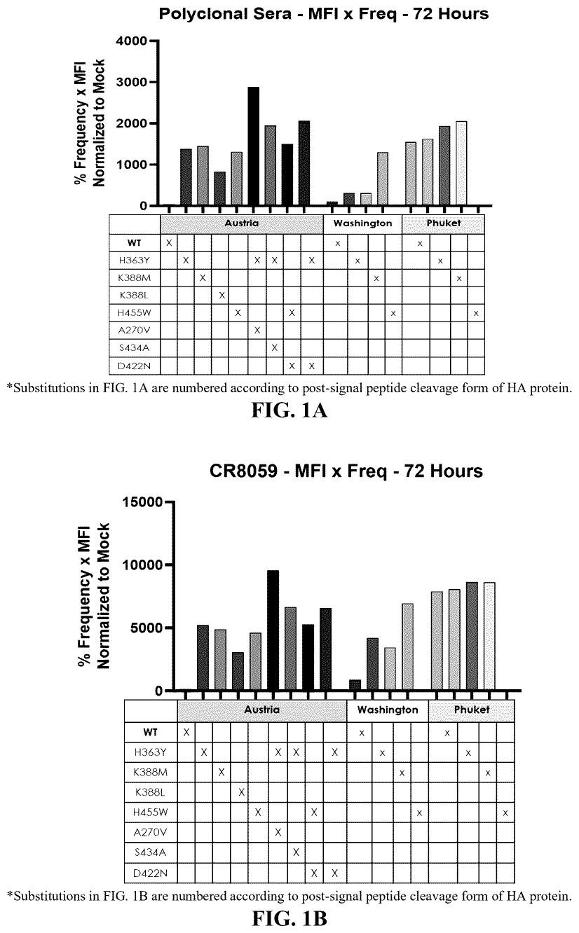

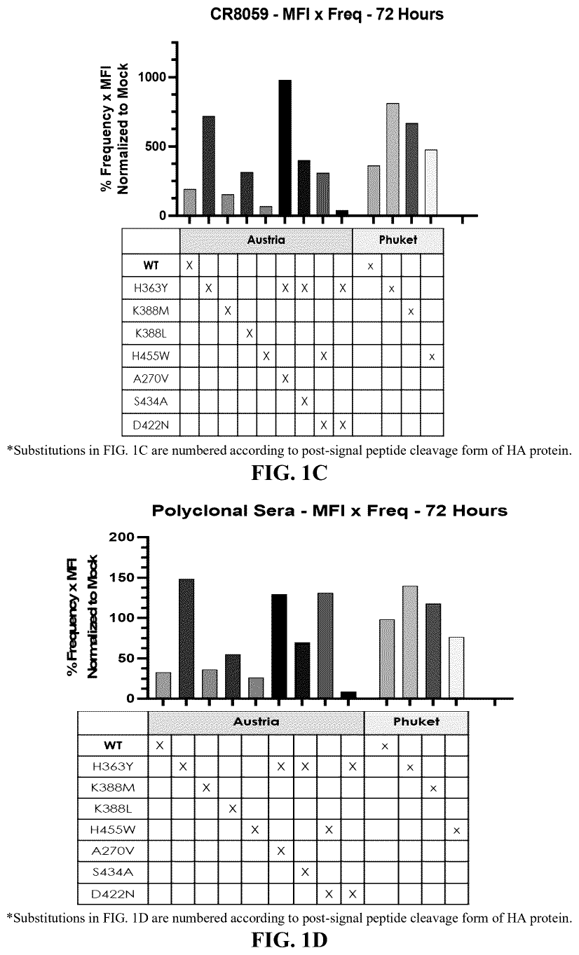

In this Example, a variety of different stabilizing mutations were made to influenza B virus hemagglutinin (HA) antigens and the effects were tested. First, mRNA encoding influenza B virus HA antigens having stabilizing mutations was transfected (250 ng/1×10⁶ cells). Expression levels were determined 72 hours later (FIGS. 1A-1B) and show that the stabilizing mutations are beneficial to in vitro expression in different influenza B virus HA backbones. CR8059 (an IBV HA strain-specific antibody) data is shown in FIG. 1A and polyclonal sera expression data is shown in FIG. 1B. The experiments were repeated for the B/Austria and B/Phuket strains using mRNA formulated in lipid nanoparticles without a transfection reagent (100 ng/1×10⁶ cells). The expression data 72 hours after transfection is shown in FIGS. 1C-1D and further demonstrates that the stabilizing mutations increase expression above levels of the mock-transfected cells.

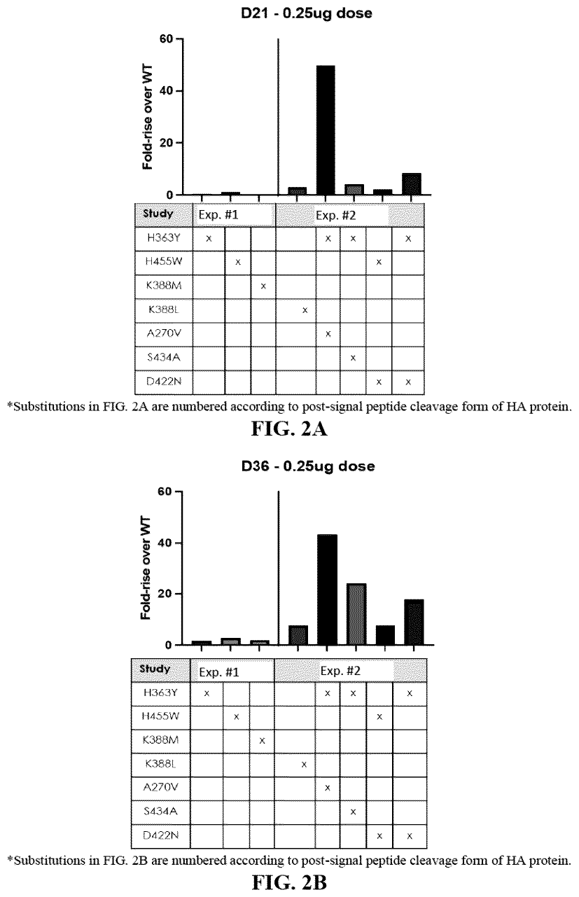

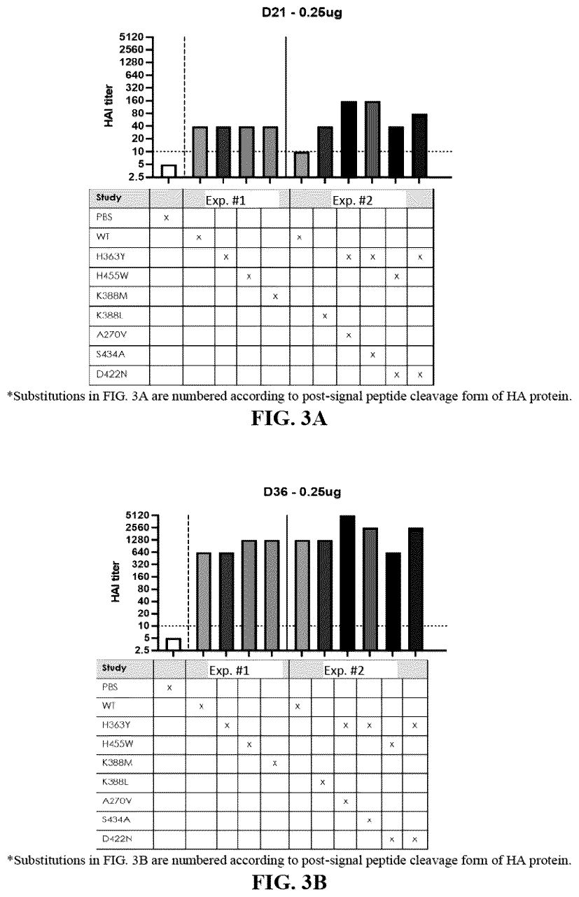

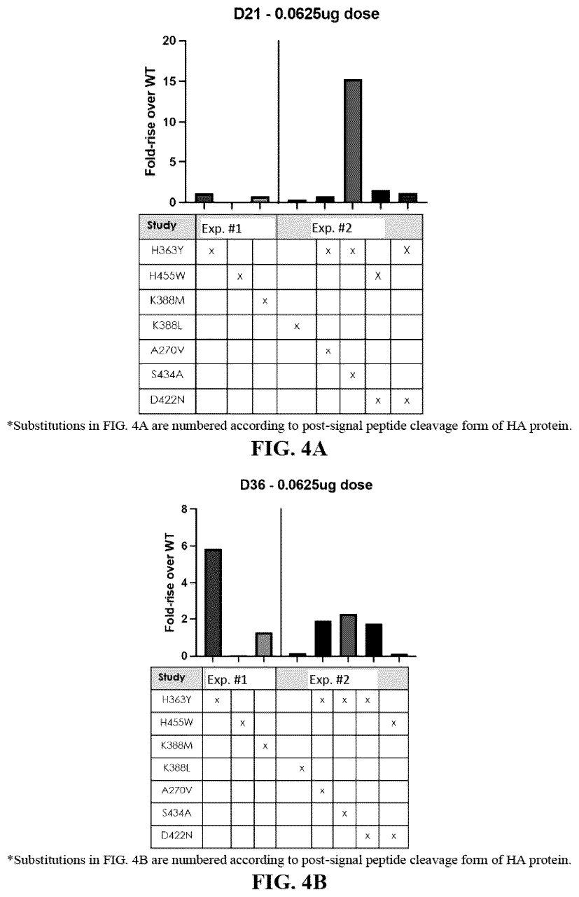

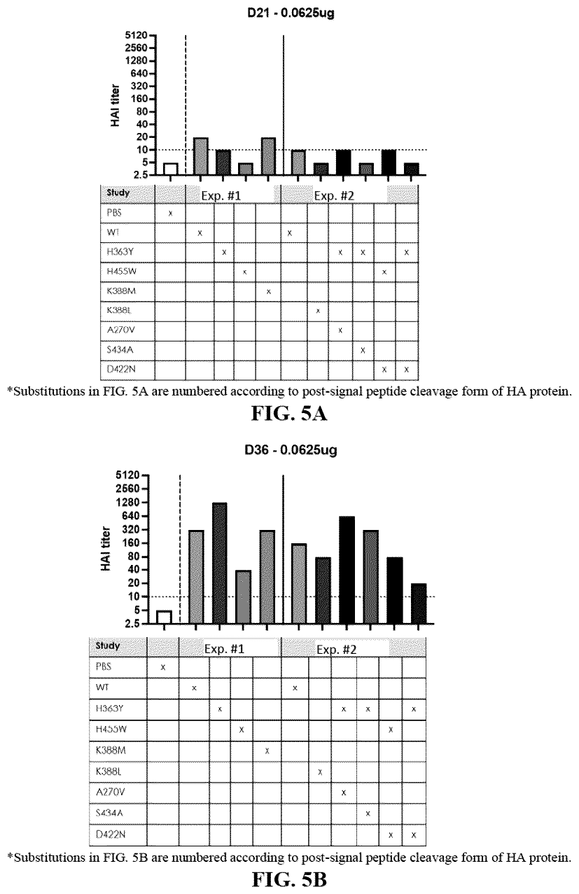

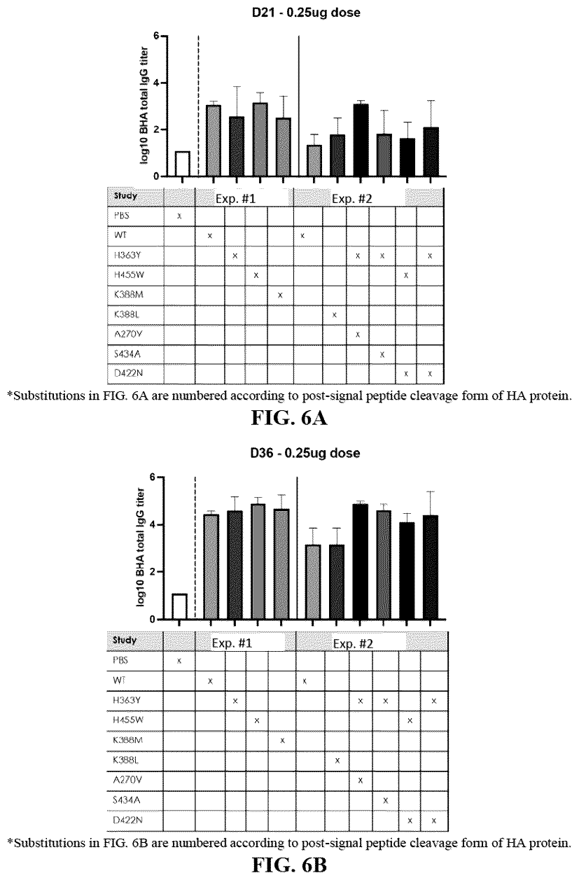

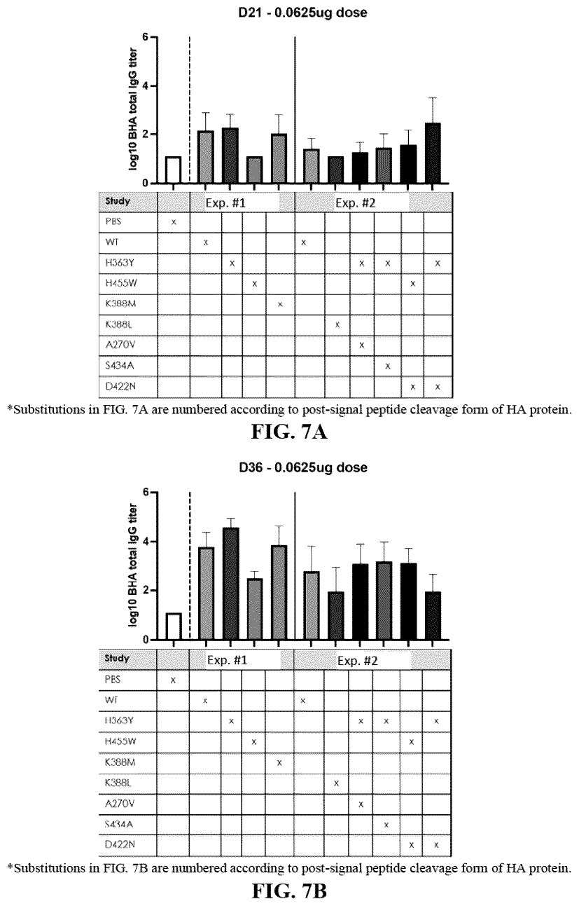

In an in vivo experiment, mice were administered mRNA encoding influenza B virus HA antigens having different stabilizing mutations at 0.25 μg/dose. Twenty-one and 36 days later, antibody-binding titers were measured by ELISA and normalized to wild-type mice. The data in FIG. 2A (Day 21) and FIG. 2B (Day 36) demonstrate that the combination of H381Y and A288V substitutions (numbered according to the full-length amino acid sequence of SEQ ID NO: 71 before signal peptide cleavage) had the greatest impact on binding titers. The ELISA titer data is provided in FIG. 6A (Day 21) and FIG. 6B (Day 36). HAI titers were measured in the same mice (FIG. 3A, Day 21; FIG. 3B, Day 36), and the combination of H381Y and A288V mutations was also found to have the greatest impact on HAI titer at Day 36. The antibody titer data for the 0.0625 μg dose is shown in FIGS. 4A-4B (ELISA data in FIGS. 7A-7B) and the HAI titers are shown in FIGS. 5A-5B. With respect to the HAI titers, the response to the 0.0625 μg dose was variable, although the H381Y and the H381Y+A288V mutations were found to induce the highest titers.

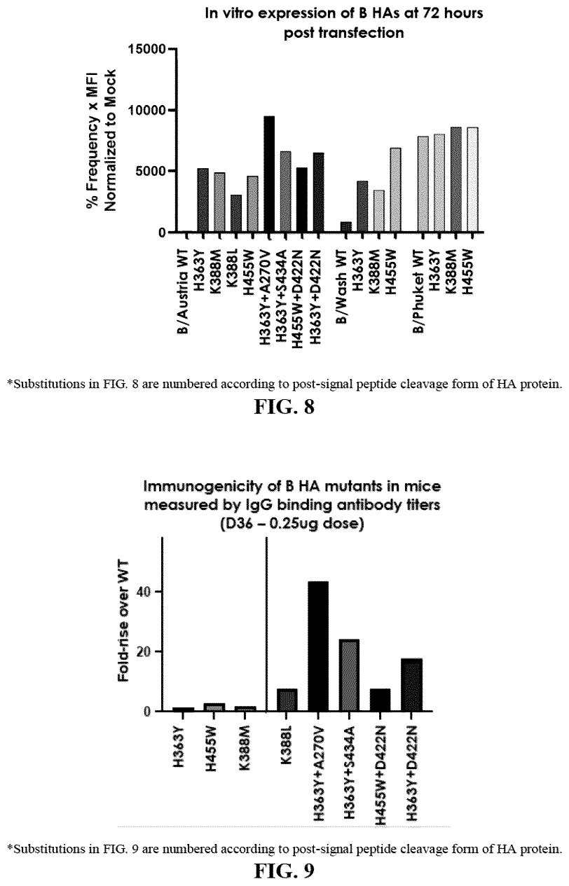

Further screening of the different mutations is shown in FIGS. 8 and 9, which depict the in vitro expression of different mutant influenza B virus HA antigens and immunogenicity (measured as IgG antibody binding titers on Day 36 after a 0.25 μg dose), respectively. Single point mutant influenza B virus HA proteins were found to have higher in vitro expression compared to wild-type and further improvements were observed for double mutants in the B/Austria (B/Victoria lineage) background. The single point mutations also improve expression for B/Washington HA and to a lesser extent B/Phuket HA proteins of the B/Yamagata lineage. In vivo, the double mutant, H381Y+A288V, showed the highest fold-rise over wild-type at a 0.25 ug dose.

### Example 2. Effects of HA Stabilization on In Vitro Expression

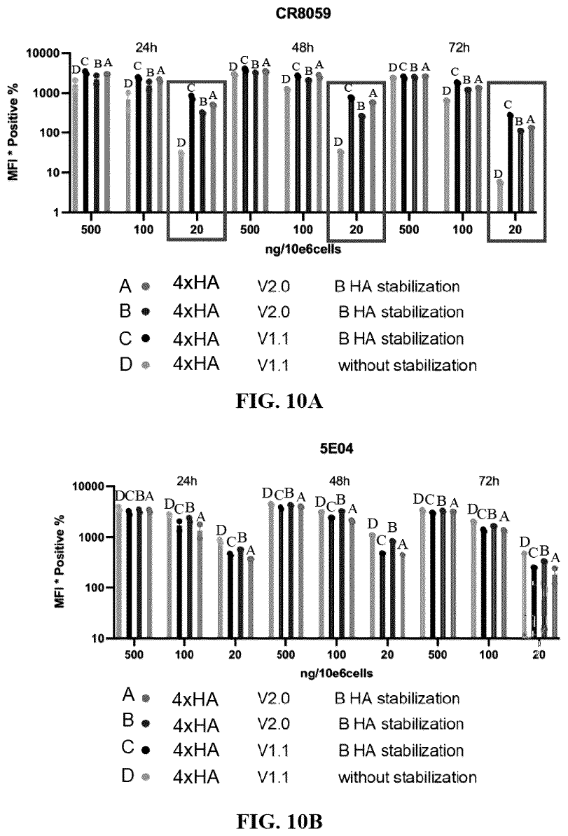

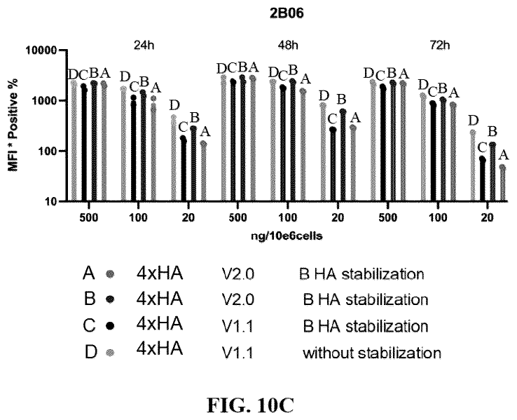

The effects of different stabilizing HA mutations on in vitro expression levels were examined. Expi293 cells were incubated with mRNA encoding four HA antigens:two HA antigens from influenza A viruses and two HA antigens from influenza B viruses. The HA antigens from influenza B viruses were either stabilized with at least one mutation, or not stabilized (wild-type HA). After incubation, the cells were stained with a IBV HA-specific antibody (CR8059), which showed that the stabilized constructs were more highly expressed at relatively lower dosage levels (FIG. 10A). Cells were also stained with an H3-specific antibody (5E04) (FIG. 10B) or an H1-specific antibody (2B06) (FIG. 10C). With respect to the A strain-specific antibodies, no differences in expression levels were noted with respect to the mutated and wild-type mRNA, demonstrating that the stabilized influenza B virus HA antigens did not affect the expression of the influenza A virus HA antigens.

### Example 3. Combination Flu/SARS-CoV-2 Vaccine in Mice

The effects of administering a combination vaccine comprising mRNA encoding influenza virus HA antigens and a SARS-CoV-2 antigen were examined. The combination vaccine was created from a combination of:
“4×HA”, an mRNA vaccine comprising four mRNAs encoding two HA antigens from influenza viruses (H1 and H3) and two HA antigens from influenza B viruses (H1 HA+H3 HA+B/Yamagata lineage HA+B/Victoria lineage HA at 1:1:1:1 ratio)
“NTD-RBD-HAtm”, an mRNA vaccine comprising mRNA encoding a SARS-CoV-2 fusion protein comprising the receptor binding domain (RBD) and N-terminal domain (NTD) of the Spike protein, linked with an influenza virus HA transmembrane sequence

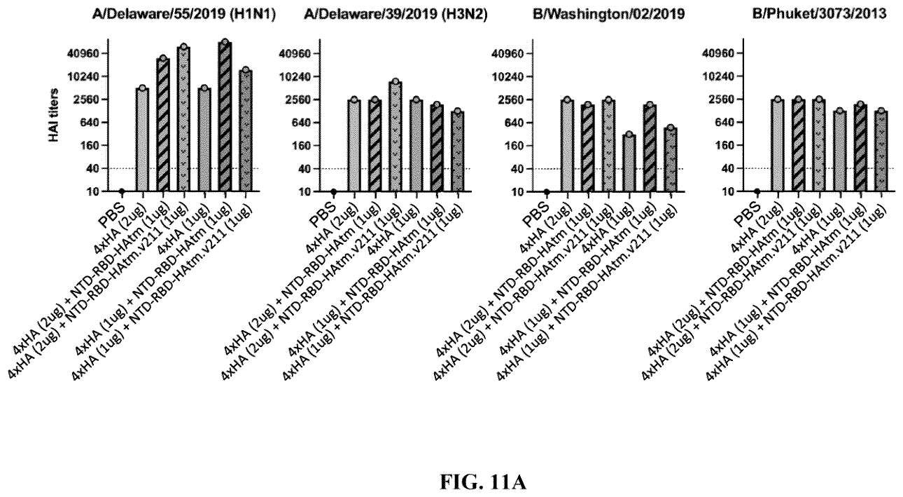

The effects of the resulting combination vaccine, “4×HA/NTD-RBD-HAtm” (4×HA and NTD-RBD-HAtm mixed at a 1:1 or 2:1 ratio) were tested. BALB/c mice were administered the different vaccines in a prime-boost protocol and the resulting HAI titers and SARS-CoV-2 neutralization titers were measured. It was found that the inclusion of additional mRNA encoding a SARS-CoV-2 antigen did not impact the resulting HAI titers compared to the group administered the flu vaccine (4×HA) alone (FIG. 11A). In addition, SARS-CoV-2 neutralization titers were reduced relative to administration of NTD-RBd-HAtm by itself, but only to levels seen in mice administered mRNA encoding a SARS-CoV-2 protein having two stabilizing proline mutations (FIG. 11B).

### Example 4. A Multi-Phase, Adaptive, Randomized, Observer-Blind, Placebo- and Active-Control Study to Evaluate the Safety, Reactogenicity, and Immunogenicity of mRNA-Based Influenza and SARS-CoV-2 Multi-Component Vaccines in Healthy Adults

4×HA/NTD-RBD-HAtm, a lipid-encapsulated mRNA-based prophylactic combination vaccine encoding influenza virus and SARS-CoV-2 antigens, is being developed. 4×HA/NTD-RBD-HAtm contains six mRNAs: four sequences encoding surface glycoprotein hemagglutinin (HA) of seasonal influenza viruses and two sequences encoding linked N terminal domain (NTD) and receptor-binding domain (RBD) of the SARS-CoV-2 spike protein. 4×HA/NTD-RBD-HAtm encodes the respective antigens also encoded by 4×HA (seasonal influenza) and NTD-RBD-HAtm (SARS-CoV-2 bivalent booster).

### Study Vaccines: 4×HA/NTD-RBD-HAtm (4×HA/NTD-RBD-HAtm.1, 4×HA/NTD-RBD-HAtm.2, and 4×HA/NTD-RBD-HAtm.3)

As noted above, 4×HA/NTD-RBD-HAtm contains 6 mRNAs: 4 mRNAs that each encode a membrane-bound hemagglutinin (HA) of one of the 4 different influenza virus strains recommended by the WHO in an equivalent mass ratio (SEQ ID NOs: 64-67) and 2 sequences that encode the N-terminal domain (NTD) and receptor-binding domain (RBD) of the SARS-CoV-2 Spike glycoprotein of the original (Wuhan-Hu-1) and BA.4/BA.5 variants in an equivalent mass ratio (SEQ ID NOs: 68-69). The NTD and RBD sequences are linked together with a short flexible linker and a transmembrane domain anchors the protein to the cell membrane. The influenza virus HA proteins encoded by 4×HA/NTD-RBD-HAtm are also encoded by 4×HA (influenza) vaccine, and the SARS-CoV-2 fusion proteins are also encoded by the NTD-RBD-HAtm (SARS-CoV-2 bivalent) vaccine, except that the influenza B virus HA antigens encoded by 4×HA/NTD-RBD-HAtm vaccine contain stabilizing point mutations. The mRNAs in 4×HA/NTD-RBD-HAtm further comprise an updated sequence in the non-coding region.

Three 4×HA/NTD-RBD-HAtm vaccine compositions will be evaluated; each contain the same 6 mRNAs, but differ by the ratio of the combined mass of the 4 influenza virus mRNAs to the combined mass of the two SARS COV 2 mRNAs:
1. 4×HA/NTD-RBD-HAtm.1:5:1 combined influenza virus mRNA to combined SARS-CoV-2 mRNA mass ratio.
2. 4×HA/NTD-RBD-HAtm.2:10:1 combined influenza virus mRNA to combined SARs-CoV-2 mRNA mass ratio.
3. 4×HA/NTD-RBD-HAtm.3:20:1 combined influenza virus mRNA to combined SARS-CoV-2 mRNA mass ratio.

4×HA, NTD-RBD-HAtm, and S-2P will be used as controls. 4×HA is an LNP-encapsulated, mRNA-based, prophylactic vaccine containing 4 mRNAs that encode membrane-bound HA of the 4 different influenza virus strains recommended by the WHO in an equivalent mRNA mass ratio. NTD-RBD-HAtm is an LNP-encapsulated, mRNA-based prophylactic vaccine that encodes the RBD and NTD of the SARS-CoV-2 spike glycoprotein. The NTD and RBD sequences are linked together with a short flexible linker and a transmembrane domain anchors the protein to the cell membrane. S-2P.222 (Bivalent vaccine) is an LNP-encapsulated, mRNA-based prophylactic vaccine that contains 2 mRNAs that encode the full-length SARS-CoV-2 prefusion-stabilized spike glycoproteins of the original (Wuhan-Hu-1) and BA.4/BA.5 variants in an equivalent mass ratio. The lipid nanoparticle comprises 48% Compound 1, 2.5% PEG-DMG, 38.5% cholesterol, and 11% DSPC.

### Cohorts and Duration

The trial will enroll approximately 1050 participants into 1 of 2 age cohorts: Cohort A for participants ≥65 to <80 years of age (approximately 550 participants) and Cohort B for participants ≥18 to <65 years of age (approximately 500 participants). In Cohort B, approximately 500 participants will be randomized (in equal allocation ratio) into the investigational vaccine arms with stratification by 2 age groups, ≥18 to <50 years and ≥50 to <65 years, and approximately 50% of the participants will be ≥50 to <65 years of age. The study intervention information, including dosages, is provided in Table E4-1 below.

All participants will have follow-up visits for 6 months after the study vaccination.

TABLE E4-1
Study Interventions and Dosages

| Cohort | Cohort | | | | |
|---|---|---|---|---|---|
| A | B | | Total | | |
| Arms: | Arms: | | mRNA | Component mRNA | |
| (65-79 | (18-64 | | Dose | Dose (μg) | |
| years) | years) | Intervention | (μg) | COVID | Flu |
| | B1 | 4xHA/NTD-RBD- | 15 | 2.5 | 12.5 |
| A2 | B2 | HAtm.1 (1:5) | 30 | 5 | 25 |
| A3 | | | 60 | 10 | 50 |
| A4 | B4 | 4xHA/NTD-RBD- | 27.5 | 2.5 | 25 |
| A5 | B5 | HAtm.2 (1:10) | 55 | 5 | 50 |
| A6 | B6 | 4xHA/NTD-RBD- | 52.5 | 2.5 | 50 |
| | | HAtm.3 (1:20) | | | |
| A7 | B7 | 4xHA.4 | 50 | — | 50 |
| A8 | B8 | NTD-RBD-HAtm | 5 | 5 | — |
| | | (Wuhan-Hu-1 and | | | |
| | | BA.4/BA.5) | | | |
| A9 | B9 | S-2P Bivalent | 50 | 50 | — |
| | | (Wuhan-Hu-1 and | | | |
| | | BA.4/BA.5) | | | |
| A10 | B10 | 4xHA | 50 | — | 50 |
| A11 | B11 | Fluaria | — | — | — |
| A12 | | Fluzone HD | — | — | — |

### Objectives and Endpoints

The primary objective of this trial is to evaluate the safety and reactogenicity of the candidate vaccines across study arms.

The secondary objectives are to evaluate the humoral immune responses to vaccine-matched strains for influenza virus and SARS-CoV-2 across study vaccine arms at Day 29 and to evaluate the humoral immune responses to vaccine-matched strains for influenza virus and SARS-CoV-2 across study vaccine arms at all evaluable humoral immunogenicity time points. These will be evidenced by the following endpoints: GMT and GMFR at Day 29 compared to Day 1 by HAI assay for influenza and by PsVNA for SARS-CoV-2; influenza: percentage of participants with seroconversion, defined as a Day 29 titer ≥1:40 if baseline is <1:10 or a 4-fold or greater rise if baseline is ≥1:10 in anti-HA antibodies measured by HAI assay; SARS-CoV-2: percentage of participants with seroresponse, defined as a Day 29 titer ≥4-fold if baseline is ≥LLOQ or ≥4×LLOQ if baseline titer is <LLOQ in nAb titers measured by PsVNA; GMT and GMFR at all evaluable time points compared to Day 1 by HAI for influenza and PsVNA for SARS-CoV-2; influenza: percentage of participants with seroconversion; and SARS-CoV-2: percentage of participants with seroresponse.

The exploratory objectives are to evaluate the humoral immune responses to vaccine-mismatched strains for influenza virus and SARS-CoV-2 across study vaccine arms; to evaluate the humoral immune responses against vaccine-matched and vaccine-mismatched strains for influenza virus and SARS-CoV-2 across study vaccine arms; to evaluate the cellular immune responses against influenza virus and SARS-CoV-2 in a subset of participants; to further characterize the immune response to influenza virus and SARS-CoV-2 across study vaccine arms; and to assess the occurrence of clinical influenza and COVID-19 in study participants and characterize their immune response to infection and viral isolates. The exploratory objectives will be measured by the following endpoints: GMT and GMFR at all evaluable time points compared to Day 1 by HAI for influenza virus and PsVNA for SARS-CoV-2; influenza: percentage of participants with seroconversion; SARS-CoV-2: percentage of participants with seroresponse; GMT and GMFR at all evaluable time points compared to Day 1 by alternative methods, including, but not limited to: microneutralization assay for influenza virus or ligand-binding assay for SARS-CoV-2; frequency, magnitude, and phenotype of virus-specific T-cell and B-cell responses measured by flow cytometry or other methods; targeted repertoire analysis of B-cells and T-cells after vaccination; frequency, specificities, or other endpoints to be determined for the further characterization of immune responses; frequency of RT-PCR-confirmed clinical influenza and COVID-19; and assessment of immune responses to infection and viral isolates.

### Example 5. Stabilizing Mutations for 4 Influenza Virus Strains

In this Example, several stabilizing mutations were made to hemagglutinin (HA) antigens of five influenza viruses: B/Austria/1359417/2021 HA (B/Victoria lineage), B/Phuket/3073/2013 (B/Yamagata lineage) HA, A/Darwin/6/2021 H3 HA, A/Wisconsin/67/2022 H1 HA, and A/Sydney H1 HA. Effects on expression, antibody binding, yield, and thermostability, as described in Example 1, were measured for each mutant HA. Briefly, plasmids encoding mRNAs encoding each candidate stabilized mutant HA were constructed and used to transfect cells. After 72 hours, surface expression levels were determined using flow cytometry on cell preparations stained with polyclonal sera antibodies, HA subtype-specific antigen antibodies, and/or HA subtype region-specific antibodies. Melting temperature, oligomerization profile, and yield were determined for soluble versions of each candidate protein using thermal shift assays, sodium dodecyl sulfate-polyacrylamide gel electrophoresis (SDS-PAGE), and AlphaLISA.

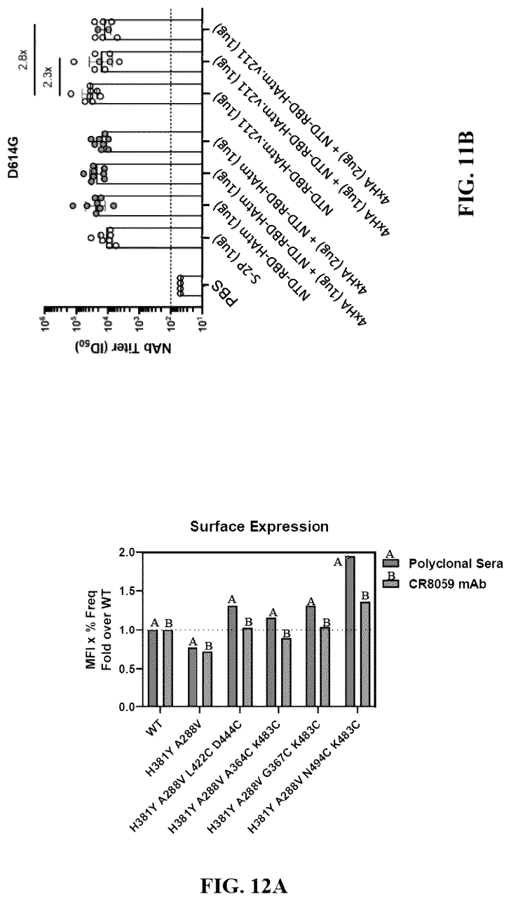

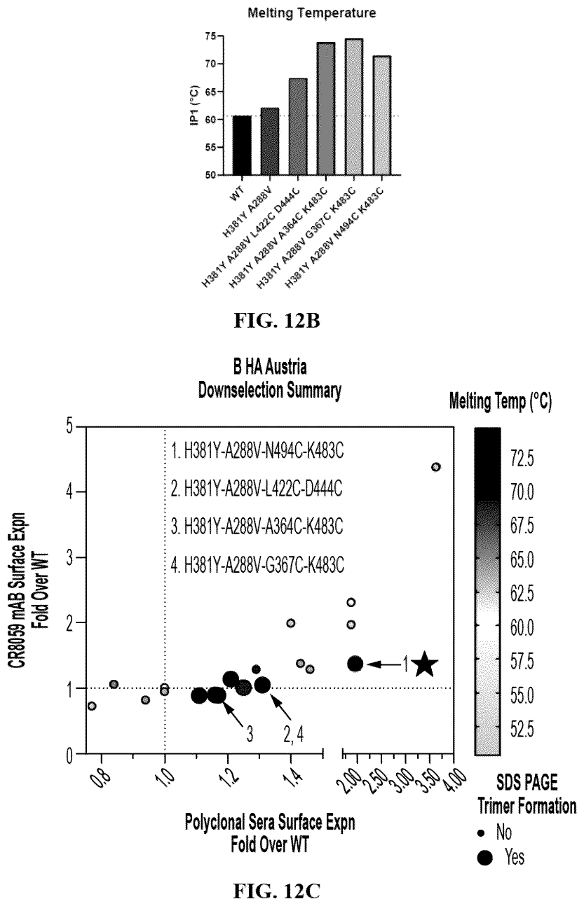

Based on surface expression and soluble protein metrics as described above, of nineteen B/Austria stabilizing mutant HA proteins (shown in Table E5-1), five candidate mutations (H381Y-A288V, H381Y-A288V-L422C-D444C, H381Y-A288V-A364C-K483C, H381Y-A288V-G367C-K483C, and H381Y-A288V-N494C-K483C relative to wild-type) were selected for further analysis. FIG. 12A shows surface expression of each mutation for each of the five B/Austria mutant HA proteins; relative to wild-type B/Austria HA, the H381Y-A288V-N494C-K483C mutant, a construct comprising an inter-protomer disulfide bond between stalk cysteines, showed beneficial surface expression based on polyclonal sera antibody and CR8059 antibody staining. FIG. 12B shows increased thermostability for four of the mutations relative to wild-type, including H381Y-A288V-N494C-K483C. As shown in FIG. 12C, the H381Y-A288V-N494C-K483C mutant HA had 2-fold surface expression as measured by polyclonal sera and +11° C. melting temperature over wild-type, and exhibited sustained formation of a trimer.

TABLE E5-1
B_Austria_HA Mutation Candidates

| | | | Surface Expression | | Soluble Protein | | |
|---|---|---|---|---|---|---|---|
| | | | Sera | CR8059 | Melt | | Melt |
| | | | Freq * | Freq * | Temp 1 | Yield | Temp 2 |
| Strain | Mutation | Construct Type | MFI/WT | MFI/WT | (° C.) | (mg) | (° C.) |
| B_Austria_HA | Wild-type (WT) | NA | 1.00 | 1.00 | 60.41 | 0.92 | 60.7 |
| B_Austria_HA | H381Y-A288V | pH Switch/ | 0.77 | 0.72 | 62.16 | NA | NA |
| | | Cavity Filling | | | | | |
| B_Austria_HA | H381Y-A288V- | Intra-protomer DS | 0.94 | 0.81 | 63.41 | 0.73 | 64.25 |
| | S27C-Y349C | (Head) | | | | | |
| B_Austria_HA | H381Y-A288V- | Intra-protomer DS | 1.40 | 1.98 | NA | 0.02 | 50.35 |
| | I295C-K328C | (Head) | | | | | |
| B_Austria_HA | H381Y-A288V- | Intra-protomer DS | 1.00 | 0.94 | 63.26 | 0.79 | 63.5 |
| | S399C-H473C | (Stalk) | | | | | |
| B_Austria_HA | H381Y-A288V- | Intra-protomer | 1.31 | 1.03 | 66.83 | 0.87 | 67.45 |
| | L422C-D444C | DS (Stalk) | | | | | |
| B_Austria_HA | H381Y-A288V- | Inter-protomer DS | 3.63 | 4.37 | 51.46 | 1.32 | 50.97 |
| | K118C-L216C | (Head) | | | | | |
| B_Austria_HA | H381Y-A288V- | Inter-protomer DS | 1.87 | 1.96 | NA | 0.17 | 55 |
| | V237C-D261C | (Head) | | | | | |
| B_Austria_HA | H381Y-A288V- | Inter-protomer DS | 1.17 | 0.88 | 70.69 | 0.67 | 71 |
| | G363C-K480C | (Stalk) | | | | | |
| B_Austria_HA | H381Y-A288V- | Intra-protomer | 1.16 | 0.89 | 72.75 | 0.68 | 73.92 |
| | A364C-K483C | DS (Stalk) | | | | | |
| B_Austria_HA | H381Y-A288V- | Inter-protomer DS | 1.11 | 0.88 | 71.78 | 0.71 | 72.03 |
| | I365C-A476C | (Stalk) | | | | | |
| B_Austria_HA | H381Y-A288V- | Inter-protomer DS | 1.25 | 1.00 | 68.56 | 0.82 | 68.84 |
| | A366C-R479C | (Stalk) | | | | | |
| B_Austria_HA | H381Y-A288V- | Intra-protomer | 1.31 | 1.04 | 73.51 | 0.61 | 74.61 |
| | G367C-K483C | DS (Stalk) | | | | | |
| B_Austria_HA | H381Y-A288V- | Inter-protomer DS | 1.21 | 1.13 | 70.37 | 0.90 | 70.59 |
| | E435C-A428C | (Stalk) | | | | | |
| B_Austria_HA | H381Y-A288V- | Intra-protomer | 1.95 | 1.36 | 71.01 | 0.26 | 71.51 |
| | N494C-K483C | DS (Stalk) | | | | | |
| B_Austria_HA | H381Y-A288V- | Inter-protomer DS | 1.29 | 1.28 | 67.33 | 0.20 | 69.11 |
| | N494C-K480C | (Stalk) | | | | | |
| B_Austria_HA | H381Y-A288V- | Proline | 1.43 | 1.37 | 63.31 | 0.79 | 64.07 |
| | E416P-L417P- | Substitution | | | | | |
| | N434P-H433P | | | | | | |
| B_Austria_HA | H381Y-A288V- | Proline | 0.84 | 1.05 | 65.24 | 0.99 | 65.17 |
| | N434P-H433P | Substitution | | | | | |
| B_Austria_HA | H381Y-A288V- | Proline | 1.87 | 2.30 | 59.29 | 0.35 | 59.49 |
| | T515P-F516P | Substitution | | | | | |
| B_Austria_HA | H381Y-A288V- | pH Switch/ | 1.46 | 1.28 | 62.86 | 0.84 | 63.78 |
| | H473F | Cavity Filling | | | | | |

Bold = Candidates pursued in FIGs. 12A-C.
MFI = Mean Fluorescence Intensity;
WT = wild-type;
DS = introduced disulfide bond

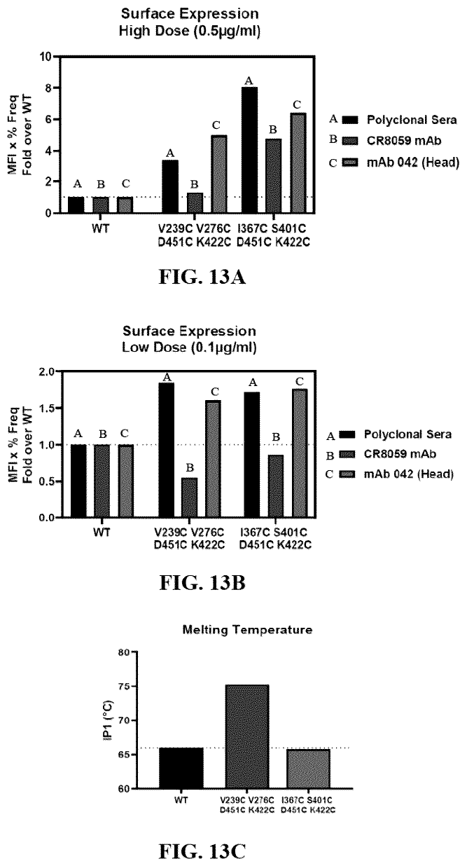

Based on surface expression and soluble protein metrics as described above, of nine B/Phuket stabilizing mutant HA proteins (shown in Table E5-2), three mutations (V239C-V276C, I367C-S401C, and D451C-K422C) relative to wild-type were combined and selected for further analysis. FIG. 13A shows surface expression of each of two B/Phuket mutant HA proteins: V239C-V276C-D451C-K422C and I367C-S401C-D451C-K422C. Both mutants showed increased binding to polyclonal sera antibodies and mAb 042 antibodies when expressed in cells transfected with high doses of mRNA (0.5 μg/mL). Only the I367C-S401C-D451C-K422C mutant, a construct comprising two inter-protomer bonds between protomer stalks, also showed increased binding to CR8059 antibodies. Similar findings were made for proteins expressed in cells transfected with low doses of mRNA (0.1 μg/mL; FIG. 13B). However, thermostability assays showed an increased melting temperature for V239C-V276C-D451C-K422C, a construct comprising an inter-protomer disulfide bond between head cysteines, and an inter-protomer disulfide bond between stalk cysteines, while the melting temperature of the I367C-S401C-D451C-K422C mutant was similar to wild-type (FIG. 13C). The V239C-V276C-D451C-K422C mutant HA exhibited 3.4-fold higher surface expression as measured by polyclonal sera and +10° C. melting temperature over wild-type, and exhibited sustained formation of a trimer.

TABLE E5-2
B_Phuket_HA Mutation Candidates

| | | | Flow Cytometry | | | | |
|---|---|---|---|---|---|---|---|
| | | | (Surface Expresesion) | | Soluble Protein | | |
| | | | Sera | CR8059 | Melt | | Melt |
| | | | Freq * | Freq * | Temp 1 | Yield | Temp 2 |
| Strain | Mutation | Construct Type | MFI/WT | MFI/WT | (° C.) | (mg) | (° C.) |
| B_Phuket_HA | Wild-type (WT) | WT | 1.000 | 1.000 | 60.43 | 1.72 | 62.52 |
| B_Phuket_HA | A231C-G273C | Intra-protomer DS | 0.903 | 0.844 | 66.28 | 1.45 | 69.01 |
| | | (Head) | | | | | |
| B_Phuket_HA | K295C-1332C | Intra-protomer DS | 4.314 | 2.347 | 59.11 | 0.30 | 61.39 |
| | | (Head) | | | | | |
| B_Phuket_HA | A396C-L510C | Intra-protomer DS | 2.655 | 1.675 | 59.97 | 0.62 | 62.61 |
| | | (Stalk) | | | | | |
| B_Phuket_HA | V239C-V276C | Inter-protomer | 3.672 | 1.784 | 61.53 | 0.59 | 63.76 |
| | | DS (Head) | | | | | |
| B_Phuket_HA | I367C-S401C | Inter-protomer | 1.203 | 0.880 | 65.48 | 2.48 | 69.61 |
| | | DS (Stalk) | | | | | |
| B_Phuket_HA | F363C-E404C | Inter-protomer DS | 1.097 | 0.866 | 65.12 | 2.41 | 68.03 |
| | | (Stalk) | | | | | |
| B_Phuket_HA | E437C-G429C | Inter-protomer DS | 1.951 | 1.504 | 62.88 | 2.98 | 65.05 |
| | | (Stalk) | | | | | |
| B_Phuket_HA | D451C-K422C | Inter-protomer | 2.068 | 1.714 | 63.40 | 2.09 | 65.01 |
| | | DS (Stalk) | | | | | |
| B_Phuket_HA | H381Y-A290V | pH Switch/Cavity | NA | NA | NA | 2.64 | 62.59 |
| | | Filling | | | | | |

Bold = Candidates pursued in FIGs. 13A-C.
MFI = Mean Fluorescence Intensity;
WT = wild-type;
DS = introduced disulfide bond

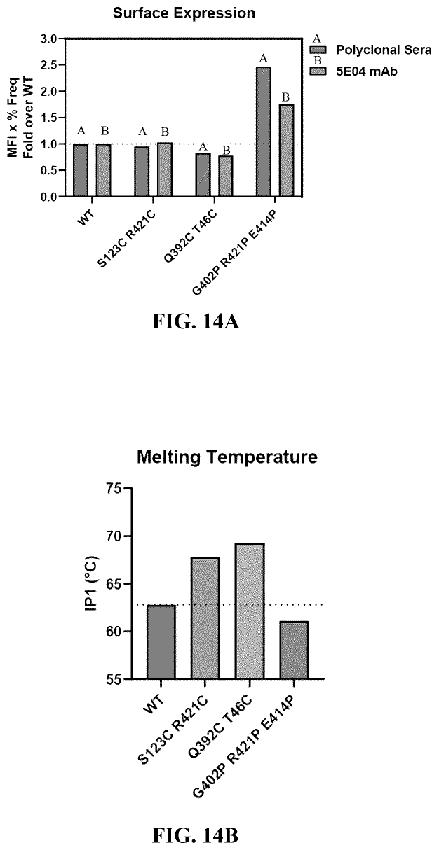

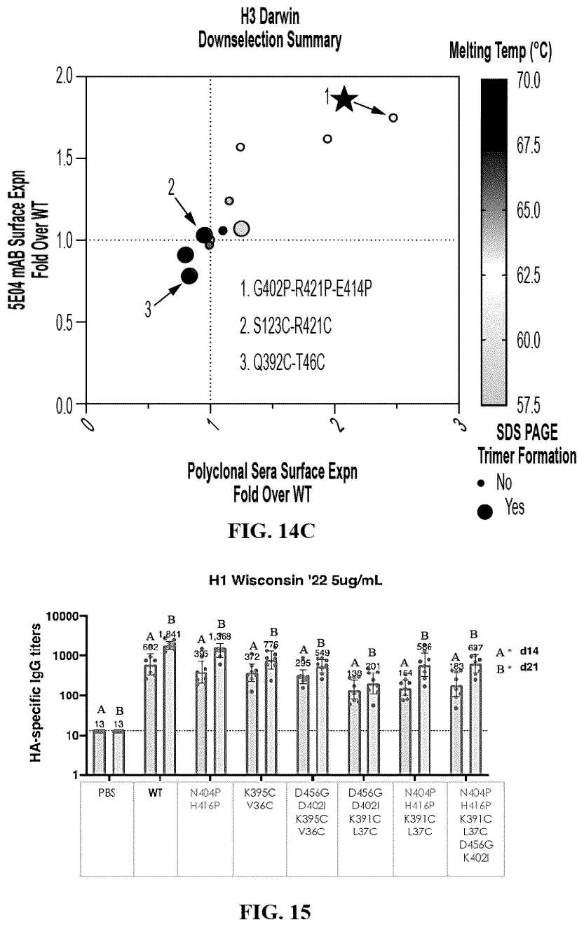

Based on surface expression and soluble protein metrics as described above, of nine A/Darwin stabilizing mutant H3 HA proteins (shown in Table E5-3), three candidate mutations (S123C-R241C, Q392C-T46C, and G402P-R421P-E414P) relative to wild-type were selected for further analysis. FIG. 14A shows surface expression of each mutation for each of the three A/Darwin mutant H3 HA proteins. Only the G402P-R421P-E414P mutant, a construct comprising proline substitution stabilizations, showed increased binding to both polyclonal sera antibodies and 5E04 antibodies. Melting temperatures of the S123C-R241C mutant and Q392C-T46C mutant, comprising interprotomer disulfide bonds between head cysteines and or stalk cysteines, respectively, were increased relative to wild-type (FIG. 14B). As shown in FIG. 14C, the G402P-R421P-E414P mutant H3 HA had 3-fold higher surface expression as measured by polyclonal sera and +1.7° C. melting temperature over wild-type.

TABLE E5-3
A_Darwin_H3 HA Mutation Candidates

| | | | Flow Cytometry | | | | |
|---|---|---|---|---|---|---|---|
| | | | (Surface Expression) | | Soluble Protein | | |
| | | | Sera | 5E04 | Melt | | Melt |
| | | | Freq * | Freq * | Temp 1 | Yield | Temp 2 |
| Strain | Mutation | Construct Type | MFI/WT | MFI/WT | (° C.) | (mg) | (° C.) |
| A_Darwin_H3 | Wild-type | WT | 1.00 | 1.00 | 60.8 | 0.88 | 62.8 |
| | (WT) | | | | | | |
| A_Darwin_H3 | T40C-A55C | Intra-protomer | 1.24 | 1.57 | 60.5 | 1.32 | 62.9 |
| | | DS (Head) | | | | | |
| A_Darwin_H3 | S123C-R421C | Inter-protomer | 0.95 | 1.03 | 67.4 | 0.67 | 67.8 |
| | | DS (Head) | | | | | |
| A_Darwin_H3 | L260C-P237C | Inter-protomer | 0.80 | 0.91 | 67.4 | 0.20 | 70.0 |
| | | DS (Head) | | | | | |
| A_Darwin_H3 | Q392C-T46C | Inter-protomer | 0.83 | 0.78 | 67.9 | 0.84 | 69.3 |
| | | DS (Stalk) | | | | | |
| A_Darwin_H3 | I411C-Y428C | Inter-protomer | 1.25 | 1.07 | 62.3 | 2.98 | 63.5 |
| | | DS (Stalk) | | | | | |
| A_Darwin_H3 | G402P-R421P- | Proline | 2.47 | 1.75 | 61.9 | 3.16 | 61.1 |
| | E414P | Substitution | | | | | |
| A_Darwin_H3 | K403G | Glycine | 1.15 | 1.24 | 61.8 | 1.32 | 63.7 |
| | | Substitution | | | | | |
| A_Darwin_H3 | F408G-H409G | Glycine | 1.94 | 1.62 | 60.4 | 2.28 | 62.6 |
| | | Substitution | | | | | |

Bold = Candidates pursued in FIGs 14A-C.
MFI = Mean Fluorescence Intensity;
WT = wild-type;
DS = introduced disulfide bond

Based on surface expression and soluble protein metrics as described above, of sixteen A/Wisconsin stabilizing mutant H1 HA proteins (shown in Table E5-4), six candidate mutations and/or combinations of mutations (N404P-H416P, K395C-V36C, D456C-D402I-K395C-V36C, D456G-D402I-K395C-V36C, N404P-H416P-K391C-L37C, and N404P-H416P-K391C-L37C-D456G-K402I) relative to wild-type were selected for further analysis. FIG. 15 shows the results of an additional in vivo experiment, in which mice were administered mRNA encoding candidate stabilized mutant H1 HA proteins, as described in Example 1. HA-specific IgG titers were measured on Day 14 and Day 21. All candidate mutant H1 HA proteins elicited sera having similar binding titers relative to wild-type H1 HA.

TABLE E5-4
A_Wisconsin_H1 HA Mutation Candidates

| | | | Soluble Protein | | |
|---|---|---|---|---|---|
| | | | Sera | 2B06 | Melt |
| | | | Freq * | Freq * | Temp |
| Strain | Mutation | Construct Type | MFI/WT | MFI/WT | (° C.) |
| A_Wisconsin_H1 | Wild-type (WT) | WT | 1.000 | 1.000 | 61.11 |
| A_Wisconsin_H1 | V410C-L462C | Intra-protomer DS | 0.884 | 1.832 | 53.07 |
| A_Wisconsin_H1 | E120C-K419C | Inter-protomer DS | 1.267 | 2.662 | 58.85 |
| A_Wisconsin_H1 | K391C-L37C | Inter-protomer | 1.005 | 1.103 | 74.27 |
| | | DS | | | |
| A_Wisconsin_H1 | K395C-V36C | Inter-protomer | 1.095 | 1.373 | 73.51 |
| | | DS | | | |
| A_Wisconsin_H1 | Q406C-D430C | Inter-protomer DS | 1.228 | 2.822 | 58.20 |
| A_Wisconsin_H1 | S457C-L346C | Inter-protomer DS | 1.050 | 1.117 | 59.29 |
| A_Wisconsin_H1 | N461C-G348C | Inter-protomer DS | 1.072 | 1.014 | 65.86 |
| A_Wisconsin_H1 | N404P-K419P | Proline | 1.582 | 1.820 | 53.36 |
| A_Wisconsin_H1 | N404P-H416P | Proline | 1.550 | 1.510 | 69.10 |
| A_Wisconsin_H1 | T405P-Q406P | Proline | 1.026 | 0.935 | 67.93 |
| A_Wisconsin_H1 | N415P-H416P | Proline | 1.319 | 3.194 | 54.79 |
| A_Wisconsin_H1 | H25Y-H45E | pH Switch/ | 0.995 | 1.026 | 61.97 |
| | | Cavity Filling | | | |
| A_Wisconsin_H1 | H370Y-K497W | pH Switch/ | 1.337 | 1.217 | 60.51 |
| | | Cavity Filling | | | |
| A_Wisconsin_H1 | K402G-T405G- | Glycine | 1.152 | 1.467 | 64.34 |
| | F407G | Substitution | | | |
| A_Wisconsin_H1 | K395I-E447I | Cavity Filling | 0.424 | 0.745 | 70.67 |
| A_Wisconsin_H1 | D456G-K402I | Cavity Filling | 1.179 | 1.201 | 65.95 |
| | | (K4021)/Glycine | | | |
| | | (D456G) | | | |

Bold = Top candidates pursued in FIG. 15.
MFI = Mean Fluorescence Intensity;
WT = wild-type;
DS = introduced disulfide bond

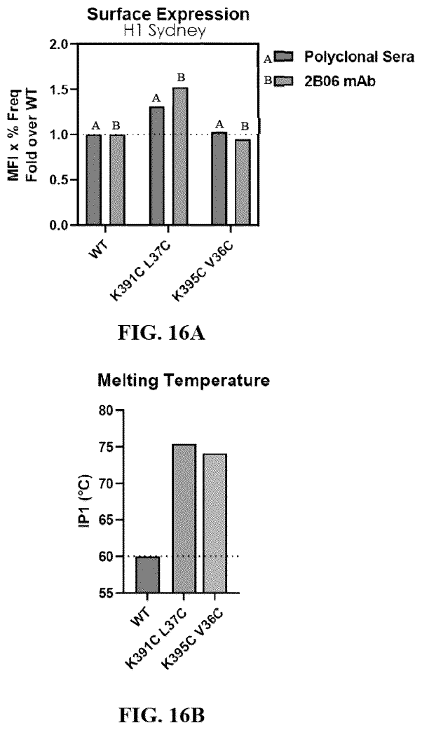

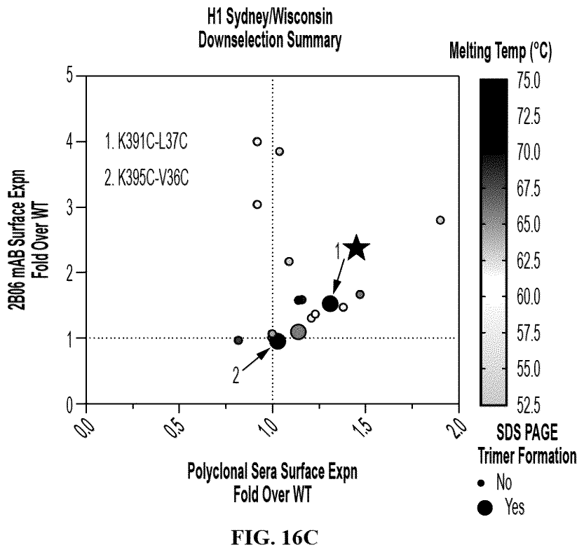

Based on surface expression and soluble protein metrics as described above, of sixteen A/Sydney stabilizing mutant H1 HA proteins (shown in Table E5-5), two candidate mutations (K391C-L37C and K395C-V36C) relative to wild-type were selected for further analysis. FIG. 16A shows surface expression of each mutation for each of the two A/Sydney candidate mutant H1 HA proteins. The K391C-L37C mutant, a construct comprising an inter-protomer disulfide bond, showed increased binding to both polyclonal sera antibodies and 2B06 antibodies. Melting temperatures for both mutants were similarly increased relative to wild-type (FIG. 16B). Ultimately, the expression of each mutation for each of the two A/Sydney mutant H1 proteins. As shown in FIG. 16C, the K391C-L³⁷C mutant H1 HA had 1.4-fold surface expression as measured by polyclonal sera and +15° C. melting temperature over wild-type, and exhibited sustained formation of a trimer.

TABLE E5-5
A_Sydney_H1 HA Mutation Candidates

| | | | | | | | Soluble |
|---|---|---|---|---|---|---|---|
| | | | Flow Cytometry (Surface Expression) | | | | Protein |
| | | | Sera | 2B06 | | Melt | Melt |
| | | | Freq * | Freq * | Yield/ | Temp - | Temp 2 |
| Strain | Mutation | Construct Type | MFI/WT | MFI/WT | WT | WT (° C.) | (° C.) |
| A_Sydney_H1 | Wild-type | WT | 1.00 | 1.00 | 1.00 | 61.53 | 60.7 |
| | (WT) | | | | | | |
| A_Sydney_H1 | V410C | Intra-protomer | 1.09 | 2.17 | 1.23 | 53.63 | NA |
| | L462C | DS | | | | | |
| A_Sydney_H1 | E120C | Inter-protomer | 0.92 | 4.00 | 2.29 | 59.32 | 64.25 |
| | K419C | DS | | | | | |
| A_Sydney_H1 | K391C | Inter-protomer | 1.31 | 1.52 | 1.24 | 73.88 | 50.35 |
| | L37C | DS | | | | | |
| A_Sydney_H1 | K395C | Inter-protomer | 1.03 | 0.95 | 1.72 | 73.07 | 63.5 |
| | V36C | DS | | | | | |
| A_Sydney_H1 | Q406C | Inter-protomer | 0.92 | 3.04 | 3.24 | 58.25 | 67.45 |
| | D430C | DS | | | | | |
| A_Sydney_H1 | S457C | Inter-protomer | 1.38 | 1.47 | 1.95 | 58.99 | 50.97 |
| | L346C | DS | | | | | |
| A_Sydney_H1 | N461C | Inter-protomer | 1.90 | 2.80 | 4.90 | 65.24 | 55 |
| | G348C | DS | | | | | |
| A_Sydney_H1 | N404P | Proline | 1.14 | 1.09 | 1.74 | 53.73 | 71 |
| | K419P | | | | | | |
| A_Sydney_H1 | N404P | Proline | 1.16 | 1.58 | 3.05 | 68.17 | 73.92 |
| | H416P | | | | | | |
| A_Sydney_H1 | T405P | Proline | 0.82 | 0.96 | 1.06 | 66.77 | 72.03 |
| | Q406P | | | | | | |
| A_Sydney_H1 | N415P | Proline | 1.04 | 3.85 | 2.89 | 55.41 | 68.84 |
| | H416P | | | | | | |
| A_Sydney_H1 | H25Y H45E | pH Switch/ | 1.21 | 1.30 | 1.14 | 58.87 | 74.61 |
| | | Cavity Filling | | | | | |
| A_Sydney_H1 | H370Y | pH Switch/ | 1.23 | 1.36 | 3.38 | 58.58 | 70.59 |
| | K497W | Cavity Filling | | | | | |
| A_Sydney_H1 | K402G | Glycine | 1.00 | 1.06 | 1.23 | 62.57 | 71.51 |
| | T405G | Substitution | | | | | |
| | F407G | | | | | | |
| A_Sydney_H1 | K395I E447I | Cavity Filling | 1.14 | 1.57 | 0.31 | 70.60 | 69.11 |
| A_Sydney_H1 | D456G | Cavity Filling | 1.47 | 1.66 | 1.04 | 66.22 | 64.07 |
| | K402I | (K402I)/ | | | | | |
| | | Glycine | | | | | |
| | | (D456G) | | | | | |

Bold = Top candidates pursued in FIGs. 16A-C.
MFI = Mean Fluorescence Intensity;
WT = wild-type;
DS = introduced disulfide bond

### Example 6. A Phase 1, Randomized, Double-Blind, Active-Control Study to Evaluate the Safety, Reactogenicity, and Immunogenicity of Octovalent mRNA-Based Influenza Vaccines in Healthy Adults

In this Phase 1 study, 4×HA/4×NA at 1:1 HA:NA and 4×HA/4×NA at 3:1 HA:NA, two octovalent lipid-encapsulated mRNA-based prophylactic combination vaccines encoding stabilized influenza virus glycoprotein hemagglutinin (HA) and neuraminidase (NA) antigens were evaluated for reactogenicity and safety. 4×HA/4×NA at 1:1 HA:NA contains 8 mRNAs: 4 mRNA sequences encoding stabilized mutant membrane-bound HA proteins (SEQ ID NOs: 85, 87, 91, and 94) and 4 mRNA sequences that encode NA proteins of the 4 different influenza viruses recommended by the WHO in an equivalent mass ratio. Substitutions present in each sequence are:
SEQ ID NO: 85-Influenza B/Phuket/3073/2013 (B/Yamagata lineage) virus HA protein with V239C, V276C, D451C, and K422C substitutions relative to SEQ ID NO: 70;
SEQ ID NO: 87-Influenza B/Austria/1359417/2021 (B/Victoria lineage) virus HA protein with H381Y, A288V, N494C, and K483C substitutions relative to SEQ ID NO: 71;
SEQ ID NO: 91-Influenza A/Darwin/6/2021(H3N2) virus HA protein with G402P, R421P, and E414P substitutions relative to SEQ ID NO: 82; and
SEQ ID NO: 94-Influenza A/Wisconsin/67/2022(H1N1)pdm09 HA protein with K391C and L37C substitutions relative to SEQ ID NO: 83.

The proteins encoded by 4×HA/4×NA at 1:1 HA:NA are also encoded by 4×HA/4×NA at 3:1 HA:NA, with the exception that 4×HA/4×NA at 3:1 HA:NA contains a 3:1 HA:NA ratio. 4×HA, and FLUBLOK®, a traditional seasonal flu vaccine, were used as controls. 4×HA is an LNP-encapsulated, mRNA-based, prophylactic vaccine containing 4 mRNAs that encode membrane-bound HA of the 4 different influenza viruses recommended by the WHO in an equivalent mRNA mass ratio. The lipid nanoparticle comprises 48% Compound 1, 2.5% PEG-DMG, 38.5% cholesterol, and 11% DSPC. FLUBLOK®, is an approved, quadrivalent recombinant full-length HA vaccine comprising HAs from A/West Virginia/30/2022 (A/Wisconsin/67/2022 pdm09-like (H1N1), virus)A/Darwin/6/2021(H3N2), B/Austria/1359417/2021 and B/Phuket/3073/2013, and a carrier comprising sodium chloride, monobasic sodium phosphate, dibasic sodium phosphate, and polysorbate.

### Cohorts and Duration

The trial enrolled 572 participants into cohorts of 2 age groups, each cohort comprising 8 vaccination groups: Cohort A for participants 18-49 years of age and Cohort B for participants 50-75 years of age (approximately 35 participants per vaccination group, per cohort). The study intervention information, including dosages, is provided in Table E6-1 below. All participants had follow-up visits for 6 months after the study vaccination.

TABLE E6-1
Study Interventions and Dosages

| Cohort | Cohort | | | | |
|---|---|---|---|---|---|
| A Arms | B Arms | | | Component | |
| (18-49 | (50-75 | | Total mRNA | mRNA dose (μg) | |
| years) | years) | Intervention | Dose (μg) | HA | NA |
| A1 | B1 | FLUBLOK ®, | — | — | — |
| A2 | B2 | 4xHA | 50 | 50 | — |
| A3 | B3 | 4xHA/4xNA at 3:1 | 25 | 18.75 | 6.25 |
| | | HA:NA | | | |
| A4 | B4 | 4xHA/4xNA at 1:1 | 50 | 25 | 25 |
| | | HA:NA | | | |
| A5 | B5 | 4xHA/4xNA at 3:1 | 50 | 37.5 | 12.5 |
| | | HA:NA | | | |
| A6 | B6 | 4xHA/4xNA at 1:1 | 100 | 50 | 50 |
| | | HA:NA | | | |
| A7 | B7 | 4xHA/4xNA at 3:1 | 100 | 75 | 25 |
| | | HA:NA | | | |
| A8 | B8 | 4xHA/4xNA at 1:1 | 150 | 75 | 75 |
| | | HA:NA | | | |

### Objectives and Endpoints

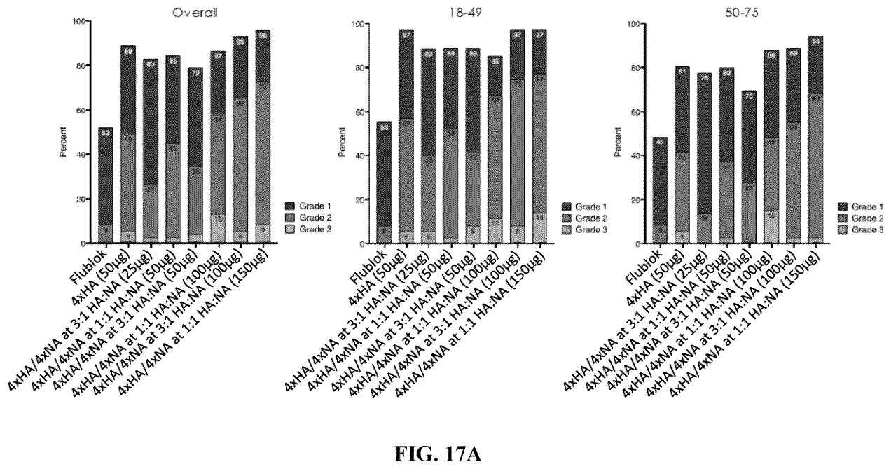

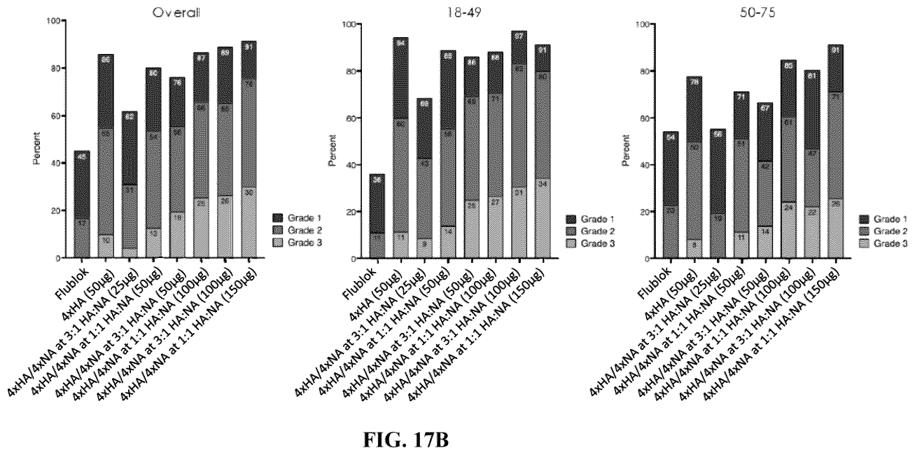

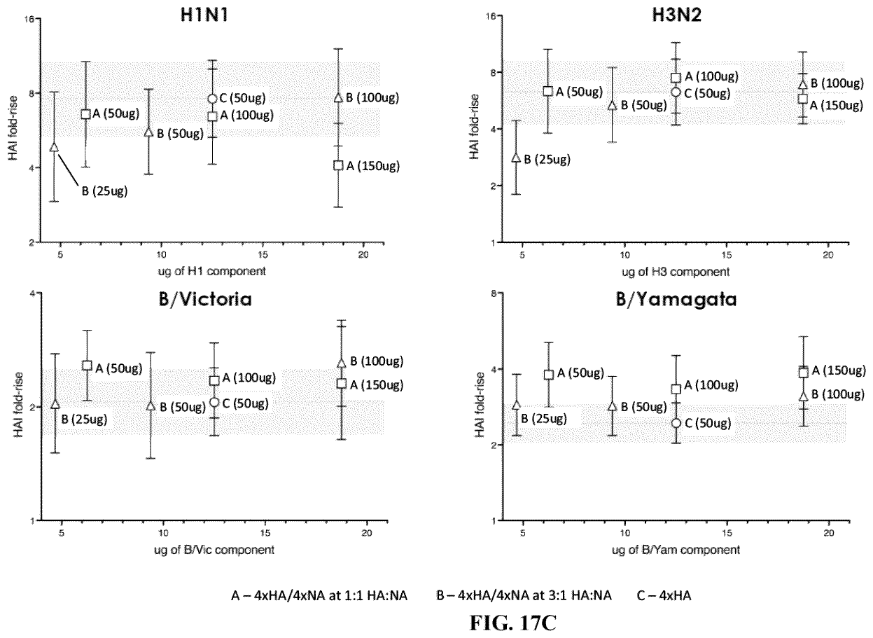

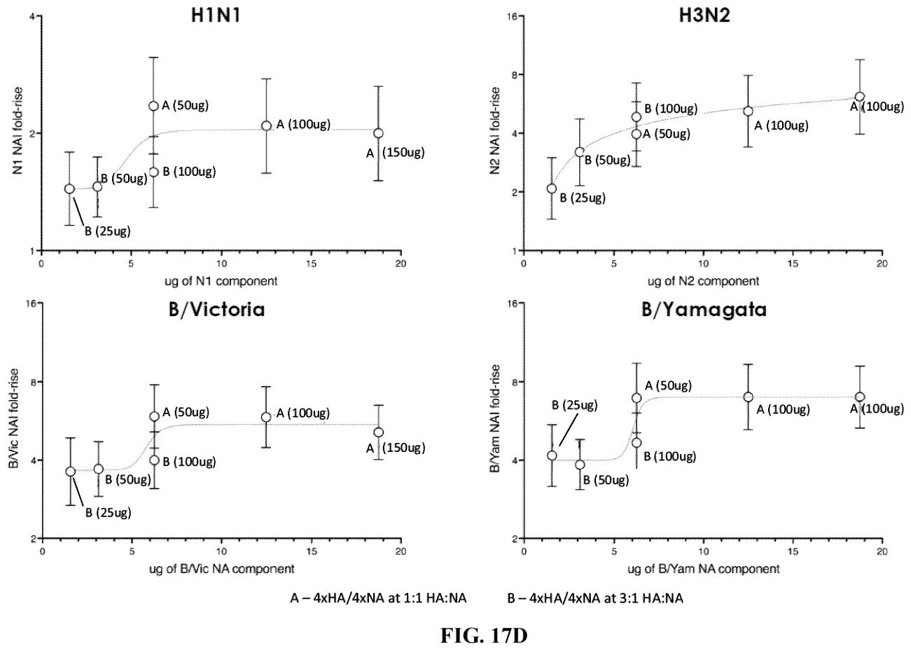

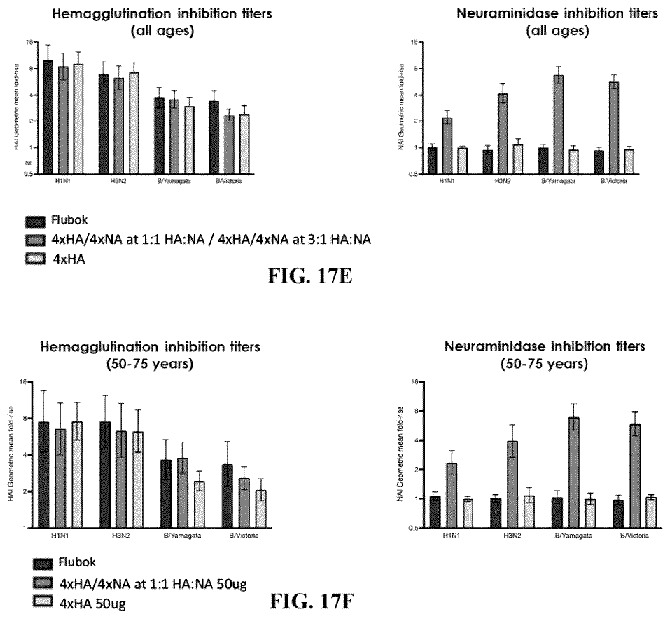

The primary objective of this trial was to evaluate the safety and reactogenicity of the candidate vaccines across study arms. As shown in FIG. 17A, 50 μg doses of 4×HA, 4×HA/4×NA at 1:1 HA:NA, and 4×HA/4×NA at 3:1 HA:NA elicited similar local reactions in all participants, regardless of composition. Importantly, introduction of NA-antigen encoding mRNA did not increase local reactogenicity. FIG. 17B shows similar systemic reactogenicity for participants to whom 50 μg doses of 4×HA/4×NA at 1:1 HA:NA and 4×HA was administered, while slightly higher systemic reactogenicity was observed in subjects treated with 50 μg doses of 4×HA/4×NA at 3:1 HA:NA. Higher doses of mRNA vaccines elicited higher rates of grade 2 and grade 3 local and systemic reactions.

The secondary objectives of this trial were to evaluate the humoral immune responses to vaccine-matched strains for influenza viruses across study vaccine arms at Day 29. GMT and GMFR at Day 29 compared to Day 1 were assessed by HAI assay and NAI assay. In subjects aged 50-75 years at Day 29 compared to Day 1 by HAI assays. 4×HA/4×NA at 1:1 HA:NA at 50 μg elicited similar HAI fold-rises as 4×HA at 50 μg, despite including only half the dose of HA-encoding mRNAs (FIG. 17C). Similarly, FIG. 17D shows that, in subjects aged 50-75 years of age, for similar dose levels of NA-encoding mRNAs, 4×HA/4×NA at 1:1 HA:NA shows a trend for higher NAI titer rises than 4×HA/4×NA at 3:1 HA:NA (except for N2). Elicited HAI and NAI titers for FLUBLOK®, 4×HA/4×NA at 1:1 HA:NA/1030, and 4×HA were compared and 4×HA/4×NA at 1:1 HA:NA (50 μg) was found to elicit both HAI titers, similar to those induced by FLUBLOK®, and robust NAI titers (FIG. 17E). These findings held for subjects aged 50-75 (FIG. 17F).

### Example 7. A Phase 2, Adaptive, Randomized, Observer-Blind, Placebo- and Active-Control Study to Evaluate the Safety and Immunogenicity of Octovalent mRNA-Based Influenza Vaccines Relative to a Quadrivalent mRNA-Based Influenza Vaccine in Healthy Adults

As described in Example 6, 4×HA/4×NA at 1:1 HA:NA is an octovalent lipid-encapsulated mRNA-based prophylactic combination vaccines encoding stabilized influenza virus glycoprotein hemagglutinin (HA) and neuraminidase (NA) antigens. 4×HA/4×NA at 1:1 HA:NA contains 8 mRNAs: 4 mRNA sequences encoding stabilized mutant membrane-bound HA proteins (SEQ ID NOs: 85, 87, 91, and 94) and 4 mRNA sequences encoding wild-type or stabilized mutant membrane-bound NA proteins of the 4 different influenza viruses recommended by the WHO in an equivalent mass ratio. In this Phase 2 trial, the safety and immunogenicity of the two 4×HA/4×NA at 1:1 HA:NA formulations will be compared to 4×HA.

### Cohorts and Duration

The trial will consist of two parts: Part A and Part B. The Part A trial will enroll approximately 900 older adult participants into 9 arms. Approximately 100 participants will be randomized (in equal allocation ratio) into investigational vaccine arms with stratification by 2 age groups, 18-49 years and 50-75 years. The study intervention information for Part A, including dosages, is provided in Table E7-1 below.

TABLE E7-1
Part A Interventions and Dosages

| | | Total | | |
|---|---|---|---|---|
| | | mRNA | Component | |
| Study | | Dose | mRNA Dose (μg) | |
| Arms | Interventions | (μg) | HA | NA |
| A1 | 4xHA (4x HA mutant) | 50 | 50 | — |
| A2 | | 25 | 25 | — |
| A3 | 4xHA/4xNA at 1:1 HA:NA | 50 | 25 | 25 |
| A4 | (4x HA mutant; | 75 | 32.5 | 32.5 |
| | 4x mutated NA) | | | |
| A5 | 4xHA/4xNA at 1:1 HA:NA | 25 | 12.5 | 12.5 |
| A6 | (4x HA mutant; | 50 | 24 | 25 |
| | 4x wild-type NA) | | | |
| A7 | Fluzone HD (comparator) | — | — | — |
| A8 | FLUAD (comparator) | — | — | — |
| A9 | 4xHA (comparator) | 50 | 25 | — |

The Part B trial will enroll approximately 500 adult participants into 5 arms. Approximately 100 participants will be randomized (in equal allocation ratio) into the same 2 age groups. Study intervention information for Part B, including dosages, is provided in Table E7-2 below.

TABLE E7-2
Part B Interventions and Dosages

| Study | | Total mRNA | |
|---|---|---|---|
| Arms | Interventions | Dose (μg) | HA |
| B1 | 4xHA (4x HA mutant) | 2.5 | 50 |
| B2 | | 5 | 25 |
| B3 | | 10 | 25 |
| B4 | 4xHA (2xHA mutant- optimized B material | 50 | 50 |
| | from Example 4) | | |
| B5 | Fluarix | — | — |

All participants will have follow-up visits for 6 months after the study vaccination.

### Objectives and Endpoints

The primary objective of this trial is to evaluate the safety and reactogenicity of the candidate vaccines across study arms.

The secondary objectives are to evaluate the humoral immune responses to vaccine-matched strains for influenza virus across study vaccine arms at Day 29. These will be evidenced by the following endpoints: GMT and GMFR at Day 29 compared to Day 1 by HAI assay and NAI assay; percentage of participants with seroconversion, defined as a Day 29 titer ≥1:40 if baseline is <1:10 or a 4-fold or greater rise if baseline is ≥1:10 in anti-HA antibodies measured by HAI assay or in anti-NA antibodies measured by NAI assay; GMT and GMFR at all evaluable time points compared to Day 1 by HAI or NAI; and percentage of participants with seroconversion.

The exploratory objectives are to evaluate the humoral immune responses to vaccine-mismatched strains for influenza virus across study vaccine arms; to evaluate the humoral immune responses against vaccine-matched and vaccine-mismatched strains for influenza virus across study vaccine arms; to evaluate the cellular immune responses against influenza virus in a subset of participants; to further characterize the immune response to influenza virus across study vaccine arms; and to assess the occurrence of clinical influenza virus in study participants and characterize their immune response to infection and viral isolates. The exploratory objectives will be measured by the following endpoints: GMT and GMFR at all evaluable time points compared to Day 1 by HAI and NAI; percentage of participants with seroconversion; GMT and GMFR at all evaluable time points compared to Day 1 by alternative methods, including, but not limited to: microneutralization assay; frequency, magnitude, and phenotype of virus-specific T-cell and B-cell responses measured by flow cytometry or other methods; targeted repertoire analysis of B-cells and T-cells after vaccination; frequency, specificities, or other endpoints to be determined for the further characterization of immune responses; frequency of RT-PCR-confirmed clinical influenza; and assessment of immune responses to infection and viral isolates.

### Example 8. A Phase 3, Randomized, Stratified, Observer-Blind, Active-Control Study to Evaluate the Immunogenicity, Reactogenicity and Safety of a Seasonal Influenza Vaccine in Adults 18 Years and Older

As described in Example 6, the 4×HA vaccine comprises 4 mRNA sequences encoding stabilized mutant membrane-bound HA proteins (SEQ ID NOs: 85, 87, 91, and 94) of the four different influenza virus strains recommended by the WHO for 2022-2023 Northern Hemisphere cell- or recombinant-based vaccines was tested. In this Phase 3 trial, the immunogenicity, reactogenicity, and safety of the mRNA vaccine were compared to that of an influenza vaccine comparator (FLUARIX® Quadrivalent, which comprises 4 HAs of the 4 different influenza virus strains recommended by the WHO for 2022-2023 NH egg-based vaccines).

### Treatment Groups and Dose Levels

Medically stable adults, aged 18 years and older, were screened and enrolled. Approximately 2400 participants were randomly assigned to treatment in this study in a 1:1: ratio to 1 of 2 treatment groups to receive either a single dose of mRNA-1010 or a single dose of a licensed seasonal influenza vaccine as an active comparator (FLUARIX® Quadrivalent) (Table E8-1 below).

TABLE E8-1
Treatment Groups and Dose Levels

| | | Total | mRNA/Antigen | | |
|---|---|---|---|---|---|
| Treatment | Study | Dose | HA (each) | | No. of |
| Group | Intervention | (μg) | (μg) | | Participants |
| 1 | 4xHA (4x HA | 50 (of | 12.5 | (mRNA) | 1200 |
| | mutant) | mRNA) | | | |
| 2 | FLUARIX ® | 60 (of | 15 | (protein) | 1200 |

| | |
|---|---|
| Quadrivalent | protein) |

### Objectives and Endpoints

The primary objectives of this trial were to evaluate the humoral immunogenicity of the mRNA vaccine relative to the influenza comparator vaccine against four vaccine-matched IAV and IBV strains at Day 29; and to evaluate the safety and reactogenicity of the mRNA vaccine. The humoral immunity was determined by the GMT at Day 29 (measured by HAI assay) and the proportion of participants reaching seroconversion at Day 29 (as measured by HAI assay).

The secondary objective was to further evaluate the humoral immunogenicity of the mRNA vaccine against four vaccine-matched IAV and IBV strains at Day 29. This was evidenced by the following endpoints: the proportion of participants with HAI titer ≥1:40 at Day 29 and the GMFR comparison between Day 29 and Day 1 (baseline) measured by HAI assay.

The exploratory objectives were to evaluate the humoral immunogenicity of the mRNA vaccine relative to that of the comparator vaccine against four vaccine-matched IAV and IBV strains at Day 181 (end of study) and to assess the occurrence of clinical influenza in study participants and characterize their immune responses to infection and viral isolates. These objectives were determined by measuring the GMT at Day 181 by HAI; determining the proportion of participants who reach seroconversion by Day 181 as measured by HAI; determining the GMT and GMFR of neutralizing antibodies by assays (e.g., microneutralization assays) or alternative methods against vaccine-matched or vaccine-mismatched strains on Day 29 compared to Day 1 (baseline); determining the GMT and GMFR of anti-HA antibodies as measured by HAI against vaccine-mismatched strains on Day 29 compared to Day 1 (baseline); and determining the frequency of RT-PCR-confirmed IL1 and assessment of immune responses in participants with RT-PCR-confirmed ILI.

### Results

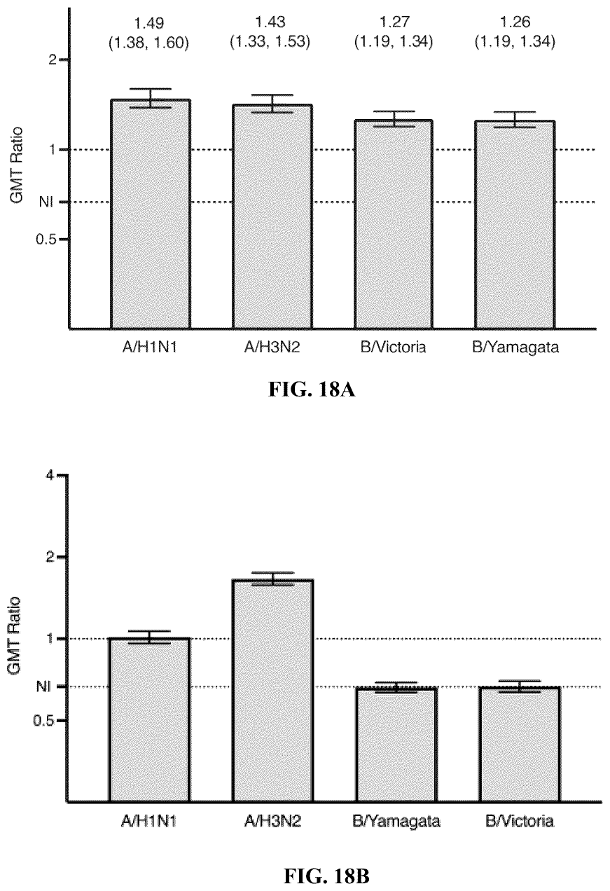

The mRNA vaccine was found to meet all primary immunogenicity endpoints, as measured by GMR and seroconversion rates. In particular, higher GMTs and seroconversion rates were observed for the mRNA vaccine for all four influenza virus strains (FIG. 18A). The increased titers against IBV HA proteins contrast with results of a previous study using mRNAs encoding non-mutant IBV HA proteins, where titers against both influenza B/Victoria lineage and influenza B/Yamagata lineage HA proteins were not superior to those elicited by comparator vaccines (FIG. 18B). Higher immunogenicity relative to the influenza vaccine comparator was consistently observed across all age groups, including adults of at least 65 years of age.

### Example 9. A Phase ½, Randomized, Observer-blind, Active-Control Study to Evaluate the Safety, Reactogenicity, and Immunogenicity of mRNA-based Influenza Vaccines in Healthy Adults

In this Phase ½ trial, the immunogenicity, reactogenicity, and safety of mRNA vaccines comprising 4 mRNAs each encoding one of: (i) an IAV H1 HA, (ii) an IAV H3 HA, (iii) an influenza B/Victoria lineage virus HA protein (with or without H381Y and A288V substitutions), and (iv) an influenza B/Yamagata lineage virus HA protein, were compared to that of comparator influenza vaccines (FLUARIX® Quadrivalent, which comprises 4 HAs of the 4 different influenza virus strains recommended by the WHO for 2022-2023 NH egg-based vaccines, and Fluzone high-dose).

### Treatment Groups and Dose Levels

Subjects aged 18 to 80 were assigned to a treatment group in Table E9-1 below.

TABLE E9-1
Interventions and Groups

| | | Number of | Vaccine | | Dose (each |
|---|---|---|---|---|---|
| Group | Age | Subjects | Components | Dose | mRNA/component) |
| A1 | 65 to <80 | 51 | Fluarix | | |
| | | | (comparator) | | |
| A2 | 65 to <80 | 50 | Fluzone high- | Standard of | Standard of care |
| | | | dose (HD) | care | |
| | | | (comparator) | | |
| A3 | 65 to <80 | 51 | 4xHA | 50 μg total | 12.5 μg each mRNA |
| | | | | mRNA | |
| A4 | 65 to <80 | 46 | 4xHA (H381Y- | 50 μg | 12.5 μg each mRNA |
| | | | A288V) | | |
| B1 | 50 to <65 | 25 | Fluarix | Standard of | Standard of care |
| | | | (comparator) | care | |
| B2 | 50 to <65 | 24 | 4xHA | 50 μg total | 12.5 μg each mRNA |
| | | | | mRNA | |
| B3 | 50 to <65 | 27 | 4xHA (H381Y- | 50 μg | 12.5 μg each mRNA |
| | | | A288V) | | |
| C1 | 18 to <50 | 24 | Fluarix | Standard of | Standard of care |
| | | | (comparator) | care | |
| C2 | 18 to <50 | 24 | 4xHA | 50 μg total | 12.5 μg each mRNA |
| | | | | mRNA | |
| C3 | 18 to <50 | 25 | 4xHA (H381Y- | 50 μg | 12.5 μg each mRNA |
| | | | A288V) | | |

### Objectives and Endpoints

One objective of this trial was to evaluate humoral immune responses to vaccine-matched strains for influenza viruses at Day 29. This was evidenced by the following endpoints: GMT and GMFR at Day 29 compared to Day 1 by HAI assay, and percentage of participants with seroconversion, defined as a Day 29 titer greater than or equal to 1:40 if Baseline titer is less than 1:10, or a 4-fold or greater rise if Baseline titer is greater than or equal to 1:10, in anti-HA antibodies measured by HAI assay.

### Results

The 4×HA mRNA vaccine in which the encoded influenza B/Victoria lineage HA protein included H381Y and A288V substitutions relative to SEQ ID NO: 71 elicited substantially higher anti-B/Victoria lineage HA antibody titers than each of the comparator mRNA vaccine (without H381Y and A288V substitutions), high-dose Fluzone, and Fluarix, as shown in Tables E9-2-E9-4:

TABLE E9-2
Immunogenicity in subjects (Cohort A, 65 to <80 years)

| | | | Fluzone HD | | | | 4xHA (H381Y- | |
|---|---|---|---|---|---|---|---|---|
| | Fluarix (A1) | | (A2) | | 4xHA (A3) | | A288V) (A4) | |
| | N = 51 | | N = 50 | | N = 46 | | N = 49 | |
| Day 1 GMT | 55.4 | (41.3, 74.3) | 49.2 | (35.8, 67.8) | 47.5 | (34.7, 65.0) | 47.4 | (35.2, 63.8) |
| Day 29 GMT | 100.7 | (70.1, 144.8) | 106.3 | (72.7, 155.3) | 89.3 | (66.4, 120.2) | 150.5 | (120.3, 188.4) |
| GMFR | 1.8 | (1.3, 2.5) | 2.2 | (1.6, 2.9) | 1.9 | (1.5, 2.4) | 3.3 | (2.4, 4.5) |
| SCR | 21.6 | (11.3, 35.3) | 18.0 | (8.6, 31.4) | 22.0 | (11.5, 36.0) | 47.7 | (32.5, 63.3) |

TABLE E9-3
Immunogenicity in subjects (Cohort B, 50 to <65 years)

| | Fluarix | | 4xHA | | 4xHA (H381Y- | |
|---|---|---|---|---|---|---|
| | (B1) | | (B2) | | A288V) (B3) | |
| | N = 25 | | N = 24 | | N = 27 | |
| Day 1 GMT | 45.9 | (28.6, 73.8) | 54.0 | (32.6, 89.7) | 39.0 | (23.3, 65.2) |
| Day 29 GMT | 104.2 | (67.7, 160.4) | 87.6 | (58.8, 130.3) | 125.4 | (79.4, 198.2) |
| GMFR | 2.3 | (1.5, 3.4) | 1.6 | (1.2, 2.2) | 3.2 | (2.2, 4.7) |
| SCR | 32.0 | (14.9, 53.5) | 8.7 | (1.1, 28.0) | 40.7 | (22.4, 61.2) |

TABLE E9-4
Immunogenicity in subjects (Cohort C, 18 to <50 years)

| | Fluarix | | 4xHA | | 4xHA (H381Y- | |
|---|---|---|---|---|---|---|
| | (C1) | | (C2) | | A288V) (C3) | |
| | N = 24 | | N = 24 | | N = 25 | |
| Day 1 GMT | 27.1 | (19.1, 38.4) | 20.3 | (13.6, 30.1) | 25.0 | (18.3, 34.0) |
| Day 29 GMT | 72.3 | (40.7, 128.5) | 40.6 | (28.5, 57.7) | 54.2 | (33.8, 87.0) |
| GMFR | 2.7 | (1.7, 4.2) | 2.2 | (1.6, 2.9) | 2.2 | (1.5, 3.1) |
| SCR | 37.5 | (18.8, 59.4) | 17.4 | (5.0, 38.8) | 36.0 | (18.0, 57.5) |

Data are shown as point estimate with 95% confidence interval in parentheses. GMT=geometric mean titer. GMFR=geometric mean fold rise. SCR=seroconversion rate. 95% CI is calculated based on the t-distribution of the log-transformed values for GM titer and GM fold rise values, respectively, then back transformed to the original scale for presentation.

Seroconversion at a participant level is defined as (a) a pre-vaccination HAI titer <1:10 and a post-vaccination titer >=1:40, or (b) a pre-vaccination HAI titer >=1:10 and a minimum 4-fold rise in post-vaccination HAI antibody titer.

### EXEMPLARY SEQUENCES

H1_Wisconsin_2019_WT_V2

| SEQ ID NO: 64 consists of from 5′ end to 3′ end: 5′ UTR SEQ ID NO: 45, | | 64 |
|---|---|---|
| mRNA ORF SEQ ID NO: 46, and 3′ UTR SEQ ID NO: 47. | | |
| Chemistry | 1-methylpseudouridine | |
| Cap | 7mG(5′)ppp(5′)NlmpNp | |
| 5′ UTR | AGGAAAUCGCAAAAUUUGCUCUUCGCGUUAGAUUUCUUUU | 45 |
| | AGUUUUCUCGCAACUAGCAAGCUUUUUGUUCUCGCC | |
| ORF of mRNA | AUGAAGGCCAUCCUGGUCGUGAUGCUGUACACCUUCACCAC | 46 |
| Construct | CGCCAACGCCGACACCCUGUGCAUCGGCUACCACGCCAACA | |
| (excluding the stop | ACAGCACCGACACCGUGGACACCGUGCUGGAGAAGAACGUG | |
| codon) | ACCGUGACCCACAGCGUGAACCUGCUGGAGGACAAGCACAA | |
| | CGGCAAGCUGUGCAAGCUGAGGGGAGUGGCACCCCUGCACC | |
| | UGGGCAAGUGCAACAUCGCCGGCUGGAUCCUGGGCAACCCC | |
| | GAGUGCGAGAGCCUGAGCACAGCCCGGAGCUGGAGCUACAU | |
| | CGUGGAGACCAGCAACAGCGACAACGGCACCUGUUACCCCG | |
| | GCGACUUCAUCAACUACGAGGAGCUGCGGGAGCAGCUGAGC | |
| | AGCGUGAGCAGCUUCGAGCGGUUCGAGAUCUUCCCCAAGAC | |
| | CAGCAGCUGGCCCAACCACGACAGCGACAACGGCGUGACAG | |
| | CAGCCUGUCCACACGCCGGAGCCAAGAGCUUCUACAAGAAC | |
| | CUGAUCUGGCUGGUGAAGAAGGGCAAGAGCUACCCCAAGA | |
| | UCAACCAGACCUACAUCAACGACAAGGGCAAGGAGGUGCUG | |
| | GUGCUGUGGGGCAUCCACCACCCACCUACCAUCGCCGACCA | |
| | GCAGAGCCUGUACCAGAACGCCGACGCCUACGUGUUCGUGG | |
| | GCACCAGCCGGUACAGCAAGAAGUUCAAGCCAGAGAUCGCC | |
| | ACCCGGCCCAAGGUGAGAGACCAGGAGGGCCGGAUGAACUA | |
| | CUACUGGACCCUGGUGGAGCCCGGAGACAAGAUUACCUUCG | |
| | AGGCCACCGGCAACCUGGUGGCCCCUCGGUACGCCUUCACC | |
| | AUGGAACGGGACGCUGGCAGCGGCAUCAUCAUCAGCGACAC | |
| | UCCCGUGCACGACUGCAACACCACCUGCCAGACUCCCGAGG | |
| | GCGCUAUCAACACCAGCCUGCCCUUCCAGAACGUGCACCCC | |
| | AUCACCAUCGGCAAGUGCCCCAAGUACGUAAAGAGCACCAA | |
| | AUUGCGGCUGGCCACCGGACUCAGGAACGUGCCCAGCAUCC | |
| | AAAGCCGGGGCCUGUUUGGCGCAAUCGCCGGCUUCAUCGAG | |
| | GGCGGCUGGACUGGCAUGGUGGACGGCUGGUACGGCUACCA | |
| | CCACCAGAACGAACAGGGGAGCGGCUACGCAGCUGACCUGA | |
| | AGAGCACCCAGAACGCCAUCGACAAGAUCACCAACAAGGUG | |
| | AACAGCGUGAUCGAGAAGAUGAACACCCAGUUCACCGCCGU | |
| | GGGCAAGGAGUUCAACCACCUGGAGAAGCGGAUCGAGAACC | |
| | UGAACAAGAAGGUGGACGACGGCUUCCUGGACAUCUGGACC | |
| | UACAACGCCGAGCUGCUGGUUCUGCUGGAGAACGAGCGGAC | |
| | CCUGGACUAUCACGACAGCAACGUGAAGAACCUGUACGAGA | |
| | AGGUGCGGAACCAGCUGAAGAACAACGCCAAGGAGAUCGGC | |
| | AACGGCUGCUUCGAGUUCUACCACAAGUGCGACAACACCUG | |
| | CAUGGAGAGCGUGAAGAACGGCACCUACGACUACCCCAAGU | |
| | ACAGCGAGGAGGCCAAGCUGAACCGGGAGAAGAUCGACGGC | |
| | GUGAAGCUGGACAGCACCCGGAUCUACCAGAUCCUGGCCAU | |
| | CUACAGCACCGUGGCCAGCAGCCUGGUGCUGGUGGUGAGCC | |
| | UGGGCGCCAUCAGCUUCUGGAUGUGCAGCAACGGCAGCCUG | |
| | CAGUGCCGGAUCUGCAUC | |
| 3′ UTR | UAAAGCUCCCCGGGGGCCUCGGUGGCCCUCCACCGAAGCAG | 47 |
| | CCAUCAGCACCCCCAGCCCCUCCUCCCCUUCCUGCAGGUCAA | |
| | AGUGGUCUUUGAAUAAAGUCUGAGUGGGCGGC | |
| Corresponding amino | MKAILVVMLYTFTTANADTLCIGYHANNSTDTVDTVLEKNVTV | 48 |
| acid sequence | THSVNLLEDKHNGKLCKLRGVAPLHLGKCNIAGWILGNPECES | |
| | LSTARSWSYIVETSNSDNGTCYPGDFINYEELREQLSSVSSFE | |
| | RFEIFPKTSSWPNHDSDNGVTAACPHAGAKSFYKNLIWLVKKG | |
| | KSYPKINQTYINDKGKEVLVLWGIHHPPTIADQQSLYQNADAY | |
| | VFVGTSRYSKKFKPEIATRPKVRDQEGRMNYYWTLVEPGDKIT | |
| | FEATGNLVAPRYAFTMERDAGSGIIISDTPVHDCNTTCQTPEG | |
| | AINTSLPFQNVHPITIGKCPKYVKSTKLRLATGLRNVPSIQSR | |
| | GLFGAIAGFIEGGWTGMVDGWYGYHHQNEQGSGYAADLKSTQN | |
| | AIDKITNKVNSVIEKMNTQFTAVGKEFNHLEKRIENLNKKVDD | |
| | GFLDIWTYNAELLVLLENERTLDYHDSNVKNLYEKVRNQLKNN | |
| | AKEIGNGCFEFYHKCDNTCMESVKNGTYDYPKYSEEAKLNREK | |
| | IDGVKLDSTRIYQILAIYSTVASSLVLVVSLGAISFWMCSNGS | |
| | LQCRICI | |
| PolyA tail | 100 nt | |

H3_A/Darwin/6/2021_G53D_V2

| SEQ ID NO: 65 consists of from 5′ end to 3′ end: 5′ UTR SEQ ID NO: 45, | | 65 |
|---|---|---|
| mRNA ORF SEQ ID NO: 49, and 3′ UTR SEQ ID NO: 50. | | |
| Chemistry | 1-methylpseudouridine | |
| Cap | 7mG(5′)ppp(5′)NlmpNp | |
| 5′ UTR | AGGAAAUCGCAAAAUUUGCUCUUCGCGUUAGAUUUUUUU | 45 |
| | AGUUUUCUCGCAACUAGCAAGCUUUUUGUUCUCGCC | |
| ORF of mRNA | AUGAAGACCAUCAUCGCCCUGAGCAACAUCCUGUGCCUGGU | 49 |
| Construct | GUUCGCCCAGAAGAUCCCCGGCAACGAUAACAGCACCGCCA | |
| (excluding the stop | CCCUGUGUCUGGGACACCACGCCGUGCCCAACGGCACCAUC | |
| codon) | GUGAAGACUAUCACCAACGACCGGAUCGAGGUGACCAACGC | |
| | CACCGAGCUGGUGCAGAACAGCAGCAUCGGCGAGAUCUGCG | |
| | ACAGCCCUCACCAGAUCCUGGACGGCGGCAACUGCACCCUG | |
| | AUCGACGCACUGCUGGGCGACCCUCAGUGCGACGGCUUUCA | |
| | GAACAAGGAGUGGGACCUGUUCGUGGAGAGAUCGCGGGCC | |
| | AACAGCAACUGCUACCCCUACGACGUCCCCGACUACGCAAG | |
| | CCUGAGAAGCCUCGUGGCCUCAAGCGGCACCCUGGAGUUCA | |
| | AGAACGAGAGCUUCAACUGGACCGGCGUGAAGCAGAACGGC | |
| | ACCUCAAGCGCCUGCAUCCGGGGCUCCAGCAGCAGCUUCUU | |
| | CUCACGGCUGAACUGGCUGACCCACCUGAACAACAUCUACC | |
| | CCGCCCAGAACGUGACCAUGCCCAACAAGGAGCAGUUCGAC | |
| | AAGCUGUACAUCUGGGGAGUGCACCAUCCCGACACCGACAA | |
| | GAACCAGAUUAGCCUGUUCGCCCAGAGCAGCGGCCGGAUCA | |
| | CCGUGAGCACCAAGCGGAGCCAGCAGGCCGUGAUCCCCAAC | |
| | AUCGGCUCUCGGCCCAGAAUCCGGGACAUCCCCAGCCGGAU | |
| | CAGCAUCUACUGGACCAUUGUGAAGCCCGGCGACAUCCUGC | |
| | UGAUCAACUCCACCGGCAACCUGAUCGCCCCUCGGGGCUAU | |
| | UUCAAGAUCCGGAGCGGCAAGAGCAGCAUCAUGCGGAGCGA | |
| | CGCCCCUAUCGGCAAGUGCAAGAGCGAGUGCAUCACACCCA | |
| | ACGGAAGCAUCCCCAACGACAAGCCCUUCCAGAACGUGAAC | |
| | CGGAUAACCUACGGCGCCUGCCCUAGAUACGUGAAGCAGAG | |
| | CACCCUGAAGCUGGCCACCGGCAUGCGGAACGUGCCCGAGA | |
| | AGCAGACUCGGGGCAUCUUCGGCGCCAUCGCCGGCUUCAUC | |
| | GAGAACGGCUGGGAGGGCAUGGUGGACGGCUGGUACGGCU | |
| | UCCGGCACCAGAACUCUGAGGGCAGAGGACAGGCCGCAGAC | |
| | CUGAAGAGCACCCAGGCCGCCAUCGACCAGAUCAACGGCAA | |
| | GCUGAACCGGCUGAUCGGCAAGACCAACGAGAAGUUCCACC | |
| | AGAUCGAGAAGGAGUUCAGCGAGGUGGAGGGCAGGGUACA | |
| | GGACCUGGAGAAGUACGUGGAGGACACCAAGAUCGACCUG | |
| | UGGAGCUACAACGCCGAGCUGCUGGUAGCCCUGGAGAACCA | |
| | GCACACCAUCGACCUGACCGACAGCGAGAUGAACAAGCUGU | |
| | UCGAGAAGACCAAGAAGCAGCUGCGGGAGAACGCCGAGGAC | |
| | AUGGGCAACGGCUGCUUCAAGAUCUACCACAAGUGCGACAA | |
| | CGCCUGCAUCGGCAGCAUCCGGAACGAGACCUACGACCACA | |
| | ACGUGUACCGGGACGAGGCCCUGAACAACCGGUUCCAGAUC | |
| | AAGGGCGUGGAGCUGAAGAGCGGCUACAAGGACUGGAUCC | |
| | UGUGGAUCAGCUUCGCCAUGUCCUGCUUCCUGCUGUGCAUC | |
| | GCCCUGCUGGGUUUCAUCAUGUGGGCCUGCCAGAAGGGCAA | |
| | CAUCCGGUGCAACAUCUGCAUC | |
| 3′ UTR | UAAAGCUCCCCGGGGGCCUCGGUGGCCCUCCGCAUCAGACC | 50 |
| | AACGGCACACCCCCAGCCCCUCCUCCCCUUCCUGCAGGUCA | |
| | AGGAGUGGUCUUUGAAUAAAGUCUGAGUGGGCGGC | |
| Corresponding amino | MKTIIALSNILCLVFAQKIPGNDNSTATLCLGHHAVPNGTIVKTIT | 51 |
| acid sequence | NDRIEVTNATELVQNSSIGEICDSPHQILDGGNCTLIDALLGDPQC | |
| | DGFQNKEWDLFVERSRANSNCYPYDVPDYASLRSLVASSGTLEF | |
| | KNESFNWTGVKQNGTSSACIRGSSSSFFSRLNWLTHLNNIYPAQ | |
| | NVTMPNKEQFDKLYIWGVHHPDTDKNQISLFAQSSGRITVSTKR | |
| | SQQAVIPNiGSRPRIRDIPSRISIYWTIVKPGDILLINSTGNLI | |
| | APRGYFKIRSGKSSIMRSDAPIGKCKSECITPNGSIPNDKPFQN | |
| | VNRITYGACPRYVKQSTLKLATGMRNVPEKQTRGIFGAIAGFIE | |
| | NGWEGMVDGWYGFRHQNSEGRGQAADLKSTQAAIDQINGKLNRL | |
| | IGKTNEKFHQIEKEFSEVEGRVQDLEKYVEDTKIDLWSYNAELL | |
| | VALENQHTIDLTDSEMNKLFEKTKKQLRENAEDMGNGCFKIYHK | |
| | CDNACIGSIRNETYDHNVYRDEALNNRFQIKGVELKSGYKDWIL | |
| | WISFAMSCFLLCIALLGFIMWACQKGNIRCNICI | |
| PolyA tail | 100 nt | |

B/Austria/1359417/2021 H363Y A270V HA_SEv4.5

| SEQ ID NO: 66 consists of from 5′ end to 3′ end: 5′ UTR SEQ ID NO: 45, | | 66 |
|---|---|---|
| mRNA ORF SEQ ID NO: 52, and 3′ UTR SEQ ID NO: 53. | | |
| Chemistry | 1-methylpseudouridine | |
| Cap | 7mG(5′)ppp(5′)NlmpNp | |
| 5′ UTR | AGGAAAUCGCAAAAUUUGCUCUUCGCGUUAGAUUUCUUUU | 45 |
| | AGUUUUCUCGCAACUAGCAAGCUUUUUGUUCUCGCC | |
| ORF of mRNA | AUGAAGGCUAUCAUCGUGCUGUUAAUGGUGGUGACCAGCA | 52 |
| Construct | ACGCCGACCGGAUCUGCACAGGCAUCACCUCUAGCAACAGC | |
| (excluding the stop | CCUCACGUGGUCAAGACCGCCACACAGGGCGAGGUGAACGU | |
| codon) | GACCGGCGUGAUUCCCCUGACCACCACCCCUACCAAGAGCC | |
| | ACUUCGCCAACCUGAAGGGAACCGAGACCCGGGGCAAGCUG | |
| | UGUCCAAAGUGCCUGAACUGUACCGACCUGGACGUGGCUCU | |
| | GGGCAGACCCAAGUGCACCGGCAAGAUCCCCAGCGCCCGGG | |
| | UGUCUAUCUUGCACGAAGUGCGGCCCGUGACUAGCGGUUGC | |
| | UUCCCCAUCAUGCACGACCGGACCAAGAUCAGACAGCUGCC | |
| | CAACCUGCUGCGGGGCUACGAGCACGUGCGGCUGUCCACCC | |
| | AUAACGUGAUCAACACCGAAGACGCACCCGGCGGCCCAUAC | |
| | GAGAUCGGCACCAGCGGCUCUUGCCUGAAUAUCACCAACGG | |
| | CAAGGGAUUCUUUGCUACCAUGGCCUGGGCCGUGCCAAAGA | |
| | ACAAGACUGCCACCAACCCUCUGACCAUCGAGGUGCCCUAC | |
| | AUCUGCACCGAGGAGGAGGACCAGAUCACCGUGUGGGGCUU | |
| | CCACAGCGACGACGAGACACAGAUGGCCAGACUGUACGGCG | |
| | ACAGCAAACCCCAGAAGUUCACCAGCAGCGCCAACGGCGUG | |
| | ACCACCCACUACGUGAGCCAGAUUGGCGGCUUCCCUAACCA | |
| | AACCGAGGACGGCGGCUUACCCCAGAGCGGCCGGAUCGUGG | |
| | UGGACUACAUGGUUCAGAAGAGCGGCAAGACCGGCACCAUC | |
| | ACCUACCAGCGGGGCAUCCUGUUACCACAGAAGGUGUGGUG | |
| | CGUGUCAGGGAAAUCAAAGGUCAUCAAGGGCUCCCUGCCAC | |
| | UGAUUGGCGAGGCCGACUGCCUGCACGAGAAGUACGGCGGC | |
| | CUGAACAAGAGCAAGCCCUACUACACCGGCGAGCACGCCAA | |
| | GGCAAUCGGCAACUGCCCCAUCUGGGUGAAGACACCCCUGA | |
| | AGCUGGCAAACGGCACCAAGUACCGGCCACCCGCCAAACUG | |
| | CUGAAGGAGCGGGGCUUCUUCGGCGCCAUUGCCGGCUUCCU | |
| | CGAAGGCGGUUGGGAGGGCAUGAUCGCAGGCUGGUACGGC | |
| | UACACUAGCCACGGCGCACACGGAGUAGCAGUGGCCGCCGA | |
| | CCUGAAGAGCACCCAGGAGGCCAUCAACAAGAUCACCAAGA | |
| | ACCUGAACAGCCUGAGCGAGCUGGAGGUGAAGAAUCUGCA | |
| | GAGGCUGUCUGGCGCUAUGGACGAGCUGCACAACGAGAUCC | |
| | UGGAGCUGGACGAGAAGGUGGACGACUUACGGGCCGACACC | |
| | AUCAGCAGCCAGAUCGAGCUGGCAGUGCUGCUGAGCAACGA | |
| | GGGCAUCAUCAACAGCGAGGACGAGCACCUGCUGGCCCUGG | |
| | AGCGGAAGCUGAAGAAGAUGCUGGGCCCUUCUGCCGUGGA | |
| | GAUCGGUAACGGCUGCUUCGAGACCAAGCACAAGUGCAACC | |
| | AGACCUGCCUGGAUCGGAUCGCAGCCGGCACCUUUGACGCC | |
| | GGGGAGUUCAGCCUGCCCACCUUCGACAGCUUGAACAUCAC | |
| | CGCCGCCAGCCUGAACGACGACGGCCUGGACAACCACACCA | |
| | UCCUGCUGUACUACUCUACAGCCGCUAGCAGCCUGGCCGUG | |
| | ACCCUGAUGAUCGCCAUCUUCGUGGUGUACAUGGUGAGCCG | |
| | GGACAACGUGAGCUGCAGCAUCUGCCUG | |
| 3′ UTR | UAAAGCUCCCCGGGGGCCUCGGUGGCCCUCAACCUACGCCG | 53 |
| | AAGACCACGCCCCCAGCCCCUCCUCCCCUUCCUGCAGGUCA | |
| | AGAGUGGUCUUUGAAUAAAGUCUGAGUGGGCGGC | |
| Corresponding amino | MKAIIVLLMVVTSNADRICTGITSSNSPHVVKTATQGEVNVTGVI | 54 |
| acid sequence | PLTTTPTKSHFANLKGTETRGKLCPKCLNCTDLDVALGRPKCTGK | |
| | IPSARVSILHEVRPVTSGCFPIMHDRTKIRQLPNLLRGYEHVRLS | |
| | THNVINTEDAPGGPYEIGTSGSCLNITNGKGFFATMAWAVPKNK | |
| | TATNPLTIEVPYICTEEEDQITVWGFHSDDETQMARLYGDSKPQ | |
| | KFTSSANGVTTHYVSQIGGFPNQTEDGGLPQSGRIVVDYMVQKS | |
| | GKTGTITYQRGILLPQKVWCVSGKSKVIKGSLPLIGEADCLHEK | |
| | YGGLNKSKPYYTGEHAKAIGNCPIWVKTPLKLANGTKYRPPAKL | |
| | LKERGFFGAIAGFLEGGWEGMIAGWYGYTSHGAHGVAVAADLK | |
| | STQEAINKITKNLNSLSELEVKNLQRLSGAMDELHNEILELDEK | |
| | VDDLRADTISSQIELAVLLSNEGIINSEDEHLLALERKLKKMLG | |
| | PSAVEIGNGCFETKHKCNQTCLDRIAAGTFDAGEFSLPTFDSLN | |
| | ITAASLNDDGLDNHTILLYYSTAASSLAVTLMIAIFVVYMVSRD | |
| | NVSCSICL | |
| PolyA tail | 100 nt | |

B/Phuket/3073/2013 H363Y A270V HA

| SEQ ID NO: 67 consists of from 5′ end to 3′ end: 5′ UTR SEQ ID NO: 45, | | 67 |
|---|---|---|
| mRNA ORF SEQ ID NO: 55, and 3′ UTR SEQ ID NO: 56. | | |
| Chemistry | 1-methylpseudouridine | |
| Cap | 7mG(5′)ppp(5′)NlmpNp | |
| 5′ UTR | AGGAAAUCGCAAAAUUUGCUCUUCGCGUUAGAUUUCUUUU | 45 |
| | AGUUUUCUCGCAACUAGCAAGCUUUUUGUUCUCGCC | |
| ORF of mRNA | AUGAAGGCUAUCAUCGUGCUACUCAUGGUGGUAACCAGCA | 55 |
| Construct | ACGCCGACCGGAUCUGCACCGGCAUCACCAGCAGCAACAGC | |
| (excluding the stop | CCGCACGUUGUGAAGACCGCCACCCAAGGCGAGGUGAACGU | |
| codon) | GACCGGCGUGAUCCCACUGACCACCACUCCCACCAAGAGCU | |
| | ACUUCGCCAACCUGAAGGGCACUCGGACUCGGGGCAAGCUC | |
| | UGCCCCGACUGCCUGAACUGCACCGACCUGGACGUGGCUCU | |
| | GGGCAGACCCAUGUGCGUGGGGACCACCCCUUCUGCCAAGG | |
| | CCUCAAUCCUGCACGAGGUCAGGCCCGUGACCAGCGGGUGC | |
| | UUCCCCAUCAUGCACGACCGGACCAAGAUCAGACAGCUGCC | |
| | CAACCUGCUGCGGGGCUACGAGAAGAUCCGGCUGAGCACAC | |
| | AGAACGUGAUCGACGCCGAGAAGGCCCCUGGAGGUCCCUAC | |
| | CGGCUGGGCACCAGCGGAAGCUGCCCCAACGCCACGAGCAA | |
| | GAUCGGCUUCUUCGCCACCAUGGCCUGGGCUGUGCCCAAGG | |
| | ACAACUACAAGAACGCCACCAAUCCCCUGACCGUGGAGGUG | |
| | CCCUACAUUUGUACCGAGGGCGAGGACCAGAUCACCGUGUG | |
| | GGGCUUCCACAGCGACAACAAGACCCAAAUGAAGAGCCUGU | |
| | ACGGCGACAGCAAUCCCCAGAAGUUCACAAGCAGCGCCAAC | |
| | GGUGUGACCACCCACUACGUGAGCCAGAUUGGCGACUUCCC | |
| | CGACCAGACCGAGGACGGAGGGCUGCCGCAGAGUGGCCGGA | |
| | UCGUGGUGGACUACAUGAUGCAGAAGCCCGGCAAGACCGGC | |
| | ACCAUAGUGUAUCAGCGGGGCGUGCUGUUGCCUCAGAAAG | |
| | UUUGGUGUGUGAGCGGCAGGAGCAAGGUGAUCAAGGGCAG | |
| | CCUUCCCCUGAUCGGCGAGGCAGACUGCCUCCACGAGGAGU | |
| | ACGGCGGCCUGAACAAGAGCAAGCCCUACUACACCGGCAAG | |
| | CACGCCAAGGCAAUCGGGAACUGCCCCAUCUGGGUCAAGAC | |
| | CCCUCUGAAGCUGGCCAACGGCACCAAGUACCGGCCACCAG | |
| | CCAAGCUGCUGAAGGAGCGGGGCUUCUUUGGCGCCAUUGCC | |
| | GGCUUCCUCGAGGGAGGCUGGGAGGGCAUGAUCGCCGGCUG | |
| | GUACGGCUACACAAGCCACGGCGCACACGGAGUGGCUGUGG | |
| | CUGCCGACCUGAAGAGCACCCAGGAGGCCAUCAACAAGAUC | |
| | ACCAAGAAUCUGAACAGCCUGAGCGAGCUGGAGGUGAAGA | |
| | ACCUGCAGCGGCUGUCAGGCGCCAUGGACGAGCUGCACAAC | |
| | GAGAUCCUGGAGCUGGACGAGAAGGUGGACGACCUGCGUG | |
| | CCGACACCAUCAGCAGCCAGAUCGAGCUGGCCGUACUGCUG | |
| | AGCAACGAGGGCAUCAUCAACAGCGAGGACGAGCACCUGCU | |
| | GGCCCUGGAGCGGAAACUGAAGAAGAUGCUGGGACCCUCUG | |
| | CCGUGGACAUCGGCAACGGCUGCUUCGAGACUAAGCACAAG | |
| | UGCAACCAGACCUGCCUGGAUCGGAUCGCCGCCGGAACCUU | |
| | CAACGCCGGCGAGUUCAGCCUGCCCACCUUCGACAGCUUAA | |
| | ACAUCACCGCCGCCAGCCUGAACGACGACGGCCUGGACAAC | |
| | CACACCAUCCUGCUGUACUACAGCACUGCCGCCUCAAGCCU | |
| | GGCCGUGACCCUGAUGCUGGCCAUCUUCAUCGUGUACAUGG | |
| | UGAGCCGGGACAACGUGAGCUGCAGCAUCUGCCUG | |
| 3′ UTR | UAAAGCUCCCCGGGGGCCUCGGUGGCCCUCAACGACCCUGC | 56 |
| | CGCAGCAAACCCCCAGCCCCUCCUCCCCUUCCUGCAGGAUC | |
| | AGUGGUCUUUGAAUAAAGUCUGAGUGGGCGGC | |
| Corresponding amino | MKAIIVLLMVVTSNADRICTGITSSNSPHVVKTATQGEVNVTGVI | 57 |
| acid sequence | PLTTTPTKSYFANLKGTRTRGKLCPDCLNCTDLDVALGRPMCVG | |
| | TTPSAKASILHEVRPVTSGCFPIMHDRTKIRQLPNLLRGYEKIRL | |
| | STQNVIDAEKAPGGPYRLGTSGSCPNATSKIGFFATMAWAVPKD | |
| | NYKNATNPLTVEVPYICTEGEDQITVWGFHSDNKTQMKSLYGD | |
| | SNPQKFTSSANGVTTHYVSQIGDFPDQTEDGGLPQSGRIVVDYM | |
| | MQKPGKTGTIVYQRGVLLPQKVWCVSGRSKVIKGSLPLIGEADC | |
| | LHEEYGGLNKSKPYYTGKHAKAIGNCPIWVKTPLKLANGTKYR | |
| | PPAKLLKERGFFGAIAGFLEGGWEGMIAGWYGYTSHGAHGVAVA | |
| | ADLKSTQEAINKITKNLNSLSELEVKNLQRLSGAMDELHNEILE | |
| | LDEKVDDLRADTISSQIELAVLLSNEGIINSEDEHLLALERKLK | |
| | KMLGPSAVDIGNGCFETKHKCNQTCLDRIAAGTFNAGEFSLPTF | |
| | DSLNITAASLNDDGLDNHTILLYYSTAASSLAVTLMLAIFIVYM | |
| | VSRDNVSCSICL | |
| PolyA tail | 100 nt | |

NTD-RBD-HATM vV2

| SEQ ID NO: 68 consists of from 5′ end to 3′ end: 5′ UTR SEQ ID NO: 45, | | 68 |
|---|---|---|
| mRNA ORF SEQ ID NO: 58, and 3′ UTR SEQ ID NO: 59. | | |
| Chemistry | 1-methylpseudouridine | |
| Cap | 7mG(5′)ppp(5′)NlmpNp | |
| 5′ UTR | AGGAAAUCGCAAAAUUUGCUCUUCGCGUUAGAUUUCUUUU | 45 |
| | AGUUUUCUCGCAACUAGCAAGCUUUUUGUUCUCGCC | |
| ORF of mRNA | AUGUUCGUGUUCCUGGUGCUGCUGCCCCUGGUGAGCAGCCA | 58 |
| Construct | GUGCGUGAACCUGACCACCCGGACCCAGCUGCCACCAGCCU | |
| (excluding the stop | ACACCAACAGCUUCACCCGGGGCGUCUACUACCCCGACAAG | |
| codon) | GUGUUCCGGAGCAGCGUCCUGCACAGCACCCAGGACCUGUU | |
| | CCUGCCCUUCUUCAGCAACGUGACCUGGUUCCACGCCAUCC | |
| | ACGUGAGCGGCACCAACGGCACCAAGCGGUUCGACAACCCC | |
| | GUGCUGCCCUUCAACGACGGCGUGUACUUCGCCAGCACCGA | |
| | GAAGAGCAACAUCAUCCGGGGCUGGAUCUUCGGCACCACCC | |
| | UGGACAGCAAGACCCAGAGCCUGCUGAUCGUGAAUAACGCC | |
| | ACCAACGUGGUGAUCAAGGUGUGCGAGUUCCAGUUCUGCA | |
| | ACGACCCCUUCCUGGGCGUGUACUACCACAAGAACAACAAG | |
| | AGCUGGAUGGAGAGCGAGUUCCGGGUGUACAGCAGCGCCA | |
| | ACAACUGCACCUUCGAGUACGUGAGCCAGCCCUUCCUGAUG | |
| | GACCUGGAGGGCAAGCAGGGCAACUUCAAGAACCUGCGGGA | |
| | GUUCGUGUUCAAGAACAUCGACGGCUACUUCAAGAUCUACA | |
| | GCAAGCACACCCCAAUCAACCUGGUGCGGGAUCUGCCCCAG | |
| | GGCUUCUCAGCCCUGGAGCCCCUGGUGGACCUGCCCAUCGG | |
| | CAUCAACAUCACCCGGUUCCAGACCCUGCUGGCCCUGCACC | |
| | GGAGCUACCUGACCCCAGGCGACAGCAGCAGCGGGUGGACA | |
| | GCAGGCGCGGCUGCUUACUACGUGGGCUACCUGCAGCCCCG | |
| | GACCUUCCUGCUGAAGUACAACGAGAACGGCACCAUCACCG | |
| | ACGCCGUGGACGGAGGCGGAUCGGGAGGCGGACCCAACAUC | |
| | ACCAACCUGUGCCCCUUCGGCGAGGUGUUCAACGCCACCCG | |
| | GUUCGCCAGCGUGUACGCCUGGAACCGGAAGCGGAUCAGCA | |
| | ACUGCGUGGCCGACUACAGCGUGCUGUACAACAGCGCCAGC | |
| | UUCAGCACCUUCAAGUGCUACGGCGUGAGCCCCACCAAGCU | |
| | GAACGACCUGUGCUUCACCAACGUGUACGCCGACAGCUUCG | |
| | UGAUCCGUGGCGACGAGGUGCGGCAGAUCGCACCCGGCCAG | |
| | ACAGGCAAGAUCGCCGACUACAACUACAAGCUGCCCGACGA | |
| | CUUCACCGGCUGCGUGAUCGCCUGGAACAGCAACAACCUCG | |
| | ACAGCAAGGUGGGCGGCAACUACAACUACCUGUACCGGCUG | |
| | UUCCGGAAGAGCAACCUGAAGCCCUUCGAGCGGGACAUCAG | |
| | CACCGAGAUCUACCAAGCCGGCUCCACCCCUUGCAACGGCG | |
| | UGGAGGGCUUCAACUGCUACUUCCCUCUGCAGAGCUACGGC | |
| | UUCCAGCCCACCAACGGCGUGGGCUACCAGCCCUACCGGGU | |
| | GGUGGUGCUGAGCUUCGAGCUGCUGCACGCCCCAGCCACCG | |
| | UGUGUGGCCCCAAGUCUGGCGGAGGCAGCAUCCUGGCCAUC | |
| | UACAGCACCGUGGCCAGCAGCCUGGUGCUGCUGGUGAGCCU | |
| | GGGCGCCAUCAGCUUC | |
| 3′ UTR | UAAAGCUCCCCGGGGGCCUCGGUGGCCUAGCUUCUUGCCCC | 59 |
| | UUGGGCCUCCCCCCAGCCCCUCCUCCCCUUCCUGCACCUGUA | |
| | CUUCCGUGGUCUUUGAAUAAAGUCUGAGUGGGCGGC | |
| Corresponding amino | MFVFLVLLPLVSSQCVNLTTRTQLPPAYTNSFTRGVYYPDKVFR | 60 |
| acid sequence | SSVLHSTQDLFLPFFSNVTWFHAIHVSGTNGTKRFDNPVLPFNDG | |
| | VYFASTEKSNIIRGWIFGTTLDSKTQSLLIVNNATNVVIKVCEFQF | |
| | CNDPFLGVYYHKNNKSWMESEFRVYSSANNCTFEYVSQPFLMD | |
| | LEGKQGNFKNLREFVFKNIDGYFKIYSKHTPINLVRDLPQGFSAL | |
| | EPLVDLPIGINITRFQTLLALHRSYLTPGDSSSGWTAGAAAYYVG | |
| | YLQPRTFLLKYNENGTITDAVDGGGSGGGPNITNLCPFGEVFNA | |
| | TRFASVYAWNRKRISNCVADYSVLYNSASFSTFKCYGVSPTKLN | |
| | DLCFTNVYADSFVIRGDEVRQIAPGQTGKIADYNYKLPDDFTGC | |
| | VIAWNSNNLDSKVGGNYNYLYRLFRKSNLKPFERDISTEIYQAG | |
| | STPCNGVEGFNCYFPLQSYGFQPTNGVGYQPYRVVVLSFELLHA | |
| | PATVCGPKSGGGSILAIYSTVASSLVLLVSLGAISF | |
| PolyA tail | 100 nt | |

BA.4.BA.5-NTD-RBD-HATM

| SEQ ID NO: 69 consists of from 5′ end to 3′ end: 5′ UTR SEQ ID NO: 45, | | 69 |
|---|---|---|
| mRNA ORF SEQ ID NO: 61, and 3′ UTR SEQ ID NO: 62. | | |
| Chemistry | 1-methylpseudouridine | |
| Cap | 7mG(5′)ppp(5′)NlmpNp | |
| 5′ UTR | AGGAAAUCGCAAAAUUUGCUCUUCGCGUUAGAUUUCUUUU | 45 |
| | AGUUUUCUCGCAACUAGCAAGCUUUUUGUUCUCGCC | |
| ORF of mRNA | AUGUUCGUGUUCCUGGUGCUGCUGCCCCUGGUGAGCAGCCA | 61 |
| Construct | GUGCGUGAACCUGAUCACCCGGACCCAGAGCUACACCAACA | |
| (excluding the stop | GCUUCACCCGGGGCGUCUACUACCCCGACAAGGUGUUCCGG | |
| codon) | AGCAGCGUCCUGCACAGCACCCAGGACCUGUUCCUGCCCUU | |
| | CUUCAGCAACGUGACCUGGUUCCACGCCAUCAGCGGCACCA | |
| | ACGGCACCAAGCGGUUCGACAACCCCGUGCUGCCCUUCAAC | |
| | GACGGCGUGUACUUCGCCAGCACCGAGAAGAGCAACAUCAU | |
| | CCGGGGCUGGAUCUUCGGCACCACCCUGGACAGCAAGACCC | |
| | AGAGCCUGCUGAUCGUGAAUAACGCCACCAACGUGGUGAUC | |
| | AAGGUGUGCGAGUUCCAGUUCUGCAACGACCCCUUCCUGGA | |
| | CGUGUACUACCACAAGAACAACAAGAGCUGGAUGGAGAGC | |
| | GAGUUCCGGGUGUACAGCAGCGCCAACAACUGCACCUUCGA | |
| | GUACGUGAGCCAGCCCUUCCUGAUGGACCUGGAGGGCAAGC | |
| | AGGGCAACUUCAAGAACCUGCGGGAGUUCGUGUUCAAGAA | |
| | CAUCGACGGCUACUUCAAGAUCUACAGCAAGCACACCCCAA | |
| | UCAACCUGGGCCGGGAUCUGCCCCAGGGCUUCUCAGCCCUG | |
| | GAGCCCCUGGUGGACCUGCCCAUCGGCAUCAACAUCACCCG | |
| | GUUCCAGACCCUGCUGGCCCUGCACCGGAGCUACCUGACCC | |
| | CAGGCGACAGCAGCAGCGGGUGGACAGCAGGCGCGGCUGCU | |
| | UACUACGUGGGCUACCUGCAGCCCCGGACCUUCCUGCUGAA | |
| | GUACAACGAGAACGGCACCAUCACCGACGCCGUGGACGGCG | |
| | GAGGCUCUGGAGGCGGCCCCAACAUCACCAACCUGUGCCCC | |
| | UUCGACGAGGUGUUCAACGCCACCCGGUUCGCCAGCGUGUA | |
| | CGCCUGGAACCGGAAGCGGAUCAGCAACUGCGUGGCCGACU | |
| | ACAGCGUGCUGUACAACUUCGCCCCAUUCUUCGCCUUCAAG | |
| | UGCUACGGCGUGAGCCCCACCAAGCUGAACGACCUGUGCUU | |
| | CACCAACGUGUACGCCGACAGCUUCGUGAUCCGUGGCAACG | |
| | AGGUGAGCCAGAUCGCACCCGGCCAGACAGGCAACAUCGCC | |
| | GACUACAACUACAAGCUGCCCGACGACUUCACCGGCUGCGU | |
| | GAUCGCCUGGAACAGCAACAAGCUCGACAGCAAGGUGGGCG | |
| | GCAACUACAACUACCGGUACCGGCUGUUCCGGAAGAGCAAC | |
| | CUGAAGCCCUUCGAGCGGGACAUCAGCACCGAGAUCUACCA | |
| | AGCCGGCAACAAGCCUUGCAACGGCGUGGCCGGCGUGAACU | |
| | GCUACUUCCCUCUGCAGAGCUACGGCUUCCGGCCCACCUAC | |
| | GGCGUGGGCCACCAGCCCUACCGGGUGGUGGUGCUGAGCUU | |
| | CGAGCUGCUGCACGCCCCAGCCACCGUGUGUGGCCCCAAGA | |
| | GCGGCGGCGGCAGCAUCCUGGCCAUCUACAGCACCGUGGCC | |
| | AGCAGCCUGGUGCUGCUGGUGAGCCUGGGCGCCAUCAGCUU | |
| | U | |
| 3′ UTR | UAAAGCUCCCCGGGGGCCUCGGUGGCCUAGCUUCUUGCCCC | 62 |
| | UUGGGCCUCCCCCCAGCCCCUCCUCCCCUUCCUGCAUUUGCC | |
| | UGUGAGCAGUGGUCUUUGAAUAAAGUCUGAGUGGGCGGC | |
| Corresponding amino | MFVFLVLLPLVSSQCVNLITRTQSYTNSFTRGVYYPDKVFRSSVL | 63 |
| acid sequence | HSTQDLFLPFFSNVTWFHAISGTNGTKRFDNPVLPFNDGVYFAST | |
| | EKSNIIRGWIFGTTLDSKTQSLLIVNNATNVVIKVCEFQFCNDPFL | |
| | DVYYHKNNKSWMESEFRVYSSANNCTFEYVSQPFLMDLEGKQ | |
| | GNFKNLREFVFKNIDGYFKIYSKHTPINLGRDLPQGFSALEPLVD | |
| | LPIGINITRFQTLLALHRSYLTPGDSSSGWTAGAAAYYVGYLQPR | |
| | TFLLKYNENGTITDAVDGGGSGGGPNITNLCPFDEVFNATRFAS | |
| | VYAWNRKRISNCVADYSVLYNFAPFFAFKCYGVSPTKLNDLCF | |
| | TNVYADSFVIRGNEVSQIAPGQTGNIADYNYKLPDDFTGCVIAW | |
| | NSNKLDSKVGGNYNYRYRLFRKSNLKPFERDISTEIYQAGNKPC | |
| | NGVAGVNCYFPLQSYGFRPTYGVGHQPYRVVVLSFELLHAPAT | |
| | VCGPKSGGGSILAIYSTVASSLVLLVSLGAISF | |
| PolyA tail | 100 nt | |

Additional Influenza Virus Sequences (mutation(s) relative to wild-type sequence are bolded and underlined)

| | | SEQ |
|---|---|---|
| Name | Sequence | ID NO: |
| B/Phuket/3 | MKAIIVLLMVVTSNADRICTGITSSNSPHVVKTATQGEVNVTGVIPLTTTPTKSY | 70 |
| 073/2013 | FANLKGTRTRGKLCPDCLNCTDLDVALGRPMCVGTTPSAKASILHEVRPVTSG | |
| (B/Yamagata | CFPIMHDRTKIRQLPNLLRGYEKIRLSTQNVIDAEKAPGGPYRLGTSGSCPNATS | |
| lineage) | KIGFFATMAWAVPKDNYKNATNPLTVEVPYICTEGEDQITVWGFHSDNKTQM | |
| HA: wild- | KSLYGDSNPQKFTSSANGVTTHYVSQIGDFPDQTEDGGLPQSGRIVVDYMMQK | |
| type | PGKTGTIVYQRGVLLPQKVWCASGRSKVIKGSLPLIGEADCLHEEYGGLNKSK | |
| | PYYTGKHAKAIGNCPIWVKTPLKLANGTKYRPPAKLLKERGFFGAIAGFLEGG | |
| | WEGMIAGWHGYTSHGAHGVAVAADLKSTQEAINKITKNLNSLSELEVKNLQR | |
| | LSGAMDELHNEILELDEKVDDLRADTISSQIELAVLLSNEGIINSEDEHLLALER | |
| | KLKKMLGPSAVDIGNGCFETKHKCNQTCLDRIAAGTFNAGEFSLPTFDSLNITA | |
| | ASLNDDGLDNHTILLYYSTAASSLAVTLMLAIFIVYMVSRDNVSCSICL | |
| B/Austria/1 | MKAIIVLLMVVTSNADRICTGITSSNSPHVVKTATQGEVNVTGVIPLTTTPTKSH | 71 |
| 359417/ | FANLKGTETRGKLCPKCLNCTDLDVALGRPKCTGKIPSARVSILHEVRPVTSGC | |
| 2021 | FPIMHDRTKIRQLPNLLRGYEHVRLSTHNVINTEDAPGGPYEIGTSGSCLNITNG | |
| (B/Victoria | KGFFATMAWAVPKNKTATNPLTIEVPYICTEEEDQITVWGFHSDDETQMARLY | |
| lineage) | GDSKPQKFTSSANGVTTHYVSQIGGFPNQTEDGGLPQSGRIVVDYMVQKSGKT | |
| HA: wild- | GTITYQRGILLPQKVWCASGKSKVIKGSLPLIGEADCLHEKYGGLNKSKPYYTG | |
| type | EHAKAIGNCPIWVKTPLKLANGTKYRPPAKLLKERGFFGAIAGFLEGGWEGMI | |
| | AGWHGYTSHGAHGVAVAADLKSTQEAINKITKNLNSLSELEVKNLQRLSGAM | |
| | DELHNEILELDEKVDDLRADTISSQIELAVLLSNEGIINSEDEHLLALERKLKKM | |
| | LGPSAVEIGNGCFETKHKCNQTCLDRIAAGTFDAGEFSLPTFDSLNITAASLND | |
| | DGLDNHTILLYYSTAASSLAVTLMIAIFVVYMVSRDNVSCSICL | |
| A/Darwin/6 | MKTIIALSNILCLVFAQKIPGNDNSTATLCLGHHAVPNGTIVKTITNDRIEVTNA | 82 |
| /2021(H3N | TELVQNSSIGEICGSPHQILDGGNCTLIDALLGDPQCDGFQNKEWDLFVERSRA | |
| 2) HA: | NSNCYPYDVPDYASLRSLVASSGTLEFKNESFNWTGVKQNGTSSACIRGSSSSF | |
| wild-type | FSRLNWLTSLNnIYPAQNVTMPNKEQFDKLYIWGVHHPDTDKNQISLFAQSSG | |
| | RITVSTKRSQQAVIPNIGSRPRIRGIPSRISIYWTIVKPGDILLINSTGNLIAPRGYF | |
| | KIRSGKSSIMRSDAPIGKCKSECITPNGSIPNDKPFQNVNRITYGACPRYVKQSTL | |
| | KLATGMRNVPEKQTRGIFGAIAGFIENGWEGMVDGWYGFRHQNSEGRGQAA | |
| | DLKSTQAAIDQINGKLNRLIGKTNEKFHQIEKEFSEVEGRVQDLEKYVEDTKID | |
| | LWSYNAELLVALENQHTIDLTDSEMNKLFEKTKKQLRENAEDMGNGCFKIYH | |
| | KCDNACIGSIRNETYDHNVYRDEALNNRFQIKGVELKSGYKDWILWISFAMSC | |
| | FLLCIALLGFIMWACQKGNIRCNICI | |
| A/Wisconsin | MKAILVVMLYTFTTANADTLCIGYHANNSTDTVDTVLEKNVTVTHSVNLLED | 83 |
| /67/2022( | KHNGKLCKLRGVAPLHLGQCNIAGWILGNPECESLSTARSWSYIVETSNSDNG | |
| H1N1) HA: | TCYPGDFINYEELREQLSSVSSFERFEIFPKTSSWPNHDSDNGVTAACSHAGARS | |
| wild-type | FYKNLIWLVKKGKSYPKINQTYINDKGKEVLVLWGIHHPPTITDQESLYQNAD | |
| | AYVFVGTSRYSKKFKPEIATRPKVRDQAGRMNYYWTLVEPGDKITFEATGNL | |
| | VAPRYAFTMEKEAGSGIIISDTPVHDCNATCQTPEGAINTSLPFQNVHPITIGKCP | |
| | KYVRSTKLRLATGLRNVPSIQSRGLFGAIAGFIEGGWTGMVDGWYGYHHQND | |
| | QGSGYAADLKSTQNAIDKITNKVNSVIEKMNTQFTAVGKEFNHLEKRIENLNK | |
| | KVDDGFLDVWTYNAELLVLLENERTLDYHDSNVKNLYEKVRHQLKNNAKEI | |
| | GNGCFEFYHKCDNTCMESVKNGTYDYPKYSEEAKLNREKIDGVKLDSTRIYQI | |
| | LAIYSTVASSLVLVVSLGAISFWMCSNGSLQCRICI | |
| A/Sydney/5 | MKAILVVMLYTFTTANADTLCIGYHANNSTDTVDTVLEKNVTVTHSVNLLED | 95 |
| /2021(H1N | KHNGKLCKLRGVAPLHLGQCNIAGWILGNPECESLSTARSWSYIVETSNSDNG | |
| 1) HA: | TCYPGNFINYEELREQLSSVSSFERFEIFPKTSSWPNHDSDNGVTAACPHAGAKS | |
| wild-type | FYKNLIWLVKKGKSYPKINQTYINDKGKEVLVLWGIHHPPTITDQESLYQNAD | |
| | AYVFVGTSRYSKKFKPEIAARPKVRDRAGRMNYYWTLVEPGDKITFEATGNL | |
| | VAPRYAFTMEKDAGSGIIISDTPVHDCNTTCQTPEGAINTSLPFQNVHPITIGKCP | |
| | KYVRSTKLRLATGLRNVPSIQSRGLFGAIAGFIEGGWTGMVDGWYGYHHQNE | |
| | QGSGYAADLKSTQNAIDKITNKVNSVIEKMNTQFTAVGKEFNHLEKRIENLNK | |
| | KVDDGFLDIWTYNAELLVLLENERTLDYHDSNVKNLYEKVRNQLKNNAKEIG | |
| | NGCFEFYHKCDNTCMESVKNGTYDYPKYSEEAKLNREKIDGVKLDSTRIYQIL | |
| | AIYSTVASSLVLVVSLGAISFWMCSNGSLQCRICI | |
| B Phuket | MKAIIVLLMVVTSNADRICTGITSSNSPHVVKTATQGEVNVTGVIPLTTTPTKSY | 72 |
| HA: Q267L | FANLKGTRTRGKLCPDCLNCTDLDVALGRPMCVGTTPSAKASILHEVRPVTSG | |
| | CFPIMHDRTKIRQLPNLLRGYEKIRLSTQNVIDAEKAPGGPYRLGTSGSCPNATS | |
| | KIGFFATMAWAVPKDNYKNATNPLTVEVPYICTEGEDQITVWGFHSDNKTQM | |
| | KSLYGDSNPQKFTSSANGVTTHYVSQIGDFPDQTEDGGLPQSGRIVVDYMMLK | |
| | PGKTGTIVYQRGVLLPQKVWCASGRSKVIKGSLPLIGEADCLHEEYGGLNKSK | |
| | PYYTGKHAKAIGNCPIWVKTPLKLANGTKYRPPAKLLKERGFFGAIAGFLEGG | |
| | WEGMIAGWHGYTSHGAHGVAVAADLKSTQEAINKITKNLNSLSELEVKNLQR | |
| | LSGAMDELHNEILELDEKVDDLRADTISSQIELAVLLSNEGIINSEDEHLLALER | |
| | KLKKMLGPSAVDIGNGCFETKHKCNQTCLDRIAAGTFNAGEFSLPTFDSLNITA | |
| | ASLNDDGLDNHTILLYYSTAASSLAVTLMLAIFIVYMVSRDNVSCSICL | |
| B_Phuket_ | MKAIIVLLMVVTSNADRICTGITSSNSPHVVKTATQGEVNVTGVIPLTTTPTKSY | 73 |
| HA: G273A | FANLKGTRTRGKLCPDCLNCTDLDVALGRPMCVGTTPSAKASILHEVRPVTSG | |
| | CFPIMHDRTKIRQLPNLLRGYEKIRLSTQNVIDAEKAPGGPYRLGTSGSCPNATS | |
| | KIGFFATMAWAVPKDNYKNATNPLTVEVPYICTEGEDQITVWGFHSDNKTQM | |
| | KSLYGDSNPQKFTSSANGVTTHYVSQIGDFPDQTEDGGLPQSGRIVVDYMMQK | |
| | PGKTATIVYQRGVLLPQKVWCASGRSKVIKGSLPLIGEADCLHEEYGGLNKSK | |
| | PYYTGKHAKAIGNCPIWVKTPLKLANGTKYRPPAKLLKERGFFGAIAGFLEGG | |
| | WEGMIAGWHGYTSHGAHGVAVAADLKSTQEAINKITKNLNSLSELEVKNLQR | |
| | LSGAMDELHNEILELDEKVDDLRADTISSQIELAVLLSNEGIINSEDEHLLALER | |
| | KLKKMLGPSAVDIGNGCFETKHKCNQTCLDRIAAGTFNAGEFSLPTFDSLNITA | |
| | ASLNDDGLDNHTILLYYSTAASSLAVTLMLAIFIVYMVSRDNVSCSICL | |
| B_Phuket_ | MKAIIVLLMVVTSNADRICTGITSSNSPHVVKTATQGEVNVTGVIPLTTTPTKSY | 74 |
| HA: H435L | FANLKGTRTRGKLCPDCLNCTDLDVALGRPMCVGTTPSAKASILHEVRPVTSG | |
| | CFPIMHDRTKIRQLPNLLRGYEKIRLSTQNVIDAEKAPGGPYRLGTSGSCPNATS | |
| | KIGFFATMAWAVPKDNYKNATNPLTVEVPYICTEGEDQITVWGFHSDNKTQM | |
| | KSLYGDSNPQKFTSSANGVTTHYVSQIGDFPDQTEDGGLPQSGRIVVDYMMQK | |
| | PGKTGTIVYQRGVLLPQKVWCASGRSKVIKGSLPLIGEADCLHEEYGGLNKSK | |
| | PYYTGKHAKAIGNCPIWVKTPLKLANGTKYRPPAKLLKERGFFGAIAGFLEGG | |
| | WEGMIAGWHGYTSHGAHGVAVAADLKSTQEAINKITKNLNSLSELEVKNLQR | |
| | LSGAMDELLNEILELDEKVDDLRADTISSQIELAVLLSNEGIINSEDEHLLALER | |
| | KLKKMLGPSAVDIGNGCFETKHKCNQTCLDRIAAGTFNAGEFSLPTFDSLNITA | |
| | ASLNDDGLDNHTILLYYSTAASSLAVTLMLAIFIVYMVSRDNVSCSICL | |
| B_Phuket_ | MKAIIVLLMVVTSNADRICTGITSSNSPHVVKTATQGEVNVTGVIPLTTTPTKSY | 75 |
| HA: | FANLKGTRTRGKLCPDCLNCTDLDVALGRPMCVGTTPSAKASILHEVRPVTSG | |
| Q267L- | CFPIMHDRTKIRQLPNLLRGYEKIRLSTQNVIDAEKAPGGPYRLGTSGSCPNATS | |
| G273A- | KIGFFATMAWAVPKDNYKNATNPLTVEVPYICTEGEDQITVWGFHSDNKTQM | |
| H435L | KSLYGDSNPQKFTSSANGVTTHYVSQIGDFPDQTEDGGLPQSGRIVVDYMMLK | |
| | PGKTATIVYQRGVLLPQKVWCASGRSKVIKGSLPLIGEADCLHEEYGGLNKSK | |
| | PYYTGKHAKAIGNCPIWVKTPLKLANGTKYRPPAKLLKERGFFGAIAGFLEGG | |
| | WEGMIAGWHGYTSHGAHGVAVAADLKSTQEAINKITKNLNSLSELEVKNLQR | |
| | LSGAMDELLNEILELDEKVDDLRADTISSQIELAVLLSNEGIINSEDEHLLALER | |
| | KLKKMLGPSAVDIGNGCFETKHKCNQTCLDRIAAGTFNAGEFSLPTFDSLNITA | |
| | ASLNDDGLDNHTILLYYSTAASSLAVTLMLAIFIVYMVSRDNVSCSICL | |
| B_Phuket_ | MKAIIVLLMVVTSNADRICTGITSSNSPHVVKTATQGEVNVTGVIPLTTTPTKSY | 85 |
| HA: | FANLKGTRTRGKLCPDCLNCTDLDVALGRPMCVGTTPSAKASILHEVRPVTSG | |
| V239C- | CFPIMHDRTKIRQLPNLLRGYEKIRLSTQNVIDAEKAPGGPYRLGTSGSCPNATS | |
| V276C- | KIGFFATMAWAVPKDNYKNATNPLTVEVPYICTEGEDQITVWGFHSDNKTQM | |
| D451C- | KSLYGDSNPQKFTSSANGVTTHYCSQIGDFPDQTEDGGLPQSGRIVVDYMMQK | |
| K422C | PGKTGTICYQRGVLLPQKVWCASGRSKVIKGSLPLIGEADCLHEEYGGLNKSK | |
| | PYYTGKHAKAIGNCPIWVKTPLKLANGTKYRPPAKLLKERGFFGAIAGFLEGG | |
| | WEGMIAGWHGYTSHGAHGVAVAADLKSTQEAINKITKNLNSLSELEVCNLQR | |
| | LSGAMDELHNEILELDEKVDDLRACTISSQIELAVLLSNEGIINSEDEHLLALER | |
| | KLKKMLGPSAVDIGNGCFETKHKCNQTCLDRIAAGTFNAGEFSLPTFDSLNITA | |
| | ASLNDDGLDNHTILLYYSTAASSLAVTLMLAIFIVYMVSRDNVSCSICL | |
| B_Phuket_ | MKAIIVLLMVVTSNADRICTGITSSNSPHVVKTATQGEVNVTGVIPLTTTPTKSY | 86 |
| HA: 1367C- | FANLKGTRTRGKLCPDCLNCTDLDVALGRPMCVGTTPSAKASILHEVRPVTSG | |
| S401C- | CFPIMHDRTKIRQLPNLLRGYEKIRLSTQNVIDAEKAPGGPYRLGTSGSCPNATS | |
| D451C- | KIGFFATMAWAVPKDNYKNATNPLTVEVPYICTEGEDQITVWGFHSDNKTQM | |
| K422C | KSLYGDSNPQKFTSSANGVTTHYVSQIGDFPDQTEDGGLPQSGRIVVDYMMQK | |
| | PGKTGTIVYQRGVLLPQKVWCASGRSKVIKGSLPLIGEADCLHEEYGGLNKSK | |
| | PYYTGKHAKAIGNCPIWVKTPLKLANGTKYRPPAKLLKERGFFGACAGFLEGG | |
| | WEGMIAGWHGYTSHGAHGVAVAADLKCTQEAINKITKNLNSLSELEVCNLQR | |
| | LSGAMDELHNEILELDEKVDDLRACTISSQIELAVLLSNEGIINSEDEHLLALER | |
| | KLKKMLGPSAVDIGNGCFETKHKCNQTCLDRIAAGTFNAGEFSLPTFDSLNITA | |
| | ASLNDDGLDNHTILLYYSTAASSLAVTLMLAIFIVYMVSRDNVSCSICL | |
| B_Austria__ | MKAIIVLLMVVTSNADRICTGITSSNSPHVVKTATQGEVNVTGVIPLTTTPTKSH | 76 |
| HA: H383Y | FANLKGTETRGKLCPKCLNCTDLDVALGRPKCTGKIPSARVSILHEVRPVTSGC | |
| | FPIMHDRTKIRQLPNLLRGYEHVRLSTHNVINTEDAPGGPYEIGTSGSCLNITNG | |
| | KGFFATMAWAVPKNKTATNPLTIEVPYICTEEEDQITVWGFHSDDETQMARLY | |
| | GDSKPQKFTSSANGVTTHYVSQIGGFPNQTEDGGLPQSGRIVVDYMVQKSGKT | |
| | GTITYQRGILLPQKVWCASGKSKVIKGSLPLIGEADCLHEKYGGLNKSKPYYTG | |
| | EHAKAIGNCPIWVKTPLKLANGTKYRPPAKLLKERGFFGAIAGFLEGGWEGMI | |
| | AGWYGYTSHGAHGVAVAADLKSTQEAINKITKNLNSLSELEVKNLQRLSGAM | |
| | DELHNEILELDEKVDDLRADTISSQIELAVLLSNEGIINSEDEHLLALERKLKKM | |
| | LGPSAVEIGNGCFETKHKCNQTCLDRIAAGTFDAGEFSLPTFDSLNITAASLND | |
| | DGLDNHTILLYYSTAASSLAVTLMIAIFVVYMVSRDNVSCSICL | |
| B_Austria_ | MKAIIVLLMVVTSNADRICTGITSSNSPHVVKTATQGEVNVTGVIPLTTTPTKSH | 77 |
| HA: A290V | FANLKGTETRGKLCPKCLNCTDLDVALGRPKCTGKIPSARVSILHEVRPVTSGC | |
| | FPIMHDRTKIRQLPNLLRGYEHVRLSTHNVINTEDAPGGPYEIGTSGSCLNITNG | |
| | KGFFATMAWAVPKNKTATNPLTIEVPYICTEEEDQITVWGFHSDDETQMARLY | |
| | GDSKPQKFTSSANGVTTHYVSQIGGFPNQTEDGGLPQSGRIVVDYMVQKSGKT | |
| | GTITYQRGILLPQKVWCVSGKSKVIKGSLPLIGEADCLHEKYGGLNKSKPYYTG | |
| | EHAKAIGNCPIWVKTPLKLANGTKYRPPAKLLKERGFFGAIAGFLEGGWEGMI | |
| | AGWYGYTSHGAHGVAVAADLKSTQEAINKITKNLNSLSELEVKNLQRLSGAM | |
| | DELHNEILELDEKVDDLRADTISSQIELAVLLSNEGIINSEDEHLLALERKLKKM | |
| | LGPSAVEIGNGCFETKHKCNQTCLDRIAAGTFDAGEFSLPTFDSLNITAASLND | |
| | DGLDNHTILLYYSTAASSLAVTLMIAIFVVYMVSRDNVSCSICL | |
| B_Austria_ | MKAIIVLLMVVTSNADRICTGITSSNSPHVVKTATQGEVNVTGVIPLTTTPTKSH | 87 |
| HA: | FANLKGTETRGKLCPKCLNCTDLDVALGRPKCTGKIPSARVSILHEVRPVTSGC | |
| H381Y- | FPIMHDRTKIRQLPNLLRGYEHVRLSTHNVINTEDAPGGPYEIGTSGSCLNITNG | |
| A288V- | KGFFATMAWAVPKNKTATNPLTIEVPYICTEEEDQITVWGFHSDDETQMARLY | |
| N494C- | GDSKPQKFTSSANGVTTHYVSQIGGFPNQTEDGGLPQSGRIVVDYMVQKSGKT | |
| K483C | GTITYQRGILLPQKVWCVSGKSKVIKGSLPLIGEADCLHEKYGGLNKSKPYYTG | |
| | EHAKAIGNCPIWVKTPLKLANGTKYRPPAKLLKERGFFGAIAGFLEGGWEGMI | |
| | AGWYGYTSHGAHGVAVAADLKSTQEAINKITKNLNSLSELEVKNLQRLSGAM | |
| | DELHNEILELDEKVDDLRADTISSQIELAVLLSNEGIINSEDEHLLALERKLKCM | |
| | LGPSAVEIGCGCFETKHKCNQTCLDRIAAGTFDAGEFSLPTFDSLNITAASLND | |
| | DGLDNHTILLYYSTAASSLAVTLMIAIFVVYMVSRDNVSCSICL | |
| B_Austria_ | MKAIIVLLMVVTSNADRICTGITSSNSPHVVKTATQGEVNVTGVIPLTTTPTKSH | 88 |
| HA: | FANLKGTETRGKLCPKCLNCTDLDVALGRPKCTGKIPSARVSILHEVRPVTSGC | |
| H381Y- | FPIMHDRTKIRQLPNLLRGYEHVRLSTHNVINTEDAPGGPYEIGTSGSCLNITNG | |
| A288V- | KGFFATMAWAVPKNKTATNPLTIEVPYICTEEEDQITVWGFHSDDETQMARLY | |
| L422C- | GDSKPQKFTSSANGVTTHYVSQIGGFPNQTEDGGLPQSGRIVVDYMVQKSGKT | |
| D444C | GTITYQRGILLPQKVWCVSGKSKVIKGSLPLIGEADCLHEKYGGLNKSKPYYTG | |
| | EHAKAIGNCPIWVKTPLKLANGTKYRPPAKLLKERGFFGAIAGFLEGGWEGMI | |
| | AGWYGYTSHGAHGVAVAADLKSTQEAINKITKNLNSLSELEVKNCQRLSGAM | |
| | DELHNEILELDEKVCDLRADTISSQIELAVLLSNEGIINSEDEHLLALERKLKKM | |
| | LGPSAVEIGNGCFETKHKCNQTCLDRIAAGTFDAGEFSLPTFDSLNITAASLND | |
| | DGLDNHTILLYYSTAASSLAVTLMIAIFVVYMVSRDNVSCSICL | |
| B_Austria_ | MKAIIVLLMVVTSNADRICTGITSSNSPHVVKTATQGEVNVTGVIPLTTTPTKSH | 89 |
| HA: | FANLKGTETRGKLCPKCLNCTDLDVALGRPKCTGKIPSARVSILHEVRPVTSGC | |
| H381Y- | FPIMHDRTKIRQLPNLLRGYEHVRLSTHNVINTEDAPGGPYEIGTSGSCLNITNG | |
| A288V- | KGFFATMAWAVPKNKTATNPLTIEVPYICTEEEDQITVWGFHSDDETQMARLY | |
| A364C- | GDSKPQKFTSSANGVTTHYVSQIGGFPNQTEDGGLPQSGRIVVDYMVQKSGKT | |
| K483C | GTITYQRGILLPQKVWCVSGKSKVIKGSLPLIGEADCLHEKYGGLNKSKPYYTG | |
| | EHAKAIGNCPIWVKTPLKLANGTKYRPPAKLLKERGFFGCIAGFLEGGWEGMI | |
| | AGWYGYTSHGAHGVAVAADLKSTQEAINKITKNLNSLSELEVKNLQRLSGAM | |
| | DELHNEILELDEKVDDLRADTISSQIELAVLLSNEGIINSEDEHLLALERKLKCM | |
| | LGPSAVEIGNGCFETKHKCNQTCLDRIAAGTFDAGEFSLPTFDSLNITAASLND | |
| | DGLDNHTILLYYSTAASSLAVTLMIAIFVVYMVSRDNVSCSICL | |
| B_Austria_ | MKAIIVLLMVVTSNADRICTGITSSNSPHVVKTATQGEVNVTGVIPLTTTPTKSH | 90 |
| HA: | FANLKGTETRGKLCPKCLNCTDLDVALGRPKCTGKIPSARVSILHEVRPVTSGC | |
| H381Y- | FPIMHDRTKIRQLPNLLRGYEHVRLSTHNVINTEDAPGGPYEIGTSGSCLNITNG | |
| A288V- | KGFFATMAWAVPKNKTATNPLTIEVPYICTEEEDQITVWGFHSDDETQMARLY | |
| G367C- | GDSKPQKFTSSANGVTTHYVSQIGGFPNQTEDGGLPQSGRIVVDYMVQKSGKT | |
| K483C | GTITYQRGILLPQKVWCVSGKSKVIKGSLPLIGEADCLHEKYGGLNKSKPYYTG | |
| | EHAKAIGNCPIWVKTPLKLANGTKYRPPAKLLKERGFFGAIACFLEGGWEGMI | |
| | AGWYGYTSHGAHGVAVAADLKSTQEAINKITKNLNSLSELEVKNLQRLSGAM | |
| | DELHNEILELDEKVDDLRADTISSQIELAVLLSNEGIINSEDEHLLALERKLKCM | |
| | LGPSAVEIGNGCFETKHKCNQTCLDRIAAGTFDAGEFSLPTFDSLNITAASLND | |
| | DGLDNHTILLYYSTAASSLAVTLMIAIFVVYMVSRDNVSCSICL | |
| A_Darwin_ | MKTIIALSNILCLVFAQKIPGNDNSTATLCLGHHAVPNGTIVKTITNDRIEVTNA | 91 |
| H3 HA: | TELVQNSSIGEICGSPHQILDGGNCTLIDALLGDPQCDGFQNKEWDLFVERSRA | |
| G402P- | NSNCYPYDVPDYASLRSLVASSGTLEFKNESFNWTGVKQNGTSSACIRGSSSSF | |
| R421P- | FSRLNWLTSLNNIYPAQNVTMPNKEQFDKLYIWGVHHPDTDKNQISLFAQSSG | |
| E414P | RITVSTKRSQQAVIPNIGSRPRIRGIPSRISIYWTIVKPGDILLINSTGNLIAPRGYF | |
| | KIRSGKSSIMRSDAPIGKCKSECITPNGSIPNDKPFQNVNRITYGACPRYVKQSTL | |
| | KLATGMRNVPEKQTRGIFGAIAGFIENGWEGMVDGWYGFRHQNSEGRGQAA | |
| | DLKSTQAAIDQINGKLNRLIPKTNEKFHQIEKPFSEVEGPVQDLEKYVEDTKIDL | |
| | WSYNAELLVALENQHTIDLTDSEMNKLFEKTKKQLRENAEDMGNGCFKIYHK | |
| | CDNACIGSIRNETYDHNVYRDEALNNRFQIKGVELKSGYKDWILWISFAMSCF | |
| | LLCIALLGFIMWACQKGNIRCNICI | |
| A_Darwin_ | MKTIIALSNILCLVFAQKIPGNDNSTATLCLGHHAVPNGTIVKTITNDRIEVTNA | 92 |
| H3 HA: | TELVQNSSIGEICGSPHQILDGGNCTLIDALLGDPQCDGFQNKEWDLFVERSRA | |
| S123C- | NSNCYPYDVPDYACLRSLVASSGTLEFKNESFNWTGVKQNGTSSACIRGSSSSF | |
| R421C | FSRLNWLTSLNNIYPAQNVTMPNKEQFDKLYIWGVHHPDTDKNQISLFAQSSG | |
| | RITVSTKRSQQAVIPNIGSRPRIRGIPSRISIYWTIVKPGDILLINSTGNLIAPRGYF | |
| | KIRSGKSSIMRSDAPIGKCKSECITPNGSIPNDKPFQNVNRITYGACPRYVKQSTL | |
| | KLATGMRNVPEKQTRGIFGAIAGFIENGWEGMVDGWYGFRHQNSEGRGQAA | |
| | DLKSTQAAIDQINGKLNRLIGKTNEKFHQIEKEFSEVEGCVQDLEKYVEDTKID | |
| | LWSYNAELLVALENQHTIDLTDSEMNKLFEKTKKQLRENAEDMGNGCFKIYH | |
| | KCDNACIGSIRNETYDHNVYRDEALNNRFQIKGVELKSGYKDWILWISFAMSC | |
| | FLLCIALLGFIMWACQKGNIRCNICI | |
| A_Darwin_ | MKTIIALSNILCLVFAQKIPGNDNSTATLCLGHHAVPNGTIVKTICNDRIEVTNA | 93 |
| H3 HA: | TELVQNSSIGEICGSPHQILDGGNCTLIDALLGDPQCDGFQNKEWDLFVERSRA | |
| Q392C- | NSNCYPYDVPDYASLRSLVASSGTLEFKNESFNWTGVKQNGTSSACIRGSSSSF | |
| T46C | FSRLNWLTSLNnIYPAQNVTMPNKEQFDKLYIWGVHHPDTDKNQISLFAQSSG | |
| | RITVSTKRSQQAVIPNIGSRPRIRGIPSRISIYWTIVKPGDILLINSTGNLIAPRGYF | |
| | KIRSGKSSIMRSDAPIGKCKSECITPNGSIPNDKPFQNVNRITYGACPRYVKQSTL | |
| | KLATGMRNVPEKQTRGIFGAIAGFIENGWEGMVDGWYGFRHQNSEGRGQAA | |
| | DLKSTQAAIDCINGKLNRLIGKTNEKFHQIEKEFSEVEGRVQDLEKYVEDTKID | |
| | LWSYNAELLVALENQHTIDLTDSEMNKLFEKTKKQLRENAEDMGNGCFKIYH | |
| | KCDNACIGSIRNETYDHNVYRDEALNNRFQIKGVELKSGYKDWILWISFAMSC | |
| | FLLCIALLGFIMWACQKGNIRCNICI | |
| A_Wisconsin_ | MKAILVVMLYTFTTANADTLCIGYHANNSTDTVDTVCEKNVTVTHSVNLLED | 94 |
| H1 HA: | KHNGKLCKLRGVAPLHLGQCNIAGWILGNPECESLSTARSWSYIVETSNSDNG | |
| K391C- | TCYPGDFINYEELREQLSSVSSFERFEIFPKTSSWPNHDSDNGVTAACSHAGARS | |
| L37C | FYKNLIWLVKKGKSyPKINQTYINDKGKEVLVLWGIHHPPTITDQESLYQNADA | |
| | YVFVGTSRYSKKFKPEIATRPKVRDQAGRMNYYWTLVEPGDKITFEATGNLV | |
| | APRYAFTMEKEAGSGIIISDTPVHDCNATCQTPEGAINTSLPFQNVHPITIGKCPK | |
| | YVRSTKLRLATGLRNVPSIQSRGLFGAIAGFIEGGWTGMVDGWYGYHHQNDQ | |
| | GSGYAADLKSTQNAIDCITNKVNSVIEKMNTQFTAVGKEFNHLEKRIENLNKK | |
| | VDDGFLDVWTYNAELLVLLENERTLDYHDSNVKNLYEKVRHQLKNNAKEIG | |
| | NGCFEFYHKCDNTCMESVKNGTYDYPKYSEEAKLNREKIDGVKLDSTRIYQIL | |
| | AIYSTVASSLVLVVSLGAISFWMCSNGSLQCRICI | |
| A_Wisconsin_ | MKAILVVMLYTFTTANADTLCIGYHANNSTDTVDTCLEKNVTVTHSVNLLED | 84 |
| H1 HA: | KHNGKLCKLRGVAPLHLGQCNIAGWILGNPECESLSTARSWSYIVETSNSDNG | |
| K395C- | TCYPGDFINYEELREQLSSVSSFERFEIFPKTSSWPNHDSDNGVTAACSHAGARS | |
| V36C | FYKNLIWLVKKGKSYPKINQTYINDKGKEVLVLWGIHHPPTITDQESLYQNAD | |
| | AYVFVGTSRYSKKFKPEIATRPKVRDQAGRMNYYWTLVEPGDKITFEATGNL | |
| | VAPRYAFTMEKEAGSGIIISDTPVHDCNATCQTPEGAINTSLPFQNVHPITIGKCP | |
| | KYVRSTKLRLATGLRNVPSIQSRGLFGAIAGFIEGGWTGMVDGWYGYHHQND | |
| | QGSGYAADLKSTQNAIDKITNCVNSVIEKMNTQFTAVGKEFNHLEKRIENLNK | |
| | KVDDGFLDVWTYNAELLVLLENERTLDYHDSNVKNLYEKVRHQLKNNAKEI | |
| | GNGCFEFYHKCDNTCMESVKNGTYDYPKYSEEAKLNREKIDGVKLDSTRIYQI | |
| | LAIYSTVASSLVLVVSLGAISFWMCSNGSLQCRICI | |
| A_Sydney_ | MKAILVVMLYTFTTANADTLCIGYHANNSTDTVDTVCEKNVTVTHSVNLLED | 96 |
| H1 HA: | KHNGKLCKLRGVAPLHLGQCNIAGWILGNPECESLSTARSWSYIVETSNSDNG | |
| K391C- | TCYPGNFINYEELREQLSSVSSFERFEIFPKTSSWPNHDSDNGVTAACPHAGAKS | |
| L37C | FYKNLIWLVKKGKSYPKINQTYINDKGKEVLVLWGIHHPPTITDQESLYQNAD | |
| | AYVFVGTSRYSKKFKPEIAARPKVRDRAGRMNYYWTLVEPGDKITFEATGNL | |
| | VAPRYAFTMEKDAGSGIIISDTPVHDCNTTCQTPEGAINTSLPFQNVHPITIGKCP | |
| | KYVRSTKLRLATGLRNVPSIQSRGLFGAIAGFIEGGWTGMVDGWYGYHHQNE | |
| | QGSGYAADLKSTQNAIDCITNKVNSVIEKMNTQFTAVGKEFNHLEKRIENLNK | |
| | KVDDGFLDIWTYNAELLVLLENERTLDYHDSNVKNLYEKVRNQLKNNAKEIG | |
| | NGCFEFYHKCDNTCMESVKNGTYDYPKYSEEAKLNREKIDGVKLDSTRIYQIL | |
| | AIYSTVASSLVLVVSLGAISFWMCSNGSLQCRICI | |
| A_Sydney_ | MKAILVVMLYTFTTANADTLCIGYHANNSTDTVDTCLEKNVTVTHSVNLLED | 97 |
| H1 HA: | KHNGKLCKLRGVAPLHLGQCNIAGWILGNPECESLSTARSWSYIVETSNSDNG | |
| K395C- | TCYPGNFINYEELREQLSSVSSFERFEIFPKTSSWPNHDSDNGVTAACPHAGAKS | |
| V36C | FYKNLIWLVKKGKSYPKINQTYINDKGKEVLVLWGIHHPPTITDQESLYQNAD | |
| | AYVFVGTSRYSKKFKPEIAARPKVRDRAGRMNYYWTLVEPGDKITFEATGNL | |
| | VAPRYAFTMEKDAGSGIIISDTPVHDCNTTCQTPEGAINTSLPFQNVHPITIGKCP | |
| | KYVRSTKLRLATGLRNVPSIQSRGLFGAIAGFIEGGWTGMVDGWYGYHHQNE | |
| | QGSGYAADLKSTQNAIDKITNCVNSVIEKMNTQFTAVGKEFNHLEKRIENLNK | |
| | KVDDGFLDIWTYNAELLVLLENERTLDYHDSNVKNLYEKVRNQLKNNAKEIG | |
| | NGCFEFYHKCDNTCMESVKNGTYDYPKYSEEAKLNREKIDGVKLDSTRIYQIL | |
| | AIYSTVASSLVLVVSLGAISFWMCSNGSLQCRICI | |

### Wild-Type Wuhan-Hu-1; USA-WA1/2020 Isolate

| | | |
|---|---|---|
| Wild-Type | MFVFLVLLPLVSSQCVNLTTRTQLPPAYTNSFTRGVYYPDKVERSSVLHSTQDLFLPFFSN | SEQ ID |
| Wuhan-Hu- | VTWFHAIHVSGTNGTKRFDNPVLPENDGVYFASTEKSNIIRGWIFGTTLDSKTQSLLIVNN | NO: 78 |
| 1; USA- | ATNVVIKVCEFQFCNDPFLGVYYHKNNKSWMESEFRVYSSANNCTFEYVSQPFLMDLEGKQ | |
| WA1/2020 | GNFKNLREFVFKNIDGYFKIYSKHTPINLVRDLPQGFSALEPLVDLPIGINITRFQTLLAL | |
| isolate | HRSYLTPGDSSSGWTAGAAAYYVGYLQPRTFLLKYNENGTITDAVDCALDPLSETKCTLKS | |
| | FTVEKGIYQTSNFRVQPTESIVRFPNITNLCPFGEVFNATRFASVYAWNRKRISNCVADYS | |
| | VLYNSASFSTFKCYGVSPTKLNDLCFTNVYADSFVIRGDEVRQIAPGQTGKIADYNYKLPD | |
| | DFTGCVIAWNSNNLDSKVGGNYNYLYRLFRKSNLKPFERDISTEIYQAGSTPCNGVEGENC | |
| | YFPLQSYGFQPTNGVGYQPYRVVVLSFELLHAPATVCGPKKSTNLVKNKCVNFNFNGLTGT | |
| | GVLTESNKKELPFQQFGRDIADTTDAVRDPQTLEILDITPCSFGGVSVITPGTNTSNQVAV | |
| | LYQDVNCTEVPVAIHADQLTPTWRVYSTGSNVFQTRAGCLIGAEHVNNSYECDIPIGAGIC | |
| | ASYQTQTNSPRRARSVASQSIIAYTMSLGAENSVAYSNNSIAIPTNFTISVTTEILPVSMT | |
| | KTSVDCTMYICGDSTECSNLLLQYGSFCTQLNRALTGIAVEQDKNTQEVFAQVKQIYKTPP | |
| | IKDFGGFNFSQILPDPSKPSKRSFIEDLLENKVTLADAGFIKQYGDCLGDIAARDLICAQK | |
| | FNGLTVLPPLLTDEMIAQYTSALLAGTITSGWTFGAGAALQIPFAMQMAYRENGIGVTQNV | |
| | LYENQKLIANQFNSAIGKIQDSLSSTASALGKLQDVVNQNAQALNTLVKQLSSNFGAISSV | |
| | LNDILSRLDKVEAEVQIDRLITGRLQSLQTYVTQQLIRAAEIRASANLAATKMSECVLGQS | |
| | KRVDFCGKGYHLMSFPQSAPHGVVFLHVTYVPAQEKNFTTAPAICHDGKAHFPREGVFVSN | |
| | GTHWFVTQRNFYEPQIITTDNTFVSGNCDVVIGIVNNTVYDPLQPELDSFKEELDKYFKNH | |
| | TSPDVDLGDISGINASVVNIQKEIDRLNEVAKNLNESLIDLQELGKYEQYIKWPWYIWLGF | |
| | IAGLIAIVMVTIMLCCMTSCCSCLKGCCSCGSCCKFDEDDSEPVLKGVKLHYT | |

### Respiratory Syncytial Virus F Glycoprotein and SARS-CoV-2 Spike Glycoprotein

| | | |
|---|---|---|
| Wild-Type | MELLIHRSSAIFLTLAINTLYLTSSQNITEEFYQSTCSAVSRGYFSALRTGWYTSVITIELSN | SEQ ID |
| RSVF | IKETKCNGTDTKVKLIKQELDKYKNAVTELQLLMQNTPAANNRARREAPQYMNYTINTTKNLN | NO: 98 |
| Glycoprotein | VSISKKRKRRFLGFLLGVGSAIASGIAVSKVLHLEGEVNKIKNALLSTNKAVVSLSNGVSVLT | |
| | SKVLDLKNYINNQLLPIVNQQSCRISNIETVIEFQQKNSRLLEITREFSVNAGVTTPLSTYML | |
| | TNSELLSLINDMPITNDQKKLMSSNVQIVRQQSYSIMSIIKEEVLAYVVQLPIYGVIDTPCWK | |
| | LHTSPLCTTNIKEGSNICLTRTDRGWYCDNAGSVSFFPQADTCKVQSNRVFCDTMNSLTLPSE | |
| | VSLCNTDIFNSKYDCKIMTSKTDISSSVITSLGAIVSCYGKTKCTASNKNRGIIKTESNGCDY | |
| | VSNKGVDTVSVGNTLYYVNKLEGKNLYVKGEPIINYYDPLVFPSDEFDASISQVNEKINQSLA | |
| | FIRRSDELLHNVNTGKSTTNIMITAIIIVIIVVLLSLIAIGLLLYCKAKNTPVTLSKDQLSGI | |
| | NNIAFSK | |
| Prefusion RSV | MELLILKANAITTILTAVTFCFASGQNITEEFYQSTCSAVSKGYLSALRTGWYTSVITIELSN | SEQ ID |
| F Glycoprotein | IKENKCNGTDAKVKLIKQELDKYKNAVTELQLLMQSTPATGSGSAICSGVAVCKVLHLEGEVN KIKSALLSTNKAVVSLSNGVSVLTFKVLDLKNYIDKQLLPILNKQSCSISNIETVIEFQQKNN | NO: 99 |
| | RLLEITREFSVNAGVTTPVSTYMLTNSELLSLINDMPITNDQKKLMSNNVQIVRQQSYSIMCI | |
| | IKEEVLAYVVQLPLYGVIDTPCWKLHTSPLCTTNTKEGSNICLTRTDRGWYCDNAGSVSFFPQ | |
| | AETCKVQSNRVFCDTMNSRTLPSEVNLCNVDIFNPKYDCKIMTSKTDVSSSVITSLGAIVSCY | |
| | GKTKCTASNKNRGIIKTFSNGCDYVSNKGVDTVSVGNTLYCVNKQEGKSLYVKGEPIINFYDP | |
| | LVFPSDEFDASISQVNEKINQSLAFIRKSDELLHNVNAGKSTTNIMITTIIIVIIVILLSLIA | |
| | VGLLLY | |
| Prefusion RSV | ATGGAGCTGCTGATCCTGAAGGCCAACGCCATCACGACCATCCTGACCGCCGTGACCTTCTGC | SEQ ID |
| F Glycoprotein | TTCGCCAGCGGGCAGAACATCACCGAGGAGTTCTACCAGTCCACCTGCTCCGCCGTGAGCAAG | NO: 113 |
| ORF (not | GGCTACCTGTCTGCCCTGAGAACCGGCTGGTACACCAGCGTGATCACCATCGAGCTGTCCAAC | |
| including | ATCAAGGAGAACAAGTGCAACGGCACCGACGCCAAGGTGAAGCTGATCAAGCAGGAGCTGGAC | |
| stop | AAGTACAAGAACGCAGTGACCGAGCTGCAGCTGCTGATGCAGAGCACACCAGCCACCGGTAGC | |
| codon) | GGGTCCGCCATTTGCTCCGGCGTGGCCGTGTGCAAGGTGCTGCACCTGGAGGGCGAGGTGAAC | |
| | AAGATCAAGAGCGCCCTGCTCTCCACCAACAAGGCCGTGGTGAGCCTGAGCAACGGGGTGAGC | |
| | GTGCTGACCTTCAAGGTGCTGGACCTGAAGAACTACATCGACAAGCAGCTGCTGCCTATCCTG | |
| | AACAAGCAGAGCTGCAGCATCAGCAACATCGAGACCGTGATCGAGTTCCAGCAGAAGAACAAC | |
| | CGGCTGCTGGAGATCACCAGGGAGTTCAGCGTGAACGCAGGGGTGACCACACCCGTGTCCACC | |
| | TACATGCTGACCAACTCCGAGCTGCTGAGCCTGATCAACGATATGCCCATCACCAACGACCAG | |
| | AAGAAGCTGATGAGCAACAACGTGCAGATCGTGCGGCAGCAGTCCTACTCCATCATGTGCATC | |
| | ATCAAGGAGGAGGTGCTGGCCTACGTGGTGCAGCTGCCCCTGTACGGCGTGATCGACACCCCT | |
| | TGCTGGAAGCTGCACACCAGCCCTCTGTGCACCACCAACACGAAGGAGGGCAGCAATATCTGC | |
| | CTGACCCGGACCGACAGGGGCTGGTACTGCGACAACGCCGGCAGCGTGTCCTTCTTTCCCCAG | |
| | GCCGAGACCTGCAAGGTGCAGTCCAACAGGGTGTTCTGCGACACCATGAACTCTCGCACCCTG | |
| | CCCAGCGAGGTGAACCTGTGCAACGTGGACATCTTCAACCCCAAGTACGACTGCAAGATCATG | |
| | ACCTCCAAGACCGACGTGTCCTCTAGCGTTATCACCTCCCTGGGCGCCATCGTGAGCTGCTAC | |
| | GGCAAGACCAAGTGCACCGCCAGCAACAAGAACAGGGGCATCATCAAGACCTTCAGCAACGGG | |
| | TGCGACTACGTGTCCAACAAGGGCGTGGACACCGTGTCCGTGGGCAACACCCTGTACTGCGTG | |
| | AACAAGCAGGAGGGCAAGAGCCTGTACGTGAAGGGCGAGCCCATCATCAACTTCTACGACCCT | |
| | CTGGTGTTCCCCAGCGACGAGTTCGACGCCAGCATCTCCCAGGTGAACGAGAAGATCAACCAG | |
| | AGCCTGGCCTTCATCCGCAAGAGCGACGAGCTGCTGCACAACGTGAACGCCGGCAAGAGCACC | |
| | ACAAACATCATGATCACCACCATCATCATCGTGATAATCGTGATCCTGCTGTCCCTGATCGCT | |
| | GTGGGCCTGCTGCTGTAC | |
| RSV F C- | CKARSTPVTLSKDQLSGINNIAFSN | SEQ ID |
| terminal 25 | | NO: 100 |
| amino acids | | |
| RSV F C- | TPVTLSKDQLSGINNIAFSN | SEQ ID |
| terminal 20 | | NO: 101 |
| amino acids | | |
| RSV-F C- | SKDQLSGINNIAFSN | SEQ ID |
| terminal 15 | | NO: 102 |
| amino acids | | |
| RSV-F C- | SGINNIAFSN | SEQ ID |
| terminal 10 | | NO: 103 |
| amino acids | | |
| Wild-type | MFVFLVLLPLVSSQCVNLTTRTQLPPAYTNSFTRGVYYPDKVFRSSVLHSTQDLFLPFFSNVT | NO: 104 |
| SARS-CoV-2 S | WFHAIHVSGTNGTKRFDNPVLPENDGVYFASTEKSNIIRGWIFGTTLDSKTQSLLIVNNATNV | SEQ ID |
| protein | VIKVCEFQFCNDPFLGVYYHKNNKSWMESEFRVYSSANNCTFEYVSQPFLMDLEGKQGNEKNL | |
| | REFVFKNIDGYFKIYSKHTPINLVRDLPQGFSALEPLVDLPIGINITRFQTLLALHRSYLTPG | |
| | DSSSGWTAGAAAYYVGYLQPRTFLLKYNENGTITDAVDCALDPLSETKCTLKSFTVEKGIYQT | |
| | SNFRVQPTESIVRFPNITNLCPFGEVENATRFASVYAWNRKRISNCVADYSVLYNSASFSTEK | |
| | CYGVSPTKLNDLCFTNVYADSFVIRGDEVRQIAPGQTGKIADYNYKLPDDFTGCVIAWNSNNL | |
| | DSKVGGNYNYLYRLFRKSNLKPFERDISTEIYQAGSTPCNGVEGENCYFPLQSYGFQPTNGVG | |
| | YQPYRVVVLSFELLHAPATVCGPKKSTNLVKNKCVNFNFNGLTGTGVLTESNKKELPFQQFGR | |
| | DIADTTDAVRDPQTLEILDITPCSFGGVSVITPGTNTSNQVAVLYQDVNCTEVPVAIHADQLT | |
| | PTWRVYSTGSNVFQTRAGCLIGAEHVNNSYECDIPIGAGICASYQTQTNSPRRARSVASQSII | |
| | AYTMSLGAENSVAYSNNSIAIPTNFTISVTTEILPVSMTKTSVDCTMYICGDSTECSNLLLQY | |
| | GSFCTQLNRALTGIAVEQDKNTQEVFAQVKQIYKTPPIKDFGGFNFSQILPDPSKPSKRSFIE | |
| | DLLFNKVTLADAGFIKQYGDCLGDIAARDLICAQKENGLTVLPPLLTDEMIAQYTSALLAGTI | |
| | TSGWTFGAGAALQIPFAMQMAYRENGIGVTQNVLYENQKLIANQFNSAIGKIQDSLSSTASAL | |
| | GKLQDVVNQNAQALNTLVKQLSSNFGAISSVLNDILSRLDKVEAEVQIDRLITGRLQSLQTYV | |
| | TQQLIRAAEIRASANLAATKMSECVLGQSKRVDFCGKGYHLMSFPQSAPHGVVFLHVTYVPAQ | |
| | EKNFTTAPAICHDGKAHFPREGVFVSNGTHWFVTQRNFYEPQIITTDNTFVSGNCDVVIGIVN | |
| | NTVYDPLQPELDSFKEELDKYFKNHTSPDVDLGDISGINASVVNIQKEIDRLNEVAKNLNESL | |
| | IDLQELGKYEQYIKWPWYIWLGFIAGLIAIVMVTIMLCCMTSCCSCLKGCCSCGSCCKFDEDD | |
| | SEPVLKGVKLHYT | |
| Prefusion | MFVFLVLLPLVSSQCVNLTTRTQLPPAYTNSFTRGVYYPDKVFRSSVLHSTQDLFLPFFSNVT | SEQ ID |
| SARS-CoV-2 S | WFHAIHVSGTNGTKRFDNPVLPFNDGVYFASTEKSNIIRGWIFGTTLDSKTQSLLIVNNATNV | NO: 105 |
| protein | VIKVCEFQFCNDPFLGVYYHKNNKSWMESEFRVYSSANNCTFEYVSQPFLMDLEGKQGNEKNL | |
| | REFVFKNIDGYFKIYSKHTPINLVRDLPQGFSALEPLVDLPIGINITRFQTLLALHRSYLTPG | |
| | DSSSGWTAGAAAYYVGYLQPRTFLLKYNENGTITDAVDCALDPLSETKCTLKSFTVEKGIYQT | |
| | SNFRVQPTESIVRFPNITNLCPFGEVENATRFASVYAWNRKRISNCVADYSVLYNSASFSTEK | |
| | CYGVSPTKLNDLCFTNVYADSFVIRGDEVRQIAPGQTGKIADYNYKLPDDFTGCVIAWNSNNL | |
| | DSKVGGNYNYLYRLFRKSNLKPFERDISTEIYQAGSTPCNGVEGENCYFPLQSYGFQPTNGVG | |
| | YQPYRVVVLSFELLHAPATVCGPKKSTNLVKNKCVNFNFNGLTGTGVLTESNKKFLPFQQFGR | |
| | DIADTTDAVRDPQTLEILDITPCSFGGVSVITPGTNTSNQVAVLYQDVNCTEVPVAIHADQLT | |
| | PTWRVYSTGSNVFQTRAGCLIGAEHVNNSYECDIPIGAGICASYQTQTNSPRRARSVASQSII | |
| | AYTMSLGAENSVAYSNNSIAIPTNFTISVTTEILPVSMTKTSVDCTMYICGDSTECSNLLLQY | |
| | GSFCTQLNRALTGIAVEQDKNTQEVFAQVKQIYKTPPIKDFGGFNFSQILPDPSKPSKRSFIE | |
| | DLLFNKVTLADAGFIKQYGDCLGDIAARDLICAQKENGLTVLPPLLTDEMIAQYTSALLAGTI | |
| | TSGWTFGAGAALQIPFAMQMAYRENGIGVTQNVLYENQKLIANQFNSAIGKIQDSLSSTASAL | |
| | GKLQDVVNQNAQALNTLVKQLSSNFGAISSVLNDILSRLDPPEAEVQIDRLITGRLQSLQTYV | |
| | TQQLIRAAEIRASANLAATKMSECVLGQSKRVDFCGKGYHLMSFPQSAPHGVVFLHVTYVPAQ | |
| | EKNFTTAPAICHDGKAHFPREGVFVSNGTHWFVTQRNFYEPQIITTDNTFVSGNCDVVIGIVN | |
| | NTVYDPLQPELDSFKEELDKYFKNHTSPDVDLGDISGINASVVNIQKEIDRLNEVAKNLNESL | |
| | IDLQELGKYEQYIKWPWYIWLGFIAGLIAIVMVTIMLCCMTSCCSCLKGCCSCGSCCKFDEDD | |
| | SEPVLKGVKLHYT | |

It should be understood that any of the mRNA sequences may include a 5′ UTR and/or a 3′ UTR. The UTR sequences may be selected from the following sequences, or other known UTR sequences may be used. It should also be understood that any of the mRNA constructs may further comprise a poly(A) tail and/or cap (e.g., 7 mG(5′)ppp(5′)NlmpNp). Further, while many of the mRNAs and encoded antigen sequences include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
(SEQ ID NO: 1)
GGGAAAUAAGAGAGAAAAGAAGAGUAAGAAGAAAUAUAAGAG
CCACC
5′ UTR:
(SEQ ID NO: 2)
GGGAAAUAAGAGAGAAAAGAAGAGUAAGAAGAAAUAUAAGACC
CCGGCGCCGCCACC
3′ UTR:
(SEQ ID NO: 3)
UGAUAAUAGGCUGGAGCCUCGGUGGCCAUGCUUCUUGCCCCUU
GGGCCUCCCCCCAGCCCCUCCUCCCCUUCCUGCACCCGUACCC
CCGUGGUCUUUGAAUAAAGUCUGAGUGGGCGGC
3′ UTR:
(SEQ ID NO: 4)
UGAUAAUAGGCUGGAGCCUCGGUGGCCUAGCUUCUUGCCCCUU
GGGCCUCCCCCCAGCCCCUCCUCCCCUUCCUGCACCCGUACCC
CCGUGGUCUUUGAAUAAAGUCUGAGUGGGCGGC

TABLE S-1
5′ UTR sequences

| SEQ ID | |
|---|---|
| NO: | Sequence |
| 5 | GGAAAUCGCAAAAUUUGCUCUUCGCGUUAGAUUUCUUUUAGUUUUCUCGCAACUAGCAAG |
| | CUUUUUGUUCUCGCC |
| 6 | GGAAAUCCCCACAACCGCCUCAUAUCCAGGCUCAAGAAUAGAGCUCAGUGUUUUGUUGUU |
| | UAAUCAUUCCGACGUGUUUUGCGAUAUUCGCGCAAAGCAGCCAGUCGCGCGCUUGCUUUU |
| | AAGUAGAGUUGUUUUUCCACCCGUUUGCCAGGCAUCUUUAAUUUAACAUAUUUUUAUUUU |
| | UCAGGCUAACCUACGCCGCCACC |
| 7 | GGAAAUAAGAGAGAAAAGAAGAGUAAGAAGAAAUAUAAGAUCUCCCUGAGCUUCAGGGAG |
| | CCCCGGCGCCGCCACC |
| 8 | GGAAACCCCCCACCCCCGUAAGAGAGAAAAGAAGAGUAAGAAGAAAUAUAAGAUCUCCCU |
| | GAGCUUCAGGGAGCCCCGGCGCCGCCACC |
| 9 | GGAGAACUUCCGCUUCCGUUGGCGCAAGCGCUUUCAUUUUUUCUGCUACCGUGACUAAG |
| 10 | GGAAAUAAGAGAGAAAAGAAGAGUAAGAAGAAAUAUAAGAGCCACC |
| 11 | GGAAAUAAGAGAGAAAAGAAGAGUAAGAAGAAAUAUAAGACCCCGGCGCCGCCACC |
| 12 | GGAAAUCGCAAAAUUUGCUCUUCGCGUUAGAUUUCUUUUAGUUUUCUCGCAACUAGCAAG |
| | CUUUUUGUUCUCGCCGCCGCC |
| 13 | GGAAAUCGCAAAAUUUUCUUUUCGCGUUAGAUUUCUUUUAGUUUUCUUUCAACUAGCAAG |
| | CUUUUUGUUCUCGCCGCCGCC |
| 14 | G G A A A U C G C A A A A (N₂)ₓ (N₃)ₓ C U (N₄)ₓ (N₅)ₓ C G C G U |
| | U A G A U U U C U U U U A G U U U U C U N N C A A C U A G C |
| | A A G C U U U U U G U U C U C G C C (N₈ C C )x |
| | (N₂)ₓ is a uracil and x is an integer from 0 to 5, e.g., wherein x = 3 or 4; |
| | (N₃)ₓ is a guanine and x is an integer from 0 to 1; |
| | (N₄)ₓ is a cytosine and x is an integer from 0 to 1; |
| | (N₅)ₓ is a uracil and x is an integer from 0 to 5, e.g., wherein x = 2 or 3; |
| | N₆ is a uracil or cytosine; |
| | N₇ is a uracil or guanine; |
| | N₈ is adenine or guanine and x is an integer from 0 to 1. |
| 15 | GGAAAAUUUUAGCCUGGAACGUUAGAUAACUGUCCUGUUGUCUUUAUAUACUUGGUCCCC |
| | AAGUAGUUUGUCUUCCAAA |
| 16 | GGAAACUUUAUUUAGUGUUACUUUAUUUUCUGUUUAUUUGUGUUUCUUCAGUGGGUUUGU |
| | UCUAAUUUCCUUGGCCGCC |
| 17 | GGAAAAUCUGUAUUAGGUUGGCGUGUUCUUUGGUCGGUUGUUAGUAUUGUUGUUGAUUCG |
| | UUUGUGGUCGGUUGCCGCC |
| 18 | GGAAAAUUAUUAACAUCUUGGUAUUCUCGAUAACCAUUCGUUGGAUUUUAUUGUAUUCGU |
| | AGUUUGGGUUCCUGCCGCC |
| 19 | GGAAAUUAUUAUUAUUUCUAGCUACAAUUUAUCAUUGUAUUAUUUUAGCUAUUCAUCAUU |
| | AUUUACUUGGUGAUCAACA |
| 20 | GGAAAUAGGUUGUUAACCAAGUUCAAGCCUAAUAAGCUUGGAUUCUGGUGACUUGCUUCA |
| | CCGUUGGCGGGCACCGAUC |
| 21 | GGAAAUCGUAGAGAGUCGUACUUAGUACAUAUCGACUAUCGGUGGACACCAUCAAGAUUA |
| | UAAACCAGGCCAGA |
| 22 | GGAAACCCGCCCAAGCGACCCCAACAUAUCAGCAGUUGCCCAAUCCCAACUCCCAACACA |
| | AUCCCCAAGCAACGCCGCC |
| 23 | GGAAAGCGAUUGAAGGCGUCUUUUCAACUACUCGAUUAAGGUUGGGUAUCGUCGUGGGAC |
| | UUGGAAAUUUGUUGUUUCC |
| 24 | GGAAACUAAUCGAAAUAAAAGAGCCCCGUACUCUUUUAUUUCUAUUAGGUUAGGAGCCUU |
| | AGCAUUUGUAUCUUAGGUA |
| 25 | GGAAAUGUGAUUUCCAGCAACUUCUUUUGAAUAUAUUGAAUUCCUAAUUCAAAGCGAACA |
| | AAUCUACAAGCCAUAUACC |
| 26 | GGAAAUCGUAGAGAGUCGUACUUACGUGGUCGCCAUUGCAUAGCGCGCGAAAGCAACAGG |
| | AACAAGAACGCGCC |
| 27 | GGAAAUCGUAGAGAGUCGUACUUAGAAUAAACAGAGUCGGGUCGACUUGUCUCUGAUACU |
| | ACGACGUCACAAUC |
| 28 | GGAAAAUUUGCCUUCGGAGUUGCGUAUCCUGAACUGCCCAGCCUCCUGAUAUACAACUGU |
| | UCCGCUUAUUCGGGCCGCC |
| 29 | GGAAAUCUGAGCAGGAAUCCUUUGUGCAUUGAAGACUUUAGAUUCCUCUCUGCGGUAGAC |
| | GUGCACUUAUAAGUAUUUG |
| 30 | GGAAAGCGAUUGAAGGCGUCUUUUCAACUACUCGAUUAAGGUUGGGUAUCGUCGUGGGAC |
| | UUGGAAAUUUGUUGCCACC |
| 31 | GGAAAUUUUUUUUUGAUAUUAUAAGAGUUUUUUUUUGAUAUUAAGAAAAUUUUUUUUUGA |
| | UAUUAGAAGAGUAAGAAGAAAUAUAAGACCCCGGCGCCGCCACC |
| 32 | GGAAAUAAGAGAGAAAAGAAGAGUAAGAAGAAAUAUAAGAGCCAAAAAAAAAAAACC |
| 33 | GGAAAUCUCCCUGAGCUUCAGGGAGUAAGAGAGAAAAGAAGAGUAAGAAGAAAUAUAAGA |
| | CCCCGGCGCCGCCACC |
| 34 | GCCRCC, wherein R = A or G |
| 35 | GGACUCACUAUUUGUUUUCGCGCCCAGUUGCAAAAA |

TABLE S-2
3′ UTR sequences (stop cassette is italicized; miR binding sites are boldened)

| SEQ ID | |
|---|---|
| NO: | |
| 36 | UAAAGCUCCCCGGGGGCCUCGGUGGCCUAGCUUCUUGCCCCUUGGGCCUCCCCCCAGCC |
| | CCUCCUCCCCUUCCUGCACCCGUACCCCCGUGGUCUUUGAAUAAAGUCUGAGUGGGGGC |
| 37 | UAAGUCUAAGCUGGAGCCUCCUGAGAGACCUGUGUGAACUAUUGAGAAGAUCGGAACAG |
| | CUCCUUACUCUGAGGAAGUUGGUACCCCCGUGGUCUUUGAAUAAAGUCUGAGUGGGCGGC |
| 38 | UAAAGCUCCCCGGGGCAAACACCAUUGUCACACUCCAGCCUCGGUGGCCUAGCUUCUUG |
| | CCCCUUGGGCCCAAACACCAUUGUCACACUCCAUCCCCCCAGCCCCUCCUCCCCUUCCUGCA |
| | CCCGUACCCCCCAAACACCAUUGUCACACUCCAGUGGUCUUUGAAUAAAGUCUGAGUGGGCG |
| | GC |
| | (miR122 binding sites boldened) |
| 39 | UAAAGCUCCCCGGGGUCCAUAAAGUAGGAAACACUACAGCUGGAGCCUCGGUGGCCUAG |
| | CUUCUUGCCCCUUGGGCCCAAACACCAUUGUCACACUCCAUCCCCCCAGCCCCUCCUCCCCU |
| | UCCUGCACCCGUACCCCCGUGGUCUUUGAAUAAAGUCUGAGUGGGGGC |
| | (miR-142-3p and miR122 binding sites boldened) |
| 40 | UAAAGCUCCCCGGGGGCCUCGGUGGCCUAGCUUCUUGCCCCUUGGGCCUCCCCCCAGCC |
| | CCUCCUCCCCUUCCUGCACCCGUACCCCCCAAACACCAUUGUCACACUCCAGUGGUCUUUGA |
| | AUAAAGUCUGAGUGGGCGGC |
| | (miR122 binding site boldened) |
| 41 | UAAGCCCCUCCGGGGCAAACACCAUUGUCACACUCCAGCCUCGGUGGCCUAGCUUCUUG |
| | CCCCUUGGGCCCAAACACCAUUGUCACACUCCAUCCCCCCAGCCCCUCCUCCCCUUCCUGCA |
| | CCCGUACCCCCCAAACACCAUUGUCACACUCCAGUGGUCUUUGAAUAAAGUCUGAGUGGGCG |
| | GC |
| | (miR122 binding sites boldened) |
| 42 | UAAGCCCCUCCGGGGUCCAUAAAGUAGGAAACACUACAGCCUCGGUGGCCUAGCUUCUU |
| | GCCCCUUGGGCCUCCAUAAAGUAGGAAACACUACAUCCCCCCAGCCCCUCCUCCCCUUCCUG |
| | CACCCGUACCCCCCGCAUUAUUACUCACGGUACGAGUGGUCUUUGAAUAAAGUCUGAGUGGG |
| | CGGC |
| | (miR-142-3p and miR-126-3p binding sites boldened) |
| 43 | UAAGCCCCUCCGGGGUCCAUAAAGUAGGAAACACUACAGCUGGAGCCUCGGUGGCCUAG |
| | CUUCUUGCCCCUUGGGCCCAAACACCAUUGUCACACUCCAUCCCCCCAGCCCCUCCUCCCCU |
| | UCCUGCACCCGUACCCCCGUGGUCUUUGAAUAAAGUCUGAGUGGGGGGC |
| | (miR-142-3p and miR122 binding sites boldened) |
| 44 | UAAGCCCCUCCGGGGGCCUCGGUGGCCUAGCUUCUUGCCCCUUGGGCCUCCCCCCAGCC |
| | CCUCCUCCCCUUCCUGCACCCGUACCCCCCAAACACCAUUGUCACACUCCAGUGGUCUUUGA |
| | AUAAAGUCUGAGUGGGCGGC |
| | (miR122 binding site boldened) |

### EQUIVALENTS

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

### ENUMERATED EMBODIMENTS

1. A messenger ribonucleic acid (mRNA) vaccine comprising a first mRNA encoding a stabilized prefusion hemagglutinin (HA) antigen of a first influenza B virus, wherein the HA antigen comprises at least one stabilizing substitution in a non-surface-exposed region of the HA antigen.
2. The mRNA vaccine of Embodiment 1, wherein the HA antigen of the first influenza B virus comprises two stabilizing mutations.
3. The mRNA vaccine of any one of the preceding Embodiments, wherein the HA antigen of the first influenza B virus comprises three stabilizing mutations.
4. The mRNA vaccine of any one of the preceding Embodiments, wherein the stabilizing substitution comprises a substitution at an amino acid position selected from the group consisting of: Q267, A288, G273, H381, K388, D422, S434, H435, and H455; wherein the amino acid position is determined by alignment to, and/or using the amino acid numbering of SEQ ID NO: 70 or 71.
5. The mRNA vaccine of Embodiment 4, wherein the stabilizing substitution is selected from the group consisting of: Q267L, A288V, G273A, H381Y, K388M, K388L, D422N, S434A, H435L, and H455W; wherein the amino acid position is determined by alignment to, and/or using the amino acid numbering of SEQ ID NO: 70 or 71.
6. The mRNA vaccine of Embodiment 4 or 5, wherein the HA antigen comprises an H381Y substitution.
7. The mRNA vaccine of Embodiment 4 or 5, wherein the HA antigen comprises an A²⁸⁸V substitution.
8. The mRNA vaccine of any one of Embodiments 4-7, wherein the HA antigen comprises an H381Y substitution and an A288V substitution.
9. The mRNA vaccine of Embodiment 4 or 5, wherein the HA antigen comprises a Q267L substitution.
10. The mRNA vaccine of Embodiment 4 or 5, wherein the HA antigen comprises a G273A substitution.
11. The mRNA vaccine of Embodiment 4 or 5, wherein the HA antigen comprises an H435L substitution.
12. The mRNA vaccine of any one of Embodiments 9-11, wherein the HA antigen comprises Q267L, G273A, and H435L substitutions.
13. The mRNA of any one of the preceding Embodiments, wherein the HA antigen comprises an H381Y substitution.
14. The mRNA of any one of the preceding Embodiments, wherein the HA antigen comprises a A288V substitution.
15. The mRNA of any one of the preceding Embodiments, wherein the HA antigen comprises V239C, V276C, D451C, and K422C substitutions.
16. The mRNA of any one of the preceding Embodiments, wherein the HA antigen comprises I367C, S401C, D451C, and K422C substitutions.
17. The mRNA of any one of the preceding Embodiments, wherein the HA antigen comprises H381Y, A288V, N494C, and K483C substitutions.
18. The mRNA of any one of the preceding Embodiments, wherein the HA antigen comprises H381Y, A288V, L422C, and D444C substitutions.
19. The mRNA of any one of the preceding Embodiments, wherein the HA antigen comprises H381Y, A288V, A364C, and K483C substitutions.
20. The mRNA of any one of the preceding Embodiments, wherein the HA antigen comprises H381Y, A288V, G367C, and K483C substitutions.
21. The mRNA vaccine of any one of the preceding Embodiments, wherein the substitution is relative to SEQ ID NO: 70.
22. The mRNA vaccine of any one of the preceding Embodiments, wherein the substitution is relative to SEQ ID NO: 71.
23. The mRNA vaccine of any one of Embodiments 1-5, wherein the HA antigen comprises an amino acid sequence that has at least 90%, at least 95%, or at least 98% identity to a sequence selected from the group consisting of SEQ ID NOs: 48, 51, 54, 57, 72-77, and 85-90.
24. The mRNA vaccine of Embodiment 23, wherein the HA antigen comprises an amino acid sequence selected from the group consisting of SEQ ID NOs: 48, 51, 54, 57, 72-77, and 85-90.
25. The mRNA vaccine of any one of Embodiments 23-24, wherein the mRNA comprises an open reading frame (ORF) that comprises a sequence that has at least 90%, at least 95%, or at least 98% identity to a sequence selected from the group consisting of SEQ ID NOs: 46, 49, 52, and 55.
26. The mRNA vaccine of any one of Embodiments 23-25, wherein the mRNA comprises an ORF that comprises a sequence selected from the group consisting of SEQ ID NOs: 46, 49, 52, and 55.
27. The mRNA vaccine of any one of Embodiments 23-26, wherein the mRNA comprises a sequence that has at least 90%, at least 95%, or at least 98% identity to a sequence selected from the group consisting of SEQ ID NOs: 64-67.
28. The mRNA vaccine of any one of Embodiments 23-24, wherein the mRNA comprises a sequence a sequence selected from the group consisting of SEQ ID NOs: 64-67.
29. The mRNA vaccine of any one of the preceding Embodiments, further comprising a second mRNA encoding an HA antigen of a second influenza B virus.
30. The mRNA vaccine of Embodiment 29, wherein the HA antigen of the second influenza B virus comprises 1, 2, or 3 stabilizing substitutions and wherein the first and second influenza B viruses are different from one another.
31. The mRNA vaccine of Embodiment 30, wherein the HA antigen comprises the substitutions of any one of Embodiments 4-20.
32. The mRNA vaccine of Embodiment 30 or 31, wherein the stabilizing substitutions of the HA antigen of the first influenza B virus are the same as the stabilizing substitutions of the HA antigen of the second influenza B virus.
33. The mRNA vaccine of Embodiment 30 or 31, wherein the stabilizing substitutions of the HA antigen of the first influenza B virus are different from the stabilizing substitutions of the HA antigen of the second influenza B virus.
34. The mRNA vaccine of any one of the preceding Embodiments, further comprising 1-4 mRNA molecules, each encoding an HA antigen of a different influenza A virus.
35. The mRNA vaccine of Embodiment 34, wherein the vaccine comprises 2 mRNA molecules, each encoding an HA antigen of a different influenza A virus.
36. The mRNA vaccine of Embodiment 34 or 35, wherein one HA antigen is an H1 HA antigen, and one HA antigen is an H3 HA antigen.
37. The mRNA vaccine of Embodiment 36, wherein the H1 HA antigen comprises:
   (i) a K391C substitution and an L37C substitution; and/or
   (ii) a K395C substitution and a V36C substitution,
   wherein the amino acid position is determined by alignment to, and/or using the amino acid numbering of SEQ ID NO: 83 or 95.
38. The mRNA vaccine of Embodiment 36 or 37, wherein the H3 HA antigen comprises:
   (i) a Q392C substitution and a T46C substitution;
   (ii) an S123C substitution and an R421C substitution; and/or
   (iii) a G402P substitution, an R421P substitution, and/or an E414P substitution,
   wherein the amino acid position is determined by alignment to, and/or using the amino acid numbering of SEQ ID NO: 82.
39. The mRNA vaccine of any one of Embodiments 36-38, wherein the H1 HA antigen comprises an amino acid sequence with at least 95% or at least 98% sequence identity to any one of SEQ ID NOs: 84, 96, or 97.
40. The mRNA vaccine of any one of Embodiments 36-38, wherein the H3 HA antigen comprises an amino acid sequence with at least 95% or at least 98% sequence identity to any one of SEQ ID NOs: 91-93.
41. The mRNA vaccine of any one of the preceding Embodiments, further comprising an mRNA encoding a first SARS-CoV-2 antigen.
42. The mRNA vaccine of Embodiment 41, further comprising an mRNA encoding a second SARS-CoV-2 antigen and wherein the first SARS-CoV-2 antigen is from wild-type SARS-CoV-2 and the second SARS-CoV-2 antigen is from a variant of the SARS-CoV-2 virus.
43. The mRNA vaccine of Embodiment 41 or 42, wherein the first SARS-CoV-2 antigen and, optionally, the second SARS-CoV-2 antigen each comprise a receptor binding domain (RBD) of a SARS-CoV-2 spike protein, a N-terminal domain (NTD), and an influenza virus hemagglutinin transmembrane (HATM) domain joined by linkers (NTD-RBD-HATM).
44. The mRNA vaccine of Embodiment 43, wherein a ratio of mRNAs encoding influenza virus antigens to mRNAs encoding SARS-CoV-2 antigens in the mRNA vaccine is 5:1, 10:1, or 20:1, optionally wherein the ratio is a molar ratio or a mass ratio.
45. The mRNA vaccine of any one of the preceding Embodiments, further comprising an mRNA encoding a human respiratory syncytial virus (hRSV) F glycoprotein.
46. The mRNA vaccine of Embodiment 45, wherein the hRSV F glycoprotein lacks a cytoplasmic tail.
47. The mRNA vaccine of Embodiment 45 or 46, wherein the hRSV F glycoprotein comprises an amino acid sequence with at least 95% to SEQ ID NO: 99.
48. The mRNA vaccine of any one of Embodiments 45-47, wherein the ratio of mRNAs encoding influenza virus antigens to mRNAs encoding hRSV F glycoproteins is 5:1, 10:1, or 20:1, optionally wherein the ratio is a molar ratio or a mass ratio.
49. The mRNA vaccine of any one of the preceding Embodiments, wherein each mRNA comprises a 5′ untranslated region (UTR), a 3′ UTR, and a polyA tail.
50. The mRNA vaccine of Embodiment 49, wherein the 5′ UTR comprises a sequence having at least 95% identity to a sequence selected from SEQ ID NOs: 1, 2, and 5-35.
51. The mRNA vaccine of Embodiment 50, wherein the 5′ UTR comprises a sequence selected from SEQ ID NOs: 1, 2, and 5-35. 52. The mRNA vaccine of any one of Embodiments 49-51, wherein the 3′ UTR comprises a sequence having at least 95% identity to a sequence selected from SEQ ID NOs: 3, 4, and 36-44.
53. The mRNA vaccine of Embodiment 52, wherein the 3′ UTR comprises a sequence selected from SEQ ID NOs: 3, 4, and 36-44.
54. The mRNA vaccine of any one of the preceding Embodiments, wherein each mRNA comprises a 5′ tetranucleotide cap.
55. The mRNA vaccine of any one of the preceding Embodiments, wherein each mRNA comprises a chemical modification.
56. The mRNA vaccine of Embodiment 55, wherein the chemical modification is 1-methylpseudouridine.
57. The mRNA vaccine of any one of the preceding Embodiments further comprising a lipid nanoparticle.
58. The mRNA vaccine of Embodiment 57, wherein the lipid nanoparticle comprises an ionizable amino lipid, a sterol, a neutral lipid, and a polyethylene glycol (PEG)-modified lipid.
59. The mRNA vaccine of Embodiment 57 or 58, wherein the lipid nanoparticle comprises 40-55 mol % ionizable amino lipid, 30-45 mol % sterol, 5-15 mol % neutral lipid, and 1-5 mol % PEG-modified lipid.
60. The mRNA vaccine of any one of Embodiments 57-59, wherein the lipid nanoparticle comprises 40-50 mol % ionizable amino lipid, 35-45 mol % sterol, 10-15 mol % neutral lipid, and 2-4 mol % PEG-modified lipid.
61. The mRNA vaccine of any one of Embodiments 57-60, wherein the lipid nanoparticle comprises 45 mol %, 46 mol %, 47 mol %, 48 mol %, 49 mol %, or 50 mol % ionizable amino lipid.
62. The mRNA vaccine of any one of Embodiments 57-61, wherein the ionizable amino lipid has the structure of Compound 1:

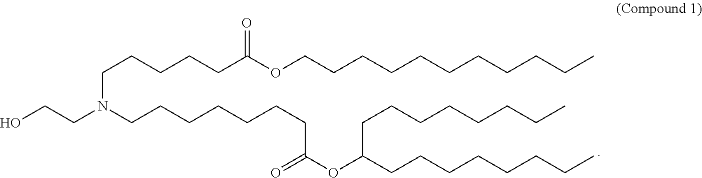 [SMILES: CCCCCCCCCCCOC(=O)CCCCCN(CCO)CCCCCCCC(=O)OC(CCCCCCCC)CCCCCCCC]

63. The mRNA vaccine of any one of Embodiments 57-62, wherein the sterol is cholesterol.
64. The mRNA vaccine of any one of Embodiments 57-63, wherein the neutral lipid is 1,2 distearoyl-sn-glycero-3-phosphocholine (DSPC).
65. The mRNA vaccine of any one of Embodiments 57-64, wherein the PEG-modified lipid is 1,2 dimyristoyl-sn-glycerol, methoxypolyethyleneglycol (PEG2000 DMG).
66. A messenger ribonucleic acid (mRNA) vaccine comprising a first mRNA encoding a stabilized prefusion hemagglutinin (HA) antigen of a first influenza B virus, a second mRNA encoding a stabilized prefusion HA antigen of a second influenza B virus, a third mRNA encoding an HA antigen of a first influenza A virus, a fourth mRNA encoding an HA antigen of a second influenza A virus, a fifth mRNA encoding a first SARS-CoV-2 antigen, and a sixth mRNA encoding a second SARS-CoV-2 antigen.
67. The mRNA vaccine of Embodiment 66, wherein:
   (a) the first mRNA encodes the HA antigen of the first influenza B virus having at least 95% or at least 98% identity to SEQ ID NO: 54;
   (b) the second mRNA encoding the HA antigen of the second influenza B virus having at least 95% or at least 98% identify to SEQ ID NO: 57;
   (c) the third mRNA encoding the HA antigen of the first influenza A virus having at least 95% or at least 98% identity to SEQ ID NO: 48;
   (d) the fourth mRNA encoding the HA antigen of the second influenza A virus having at least 95% or 98% identity to SEQ ID NO: 51;
   (e) the fifth mRNA encoding a first SARS-CoV-2 antigen having at least 95% or at least 98% identity to SEQ ID NO: 60; and/or
   (f) the sixth mRNA encoding a second SARS-CoV antigen having at least 95% or at least 98% identity to SEQ ID NO: 63.
68. The mRNA vaccine of Embodiment 67, wherein:
   (a) the first mRNA encodes the HA antigen of the first influenza B virus comprising SEQ ID NO: 54;
   (b) the second mRNA encoding the HA antigen of the second influenza B virus comprising SEQ ID NO: 57;
   (c) the third mRNA encoding the HA antigen of the first influenza A virus comprising SEQ ID NO: 48;
   (d) the fourth mRNA encoding the HA antigen of the second influenza A virus comprising SEQ ID NO: 51;
   (e) the fifth mRNA encoding a first SARS-CoV-2 antigen comprising SEQ ID NO: 60; and/or
   (f) the sixth mRNA encoding a second SARS-CoV antigen comprising SEQ ID NO: 63.
69. The mRNA vaccine of any one of Embodiments 66-68, wherein:
   (a) the first mRNA comprises an open reading frame (ORF) comprising a sequence having at least 90%, at least 95%, or at least 98% identity to SEQ ID NO: 52;
   (b) the second mRNA comprises an ORF comprising a sequence having at least 90%, at least 95%, or at least 98% identity to SEQ ID NO: 55;
   (c) the third mRNA comprises an ORF comprising a sequence having at least 90%, at least 95%, or at least 98% identity to SEQ ID NO: 46;
   (d) the fourth mRNA comprises an ORF comprising a sequence having at least 90%, at least 95%, or at least 98% identity to SEQ ID NO: 49;
   (e) the fifth mRNA comprises an ORF comprising a sequence having at least 90%, at least 95%, or at least 98% identity to SEQ ID NO: 58; and/or
   (f) the sixth mRNA comprises an ORF comprising a sequence having at least 90%, at least 95%, or at least 98% identity to SEQ ID NO: 61.
70. The mRNA vaccine of Embodiment 69, wherein:
   (a) the first mRNA comprises an ORF comprising SEQ ID NO: 52;
   (b) the second mRNA comprises an ORF comprising SEQ ID NO: 55;
   (c) the third mRNA comprises an ORF comprising SEQ ID NO: 46;
   (d) the fourth mRNA comprises an ORF comprising SEQ ID NO: 49;
   (e) the fifth mRNA comprises an ORF comprising SEQ ID NO: 58; and/or
   (f) the sixth mRNA comprises an ORF comprising SEQ ID NO: 61.
71. The mRNA vaccine of any one of Embodiments 66-70, wherein:
   (a) the first mRNA comprises a sequence having at least 90%, at least 95%, or at least 98% identity to SEQ ID NO: 66;
   (b) the second mRNA comprises a sequence having at least 90%, at least 95%, or at least 98% identity to SEQ ID NO: 67;
   (c) the third mRNA comprises a sequence having at least 90%, at least 95%, or at least 98% identity to SEQ ID NO: 64;
   (d) the fourth mRNA comprises a sequence having at least 90%, at least 95%, or at least 98% identity to SEQ ID NO: 65;
   (e) the fifth mRNA comprises a sequence having at least 90%, at least 95%, or at least 98% identity to SEQ ID NO: 68; and/or
   (f) the sixth mRNA comprises a sequence having at least 90%, at least 95%, or at least 98% identity to SEQ ID NO: 69.
72. The mRNA vaccine of Embodiment 71, wherein:
   (a) the first mRNA comprises SEQ ID NO: 66;
   (b) the second mRNA comprises SEQ ID NO: 67;
   (c) the third mRNA comprises SEQ ID NO: 64;
   (d) the fourth mRNA comprises SEQ ID NO: 65;
   (e) the fifth mRNA comprises SEQ ID NO: 68; and/or
   (f) the sixth mRNA comprises SEQ ID NO: 69.
73. The mRNA vaccine of any one of Embodiments 66-72, wherein a ratio of the first mRNA to the second mRNA to the third mRNA to the fourth mRNA is 1:1:1:1, optionally wherein the ratio is a molar ratio or a mass ratio.
74. The mRNA vaccine of any one of Embodiments 66-73, wherein a ratio of the fifth mRNA to the sixth mRNA is 1:1, optionally wherein the ratio is a molar ratio or a mass ratio.
75. The mRNA vaccine of any one of Embodiments 66-74, wherein the ratio of the (first mRNA, second mRNA, third mRNA, and fourth mRNA) to the (fifth mRNA and sixth mRNA) is 5:1, optionally wherein the ratio is a molar ratio or a mass ratio.
76. The mRNA vaccine of any one of Embodiments 66-74, wherein the ratio of the (first mRNA, second mRNA, third mRNA, and fourth mRNA) to the (fifth mRNA and sixth mRNA) is 10:1, optionally wherein the ratio is a molar ratio or a mass ratio.
77. The mRNA vaccine of any one of Embodiments 66-74, wherein the ratio of the (first mRNA, second mRNA, third mRNA, and fourth mRNA) to the (fifth mRNA and sixth mRNA) is 20:1, optionally wherein the ratio is a molar ratio or a mass ratio.
78. The mRNA vaccine of any one of Embodiments 66-77, comprising 10 μg-100 μg total mRNA.
79. The mRNA vaccine of Embodiment 78, comprising 15 μg total mRNA.
80 The mRNA vaccine of Embodiment 78, comprising 27.5 μg total mRNA.
81. The mRNA vaccine of Embodiment 78, comprising 30 μg total mRNA.
82. The mRNA vaccine of Embodiment 78, comprising 52.5 μg total mRNA.
83. The mRNA vaccine of Embodiment 78, comprising 55 μg total mRNA.
84. The mRNA vaccine of Embodiment 78, comprising 60 μg total mRNA.
85 The mRNA vaccine of any one of Embodiments 66-84, wherein each mRNA comprises a 5′ untranslated region (UTR), a 3′ UTR, and a polyA tail.
86. The mRNA vaccine of any one of Embodiments 66-85, wherein each mRNA comprises a 5′ tetranucleotide cap.
87. The mRNA vaccine of any one of Embodiments 66-86, wherein each mRNA comprises a chemical modification.
88. The mRNA vaccine of Embodiment 87, wherein the chemical modification is 1-methylpseudouridine.
89. The mRNA vaccine of any one of Embodiments 66-88, further comprising a lipid nanoparticle.
90. The mRNA vaccine of Embodiment 89, wherein the lipid nanoparticle comprises an ionizable amino lipid, a sterol, a neutral lipid, and a polyethylene glycol (PEG)-modified lipid.
91. The mRNA vaccine of Embodiment 89 or 90, wherein the lipid nanoparticle comprises 40-55 mol % ionizable amino lipid, 30-45 mol % sterol, 5-15 mol % neutral lipid, and 1-5 mol % PEG-modified lipid.
92. The mRNA vaccine of any one of Embodiments 89-91, wherein the lipid nanoparticle comprises 40-50 mol % ionizable amino lipid, 35-45 mol % sterol, 10-15 mol % neutral lipid, and 2-4 mol % PEG-modified lipid.
93. The mRNA vaccine of any one of Embodiments 89-92, wherein the lipid nanoparticle comprises 45 mol %, 46 mol %, 47 mol %, 48 mol %, 49 mol %, or 50 mol % ionizable amino lipid.
94. The mRNA vaccine of any one of Embodiments 89-93, wherein the ionizable amino lipid has the structure of Compound 1:

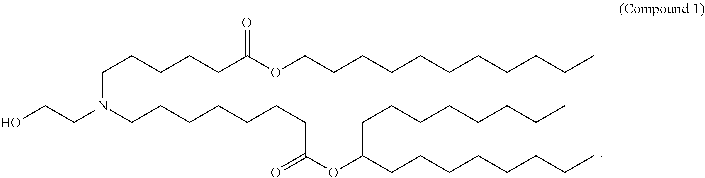 [SMILES: CCCCCCCCCCCOC(=O)CCCCCN(CCO)CCCCCCCC(=O)OC(CCCCCCCC)CCCCCCCC]

95. The mRNA vaccine of any one of Embodiments 89-94, wherein the sterol is cholesterol.
96. The mRNA vaccine of any one of Embodiments 89-95, wherein the neutral lipid is 1,2 distearoyl-sn-glycero-3-phosphocholine (DSPC).
97. The mRNA vaccine of any one of Embodiments 89-96, wherein the PEG-modified lipid is 1,2 dimyristoyl-sn-glycerol, methoxypolyethyleneglycol (PEG2000 DMG).
98. A method comprising administering to a subject in need thereof the mRNA vaccine of any one of the preceding Embodiments.
99. A composition comprising a lipid delivery vehicle and a messenger ribonucleic acid (mRNA) comprising an open reading frame (ORF) encoding a hemagglutinin (HA) protein of an influenza virus, wherein the hemagglutinin protein comprises one or more mutations selected from the group consisting of:
   (i) one or more introduced cysteines, wherein the HA protein comprises a disulfide bond formed by at least one of the one or more introduced cysteines;
   (ii) a proline or glycine substitution in a B loop of the HA protein;
   (iii) one or more substitutions of a cavity-lining residue with a larger hydrophobic residue;
   (iv) one or more substitutions of a pH-sensitive histidine in the HA protein, and
   (v) removal of a polybasic cleavage site.
100. The composition of Embodiment 99, wherein one or more introduced cysteines are present in a stalk region of the HA protein.
101. The composition of Embodiment 99, wherein one or more introduced cysteines are present in a head region of the HA protein.
102. The composition of any one of Embodiments 99-101, wherein two or more protomers of the HA protein are connected by a disulfide bond comprising the introduced cysteines.
103. The composition of any one of Embodiments 99-102, wherein the introduced cysteines are present at one or more of positions 36, 37, 46, 123, 239, 276, 364, 367, 391, 392, 395, 401, 422, 444, 451, 483, 494 relative to a reference influenza virus HA amino acid sequence, wherein the numbering of amino acids is according to any one of SEQ ID NOs: 70, 71, 82, 83, and 95.
104. The composition of any one of Embodiments 99-103, wherein the HA protein comprises one or more proline substitutions in the B loop.
105. The composition of any one of Embodiments 99-104, wherein the HA protein comprises one or more glycine substitutions in the B loop.
106. The composition of any one of Embodiments 99-105, wherein the proline or glycine substitutions are present at one or more of positions 402, 404, 405, 406, 414, 415, 416, 419, or 421 relative to a reference influenza virus HA amino acid sequence, wherein the numbering of amino acids is according to any one of SEQ ID NOs: 70, 71, 82, 83, and 95.
107. The composition of any one of Embodiments 99-106, wherein one or more of the cavity-lining residues are selected from serine, alanine, and valine.
108. The composition of any one of Embodiments 99-107, wherein one of more the larger hydrophobic residues are selected from phenylalanine, histidine, leucine, methionine, valine, and tryptophan.
109. The composition of any one of Embodiments 99-108, wherein the pH-sensitive histidine is uncharged at neutral pH in a reference influenza virus HA protein.
110. The composition of any one of Embodiments 99-109, wherein the pH-sensitive histidine is present at position 381, relative to a reference influenza virus HA amino acid sequence, wherein the numbering of amino acids is according to any one of SEQ ID NOs: 70, 71, 82, 83, and 95.
111. The composition of any one of Embodiments 99-110, wherein one or more arginines of the polybasic cleavage site are substituted with a non-basic residue that is not lysine or histidine.
112. The composition of any one of Embodiments 99-111, wherein the HA protein does not comprise a polybasic cleavage site of a highly pathogenic avian influenza (HPAI) virus.
113. The composition of any one of Embodiments 99-112, wherein the HA protein comprises an amino acid sequence with at least 90% sequence identity to any one of SEQ ID NOs: 84-94, 96, and 97.
114. A messenger ribonucleic acid (mRNA) vaccine comprising an mRNA polynucleotide comprising an open reading frame (ORF) encoding an influenza B/Victoria lineage virus hemagglutinin (HA) antigen comprising H381Y, A288V, N494C, and K483C mutations relative to a wild-type influenza B/Victoria lineage HA protein.
115. The mRNA vaccine of Embodiment 114, wherein the influenza B/Victoria lineage virus HA antigen comprises an amino acid sequence that has at least 90%, at least 95%, or at least 98% identity to SEQ ID NO: 87.
116. A messenger ribonucleic acid (mRNA) vaccine comprising an mRNA polynucleotide comprising an open reading frame (ORF) encoding an influenza B/Yamagata lineage virus hemagglutinin (HA) antigen comprising V239C, V276C, D451C, and K422C mutations relative to a wild-type influenza B/Yamagata lineage HA protein.
117. The mRNA vaccine of Embodiment 116, wherein the influenza B/Yamagata lineage virus HA antigen comprises an amino acid sequence that has at least 90%, at least 95%, or at least 98% identity to SEQ ID NO: 85.
118. The mRNA vaccine of any one of Embodiments 114-117, further comprising an mRNA encoding an influenza A virus (IAV) H1 HA antigen comprising K391C and L37C mutations relative to a wild-type influenza A/(H1N1) virus HA protein.
119. The mRNA vaccine of Embodiment 118, wherein the IAV H1 HA antigen comprises an amino acid sequence that has at least 90%, at least 95%, or at least 98% identity to SEQ ID NO: 94.
120. The mRNA vaccine of any one of Embodiments 114-119, further comprising an mRNA encoding an influenza A virus (IAV) H3 HA antigen comprising G402P, R421P, E414P mutations relative to a wild-type influenza A/(H3N2) virus HA protein.
121. The mRNA vaccine of Embodiment 120, wherein the IAV H3 HA antigen comprises an amino acid sequence that has at least 90%, at least 95%, or at least 98% identity to SEQ ID NO: 91.
122. The mRNA vaccine of any one of Embodiments 114-121, further comprising an mRNA encoding a SARS-CoV-2 antigen.
123. The mRNA vaccine of Embodiment 122, wherein the SARS-CoV-2 antigen comprises a receptor binding domain (RBD) of a SARS-CoV-2 Spike (S) protein, a N-terminal domain (NTD), and an influenza virus hemagglutinin transmembrane (HATM) domain joined by linkers (NTD-RBD-HATM).
124. The mRNA vaccine of any one of Embodiments 114-123, further comprising an mRNA encoding a human respiratory syncytial virus (hRSV) F glycoprotein.
125. The mRNA vaccine of Embodiment 124, wherein the hRSV F glycoprotein lacks a cytoplasmic tail.
126. The mRNA vaccine of Embodiment 125, wherein the hRSV F glycoprotein comprises an amino acid sequence with at least 95% to SEQ ID NO: 99.
127. The mRNA vaccine of any one of Embodiments 114-126, wherein each mRNA comprises a chemical modification.
128. The mRNA vaccine of Embodiment 127, wherein the chemical modification is N1-methylpseudouridine.
129. The mRNA vaccine of any one of Embodiments 114-128, further comprising a lipid nanoparticle.
130. The mRNA vaccine of Embodiment 129, wherein the lipid nanoparticle comprises an ionizable amino lipid, a sterol, a neutral lipid, and a polyethylene glycol (PEG)-modified lipid.
131. The mRNA vaccine of Embodiment 130, wherein the ionizable amino lipid has the structure of Compound 1:

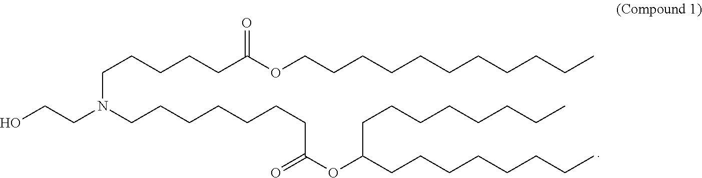 [SMILES: CCCCCCCCCCCOC(=O)CCCCCN(CCO)CCCCCCCC(=O)OC(CCCCCCCC)CCCCCCCC]

132. A method comprising administering the mRNA vaccine of any one of Embodiments 114-131 to a subject in need thereof.

What is claimed is:

1. A mutant influenza B/Victoria lineage virus hemagglutinin (HA) protein having an amino acid sequence comprising an amino acid substitution relative to a reference influenza B/Victoria lineage virus HA protein amino acid sequence, wherein the amino acid sequence of the mutant influenza B/Victoria lineage virus HA protein comprises one or more of (a)-(s):
(a) a tyrosine at position 381 and a valine at position 288;
(b) a cysteine at position 27 and a cysteine at position 349;
(c) a cysteine at position 295 and a cysteine at position 328;
(d) a cysteine at position 399 and a cysteine at position 473;
(e) a cysteine at position 422 and a cysteine at position 444;
(f) a cysteine at position 118 and a cysteine at position 216;
(g) a cysteine at position 237 and a cysteine at position 261;
(h) a cysteine at position 363 and a cysteine at position 480;
(i) a cysteine at position 364 and a cysteine at position 483;
(j) a cysteine at position 365 and a cysteine at position 476;
(k) a cysteine at position 366 and a cysteine at position 479;
(l) a cysteine at position 367 and a cysteine at position 483;
(m) a cysteine at position 435 and a cysteine at position 428;
(n) a cysteine at position 494 and a cysteine at position 483;
(o) a cysteine at position 494 and a cysteine at position 480;
(p) a proline at position 416, a proline at position 417, a proline at position 434, and a proline at position 433;
(q) a proline at position 434 and a proline at position 433;
(r) a proline at position 515, and a proline at position 516;
(s) a phenylalanine at position 473;
wherein the positions of (a)-(s) are numbered by alignment to SEQ ID NO: 71.

2. The mutant influenza B/Victoria lineage virus HA protein of claim 1, wherein the amino acid sequence of the mutant influenza B/Victoria lineage virus HA protein comprises one or more of substitutions (a)-(s) relative to the reference influenza B/Victoria lineage virus HA protein amino acid sequence:
(a) H381Y and A288V substitutions;
(b) S27C and Y349C substitutions;
(c) I295C and K328C substitutions;
(d) S399C and H473C substitutions;
(e) L422C and D444C substitutions;
(f) K118C and L216C substitutions;
(g) V237C and D261C substitutions;
(h) G363C and K480C substitutions;
(i) A364C and K483C substitutions;
(j) I365C and A476C substitutions;
(k) A366C and R479C substitutions;
(l) G367C and K483C substitutions;
(m) E435C and A428C substitutions;
(n) N494C and K483C substitutions;
(o) N494C and K480C substitutions;
(p) E416P, L417P, N434P, and H433P substitutions;
(q) N434P and H433P substitutions;
(r) T515P and F516P substitutions; and
(s) an H473F substitution,
wherein the positions of (a)-(s) are numbered by alignment to SEQ ID NO: 71.

3. The mutant influenza B/Victoria lineage virus HA protein of claim 1, wherein the amino acid sequence of the mutant influenza B/Victoria lineage virus HA protein comprises tyrosine at position 381 and valine at position 288.

4. The mutant influenza B/Victoria lineage HA protein of claim 2, wherein the amino acid sequence of the mutant influenza B/Victoria lineage HA protein comprises H381Y and A288V substitutions relative to the reference influenza B/Victoria lineage virus HA protein amino acid sequence.

5. The mutant influenza B/Victoria lineage virus HA protein of claim 4, wherein the amino acid sequence of the mutant influenza B/Victoria lineage virus HA protein further comprises one or more of substitutions (a)-(r) relative to the reference influenza B/Victoria lineage virus HA protein amino acid sequence:
(a) S27C and Y349C substitutions;
(b) I295C and K328C substitutions;
(c) S399C and H473C substitutions;
(d) L422C and D444C substitutions;
(e) K118C and L216C substitutions;
(f) V237C and D261C substitutions;
(g) G363C and K480C substitutions;
(h) A364C and K483C substitutions;
(i) I365C and A476C substitutions;
(j) A366C and R479C substitutions;
(k) G367C and K483C substitutions;
(l) E435C and A428C substitutions;
(m) N494C and K483C substitutions;
(n) N494C and K480C substitutions;
(o) E416P, L417P, N434P, and H433P substitutions;
(p) N434P and H433P substitutions;
(q) T515P and F516P substitutions;
(r) an H473F substitution,
wherein the positions of (a)-(r) are numbered by alignment to SEQ ID NO: 71.

6. The mutant influenza B/Victoria lineage virus HA protein of claim 1, wherein the amino acid sequence of the mutant influenza B/Victoria lineage virus HA protein comprises (a) tyrosine at position 381, (b) valine at position 288, and (c) cysteine at position 422 and cysteine at position 444, cysteine at position 364 and cysteine at position 483, cysteine at position 367 and cysteine at position 483, or cysteine at position 494 and cysteine at position 483.

7. The mutant influenza B/Victoria lineage virus HA protein of claim 1, wherein the mutant influenza B/Victoria lineage virus HA protein comprises an amino acid sequence with at least 90% sequence identity to the amino acid sequence of SEQ ID NO: 71.

8. A messenger ribonucleic acid (mRNA) comprising an open reading frame (ORF) encoding a mutant influenza B/Victoria lineage virus hemagglutinin (HA) protein according to claim 1.

9. The mRNA of claim 8, wherein the amino acid sequence of the mutant influenza B/Victoria lineage virus HA protein comprises tyrosine at position 381 and valine at position 288.

10. The mRNA of claim 8, wherein the amino acid sequence of the mutant influenza B/Victoria lineage virus HA protein comprises (a) tyrosine at position 381, (b) valine at position 288, and (c) cysteine at position 422 and cysteine at position 444, cysteine at position 364 and cysteine at position 483, cysteine at position 367 and cysteine at position 483, or cysteine at position 494 and cysteine at position 483.

11. The mRNA of claim 8, wherein the amino acid sequence of the mutant influenza B/Victoria lineage HA protein comprises H381Y and A288V substitutions relative to the reference influenza B/Victoria lineage virus HA protein amino acid sequence.

12. A vaccine comprising the mRNA of claim 11.

13. The vaccine of claim 12, wherein the vaccine further comprises (a) an mRNA comprising an ORF encoding an influenza A virus (IAV) H1 hemagglutinin (HA) protein, and (b) an mRNA comprising an ORF encoding an influenza A virus (IAV) H3 hemagglutinin (HA) protein.

14. The vaccine of claim 13, wherein the ORF of each mRNA comprises nucleosides consisting of N1-methylpseudouridine, adenosine, guanosine, and cytidine.

15. The vaccine of claim 14, wherein the mRNA is formulated in a lipid nanoparticle.

16. The vaccine of claim 15, wherein the lipid nanoparticle comprises 20-60 mol % ionizable lipid, 5-25 mol % non-cationic lipid, 2-4 mol % PEG-modified lipid, and 25-55 mol % sterol.

17. The vaccine of claim 16, wherein the ionizable lipid is a compound of Formula (IL*): 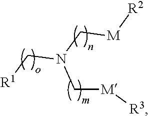
or a salt thereof, wherein:
R¹ is —OH, —NR^{N}—C₄₋₁₀ cycloalkenyl optionally substituted with one or more oxo, or —N(R^{N′}R^{N″});
R^{N} is H or C₁₋₆ alkyl;
R^{N′} is H or C₁₋₆ alkyl;
R^{N″} is H or C₁₋₆ alkyl;
o is 1, 2, 3, or 4;
n is 4, 5, 6, 7, or 8;
m is 4, 5, 6, 7, or 8;
M is —C(═O)—O—* or —O—C(═O)—*, wherein * indicates attachment to R²;
M′ is —C(═O)—O—* or —O—C(═O)—*, wherein * indicates attachment to R³;
R² is 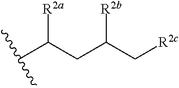
or —(C₁₋₆ alkylene)-(C₃₋₈ cycloalkyl)-C₁₋₆ alkyl;
R^{2a} is —H or C₁₋₁₀ alkyl;
R^{2b} is —H or C₁₋₁₀ alkyl;
R^{2c} is C₁₋₈ alkyl or C₂₋₈ alkenyl;
R³ is 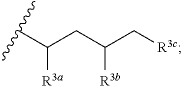
R^{3a} is H or C₁₋₁₀ alkyl;
R^{3b} is H or C₁₋₈ alkyl; and
R^{3c} is C₁₋₁₀ alkyl or C₂₋₈ alkenyl.

18. The vaccine of claim 17, wherein 0.25 mol % to 1.0 mol % of the PEG-modified lipid is present in a core of the lipid nanoparticle, and wherein 2.0 mol % to 2.75 mol % of the PEG-modified lipid is not in the core of the lipid nanoparticle.

19. The vaccine of claim 15, wherein the vaccine comprises from 25 μg-50 μg total mRNA.

20. The vaccine of claim 19, wherein the vaccine comprises 12.5 μg of each mRNA.


SEQUENCE LISTING (233 sequences)

SEQ ID NO: 1 - RNA, 47 nt, synthetic construct
  source 1..47: mol_type = other RNA
  5'UTR 1..47
gggaaataagagagaaaagaagagtaagaagaaatataagagccacc

SEQ ID NO: 2 - RNA, 57 nt, synthetic construct
  source 1..57: mol_type = other RNA
  5'UTR 1..57
gggaaataagagagaaaagaagagtaagaagaaatataagaccccggcgccgccacc

SEQ ID NO: 3 - RNA, 119 nt, synthetic construct
  source 1..119: mol_type = other RNA
  5'UTR 1..119
tgataataggctggagcctcggtggccatgcttcttgccccttgggcctccccccagcccctcctccccttcctgcacccgtacccccgtggtctttgaataaagtctgagtgggcggc

SEQ ID NO: 4 - RNA, 119 nt, synthetic construct
  source 1..119: mol_type = other RNA
  5'UTR 1..119
tgataataggctggagcctcggtggcctagcttcttgccccttgggcctccccccagcccctcctccccttcctgcacccgtacccccgtggtctttgaataaagtctgagtgggcggc

SEQ ID NO: 5 - RNA, 75 nt, synthetic construct
  source 1..75: mol_type = other RNA
  5'UTR 1..75
ggaaatcgcaaaatttgctcttcgcgttagatttcttttagttttctcgcaactagcaagctttttgttctcgcc

SEQ ID NO: 6 - RNA, 203 nt, synthetic construct
  source 1..203: mol_type = other RNA
  5'UTR 1..203
ggaaatccccacaaccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctacgccgccacc

SEQ ID NO: 7 - RNA, 76 nt, synthetic construct
  source 1..76: mol_type = other RNA
  5'UTR 1..76
ggaaataagagagaaaagaagagtaagaagaaatataagatctccctgagcttcagggagccccggcgccgccacc

SEQ ID NO: 8 - RNA, 89 nt, synthetic construct
  source 1..89: mol_type = other RNA
  5'UTR 1..89
ggaaaccccccacccccgtaagagagaaaagaagagtaagaagaaatataagatctccctgagcttcagggagccccggcgccgccacc

SEQ ID NO: 9 - RNA, 59 nt, synthetic construct
  source 1..59: mol_type = other RNA
  5'UTR 1..59
ggagaacttccgcttccgttggcgcaagcgctttcattttttctgctaccgtgactaag

SEQ ID NO: 10 - RNA, 46 nt, synthetic construct
  source 1..46: mol_type = other RNA
  5'UTR 1..46
ggaaataagagagaaaagaagagtaagaagaaatataagagccacc

SEQ ID NO: 11 - RNA, 56 nt, synthetic construct
  source 1..56: mol_type = other RNA
  5'UTR 1..56
ggaaataagagagaaaagaagagtaagaagaaatataagaccccggcgccgccacc

SEQ ID NO: 12 - RNA, 81 nt, synthetic construct
  source 1..81: mol_type = other RNA
  5'UTR 1..81
ggaaatcgcaaaatttgctcttcgcgttagatttcttttagttttctcgcaactagcaagctttttgttctcgccgccgcc

SEQ ID NO: 13 - RNA, 81 nt, synthetic construct
  source 1..81: mol_type = other RNA
  5'UTR 1..81
ggaaatcgcaaaattttcttttcgcgttagatttcttttagttttctttcaactagcaagctttttgttctcgccgccgcc

SEQ ID NO: 14 - RNA, 75 nt, synthetic construct
  source 1..75: mol_type = other RNA
  5'UTR 1..75
ggaaatcgcaaaatgctctcgcgttagatttcttttagttttctykcaactagcaagctttttgttctcgccrcc

SEQ ID NO: 15 - RNA, 79 nt, synthetic construct
  source 1..79: mol_type = other RNA
  5'UTR 1..79
ggaaaattttagcctggaacgttagataactgtcctgttgtctttatatacttggtccccaagtagtttgtcttccaaa

SEQ ID NO: 16 - RNA, 79 nt, synthetic construct
  source 1..79: mol_type = other RNA
  5'UTR 1..79
ggaaactttatttagtgttactttattttctgtttatttgtgtttcttcagtgggtttgttctaatttccttggccgcc

SEQ ID NO: 17 - RNA, 79 nt, synthetic construct
  source 1..79: mol_type = other RNA
  5'UTR 1..79
ggaaaatctgtattaggttggcgtgttctttggtcggttgttagtattgttgttgattcgtttgtggtcggttgccgcc

SEQ ID NO: 18 - RNA, 79 nt, synthetic construct
  source 1..79: mol_type = other RNA
  5'UTR 1..79
ggaaaattattaacatcttggtattctcgataaccattcgttggattttattgtattcgtagtttgggttcctgccgcc

SEQ ID NO: 19 - RNA, 79 nt, synthetic construct
  source 1..79: mol_type = other RNA
  5'UTR 1..79
ggaaattattattatttctagctacaatttatcattgtattattttagctattcatcattatttacttggtgatcaaca

SEQ ID NO: 20 - RNA, 79 nt, synthetic construct
  source 1..79: mol_type = other RNA
  5'UTR 1..79
ggaaataggttgttaaccaagttcaagcctaataagcttggattctggtgacttgcttcaccgttggcgggcaccgatc

SEQ ID NO: 21 - RNA, 74 nt, synthetic construct
  source 1..74: mol_type = other RNA
  5'UTR 1..74
ggaaatcgtagagagtcgtacttagtacatatcgactatcggtggacaccatcaagattataaaccaggccaga

SEQ ID NO: 22 - RNA, 79 nt, synthetic construct
  source 1..79: mol_type = other RNA
  5'UTR 1..79
ggaaacccgcccaagcgaccccaacatatcagcagttgcccaatcccaactcccaacacaatccccaagcaacgccgcc

SEQ ID NO: 23 - RNA, 79 nt, synthetic construct
  source 1..79: mol_type = other RNA
  5'UTR 1..79
ggaaagcgattgaaggcgtcttttcaactactcgattaaggttgggtatcgtcgtgggacttggaaatttgttgtttcc

SEQ ID NO: 24 - RNA, 79 nt, synthetic construct
  source 1..79: mol_type = other RNA
  5'UTR 1..79
ggaaactaatcgaaataaaagagccccgtactcttttatttctattaggttaggagccttagcatttgtatcttaggta

SEQ ID NO: 25 - RNA, 79 nt, synthetic construct
  source 1..79: mol_type = other RNA
  5'UTR 1..79
ggaaatgtgatttccagcaacttcttttgaatatattgaattcctaattcaaagcgaacaaatctacaagccatatacc

SEQ ID NO: 26 - RNA, 74 nt, synthetic construct
  source 1..74: mol_type = other RNA
  5'UTR 1..74
ggaaatcgtagagagtcgtacttacgtggtcgccattgcatagcgcgcgaaagcaacaggaacaagaacgcgcc

SEQ ID NO: 27 - RNA, 74 nt, synthetic construct
  source 1..74: mol_type = other RNA
  5'UTR 1..74
ggaaatcgtagagagtcgtacttagaataaacagagtcgggtcgacttgtctctgatactacgacgtcacaatc

SEQ ID NO: 28 - RNA, 79 nt, synthetic construct
  source 1..79: mol_type = other RNA
  5'UTR 1..79
ggaaaatttgccttcggagttgcgtatcctgaactgcccagcctcctgatatacaactgttccgcttattcgggccgcc

SEQ ID NO: 29 - RNA, 79 nt, synthetic construct
  source 1..79: mol_type = other RNA
  5'UTR 1..79
ggaaatctgagcaggaatcctttgtgcattgaagactttagattcctctctgcggtagacgtgcacttataagtatttg

SEQ ID NO: 30 - RNA, 79 nt, synthetic construct
  source 1..79: mol_type = other RNA
  5'UTR 1..79
ggaaagcgattgaaggcgtcttttcaactactcgattaaggttgggtatcgtcgtgggacttggaaatttgttgccacc

SEQ ID NO: 31 - RNA, 104 nt, synthetic construct
  source 1..104: mol_type = other RNA
  5'UTR 1..104
ggaaatttttttttgatattataagagtttttttttgatattaagaaaatttttttttgatattagaagagtaagaagaaatataagaccccggcgccgccacc

SEQ ID NO: 32 - RNA, 57 nt, synthetic construct
  source 1..57: mol_type = other RNA
  5'UTR 1..57
ggaaataagagagaaaagaagagtaagaagaaatataagagccaaaaaaaaaaaacc

SEQ ID NO: 33 - RNA, 76 nt, synthetic construct
  source 1..76: mol_type = other RNA
  5'UTR 1..76
ggaaatctccctgagcttcagggagtaagagagaaaagaagagtaagaagaaatataagaccccggcgccgccacc

SEQ ID NO: 34 - skipped

SEQ ID NO: 35 - RNA, 36 nt, synthetic construct
  source 1..36: mol_type = other RNA
  3'UTR 1..36
ggactcactatttgttttcgcgcccagttgcaaaaa

SEQ ID NO: 36 - RNA, 119 nt, synthetic construct
  source 1..119: mol_type = other RNA
  3'UTR 1..119
taaagctccccgggggcctcggtggcctagcttcttgccccttgggcctccccccagcccctcctccccttcctgcacccgtacccccgtggtctttgaataaagtctgagtgggcggc

SEQ ID NO: 37 - RNA, 119 nt, synthetic construct
  source 1..119: mol_type = other RNA
  3'UTR 1..119
taagtctaagctggagcctcctgagagacctgtgtgaactattgagaagatcggaacagctccttactctgaggaagttggtacccccgtggtctttgaataaagtctgagtgggcggc

SEQ ID NO: 38 - RNA, 185 nt, synthetic construct
  source 1..185: mol_type = other RNA
  3'UTR 1..185
taaagctccccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 39 - RNA, 170 nt, synthetic construct
  source 1..170: mol_type = other RNA
  3'UTR 1..170
taaagctccccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 40 - RNA, 141 nt, synthetic construct
  source 1..141: mol_type = other RNA
  3'UTR 1..141
taaagctccccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 41 - RNA, 185 nt, synthetic construct
  source 1..185: mol_type = other RNA
  3'UTR 1..185
taagcccctccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 42 - RNA, 187 nt, synthetic construct
  source 1..187: mol_type = other RNA
  3'UTR 1..187
taagcccctccggggtccataaagtaggaaacactacagcctcggtggcctagcttcttgccccttgggcctccataaagtaggaaacactacatccccccagcccctcctccccttcctgcacccgtaccccccgcattattactcacggtacgagtggtctttgaataaagtctgagtgggcggc

SEQ ID NO: 43 - RNA, 170 nt, synthetic construct
  source 1..170: mol_type = other RNA
  3'UTR 1..170
taagcccctccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 44 - RNA, 141 nt, synthetic construct
  source 1..141: mol_type = other RNA
  3'UTR 1..141
taagcccctccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 45 - RNA, 76 nt, synthetic construct
  source 1..76: mol_type = other RNA
  5'UTR 1..76: note = H1_Wisconsin_2019_WT_V2
aggaaatcgcaaaatttgctcttcgcgttagatttcttttagttttctcgcaactagcaagctttttgttctcgcc

SEQ ID NO: 46 - RNA, 1698 nt, synthetic construct
  source 1..1698: mol_type = other RNA
  misc_feature 1..1698: note = H1_Wisconsin_2019_WT_V2 ORF
atgaaggccatcctggtcgtgatgctgtacaccttcaccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgaggggagtggcacccctgcacctgggcaagtgcaacatcgccggctggatcctgggcaaccccgagtgcgagagcctgagcacagcccggagctggagctacatcgtggagaccagcaacagcgacaacggcacctgttaccccggcgacttcatcaactacgaggagctgcgggagcagctgagcagcgtgagcagcttcgagcggttcgagatcttccccaagaccagcagctggcccaaccacgacagcgacaacggcgtgacagcagcctgtccacacgccggagccaagagcttctacaagaacctgatctggctggtgaagaagggcaagagctaccccaagatcaaccagacctacatcaacgacaagggcaaggaggtgctggtgctgtggggcatccaccacccacctaccatcgccgaccagcagagcctgtaccagaacgccgacgcctacgtgttcgtgggcaccagccggtacagcaagaagttcaagccagagatcgccacccggcccaaggtgagagaccaggagggccggatgaactactactggaccctggtggagcccggagacaagattaccttcgaggccaccggcaacctggtggcccctcggtacgccttcaccatggaacgggacgctggcagcggcatcatcatcagcgacactcccgtgcacgactgcaacaccacctgccagactcccgagggcgctatcaacaccagcctgcccttccagaacgtgcaccccatcaccatcggcaagtgccccaagtacgtaaagagcaccaaattgcggctggccaccggactcaggaacgtgcccagcatccaaagccggggcctgtttggcgcaatcgccggcttcatcgagggcggctggactggcatggtggacggctggtacggctaccaccaccagaacgaacaggggagcggctacgcagctgacctgaagagcacccagaacgccatcgacaagatcaccaacaaggtgaacagcgtgatcgagaagatgaacacccagttcaccgccgtgggcaaggagttcaaccacctggagaagcggatcgagaacctgaacaagaaggtggacgacggcttcctggacatctggacctacaacgccgagctgctggttctgctggagaacgagcggaccctggactatcacgacagcaacgtgaagaacctgtacgagaaggtgcggaaccagctgaagaacaacgccaaggagatcggcaacggctgcttcgagttctaccacaagtgcgacaacacctgcatggagagcgtgaagaacggcacctacgactaccccaagtacagcgaggaggccaagctgaaccgggagaagatcgacggcgtgaagctggacagcacccggatctaccagatcctggccatctacagcaccgtggccagcagcctggtgctggtggtgagcctgggcgccatcagcttctggatgtgcagcaacggcagcctgcagtgccggatctgcatc

SEQ ID NO: 47 - RNA, 115 nt, synthetic construct
  source 1..115: mol_type = other RNA
  3'UTR 1..115: note = H1_Wisconsin_2019_WT_V2
taaagctccccgggggcctcggtggccctccaccgaagcagccatcagcacccccagcccctcctccccttcctgcaggtcaaagtggtctttgaataaagtctgagtgggcggc

SEQ ID NO: 48 - protein, 566 aa, unidentified
  source 1..566: note = Influenza A virus
  REGION 1..566: note = H1_Wisconsin_2019_WT_V2 AA
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 49 - RNA, 1698 nt, synthetic construct
  source 1..1698: mol_type = other RNA
  misc_feature 1..1698: note = H3_A/Darwin/6/2021_G53D_V2 ORF
atgaagaccatcatcgccctgagcaacatcctgtgcctggtgttcgcccagaagatccccggcaacgataacagcaccgccaccctgtgtctgggacaccacgccgtgcccaacggcaccatcgtgaagactatcaccaacgaccggatcgaggtgaccaacgccaccgagctggtgcagaacagcagcatcggcgagatctgcgacagccctcaccagatcctggacggcggcaactgcaccctgatcgacgcactgctgggcgaccctcagtgcgacggctttcagaacaaggagtgggacctgttcgtggagagatcgcgggccaacagcaactgctacccctacgacgtccccgactacgcaagcctgagaagcctcgtggcctcaagcggcaccctggagttcaagaacgagagcttcaactggaccggcgtgaagcagaacggcacctcaagcgcctgcatccggggctccagcagcagcttcttctcacggctgaactggctgacccacctgaacaacatctaccccgcccagaacgtgaccatgcccaacaaggagcagttcgacaagctgtacatctggggagtgcaccatcccgacaccgacaagaaccagattagcctgttcgcccagagcagcggccggatcaccgtgagcaccaagcggagccagcaggccgtgatccccaacatcggctctcggcccagaatccgggacatccccagccggatcagcatctactggaccattgtgaagcccggcgacatcctgctgatcaactccaccggcaacctgatcgcccctcggggctatttcaagatccggagcggcaagagcagcatcatgcggagcgacgcccctatcggcaagtgcaagagcgagtgcatcacacccaacggaagcatccccaacgacaagcccttccagaacgtgaaccggataacctacggcgcctgccctagatacgtgaagcagagcaccctgaagctggccaccggcatgcggaacgtgcccgagaagcagactcggggcatcttcggcgccatcgccggcttcatcgagaacggctgggagggcatggtggacggctggtacggcttccggcaccagaactctgagggcagaggacaggccgcagacctgaagagcacccaggccgccatcgaccagatcaacggcaagctgaaccggctgatcggcaagaccaacgagaagttccaccagatcgagaaggagttcagcgaggtggagggcagggtacaggacctggagaagtacgtggaggacaccaagatcgacctgtggagctacaacgccgagctgctggtagccctggagaaccagcacaccatcgacctgaccgacagcgagatgaacaagctgttcgagaagaccaagaagcagctgcgggagaacgccgaggacatgggcaacggctgcttcaagatctaccacaagtgcgacaacgcctgcatcggcagcatccggaacgagacctacgaccacaacgtgtaccgggacgaggccctgaacaaccggttccagatcaagggcgtggagctgaagagcggctacaaggactggatcctgtggatcagcttcgccatgtcctgcttcctgctgtgcatcgccctgctgggtttcatcatgtgggcctgccagaagggcaacatccggtgcaacatctgcatc

SEQ ID NO: 50 - RNA, 117 nt, synthetic construct
  source 1..117: mol_type = other RNA
  3'UTR 1..117: note = H3_A/Darwin/6/2021_G53D_V2
taaagctccccgggggcctcggtggccctccgcatcagaccaacggcacacccccagcccctcctccccttcctgcaggtcaaggagtggtctttgaataaagtctgagtgggcggc

SEQ ID NO: 51 - protein, 566 aa, unidentified
  source 1..566: note = Influenza A virus
  REGION 1..566: note = H3_A/Darwin/6/2021_G53D_V2 AA
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTHLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 52 - RNA, 1746 nt, synthetic construct
  source 1..1746: mol_type = other RNA
  misc_feature 1..1746: note = B/Austria/1359417/2021 H363Y A270V HA_SEv4.5 ORF
atgaaggctatcatcgtgctgttaatggtggtgaccagcaacgccgaccggatctgcacaggcatcacctctagcaacagccctcacgtggtcaagaccgccacacagggcgaggtgaacgtgaccggcgtgattcccctgaccaccacccctaccaagagccacttcgccaacctgaagggaaccgagacccggggcaagctgtgtccaaagtgcctgaactgtaccgacctggacgtggctctgggcagacccaagtgcaccggcaagatccccagcgcccgggtgtctatcttgcacgaagtgcggcccgtgactagcggttgcttccccatcatgcacgaccggaccaagatcagacagctgcccaacctgctgcggggctacgagcacgtgcggctgtccacccataacgtgatcaacaccgaagacgcacccggcggcccatacgagatcggcaccagcggctcttgcctgaatatcaccaacggcaagggattctttgctaccatggcctgggccgtgccaaagaacaagactgccaccaaccctctgaccatcgaggtgccctacatctgcaccgaggaggaggaccagatcaccgtgtggggcttccacagcgacgacgagacacagatggccagactgtacggcgacagcaaaccccagaagttcaccagcagcgccaacggcgtgaccacccactacgtgagccagattggcggcttccctaaccaaaccgaggacggcggcttaccccagagcggccggatcgtggtggactacatggttcagaagagcggcaagaccggcaccatcacctaccagcggggcatcctgttaccacagaaggtgtggtgcgtgtcagggaaatcaaaggtcatcaagggctccctgccactgattggcgaggccgactgcctgcacgagaagtacggcggcctgaacaagagcaagccctactacaccggcgagcacgccaaggcaatcggcaactgccccatctgggtgaagacacccctgaagctggcaaacggcaccaagtaccggccacccgccaaactgctgaaggagcggggcttcttcggcgccattgccggcttcctcgaaggcggttgggagggcatgatcgcaggctggtacggctacactagccacggcgcacacggagtagcagtggccgccgacctgaagagcacccaggaggccatcaacaagatcaccaagaacctgaacagcctgagcgagctggaggtgaagaatctgcagaggctgtctggcgctatggacgagctgcacaacgagatcctggagctggacgagaaggtggacgacttacgggccgacaccatcagcagccagatcgagctggcagtgctgctgagcaacgagggcatcatcaacagcgaggacgagcacctgctggccctggagcggaagctgaagaagatgctgggcccttctgccgtggagatcggtaacggctgcttcgagaccaagcacaagtgcaaccagacctgcctggatcggatcgcagccggcacctttgacgccggggagttcagcctgcccaccttcgacagcttgaacatcaccgccgccagcctgaacgacgacggcctggacaaccacaccatcctgctgtactactctacagccgctagcagcctggccgtgaccctgatgatcgccatcttcgtggtgtacatggtgagccgggacaacgtgagctgcagcatctgcctg

SEQ ID NO: 53 - RNA, 116 nt, synthetic construct
  source 1..116: mol_type = other RNA
  3'UTR 1..116: note = B/Austria/1359417/2021 H363Y A270V HA_SEv4.5
taaagctccccgggggcctcggtggccctcaacctacgccgaagaccacgcccccagcccctcctccccttcctgcaggtcaagagtggtctttgaataaagtctgagtgggcggc

SEQ ID NO: 54 - protein, 582 aa, synthetic construct
  REGION 1..582: note = B/Austria/1359417/2021 H363Y A270V HA_SEv4.5 AA
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCVSGKSKVIKGSLPLIGEADCLHEKYGGLNKSKPYYTGEHAKAIGNCPIWVKTPLKLANGTKYRPPAKLLKERGFFGAIAGFLEGGWEGMIAGWYGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 55 - RNA, 1752 nt, synthetic construct
  source 1..1752: mol_type = other RNA
  misc_feature 1..1752: note = B/Phuket/3073/2013 H363Y A270V HA ORF
atgaaggctatcatcgtgctactcatggtggtaaccagcaacgccgaccggatctgcaccggcatcaccagcagcaacagcccgcacgttgtgaagaccgccacccaaggcgaggtgaacgtgaccggcgtgatcccactgaccaccactcccaccaagagctacttcgccaacctgaagggcactcggactcggggcaagctctgccccgactgcctgaactgcaccgacctggacgtggctctgggcagacccatgtgcgtggggaccaccccttctgccaaggcctcaatcctgcacgaggtcaggcccgtgaccagcgggtgcttccccatcatgcacgaccggaccaagatcagacagctgcccaacctgctgcggggctacgagaagatccggctgagcacacagaacgtgatcgacgccgagaaggcccctggaggtccctaccggctgggcaccagcggaagctgccccaacgccacgagcaagatcggcttcttcgccaccatggcctgggctgtgcccaaggacaactacaagaacgccaccaatcccctgaccgtggaggtgccctacatttgtaccgagggcgaggaccagatcaccgtgtggggcttccacagcgacaacaagacccaaatgaagagcctgtacggcgacagcaatccccagaagttcacaagcagcgccaacggtgtgaccacccactacgtgagccagattggcgacttccccgaccagaccgaggacggagggctgccgcagagtggccggatcgtggtggactacatgatgcagaagcccggcaagaccggcaccatagtgtatcagcggggcgtgctgttgcctcagaaagtttggtgtgtgagcggcaggagcaaggtgatcaagggcagccttcccctgatcggcgaggcagactgcctccacgaggagtacggcggcctgaacaagagcaagccctactacaccggcaagcacgccaaggcaatcgggaactgccccatctgggtcaagacccctctgaagctggccaacggcaccaagtaccggccaccagccaagctgctgaaggagcggggcttctttggcgccattgccggcttcctcgagggaggctgggagggcatgatcgccggctggtacggctacacaagccacggcgcacacggagtggctgtggctgccgacctgaagagcacccaggaggccatcaacaagatcaccaagaatctgaacagcctgagcgagctggaggtgaagaacctgcagcggctgtcaggcgccatggacgagctgcacaacgagatcctggagctggacgagaaggtggacgacctgcgtgccgacaccatcagcagccagatcgagctggccgtactgctgagcaacgagggcatcatcaacagcgaggacgagcacctgctggccctggagcggaaactgaagaagatgctgggaccctctgccgtggacatcggcaacggctgcttcgagactaagcacaagtgcaaccagacctgcctggatcggatcgccgccggaaccttcaacgccggcgagttcagcctgcccaccttcgacagcttaaacatcaccgccgccagcctgaacgacgacggcctggacaaccacaccatcctgctgtactacagcactgccgcctcaagcctggccgtgaccctgatgctggccatcttcatcgtgtacatggtgagccgggacaacgtgagctgcagcatctgcctg

SEQ ID NO: 56 - RNA, 114 nt, synthetic construct
  source 1..114: mol_type = other RNA
  3'UTR 1..114: note = B/Phuket/3073/2013 H363Y A270V HA
taaagctccccgggggcctcggtggccctcaacgaccctgccgcagcaaacccccagcccctcctccccttcctgcaggatcagtggtctttgaataaagtctgagtgggcggc

SEQ ID NO: 57 - protein, 584 aa, synthetic construct
  REGION 1..584: note = B/Phuket/3073/2013 H363Y A270V HA AA
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCVSGRSKVIKGSLPLIGEADCLHEEYGGLNKSKPYYTGKHAKAIGNCPIWVKTPLKLANGTKYRPPAKLLKERGFFGAIAGFLEGGWEGMIAGWYGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 58 - RNA, 1572 nt, synthetic construct
  source 1..1572: mol_type = other RNA
  misc_feature 1..1572: note = NTD-RBD-HATM_V2 ORF
atgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggacggaggcggatcgggaggcggacccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagtctggcggaggcagcatcctggccatctacagcaccgtggccagcagcctggtgctgctggtgagcctgggcgccatcagcttc

SEQ ID NO: 59 - RNA, 119 nt, synthetic construct
  source 1..119: mol_type = other RNA
  3'UTR 1..119: note = NTD-RBD-HATM_V2
taaagctccccgggggcctcggtggcctagcttcttgccccttgggcctccccccagcccctcctccccttcctgcacctgtacttccgtggtctttgaataaagtctgagtgggcggc

SEQ ID NO: 60 - protein, 524 aa, synthetic construct
  REGION 1..524: note = NTD-RBD-HATM_V2 AA
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKSGGGSILAIYSTVASSLVLLVSLGAISF

SEQ ID NO: 61 - RNA, 1557 nt, synthetic construct
  source 1..1557: mol_type = other RNA
  misc_feature 1..1557: note = BA.4.BA.5-NTD-RBD-HATM ORF
atgttcgtgttcctggtgctgctgcccctggtgagcagccagtgcgtgaacctgatcacccggacccagagctacaccaacagcttcacccggggcgtctactaccccgacaaggtgttccggagcagcgtcctgcacagcacccaggacctgttcctgcccttcttcagcaacgtgacctggttccacgccatcagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggacgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctgggccgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggacggcggaggctctggaggcggccccaacatcaccaacctgtgccccttcgacgaggtgttcaacgccacccggttcgccagcgtgtacgcctggaaccggaagcggatcagcaactgcgtggccgactacagcgtgctgtacaacttcgccccattcttcgccttcaagtgctacggcgtgagccccaccaagctgaacgacctgtgcttcaccaacgtgtacgccgacagcttcgtgatccgtggcaacgaggtgagccagatcgcacccggccagacaggcaacatcgccgactacaactacaagctgcccgacgacttcaccggctgcgtgatcgcctggaacagcaacaagctcgacagcaaggtgggcggcaactacaactaccggtaccggctgttccggaagagcaacctgaagcccttcgagcgggacatcagcaccgagatctaccaagccggcaacaagccttgcaacggcgtggccggcgtgaactgctacttccctctgcagagctacggcttccggcccacctacggcgtgggccaccagccctaccgggtggtggtgctgagcttcgagctgctgcacgccccagccaccgtgtgtggccccaagagcggcggcggcagcatcctggccatctacagcaccgtggccagcagcctggtgctgctggtgagcctgggcgccatcagcttt

SEQ ID NO: 62 - RNA, 122 nt, synthetic construct
  source 1..122: mol_type = other RNA
  3'UTR 1..122: note = BA.4.BA.5-NTD-RBD-HATM
taaagctccccgggggcctcggtggcctagcttcttgccccttgggcctccccccagcccctcctccccttcctgcatttgcctgtgagcagtggtctttgaataaagtctgagtgggcggc

SEQ ID NO: 63 - protein, 519 aa, synthetic construct
  REGION 1..519: note = BA.4.BA.5-NTD-RBD-HATM AA
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDGGGSGGGPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKSGGGSILAIYSTVASSLVLLVSLGAISF

SEQ ID NO: 64 - RNA, 1889 nt, synthetic construct
  source 1..1889: mol_type = other RNA
  misc_feature 1..1889: note = H1_Wisconsin_2019_WT_V2
aggaaatcgcaaaatttgctcttcgcgttagatttcttttagttttctcgcaactagcaagctttttgttctcgccatgaaggccatcctggtcgtgatgctgtacaccttcaccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgaggggagtggcacccctgcacctgggcaagtgcaacatcgccggctggatcctgggcaaccccgagtgcgagagcctgagcacagcccggagctggagctacatcgtggagaccagcaacagcgacaacggcacctgttaccccggcgacttcatcaactacgaggagctgcgggagcagctgagcagcgtgagcagcttcgagcggttcgagatcttccccaagaccagcagctggcccaaccacgacagcgacaacggcgtgacagcagcctgtccacacgccggagccaagagcttctacaagaacctgatctggctggtgaagaagggcaagagctaccccaagatcaaccagacctacatcaacgacaagggcaaggaggtgctggtgctgtggggcatccaccacccacctaccatcgccgaccagcagagcctgtaccagaacgccgacgcctacgtgttcgtgggcaccagccggtacagcaagaagttcaagccagagatcgccacccggcccaaggtgagagaccaggagggccggatgaactactactggaccctggtggagcccggagacaagattaccttcgaggccaccggcaacctggtggcccctcggtacgccttcaccatggaacgggacgctggcagcggcatcatcatcagcgacactcccgtgcacgactgcaacaccacctgccagactcccgagggcgctatcaacaccagcctgcccttccagaacgtgcaccccatcaccatcggcaagtgccccaagtacgtaaagagcaccaaattgcggctggccaccggactcaggaacgtgcccagcatccaaagccggggcctgtttggcgcaatcgccggcttcatcgagggcggctggactggcatggtggacggctggtacggctaccaccaccagaacgaacaggggagcggctacgcagctgacctgaagagcacccagaacgccatcgacaagatcaccaacaaggtgaacagcgtgatcgagaagatgaacacccagttcaccgccgtgggcaaggagttcaaccacctggagaagcggatcgagaacctgaacaagaaggtggacgacggcttcctggacatctggacctacaacgccgagctgctggttctgctggagaacgagcggaccctggactatcacgacagcaacgtgaagaacctgtacgagaaggtgcggaaccagctgaagaacaacgccaaggagatcggcaacggctgcttcgagttctaccacaagtgcgacaacacctgcatggagagcgtgaagaacggcacctacgactaccccaagtacagcgaggaggccaagctgaaccgggagaagatcgacggcgtgaagctggacagcacccggatctaccagatcctggccatctacagcaccgtggccagcagcctggtgctggtggtgagcctgggcgccatcagcttctggatgtgcagcaacggcagcctgcagtgccggatctgcatctaaagctccccgggggcctcggtggccctccaccgaagcagccatcagcacccccagcccctcctccccttcctgcaggtcaaagtggtctttgaataaagtctgagtgggcggc

SEQ ID NO: 65 - RNA, 1891 nt, synthetic construct
  source 1..1891: mol_type = other RNA
  misc_feature 1..1891: note = H3_A/Darwin/6/2021_G53D_V2
aggaaatcgcaaaatttgctcttcgcgttagatttcttttagttttctcgcaactagcaagctttttgttctcgccatgaagaccatcatcgccctgagcaacatcctgtgcctggtgttcgcccagaagatccccggcaacgataacagcaccgccaccctgtgtctgggacaccacgccgtgcccaacggcaccatcgtgaagactatcaccaacgaccggatcgaggtgaccaacgccaccgagctggtgcagaacagcagcatcggcgagatctgcgacagccctcaccagatcctggacggcggcaactgcaccctgatcgacgcactgctgggcgaccctcagtgcgacggctttcagaacaaggagtgggacctgttcgtggagagatcgcgggccaacagcaactgctacccctacgacgtccccgactacgcaagcctgagaagcctcgtggcctcaagcggcaccctggagttcaagaacgagagcttcaactggaccggcgtgaagcagaacggcacctcaagcgcctgcatccggggctccagcagcagcttcttctcacggctgaactggctgacccacctgaacaacatctaccccgcccagaacgtgaccatgcccaacaaggagcagttcgacaagctgtacatctggggagtgcaccatcccgacaccgacaagaaccagattagcctgttcgcccagagcagcggccggatcaccgtgagcaccaagcggagccagcaggccgtgatccccaacatcggctctcggcccagaatccgggacatccccagccggatcagcatctactggaccattgtgaagcccggcgacatcctgctgatcaactccaccggcaacctgatcgcccctcggggctatttcaagatccggagcggcaagagcagcatcatgcggagcgacgcccctatcggcaagtgcaagagcgagtgcatcacacccaacggaagcatccccaacgacaagcccttccagaacgtgaaccggataacctacggcgcctgccctagatacgtgaagcagagcaccctgaagctggccaccggcatgcggaacgtgcccgagaagcagactcggggcatcttcggcgccatcgccggcttcatcgagaacggctgggagggcatggtggacggctggtacggcttccggcaccagaactctgagggcagaggacaggccgcagacctgaagagcacccaggccgccatcgaccagatcaacggcaagctgaaccggctgatcggcaagaccaacgagaagttccaccagatcgagaaggagttcagcgaggtggagggcagggtacaggacctggagaagtacgtggaggacaccaagatcgacctgtggagctacaacgccgagctgctggtagccctggagaaccagcacaccatcgacctgaccgacagcgagatgaacaagctgttcgagaagaccaagaagcagctgcgggagaacgccgaggacatgggcaacggctgcttcaagatctaccacaagtgcgacaacgcctgcatcggcagcatccggaacgagacctacgaccacaacgtgtaccgggacgaggccctgaacaaccggttccagatcaagggcgtggagctgaagagcggctacaaggactggatcctgtggatcagcttcgccatgtcctgcttcctgctgtgcatcgccctgctgggtttcatcatgtgggcctgccagaagggcaacatccggtgcaacatctgcatctaaagctccccgggggcctcggtggccctccgcatcagaccaacggcacacccccagcccctcctccccttcctgcaggtcaaggagtggtctttgaataaagtctgagtgggcggc

SEQ ID NO: 66 - RNA, 1938 nt, synthetic construct
  source 1..1938: mol_type = other RNA
  misc_feature 1..1938: note = B/Austria/1359417/2021 H363Y A270V HA_SEv4.5
aggaaatcgcaaaatttgctcttcgcgttagatttcttttagttttctcgcaactagcaagctttttgttctcgccatgaaggctatcatcgtgctgttaatggtggtgaccagcaacgccgaccggatctgcacaggcatcacctctagcaacagccctcacgtggtcaagaccgccacacagggcgaggtgaacgtgaccggcgtgattcccctgaccaccacccctaccaagagccacttcgccaacctgaagggaaccgagacccggggcaagctgtgtccaaagtgcctgaactgtaccgacctggacgtggctctgggcagacccaagtgcaccggcaagatccccagcgcccgggtgtctatcttgcacgaagtgcggcccgtgactagcggttgcttccccatcatgcacgaccggaccaagatcagacagctgcccaacctgctgcggggctacgagcacgtgcggctgtccacccataacgtgatcaacaccgaagacgcacccggcggcccatacgagatcggcaccagcggctcttgcctgaatatcaccaacggcaagggattctttgctaccatggcctgggccgtgccaaagaacaagactgccaccaaccctctgaccatcgaggtgccctacatctgcaccgaggaggaggaccagatcaccgtgtggggcttccacagcgacgacgagacacagatggccagactgtacggcgacagcaaaccccagaagttcaccagcagcgccaacggcgtgaccacccactacgtgagccagattggcggcttccctaaccaaaccgaggacggcggcttaccccagagcggccggatcgtggtggactacatggttcagaagagcggcaagaccggcaccatcacctaccagcggggcatcctgttaccacagaaggtgtggtgcgtgtcagggaaatcaaaggtcatcaagggctccctgccactgattggcgaggccgactgcctgcacgagaagtacggcggcctgaacaagagcaagccctactacaccggcgagcacgccaaggcaatcggcaactgccccatctgggtgaagacacccctgaagctggcaaacggcaccaagtaccggccacccgccaaactgctgaaggagcggggcttcttcggcgccattgccggcttcctcgaaggcggttgggagggcatgatcgcaggctggtacggctacactagccacggcgcacacggagtagcagtggccgccgacctgaagagcacccaggaggccatcaacaagatcaccaagaacctgaacagcctgagcgagctggaggtgaagaatctgcagaggctgtctggcgctatggacgagctgcacaacgagatcctggagctggacgagaaggtggacgacttacgggccgacaccatcagcagccagatcgagctggcagtgctgctgagcaacgagggcatcatcaacagcgaggacgagcacctgctggccctggagcggaagctgaagaagatgctgggcccttctgccgtggagatcggtaacggctgcttcgagaccaagcacaagtgcaaccagacctgcctggatcggatcgcagccggcacctttgacgccggggagttcagcctgcccaccttcgacagcttgaacatcaccgccgccagcctgaacgacgacggcctggacaaccacaccatcctgctgtactactctacagccgctagcagcctggccgtgaccctgatgatcgccatcttcgtggtgtacatggtgagccgggacaacgtgagctgcagcatctgcctgtaaagctccccgggggcctcggtggccctcaacctacgccgaagaccacgcccccagcccctcctccccttcctgcaggtcaagagtggtctttgaataaagtctgagtgggcggc

SEQ ID NO: 67 - RNA, 1942 nt, synthetic construct
  source 1..1942: mol_type = other RNA
  misc_feature 1..1942: note = B/Phuket/3073/2013 H363Y A270V HA
aggaaatcgcaaaatttgctcttcgcgttagatttcttttagttttctcgcaactagcaagctttttgttctcgccatgaaggctatcatcgtgctactcatggtggtaaccagcaacgccgaccggatctgcaccggcatcaccagcagcaacagcccgcacgttgtgaagaccgccacccaaggcgaggtgaacgtgaccggcgtgatcccactgaccaccactcccaccaagagctacttcgccaacctgaagggcactcggactcggggcaagctctgccccgactgcctgaactgcaccgacctggacgtggctctgggcagacccatgtgcgtggggaccaccccttctgccaaggcctcaatcctgcacgaggtcaggcccgtgaccagcgggtgcttccccatcatgcacgaccggaccaagatcagacagctgcccaacctgctgcggggctacgagaagatccggctgagcacacagaacgtgatcgacgccgagaaggcccctggaggtccctaccggctgggcaccagcggaagctgccccaacgccacgagcaagatcggcttcttcgccaccatggcctgggctgtgcccaaggacaactacaagaacgccaccaatcccctgaccgtggaggtgccctacatttgtaccgagggcgaggaccagatcaccgtgtggggcttccacagcgacaacaagacccaaatgaagagcctgtacggcgacagcaatccccagaagttcacaagcagcgccaacggtgtgaccacccactacgtgagccagattggcgacttccccgaccagaccgaggacggagggctgccgcagagtggccggatcgtggtggactacatgatgcagaagcccggcaagaccggcaccatagtgtatcagcggggcgtgctgttgcctcagaaagtttggtgtgtgagcggcaggagcaaggtgatcaagggcagccttcccctgatcggcgaggcagactgcctccacgaggagtacggcggcctgaacaagagcaagccctactacaccggcaagcacgccaaggcaatcgggaactgccccatctgggtcaagacccctctgaagctggccaacggcaccaagtaccggccaccagccaagctgctgaaggagcggggcttctttggcgccattgccggcttcctcgagggaggctgggagggcatgatcgccggctggtacggctacacaagccacggcgcacacggagtggctgtggctgccgacctgaagagcacccaggaggccatcaacaagatcaccaagaatctgaacagcctgagcgagctggaggtgaagaacctgcagcggctgtcaggcgccatggacgagctgcacaacgagatcctggagctggacgagaaggtggacgacctgcgtgccgacaccatcagcagccagatcgagctggccgtactgctgagcaacgagggcatcatcaacagcgaggacgagcacctgctggccctggagcggaaactgaagaagatgctgggaccctctgccgtggacatcggcaacggctgcttcgagactaagcacaagtgcaaccagacctgcctggatcggatcgccgccggaaccttcaacgccggcgagttcagcctgcccaccttcgacagcttaaacatcaccgccgccagcctgaacgacgacggcctggacaaccacaccatcctgctgtactacagcactgccgcctcaagcctggccgtgaccctgatgctggccatcttcatcgtgtacatggtgagccgggacaacgtgagctgcagcatctgcctgtaaagctccccgggggcctcggtggccctcaacgaccctgccgcagcaaacccccagcccctcctccccttcctgcaggatcagtggtctttgaataaagtctgagtgggcggc

SEQ ID NO: 68 - RNA, 1767 nt, synthetic construct
  source 1..1767: mol_type = other RNA
  misc_feature 1..1767: note = NTD-RBD-HATM_V2
aggaaatcgcaaaatttgctcttcgcgttagatttcttttagttttctcgcaactagcaagctttttgttctcgccatgttcgtgttcctggtgctgctgcccctggtgagcagccagtgcgtgaacctgaccacccggacccagctgccaccagcctacaccaacagcttcacccggggcgtctactaccccgacaaggtgttccggagcagcgtcctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctggtgcgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggacggaggcggatcgggaggcggacccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccgtggcgacgaggtgcggcagatcgcacccggccagacaggcaagatcgccgactacaactacaagctgcccgacgacttcaccggctgcgtgatcgcctggaacagcaacaacctcgacagcaaggtgggcggcaactacaactacctgtaccggctgttccggaagagcaacctgaagcccttcgagcgggacatcagcaccgagatctaccaagccggctccaccccttgcaacggcgtggagggcttcaactgctacttccctctgcagagctacggcttccagcccaccaacggcgtgggctaccagccctaccgggtggtggtgctgagcttcgagctgctgcacgccccagccaccgtgtgtggccccaagtctggcggaggcagcatcctggccatctacagcaccgtggccagcagcctggtgctgctggtgagcctgggcgccatcagcttctaaagctccccgggggcctcggtggcctagcttcttgccccttgggcctccccccagcccctcctccccttcctgcacctgtacttccgtggtctttgaataaagtctgagtgggcggc

SEQ ID NO: 69 - RNA, 1755 nt, synthetic construct
  source 1..1755: mol_type = other RNA
  misc_feature 1..1755: note = BA.4.BA.5-NTD-RBD-HATM
aggaaatcgcaaaatttgctcttcgcgttagatttcttttagttttctcgcaactagcaagctttttgttctcgccatgttcgtgttcctggtgctgctgcccctggtgagcagccagtgcgtgaacctgatcacccggacccagagctacaccaacagcttcacccggggcgtctactaccccgacaaggtgttccggagcagcgtcctgcacagcacccaggacctgttcctgcccttcttcagcaacgtgacctggttccacgccatcagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaataacgccaccaacgtggtgatcaaggtgtgcgagttccagttctgcaacgaccccttcctggacgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacaccccaatcaacctgggccgggatctgccccagggcttctcagccctggagcccctggtggacctgcccatcggcatcaacatcacccggttccagaccctgctggccctgcaccggagctacctgaccccaggcgacagcagcagcgggtggacagcaggcgcggctgcttactacgtgggctacctgcagccccggaccttcctgctgaagtacaacgagaacggcaccatcaccgacgccgtggacggcggaggctctggaggcggccccaacatcaccaacctgtgccccttcgacgaggtgttcaacgccacccggttcgccagcgtgtacgcctggaaccggaagcggatcagcaactgcgtggccgactacagcgtgctgtacaacttcgccccattcttcgccttcaagtgctacggcgtgagccccaccaagctgaacgacctgtgcttcaccaacgtgtacgccgacagcttcgtgatccgtggcaacgaggtgagccagatcgcacccggccagacaggcaacatcgccgactacaactacaagctgcccgacgacttcaccggctgcgtgatcgcctggaacagcaacaagctcgacagcaaggtgggcggcaactacaactaccggtaccggctgttccggaagagcaacctgaagcccttcgagcgggacatcagcaccgagatctaccaagccggcaacaagccttgcaacggcgtggccggcgtgaactgctacttccctctgcagagctacggcttccggcccacctacggcgtgggccaccagccctaccgggtggtggtgctgagcttcgagctgctgcacgccccagccaccgtgtgtggccccaagagcggcggcggcagcatcctggccatctacagcaccgtggccagcagcctggtgctgctggtgagcctgggcgccatcagcttttaaagctccccgggggcctcggtggcctagcttcttgccccttgggcctccccccagcccctcctccccttcctgcatttgcctgtgagcagtggtctttgaataaagtctgagtgggcggc

SEQ ID NO: 70 - protein, 584 aa, unidentified
  source 1..584: note = Influenza B virus
  REGION 1..584: note = B/Phuket/3073/2013 (B/Yamagata lineage) HA: wild-type
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 71 - protein, 582 aa, unidentified
  source 1..582: note = Influenza B virus
  REGION 1..582: note = B/Austria/1359417/2021 (B/Victoria lineage) HA: wild-type
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 72 - protein, 584 aa, synthetic construct
  REGION 1..584: note = B_Phuket_HA: Q267L
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMLKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 73 - protein, 584 aa, synthetic construct
  REGION 1..584: note = B_Phuket_HA: G273A
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTATIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 74 - protein, 584 aa, synthetic construct
  REGION 1..584: note = B_Phuket_HA: H435L
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELLNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 75 - protein, 584 aa, synthetic construct
  REGION 1..584: note = B_Phuket_HA: Q267L-G273A-H435L
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMLKPGKTATIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELLNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 76 - protein, 582 aa, synthetic construct
  REGION 1..582: note = B_Austria_HA: H383Y
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWYGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 77 - protein, 582 aa, synthetic construct; identical to SEQ ID NO: 54
  REGION 1..582: note = B_Austria_HA: A290V

SEQ ID NO: 78 - protein, 1273 aa, synthetic construct
  REGION 1..1273: note = Wild-Type Wuhan-Hu-1; USA-WA1/2020 isolate S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 79 - skipped

SEQ ID NO: 80 - RNA, 11 nt, synthetic construct
  source 1..11: mol_type = other RNA
gggatcctacc

SEQ ID NO: 81 - protein, 883 aa, unidentified
  source 1..883: note = Bacteriophage T7
  REGION 1..883: note = WT T7 RNA polymerase
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 82 - protein, 566 aa, unidentified
  source 1..566: note = Influenza A virus
  REGION 1..566: note = A/Darwin/6/2021(H3N2) HA: wild-type
MKTIIALSNILCLVFAQKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRGIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 83 - protein, 566 aa, unidentified
  source 1..566: note = Influenza A virus
  REGION 1..566: note = A/Wisconsin/67/2022(H1N1) HA: wild-type
MKAILVVMLYTFTTANADTLCIGYHANNSTDTVDTVLEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIATRPKVRDQAGRMNYYWTLVEPGDKITFEATGNLVAPRYAFTMEKEAGSGIIISDTPVHDCNATCQTPEGAINTSLPFQNVHPITIGKCPKYVRSTKLRLATGLRNVPSIQSRGLFGAIAGFIEGGWTGMVDGWYGYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 84 - protein, 566 aa, synthetic construct
  REGION 1..566: note = A_Wisconsin_H1 HA: K395C-V36C
MKAILVVMLYTFTTANADTLCIGYHANNSTDTVDTCLEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIATRPKVRDQAGRMNYYWTLVEPGDKITFEATGNLVAPRYAFTMEKEAGSGIIISDTPVHDCNATCQTPEGAINTSLPFQNVHPITIGKCPKYVRSTKLRLATGLRNVPSIQSRGLFGAIAGFIEGGWTGMVDGWYGYHHQNDQGSGYAADLKSTQNAIDKITNCVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 85 - protein, 584 aa, synthetic construct
  REGION 1..584: note = B_Phuket_HA: V239C-V276C-D451C-K422C
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYCSQIGDFPDQTEDGGLPQSGRIVVDYMMQKPGKTGTICYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVCNLQRLSGAMDELHNEILELDEKVDDLRACTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 86 - protein, 584 aa, synthetic construct
  REGION 1..584: note = B_Phuket_HA: I367C-S401C-D451C-K422C
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGACAGFLEGGWEGMIAGWHGYTSHGAHGVAVAADLKCTQEAINKITKNLNSLSELEVCNLQRLSGAMDELHNEILELDEKVDDLRACTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 87 - protein, 582 aa, synthetic construct
  REGION 1..582: note = B_Austria_HA: H381Y-A288V-N494C-K483C
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCVSGKSKVIKGSLPLIGEADCLHEKYGGLNKSKPYYTGEHAKAIGNCPIWVKTPLKLANGTKYRPPAKLLKERGFFGAIAGFLEGGWEGMIAGWYGYTSHGAHGVAVAADLKSTQEAINKITKNLNSLSELEVKNLQRLSGAMDELHNEILELDEKVDDLRADTISSQIELAVLLSNEGIINSEDEHLLALERKLKCMLGPSAVEIGCGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 88 - protein, 582 aa, synthetic construct
  REGION 1..582: note = B_Austria_HA: H381Y-A288V-L422C-D444C
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCVSGKSKVIKGSLPLIGEADCLHEKYGGLNKSKPYYTGEHAKAIGNCPIWVKTPLKLANGTKYRPPAKLLKERGFFGAIAGFLEGGWEGMIAGWYGYTSHGAHGVAVAADLKSTQEAINKITKNLNSLSELEVKNCQRLSGAMDELHNEILELDEKVCDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 89 - protein, 582 aa, synthetic construct
  REGION 1..582: note = B_Austria_HA: H381Y-A288V-A364C-K483C
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCVSGKSKVIKGSLPLIGEADCLHEKYGGLNKSKPYYTGEHAKAIGNCPIWVKTPLKLANGTKYRPPAKLLKERGFFGCIAGFLEGGWEGMIAGWYGYTSHGAHGVAVAADLKSTQEAINKITKNLNSLSELEVKNLQRLSGAMDELHNEILELDEKVDDLRADTISSQIELAVLLSNEGIINSEDEHLLALERKLKCMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 90 - protein, 582 aa, synthetic construct
  REGION 1..582: note = B_Austria_HA: H381Y-A288V-G367C-K483C
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCVSGKSKVIKGSLPLIGEADCLHEKYGGLNKSKPYYTGEHAKAIGNCPIWVKTPLKLANGTKYRPPAKLLKERGFFGAIACFLEGGWEGMIAGWYGYTSHGAHGVAVAADLKSTQEAINKITKNLNSLSELEVKNLQRLSGAMDELHNEILELDEKVDDLRADTISSQIELAVLLSNEGIINSEDEHLLALERKLKCMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 91 - protein, 566 aa, synthetic construct
  REGION 1..566: note = A_Darwin_H3 HA: G402P-R421P-E414P
MKTIIALSNILCLVFAQKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRGIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIPKTNEKFHQIEKPFSEVEGPVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 92 - protein, 566 aa, synthetic construct
  REGION 1..566: note = A_Darwin_H3 HA: S123C-R421C
MKTIIALSNILCLVFAQKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPDYACLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRGIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGCVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 93 - protein, 566 aa, synthetic construct
  REGION 1..566: note = A_Darwin_H3 HA: Q392C-T46C
MKTIIALSNILCLVFAQKIPGNDNSTATLCLGHHAVPNGTIVKTICNDRIEVTNATELVQNSSIGEICGSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRGIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDCINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 94 - protein, 566 aa, synthetic construct
  REGION 1..566: note = A_Wisconsin_H1 HA: K391C-L37C
MKAILVVMLYTFTTANADTLCIGYHANNSTDTVDTVCEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIATRPKVRDQAGRMNYYWTLVEPGDKITFEATGNLVAPRYAFTMEKEAGSGIIISDTPVHDCNATCQTPEGAINTSLPFQNVHPITIGKCPKYVRSTKLRLATGLRNVPSIQSRGLFGAIAGFIEGGWTGMVDGWYGYHHQNDQGSGYAADLKSTQNAIDCITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 95 - protein, 566 aa, unidentified
  source 1..566: note = Influenza A virus
  REGION 1..566: note = A/Sydney/5/2021(H1N1) HA: wild-type
MKAILVVMLYTFTTANADTLCIGYHANNSTDTVDTVLEKNVTVTHSVNLLEDKHNGKLCKLRGVAPLHLGQCNIAGWILGNPECESLSTARSWSYIVETSNSDNGTCYPGNFINYEELREQLSSVSSFERFEIFPKTSSWPNHDSDNGVTAACPHAGAKSFYKNLIWLVKKGKSYPKINQTYINDKGKEVLVLWGIHHPPTITDQESLYQNADAYVFVGTSRYSKKFKPEIAARPKVRDRAGRMNYYWTLVEPGDKITFEATGNLVAPRYAFTMEKDAGSGIIISDTPVHDCNTTCQTPEGAINTSLPFQNVHPITIGKCPKYVRSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 96 - protein, 566 aa, synthetic construct
  REGION 1..566: note = A_Sydney_H1 HA: K391C-L37C
MKAILVVMLYTFTTANADTLCIGYHANNSTDTVDTVCEKNVTVTHSVNLLEDKHNGKLCKLRGVAPLHLGQCNIAGWILGNPECESLSTARSWSYIVETSNSDNGTCYPGNFINYEELREQLSSVSSFERFEIFPKTSSWPNHDSDNGVTAACPHAGAKSFYKNLIWLVKKGKSYPKINQTYINDKGKEVLVLWGIHHPPTITDQESLYQNADAYVFVGTSRYSKKFKPEIAARPKVRDRAGRMNYYWTLVEPGDKITFEATGNLVAPRYAFTMEKDAGSGIIISDTPVHDCNTTCQTPEGAINTSLPFQNVHPITIGKCPKYVRSTKLRLATGLRNVPSIQSRGLFGAIAGFIEGGWTGMVDGWYGYHHQNEQGSGYAADLKSTQNAIDCITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 97 - protein, 566 aa, synthetic construct
  REGION 1..566: note = A_Sydney_H1 HA: K395C-V36C
MKAILVVMLYTFTTANADTLCIGYHANNSTDTVDTCLEKNVTVTHSVNLLEDKHNGKLCKLRGVAPLHLGQCNIAGWILGNPECESLSTARSWSYIVETSNSDNGTCYPGNFINYEELREQLSSVSSFERFEIFPKTSSWPNHDSDNGVTAACPHAGAKSFYKNLIWLVKKGKSYPKINQTYINDKGKEVLVLWGIHHPPTITDQESLYQNADAYVFVGTSRYSKKFKPEIAARPKVRDRAGRMNYYWTLVEPGDKITFEATGNLVAPRYAFTMEKDAGSGIIISDTPVHDCNTTCQTPEGAINTSLPFQNVHPITIGKCPKYVRSTKLRLATGLRNVPSIQSRGLFGAIAGFIEGGWTGMVDGWYGYHHQNEQGSGYAADLKSTQNAIDKITNCVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 98 - protein, 574 aa, unidentified
  source 1..574: note = Human respiratory syncytial virus
  REGION 1..574: note = Wild-Type RSV F Glycoprotein
MELLIHRSSAIFLTLAINTLYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 99 - protein, 510 aa, synthetic construct
  REGION 1..510: note = Prefusion RSV F Glycoprotein
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLY

SEQ ID NO: 100 - protein, 25 aa, unidentified
  source 1..25: note = Human respiratory syncytial virus
  REGION 1..25: note = RSV F C-terminal 25 amino acids
CKARSTPVTLSKDQLSGINNIAFSN

SEQ ID NO: 101 - protein, 20 aa, unidentified
  source 1..20: note = Human respiratory syncytial virus
  REGION 1..20: note = RSV F C-terminal 20 amino acids
TPVTLSKDQLSGINNIAFSN

SEQ ID NO: 102 - protein, 15 aa, unidentified
  source 1..15: note = Human respiratory syncytial virus
  REGION 1..15: note = RSV-F C-terminal 15 amino acids
SKDQLSGINNIAFSN

SEQ ID NO: 103 - protein, 10 aa, unidentified
  source 1..10: note = Human respiratory syncytial virus
  REGION 1..10: note = RSV-F C-terminal 10 amino acids
SGINNIAFSN

SEQ ID NO: 104 - protein, 1273 aa, unidentified; identical to SEQ ID NO: 78
  source 1..1273: note = SARS-CoV-2
  REGION 1..1273: note = Wild-type SARS-CoV-2 S protein

SEQ ID NO: 105 - protein, 1273 aa, synthetic construct
  REGION 1..1273: note = Prefusion SARS-CoV-2 S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 106 - protein, 84 aa, synthetic construct
  REGION 1..84: note = RSV F1 subunit
QNITEEFYQSTCSAVSKGYLSALRTGWYTSVITIELSNIKENKCNGTDAKVKLIKQELDKYKNAVTELQLLMQSTPACNSRARR

SEQ ID NO: 107 - protein, 377 aa, synthetic construct
  REGION 1..377: note = RSV F2 subunit
FLGFLLGVGSACASGIAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSLAFIRKSDELL

SEQ ID NO: 108 - protein, 461 aa, synthetic construct
  REGION 1..461: note = RSV F protein
QNITEEFYQSTCSAVSKGYLSALRTGWYTSVITIELSNIKENKCNGTDAKVKLIKQELDKYKNAVTELQLLMQSTPACNSRARRFLGFLLGVGSACASGIAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSLAFIRKSDELL

SEQ ID NO: 109 - protein, 84 aa, synthetic construct
  REGION 1..84: note = RSV F1 subunit
QNITEEFYQSTCSAVSRGYFSALRTGWYTSVITIELSNIKETKCNGTDTKVKLIKQELDKYKNAVTELQLLMQNTPACNNRARR

SEQ ID NO: 110 - protein, 377 aa, synthetic construct
  REGION 1..377: note = RSV F2 subunit
FLGFLLGVGSACASGIAVSKVLHLEGEVNKIKNALLSTNKAVVSLSNGVSVLTIKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSSEFDASISQVNEKINQSLAFIRRSDELL

SEQ ID NO: 111 - protein, 461 aa, synthetic construct
  REGION 1..461: note = RSV F protein
QNITEEFYQSTCSAVSRGYFSALRTGWYTSVITIELSNIKETKCNGTDTKVKLIKQELDKYKNAVTELQLLMQNTPACNNRARRFLGFLLGVGSACASGIAVSKVLHLEGEVNKIKNALLSTNKAVVSLSNGVSVLTIKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSSEFDASISQVNEKINQSLAFIRRSDELL

SEQ ID NO: 112 - protein, 517 aa, synthetic construct
  REGION 1..517: note = RSV F protein
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 113 - DNA, 1530 bp, synthetic construct
  source 1..1530: mol_type = other DNA
  misc_feature 1..1530: note = Prefusion RSV F protein ORF
atggagctgctgatcctgaaggccaacgccatcacgaccatcctgaccgccgtgaccttctgcttcgccagcgggcagaacatcaccgaggagttctaccagtccacctgctccgccgtgagcaagggctacctgtctgccctgagaaccggctggtacaccagcgtgatcaccatcgagctgtccaacatcaaggagaacaagtgcaacggcaccgacgccaaggtgaagctgatcaagcaggagctggacaagtacaagaacgcagtgaccgagctgcagctgctgatgcagagcacaccagccaccggtagcgggtccgccatttgctccggcgtggccgtgtgcaaggtgctgcacctggagggcgaggtgaacaagatcaagagcgccctgctctccaccaacaaggccgtggtgagcctgagcaacggggtgagcgtgctgaccttcaaggtgctggacctgaagaactacatcgacaagcagctgctgcctatcctgaacaagcagagctgcagcatcagcaacatcgagaccgtgatcgagttccagcagaagaacaaccggctgctggagatcaccagggagttcagcgtgaacgcaggggtgaccacacccgtgtccacctacatgctgaccaactccgagctgctgagcctgatcaacgatatgcccatcaccaacgaccagaagaagctgatgagcaacaacgtgcagatcgtgcggcagcagtcctactccatcatgtgcatcatcaaggaggaggtgctggcctacgtggtgcagctgcccctgtacggcgtgatcgacaccccttgctggaagctgcacaccagccctctgtgcaccaccaacacgaaggagggcagcaatatctgcctgacccggaccgacaggggctggtactgcgacaacgccggcagcgtgtccttctttccccaggccgagacctgcaaggtgcagtccaacagggtgttctgcgacaccatgaactctcgcaccctgcccagcgaggtgaacctgtgcaacgtggacatcttcaaccccaagtacgactgcaagatcatgacctccaagaccgacgtgtcctctagcgttatcacctccctgggcgccatcgtgagctgctacggcaagaccaagtgcaccgccagcaacaagaacaggggcatcatcaagaccttcagcaacgggtgcgactacgtgtccaacaagggcgtggacaccgtgtccgtgggcaacaccctgtactgcgtgaacaagcaggagggcaagagcctgtacgtgaagggcgagcccatcatcaacttctacgaccctctggtgttccccagcgacgagttcgacgccagcatctcccaggtgaacgagaagatcaaccagagcctggccttcatccgcaagagcgacgagctgctgcacaacgtgaacgccggcaagagcaccacaaacatcatgatcaccaccatcatcatcgtgataatcgtgatcctgctgtccctgatcgctgtgggcctgctgctgtac

SEQ ID NO: 114 - protein, 15 aa, synthetic construct
  REGION 1..15: note = Linker
GGSGGSGGSGGSGGG

SEQ ID NO: 115 - protein, 8 aa, synthetic construct
  REGION 1..8: note = Linker
GGGSGGGS

SEQ ID NO: 116 - protein, 7 aa, synthetic construct
  REGION 1..7: note = Linker
GGGSGGG

SEQ ID NO: 117 - protein, 6 aa, synthetic construct
  REGION 1..6: note = Linker
GGGSGG

SEQ ID NO: 118 - protein, 4 aa, synthetic construct
  REGION 1..4: note = Linker
GGGS

SEQ ID NO: 119 - protein, 4 aa, synthetic construct
  REGION 1..4: note = Linker
GSGG

SEQ ID NO: 120 - protein, 4 aa, unidentified
  source 1..4: note = Influenza A virus
  REGION 1..4: note = Polybasic cleavage site
RKTR

SEQ ID NO: 121 - protein, 4 aa, unidentified
  source 1..4: note = Influenza A virus
  REGION 1..4: note = Polybasic cleavage site
RKKR

SEQ ID NO: 122 - protein, 565 aa, unidentified
  source 1..565: note = Influenza A virus
  REGION 1..565: note = A/Beijing/262/1995 (H1N1) HA
MKAKLLVLLCTFTATYADTICIGYHANNSTDTVDTVLEKNVTVTHSVNLLEDSHNGKLCLLKGIAPLQLGNCSVAGWILGNPECESLISKESWSYIVETPNPENGTCYPGYFADYEELREQLSSVSSFERFEIFPKESSWPNHTVTGVTASCSHNGKSSFYRNLLWLTEKNGLYPNLSNSYVNNKEKEVLVLWGVHHPSNIRDQRAIYHTENAYVSVVSSHYSRRFTPEIAKRPKVRGQEGRINYYWTLLEPGDTIIFEANGNLIAPWYAFALSRGFGSGIITSNAPMNECDAKCQTPQGAINSSLPFQNVHPVTIGECPKYVRSTKLRMVTGLRNIPSIQSRGLFGAIAGFIEGGWTGMMDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 123 - protein, 565 aa, unidentified
  source 1..565: note = Influenza A virus
  REGION 1..565: note = A/New Caledonia/20/1999 (H1N1) HA
MKAKLLVLLCTFTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 124 - protein, 565 aa, unidentified
  source 1..565: note = Influenza A virus
  REGION 1..565: note = A/Solomon Islands/3/2006 (H1N1) HA
MKVKLLVLLCTFTATYADTICIGYHANNSTDTVDTVLEKNVTVTHSVNLLEDSHNGKLCLLKGIAPLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGLYPNLSKSYANNKEKEVLVLWGVHHPPNIGDQRALYHTENAYVSVVSSHYSRKFTPEIAKRPKVRDREGRINYYWTLLEPGDTIIFEANGNLIAPRYAFALSRGFGSGIINSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 125 - protein, 565 aa, unidentified
  source 1..565: note = Influenza A virus
  REGION 1..565: note = A/Brisbane/59/2007 (H1N1) HA
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGNQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 126 - skipped

SEQ ID NO: 127 - protein, 566 aa, unidentified
  source 1..566: note = Influenza A virus
  REGION 1..566: note = A/California/7/2009 (H1N1)pdm09 HA
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGXSYPKLSKSYINDKGKEVLVLWGIHHPSTSADQQSLYQNADAYVFVGSSRYSKKFKPEIAIRPKVRXXEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 128 - protein, 566 aa, unidentified
  source 1..566: note = Influenza A virus
  REGION 1..566: note = A/Michigan/45/2015 (H1N1)pdm09 HA
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDREGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 129 - protein, 566 aa, unidentified
  source 1..566: note = Influenza A virus
  REGION 1..566: note = A/Brisbane/02/2018 (H1N1)pdm09 HA
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 130 - protein, 566 aa, unidentified
  source 1..566: note = Influenza A virus
  REGION 1..566: note = A/Guangdong-Maonan/SWL1536/2019 (H1N1)pdm09 HA
MKAILVVLLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDKGVTAACPHAGAKSFYKNLIWLVKKGNSYPKLNQTYINDKGKEVLVLWGIHHPPTIAAQESLYQNADAYVFVGTSRYSKKFKPEIATRPKVRDQEGRMNYYWTLVEPGDKITFEATGNLVVPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 131 - protein, 566 aa, unidentified; identical to SEQ ID NO: 130
  source 1..566: note = Influenza A virus
  REGION 1..566: note = A/Hawaii/70/2019 (H1N1)pdm09 HA

SEQ ID NO: 132 - protein, 566 aa, unidentified
  source 1..566: note = Influenza A virus
  REGION 1..566: note = A/Victoria/2570/2019 (H1N1)pdm09 HA
MKAILVVMLYTFTTANADTLCIGYHANNSTDTVDTVLEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIAARPKVRDQAGRMNYYWTLVEPGDKITFEATGNLVAPRYAFTMEKEAGSGIIISDTPVHDCNATCQTPEGAINTSLPFQNVHPITIGKCPKYVRSTKLRLATGLRNVPSIQSRGLFGAIAGFIEGGWTGMVDGWYGYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 133 - protein, 566 aa, unidentified; identical to SEQ ID NO: 48
  source 1..566: note = Influenza A virus
  REGION 1..566: note = A/Wisconsin/588/2019 (H1N1)pdm09 HA

SEQ ID NO: 134 - protein, 566 aa, unidentified
  source 1..566: note = Influenza A virus
  REGION 1..566: note = A/Victoria/4897/2022 (H1N1)pdm09 HA
MKAILVVMLYTFTTANADTLCIGYHANNSTDTVDTVLEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGAKSFYKNLIWLVKKGKSYPKINQTYINDKGKEVLVLWGIHHPPTITDQESLYQNADAYVFVGTSRYSKKFKPEIAARPKVRDRAGRMNYYWTLVEPGDKITFEATGNLVAPRYAFTMEKEAGSGIIISDTPVHDCNATCQTPEGAINTSLPFQNVHPITIGKCPKYVRSTKLRLATGLRNVPSIQSRGLFGAIAGFIEGGWTGMVDGWYGYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 135 - protein, 566 aa, unidentified; identical to SEQ ID NO: 83
  source 1..566: note = Influenza A virus
  REGION 1..566: note = A/Wisconsin/67/2022 (H1N1)pdm09 HA

SEQ ID NO: 136 - protein, 566 aa, unidentified
  source 1..566: note = Influenza A virus
  REGION 1..566: note = A/Sydney/5/97 (H3N2) HA
MKTIIALSYILCLVFAQKIPGNDNSTATLCLGHHAVPNGTLVKTITNDQIEVTNATELVQSSSTGRICDSPHRILDGENCTLIDALLGDPHCDGFQNKEWDLFVERSKAYSNCYPYDVPDYASLRSLVASSGTLEFNNESFNWTGVAQNGTSYACKRSSIKSFFSRLNWLHQLKYKYPALNVTMPNNDKFDKLYIWGVHHPSTDSDQTSIYAQASGRVTVSTKRSQQTVIPNIGSRPWVRGISSRISIHWTIVKPGDILLINSKGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIEKTNEKFHQIEKEFSEVEGRIQDLEKYVEDTKIDLWSYNAELLVALENQHTIDLTDSEMNKLFERTRKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 137 - protein, 566 aa, unidentified
  source 1..566: note = Influenza A virus
  REGION 1..566: note = A/Moscow/10/1999 (H3N2) HA
MKTIIALSYILCLVFAQKLPGNDNSTATLCLGHHAVPNGTLVKTITNDQIEVTNATELVQSSSTGRICDSPHQILDGENCTLIDALLGDPHCDGFQNKEWDLFVERSKAYSNCYPYDVPDYASLRSLVASSGTLEFNNESFNWTGVAQNGTSSACKRRSINSFFSRLNWLHQLKYRYPALNVTMPNNDKFDKLYIWGVHHPSTDSDQTSLYTQASGRVTVSTKRSQQTVIPNIGSRPWVRGISSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMMDGWYGFRHQNSEGTGQAADLKSTQAAINQINGKLNRLIEKTNEKFHQIEKEFSEVEGRIQDLEKYVEDTKIDLWSYNAELLVALENQHTIDLTDSEMNKLFERTRKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 138 - protein, 566 aa, unidentified
  source 1..566: note = Influenza A virus
  REGION 1..566: note = A/Fujian/411/2002 (H3N2) HA
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNKSFFSRLNWLTHLKYKYPALNVTMPNNEKFDKLYIWGVLHPGTDSDQISLYAQASGRITVSTKRSQQTVIPNIGSRPRVRGVSSRISIYWTIVKPGDILLINSTGNLIAPRGYFKIRSGKSSIMRSDAPIGKCNSECITPNGSIPNDKPFQNVNRITYGACPRYIKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 139 - protein, 566 aa, unidentified
  source 1..566: note = Influenza A virus
  REGION 1..566: note = A/California/7/2004 (H3N2) HA
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSSCKRRSNNSFFSRLNWLTHLKFKYPALNVTMPNNEKFDKLYIWGVHHPGTNNDQISLYTQASGRITVSTKRSQQTVIPNIGSRPRVRDIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 140 - protein, 566 aa, unidentified
  source 1..566: note = Influenza A virus
  REGION 1..566: note = A/Wisconsin/67/2005 (H3N2) HA
MKTIIALSYILCLVFAQKLPGNDDSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNDESFNWTGVTQNGTSSSCKRRSNNSFFSRLNWLTHLKFKYPALNVTMPNNEKFDKLYIWGVHHPVTDNDQIFLYAQASGRITVSTKRSQQTVIPNIGSRPRIRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 141 - protein, 566 aa, unidentified
  source 1..566: note = Influenza A virus
  REGION 1..566: note = A/Brisbane/10/2007 (H3N2) HA
MKTIIALSYILCLVFTQKLPGNDNSTATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLKFKYPALNVTMPNNEKFDKLYIWGVHHPGTDNDQIFLYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 142 - protein, 566 aa, unidentified
  source 1..566: note = Influenza A virus
  REGION 1..566: note = A/Perth/16/2009 (H3N2) HA
MKTIIALSYILCLVFAQKLPGNDNSTATLCLGHHAVPNGTIVKTITNDQIEVTNATELVQSSSTGEICDSPHQILDGKNCTLIDALLGDPQCDGFQNKKWDLFVERSKAYSNCYPYDVPDYASLRSLVASSGTLEFNNESFNWTGVTQNGTSSACIRRSKNSFFSRLNWLTHLNFKYPALNVTMPNNEQFDKLYIWGVHHPGTDKDQIFLYAQASGRITVSTKRSQQTVSPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 143 - protein, 566 aa, unidentified
  source 1..566: note = Influenza A virus
  REGION 1..566: note = A/Victoria/361/2011 (H3N2) HA
MKTIIALSHILCLVFAQKLPGNDNSTATLCLGHHAVPNGTIVKTITNDQIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLNFKYPALNVTMPNNEQFDKLYIWGVHHPGTDKDQIFLYAQSSGRITVSTKRSQQAVIPNIGSRPRIRNIPSRISIYWTIVKPGDILLINSTGNLIAPRGYFKIRSGKSSIMRSDAPIGKCNSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 144 - protein, 566 aa, unidentified
  source 1..566: note = Influenza A virus
  REGION 1..566: note = A/Texas/50/2012 (H3N2) HA
MKTIIALSYILCLVFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWNGVTQNGTSSACIRRSNNSFFSRLNWLTHLNFKYPALNVTMPNNEQFDKLYIWGVHHPGTDKDQIFLYAQPSGRITVSTKRSQQAVIPNIGSRPRIRNIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 145 - protein, 566 aa, unidentified
  source 1..566: note = Influenza A virus
  REGION 1..566: note = A/Switzerland/9715293/2013 (H3N2) HA
MKTIIALSYILCLVFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWAGVTQNGTSSSCIRGSNSSFFSRLNWLTHLNSKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 146 - protein, 566 aa, unidentified
  source 1..566: note = Influenza A virus
  REGION 1..566: note = A/Hong Kong/4801/2014 (H3N2) HA
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 147 - protein, 566 aa, unidentified
  source 1..566: note = Influenza A virus
  REGION 1..566: note = A/Singapore/INFIMH-16-0019/2016 (H3N2) HA
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRGSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIESIRNETYDHNVYRDEALNNRFQIKGVELKSGYKDWILWISFAISCFLLCVALLGFIMWACQKGNIRCNICI

SEQ ID NO: 148 - protein, 566 aa, unidentified
  source 1..566: note = Influenza A virus
  REGION 1..566: note = A/Kansas/14/2017 (H3N2) HA
MKTIIALSCILCLVFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCYPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISCFLLCVALLGFIMWACQKGNIRCNICI

SEQ ID NO: 149 - protein, 566 aa, unidentified
  source 1..566: note = Influenza A virus
  REGION 1..566: note = A/Hong Kong/2671/2019 (H3N2) HA
MKTIIALSYILCLVFTQKIPGNDNSTATLCLGHHAVPNGTIVKTITNDRIEVTNATELVQNSSIGEICDSPHQILDGGNCTLIDALLGDPQCDGFQNKKWDLFVERSRAYSNCYPYDVPDYASLRSLVASSGTLEFKNESFNWAGVTQNGKSFSCIRGSSSSFFSRLNWLTHLNYTYPALNVTMPNKEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 150 - protein, 566 aa, unidentified
  source 1..566: note = Influenza A virus
  REGION 1..566: note = A/Hong Kong/45/2019 (H3N2) HA
MKTIIALSYILCLGFTQKIPGNDNSTATLCLGHHAVPNGTIVKTITNDRIEVTNATELVQNSSIGEICDSPHQILDGGNCTLIDALLGDPQCDGFQNKKWDLFVERSRAYSNCYPYDVPDYASLRSLVASSGTLEFKNESFNWAGVTQNGKSFSCIRGSSSSFFSRLNWLTHLNYTYPALNVTMPNKEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQNTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 151 - protein, 566 aa, unidentified [sequence omitted]

SEQ ID NO: 152 - protein, 566 aa, unidentified
  source 1..566: note = Influenza A virus
  REGION 1..566: note = A/Darwin/9/2021 (H3N2) HA
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 153 - protein, 566 aa, unidentified; identical to SEQ ID NO: 51
  source 1..566: note = Influenza A virus
  REGION 1..566: note = A/Darwin/6/2021 (H3N2) HA

SEQ ID NO: 154 - protein, 585 aa, unidentified
  source 1..585: note = Influenza B virus
  REGION 1..585: note = B/Shangdong/7/1997 HA
MKAIIVLLMVVTSNADRICTGITSSNSPHVVKTATQGEVNVTGVIPLTTTPTKSHFANLKGTKTRGKLCPKCLNCTDLDVALGRPKCTGNIPSAKVSILHEVRPVTSGCFPIMHDRTKIRQLPNLLRGYEHIRLSIHNVINAEKAPGGPYKIGTSGSCPNVTNGNGFFATMAWAVPKNDNNKTATNSLTIEVPYICTEGEDQITVWGFHSDNENQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFNAGEFSLPTFDSLNITAASLNDDGLDNHTILLYYSTAASSLAVTLMIAIFIVYMVSRDNVSCSICL

SEQ ID NO: 155 - protein, 585 aa, unidentified
  source 1..585: note = Influenza B virus
  REGION 1..585: note = B/Hong Kong/330/2001 HA
MKAIIVLLMVVTSNADRICTGITSSNSPHVVKTATQGEVNVTGVIPLTTTPTKSHFANLKGTKTRGKLCPKCLNCTDLDVALGRPKCTGNIPSAKVSILHEVRPVTSGCFPIMHDRTKIRQLPNLLRGYERIRLSNHNVINAEKAPGGPYKIGTSGSCPNVTNGNGFFATMAWAVPKNENNKTATNSLTIEVPYICTEGEDQITVWGFHSDSETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFNAGEFSLPTFDSLNITAASLNDDGLDNHTILLYYSTAASSLAVTLMIAIFVVYMVSRDNVSCSICL

SEQ ID NO: 156 - protein, 585 aa, unidentified
  source 1..585: note = Influenza B virus
  REGION 1..585: note = B/Malaysia/2506/2004 HA
MKAIIVLLMVVTSNADRICTGITSSNSPHVVKTATQGEVNVTGVIPLTTTPTKSHFANLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDNEIQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 157 - protein, 585 aa, unidentified
  source 1..585: note = Influenza B virus
  REGION 1..585: note = B/Brisbane/60/2008 HA
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHIRLSTHNVINAENAPGGPYKIGTSGSCPNITNGNGFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 158 - protein, 583 aa, unidentified
  source 1..583: note = Influenza B virus
  REGION 1..583: note = B/Colorado/06/2017 HA
MKAIIVLLMVVTSSADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEGAPGGPYKIGTSGSCPNITNGNGFFATMAWAVPDKNKTATNPLTIEVPYVCTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDKIAAGTFDAGEFSLPTFDSLNITAASLNDDGLDNHTILLYYSTAASSLAVTLMIAIFVVYMVSRDNVSCSICL

SEQ ID NO: 159 - protein, 582 aa, unidentified
  source 1..582: note = Influenza B virus
  REGION 1..582: note = B/Washington/02/2019 HA
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDSETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 160 - protein, 582 aa, unidentified; identical to SEQ ID NO: 71
  source 1..582: note = Influenza B virus
  REGION 1..582: note = B/Austria/1359417/2021 HA

SEQ ID NO: 161-163 - skipped (3 entries)

SEQ ID NO: 164 - protein, 584 aa, unidentified
  source 1..584: note = Influenza B virus
  REGION 1..584: note = B/Florida/4/2006 HA
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 165 - protein, 584 aa, unidentified
  source 1..584: note = Influenza B virus
  REGION 1..584: note = B/Wisconsin/1/2010 HA
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYENIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 166 - protein, 584 aa, unidentified
  source 1..584: note = Influenza B virus
  REGION 1..584: note = B/Massachusetts/2/2012 HA
MKAIIVLLMVVTSNADRICTGITSSNSPHVVKTATQGEVNVTGVIPLTTTPTKSYFANLKGTKTRGKLCPDCLNCTDLDVALGRPMCVGTTPSAKASILHEVRPVTSGCFPIMHDRTKIRQLANLLRGYENIRLSTQNVIDAEKAPGGPYRLGTSGSCPNATSKSGFFATMAWAVPKDNNKNATNPLTVEVPYICAEGEDQITVWGFHSDDKTQMKNLYGDSNPQKFTSSANGVTTHYVSQIGGFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 167 - protein, 584 aa, unidentified; identical to SEQ ID NO: 70
  source 1..584: note = Influenza B virus
  REGION 1..584: note = B/Phuket/3073/2013 HA

SEQ ID NO: 168 - protein, 17 aa, unidentified
  source 1..17: note = Influenza A virus
  REGION 1..17: note = A/Wisconsin/67/2022 (H1N1)pdm09 H1 HA Signal Peptide
MKAILVVMLYTFTTANA

SEQ ID NO: 169 - protein, 16 aa, unidentified
  source 1..16: note = Influenza A virus
  REGION 1..16: note = A/Darwin/6/2021 (H3N2) H3 HA Signal Peptide
MKTIIALSNILCLVFA

SEQ ID NO: 170 - protein, 15 aa, unidentified
  source 1..15: note = Influenza B virus
  REGION 1..15: note = B/Austria/1359417/2021 B/Victoria lineage HA Signal Peptide
MKAIIVLLMVVTSNA

SEQ ID NO: 171 - protein, 15 aa, unidentified
  source 1..15: note = Influenza B virus
  REGION 1..15: note = B/Phuket/3073/2013 B/Yamagata lineage HA Signal Peptide
MKAIIVLLMVVTSNA

SEQ ID NO: 172 - protein, 549 aa, unidentified
  source 1..549: note = Influenza A virus
  REGION 1..549: note = A/Wisconsin/67/2022 (H1N1)pdm09 H1 HA without SP
DTLCIGYHANNSTDTVDTVLEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIATRPKVRDQAGRMNYYWTLVEPGDKITFEATGNLVAPRYAFTMEKEAGSGIIISDTPVHDCNATCQTPEGAINTSLPFQNVHPITIGKCPKYVRSTKLRLATGLRNVPSIQSRGLFGAIAGFIEGGWTGMVDGWYGYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 173 - protein, 550 aa, unidentified
  source 1..550: note = Influenza A virus
  REGION 1..550: note = A/Darwin/6/2021 (H3N2) H3 HA without SP
QKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRGIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 174 - protein, 567 aa, unidentified
  source 1..567: note = Influenza B virus
  REGION 1..567: note = B/Austria/1359417/2021 B/Victoria lineage HA without SP
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 175 - protein, 569 aa, unidentified
  source 1..569: note = Influenza B virus
  REGION 1..569: note = B/Austria/1359417/2021 B/Yamagata lineage HA without SP
DRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 176 - protein, 884 aa, synthetic construct
  REGION 1..884: note = T7 RNA Polymerase Variant 1
MNTINIAKNDFSDIELAAIPFNTLADHYGERLAREQLALEHESYEMAEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFAG

SEQ ID NO: 177 - protein, 884 aa, synthetic construct
  REGION 1..884: note = T7 RNA Polymerase Variant 2
MNTINIAKNDFSDIELAAIPFNTLADHYGERLAREQLALEHESYEMAEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFAG

SEQ ID NO: 178 - protein, 883 aa, synthetic construct
  REGION 1..883: note = T7 RNA Polymerase Variant 3
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 179 - protein, 883 aa, synthetic construct; identical to SEQ ID NO: 178
  REGION 1..883: note = T7 RNA Polymerase Variant 4

SEQ ID NO: 180 - protein, 470 aa, unidentified
  source 1..470: note = Influenza A virus
  REGION 1..470: note = A/Beijing/262/1995 (H1N1) NA
MNPNQKIITIGSISIVIGIISLMLQIGNIISIWASHSIQTGSQNHTGICNQRIITYENSTWVNHTYVNINNTNVVAGKDKTSVTLAGNSSLCSISGWAIYTKDNSIRIGSKGDVFVIREPFISCSHLECRTFFLTQGALLNDKHSNGTVKDRSPYRALMSCPLGEAPSPYNSKFESVAWSASACHDGMGWLTIGISGPDNGAVAVLKYNGIITETIKSWKKRILRTQESECVCVNGSCFTIMTDGPSNGAASYKIFKIEKGKVTKSIELNAPNSHYEECSCYPDTGTVMCVCRDNWHGSNRPWVSFNQNLDYQIGYICSGVFGDNPRPKDGEGSCNPVTVDGADGVKGFSYRYGNGVWIGRTKSNRLRKGFEMIWDPNGWTDTDSDFSVKQDVVAMTDWSGYSGSFVQHPELTGLDCIRPCFWVELVRGRPRENTTIWTSGSSISFCGVNSDTANWSWPDGAELPFTIDK

SEQ ID NO: 181 - protein, 470 aa, unidentified
  source 1..470: note = Influenza A virus
  REGION 1..470: note = A/New Caledonia/20/1999 (H1N1) NA
MNPNQKIITIGSISIAIGIISLMLQIGNIISIWASHSIQTGSQNHTGVCNQRIITYENSTWVNHTYVNINNTNVVAGKDKTSVTLAGNSSLCSISGWAIYTKDNSIRIGSKGDVFVIREPFISCSHLECRTFFLTQGALLNDKHSNGTVKDRSPYRALMSCPLGEAPSPYNSKFESVAWSASACHDGMGWLTIGISGPDNGAVAVLKYNGIITETIKSWKKRILRTQESECVCVNGSCFTIMTDGPSNGAASYKIFKIEKGKVTKSIELNAPNFHYEECSCYPDTGTVMCVCRDNWHGSNRPWVSFNQNLDYQIGYICSGVFGDNPRPKDGEGSCNPVTVDGADGVKGFSYKYGNGVWIGRTKSNRLRKGFEMIWDPNGWTDTDSDFSVKQDVVAITDWSGYSGSFVQHPELTGLDCIRPCFWVELVRGLPRENTTIWTSGSSISFCGVNSDTANWSWPDGAELPFTIDK

SEQ ID NO: 182 - protein, 470 aa, unidentified
  source 1..470: note = Influenza A virus
  REGION 1..470: note = A/Solomon Islands/3/2006 (H1N1) NA
MNPNQKIITIGSISIAIGIISLILQIGNIISIWASHSIQTGSQNHTGICNQRIITYENSTWVNNTYVNINNTNVVAEKDKTSVTLAGNSSLCSISGWAIYTKDNSIRIGSKGDVFVIREPFISCSHLECRTFFLTQGALLNDKHSNGTVKDRSPYRALMSCPLGEAPSPYNSRFESVAWSASACHDGMGWLTIGISGPDNGAVAVLKYNGIITETIKSWKKRILRTQESECVCMNGSCFTIMTDGPSNGAASYKIFKIEKGKVTKTIELNAPNFHYEECSCYPDTGTVMCVCRDNWHGSNRPWVSFNQNLDYQIGYICSGVFGDNPRPKDGEGSCNPVTVDGADGVKGFSYKYGNGVWIGRTKSNRLRKGFEMIWDPNGWTNTDSDFSVKQDVVAITDWSGYSGSFVQHPELTGLDCIRPCFWVELVRGLPRENTTIWTSGSSISFCGVNSGTANWSWPDGAELPFTIDK

SEQ ID NO: 183 - protein, 470 aa, unidentified
  source 1..470: note = Influenza A virus
  REGION 1..470: note = A/Brisbane/59/2007 (H1N1) NA
MNPNQKIITIGSISIAIGIISLMLQIGNIISIWASHSIQTGSQNNTGICNQRIITYENSTWVNHTYVNINNTNVVAGEDKTSVTLAGNSSLCSISGWAIYTKDNSIRIGSKGDVFVIREPFISCSHLECRTFFLTQGALLNDKHSNGTVKDRSPYRALMSCPLGEAPSPYNSKFESVAWSASACHDGMGWLTIGISGPDNGAVAVLKYNGIITGTIKSWKKQILRTQESECVCMNGSCFTIMTDGPSNKAASYKIFKIEKGKVTKSIELNAPNFHYEECSCYPDTGIVMCVCRDNWHGSNRPWVSFNQNLDYQIGYICSGVFGDNPRPEDGEGSCNPVTVDGANGVKGFSYKYDNGVWIGRTKSNRLRKGFEMIWDPNGWTNTDSDFSVKQDVVAITDWSGYSGSFVQHPELTGLDCIRPCFWVELVRGLPRENTTIWTSGSSISFCGVNSDTANWSWPDGAELPFTIDK

SEQ ID NO: 184 - skipped

SEQ ID NO: 185 - protein, 469 aa, unidentified
  source 1..469: note = Influenza A virus
  REGION 1..469: note = A/California/7/2009 (H1N1)pdm09 NA
MNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 186 - protein, 469 aa, unidentified
  source 1..469: note = Influenza A virus
  REGION 1..469: note = A/Michigan/45/2015 (H1N1)pdm09 NA
MNPNQKIITIGSICMTIGMANLILQIGNIISIWVSHSIQIGNQSQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 187 - protein, 469 aa, unidentified
  source 1..469: note = Influenza A virus
  REGION 1..469: note = A/Brisbane/02/2018 (H1N1)pdm09 NA
MNPNQKIITIGSTCMTIGMANLILQIGNIISIWVSHSIQIGNQSQIETCNQSVITYENNTWVNQTYVNISNTNFAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVDSDTVGWSWPDGAELPFTIDK

SEQ ID NO: 188 - protein, 469 aa, unidentified
  source 1..469: note = Influenza A virus
  REGION 1..469: note = A/Guangdong-Maonan/SWL1536/2019 (H1N1)pdm09 NA
MNPNQKIITIGSICMTIGMANLILQIGNIISIWVSHSIQIGNQSQIETCNKNVITYENNTWVNQTYVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 189 - protein, 469 aa, unidentified; identical to SEQ ID NO: 188
  source 1..469: note = Influenza A virus
  REGION 1..469: note = A/Hawaii/70/2019 (H1N1)pdm09 NA

SEQ ID NO: 190 - protein, 469 aa, unidentified
  source 1..469: note = Influenza A virus
  REGION 1..469: note = A/Victoria/2570/2019 (H1N1)pdm09 NA
MNPNQKIITIGSICMTIGTANLILQIGNIISIWVSHSIQIGNQSQIETCDKSVITYENNTWVNQTFVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTETDNKFSKKQDIVGINEWSGYSGSFVQHPELTGLNCIRPCFWVELIRGRPEENTIWTSGSSISFCGVDSDIMGWSWPDGAELPFTIDN

SEQ ID NO: 191 - protein, 469 aa, unidentified
  source 1..469: note = Influenza A virus
  REGION 1..469: note = A/Wisconsin/588/2019 (H1N1)pdm09 NA
MNPNQKIITIGSICMTIGTANLILQIGNIISIWVSHSIQIGNQSQIETCNKSVITYENNTWVNQTFVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 192 - protein, 469 aa, unidentified; identical to SEQ ID NO: 190
  source 1..469: note = Influenza A virus
  REGION 1..469: note = A/Victoria/4897/2022 (H1N1)pdm09 NA

SEQ ID NO: 193 - protein, 469 aa, unidentified
  source 1..469: note = Influenza A virus
  REGION 1..469: note = A/Wisconsin/67/2022 (H1N1)pdm09 NA
MNPNQKIITIGSVCMTIGTANLILQIGNIISIWVSHSIQIGNQSQIETCNKSVITYENNTWVNQTFVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDN

SEQ ID NO: 194 - protein, 469 aa, unidentified
  source 1..469: note = Influenza A virus
  REGION 1..469: note = A/Sydney/5/97 (H3N2) NA
MNPNQKIITIGSVSLTIATICFLMQIAILVTTVTLHFKQYECSSPPNNQVMLCEPTIIERNITEIVYLTNTTIEKEICPKLAEYRNWSKPQCKITGFAPFSKDNSIRLSAGGDIWVTREPYVSCDPDKCYQFALGQGTTLNNRHSNDTVHDRTPYRTLLMNELGVPFHLGTKQVCIAWSSSSCHDGKAWLHVCVTGHDENATASFIYDGRLVDSIGSWSKKILRTQESECVCINGTCTVVMTDGSASGRADTKILFIEEGKIVHISPLSGSAQHVEECSCYPRYPGVRCVCRDNWKGSNRPIVDINVKDYSIVSSYVCSGLVGDTPRKNDSSSSSHCLNPNNEEGGHGVKGWAFDDGNDVWMGRTISEKFRSGYETFKVIEGWSKPNSKLQINRQVIVDRGNRSGYSGIFSVEGKSCINRCFYVELIRGRKQETEVWWTSNSIVVFCGTSGTYGTGSWPDGADINLMPI

SEQ ID NO: 195 - protein, 469 aa, unidentified
  source 1..469: note = Influenza A virus
  REGION 1..469: note = A/Moscow/10/1999 (H3N2) NA
MNPNQKIITIGSVSLTIATICFLMQIAILVTTVTLHFKQYECNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNGHSNDTVHDRTPYRTLLMNELGVPFHLGTKQVCIAWSSSSCHDGKAWLHVCVTGDDENATASFIYNGRLVDSIGSWSKKILRTQESECVCINGTCTVVMTDGSASGKADTKILFIEEGKIVHTSPLSGSAQHVEECSCYPRYPGVRCVCRDNWKGSNRPIVDINVKDYSIVSSYVCSGLVGDTPRKNDSSSSSHCLDPNNEEGGHGVKGWAFDDGNDVWMGRTISEKLRSGYETFKVIEGWSKPNSKLQINRQVIVDRGNRSGYSGIFSVEGKSCINRCFYVELIRGRKQETEVLWTSNSIVVFCGTSGTYGTGSWPDGADINLMPI

SEQ ID NO: 196 - protein, 469 aa, unidentified
  source 1..469: note = Influenza A virus
  REGION 1..469: note = A/Fujian/411/2002 (H3N2) NA
MNPNQKIITIGSVSLTISTICFFMQIAILITTVTLHFKQYEFNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDENATASFIYNGRLVDSIVSWSKKILRTQESECVCINGTCTVVMTDGSASGKADTKILFIEEGKIVHTSTLSGSAQHVEECSCYPRYPGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKQETEVLWTSNSIVVFCGTSGTYGTGSWPDGADINLMPI

SEQ ID NO: 197 - protein, 469 aa, unidentified
  source 1..469: note = Influenza A virus
  REGION 1..469: note = A/California/7/2004 (H3N2) NA
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYPGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 198 - protein, 469 aa, unidentified
  source 1..469: note = Influenza A virus
  REGION 1..469: note = A/Wisconsin/67/2005 (H3N2) NA
MNPNQKIITIGSVSLTISTICFFMQIAILITTVTLHFKQYEFNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 199 - protein, 469 aa, unidentified
  source 1..469: note = Influenza A virus
  REGION 1..469: note = A/Brisbane/10/2007 (H3N2) NA
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNDTVRDRTPYRTLLMNELGVPFHLGTKQVCIAWSSSSCHDGKAWLHVCITGDDKNATASFIYNGRLVDSIVSWSKEILRTQESECVCINGTCTVVMTDGSASGKADTKILFIEEGKIVHTSTLSGSAQHVEECSCYPRYPGVRCVCRDNWKGSNRPIVDINIKDHSTVSSYVCSGLVGDTPRKNDSSSSSHCLDPNNEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGTYGTGSWPDGADINLMPI

SEQ ID NO: 200 - protein, 469 aa, unidentified
  source 1..469: note = Influenza A virus
  REGION 1..469: note = A/Perth/16/2009 (H3N2) NA
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGTYGTGSWPDGADINLMPI

SEQ ID NO: 201 - protein, 469 aa, unidentified
  source 1..469: note = Influenza A virus
  REGION 1..469: note = A/Victoria/361/2011 (H3N2) NA
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIKEGKIVHTSTLSGSAQHVEECSCYPRYPGVRCVCRDNWKGSNRPIVDINIKDHSIVSSYVCSGLVGDTPRKTDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 202 - protein, 469 aa, unidentified
  source 1..469: note = Influenza A virus
  REGION 1..469: note = A/Texas/50/2012 (H3N2) NA
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVHDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 203 - protein, 469 aa, unidentified
  source 1..469: note = Influenza A virus
  REGION 1..469: note = A/Switzerland/9715293/2013 (H3N2) NA
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 204 - protein, 469 aa, unidentified
  source 1..469: note = Influenza A virus
  REGION 1..469: note = A/Hong Kong/4801/2014 (H3N2) NA
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKDILRTQESECICINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQTNRQVIVDRGDRSGYSGIFSVEGKSCINRCFYVELIRGRKEETEVLWTSNSIVVFCGTSGTYGTGSWPDGADLNLMPI

SEQ ID NO: 205 - protein, 469 aa, unidentified
  source 1..469: note = Influenza A virus
  REGION 1..469: note = A/Singapore/INFIMH-16-0019/2016 (H3N2) NA
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLIDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 206 - protein, 469 aa, unidentified
  source 1..469: note = Influenza A virus
  REGION 1..469: note = A/Kansas/14/2017 (H3N2) NA
MNPNQKIITIGSVSLTISTICFFMQIAILITTVTLHFKQYEFNSPPNNQVMLCEPTIIERNITEIVYLTNTTIEREICPKPAEYRNWSKPQCGITGFAPFSKDNSIRLSAGGDIWVTREPYVSCDPDKCYQFALGQGTTINNVHSNNTARDRTPHRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKTDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 207 - protein, 469 aa, unidentified
  source 1..469: note = Influenza A virus
  REGION 1..469: note = A/Hong Kong/2671/2019 (H3N2) NA
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSSYVCSGLVGDTPRKSDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHT

SEQ ID NO: 208 - protein, 469 aa, unidentified
  source 1..469: note = Influenza A virus
  REGION 1..469: note = A/Hong Kong/45/2019 (H3N2) NA
MNPNQKIITIGSVSLTISTICFFMQIAILITTVTLHFKQYEFNSLPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSSYVCSGLVGDTPRKSDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 209 - protein, 469 aa, unidentified
  source 1..469: note = Influenza A virus
  REGION 1..469: note = A/Cambodia/e0826360/2020 (H3N2) NA
MNPNQKIITIGSVSLTISTICFFMQIAILITTVTLHFKQYEFNSPPNNQVMLCEPTIIERNMTEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 210 - protein, 469 aa, unidentified
  source 1..469: note = Influenza A virus
  REGION 1..469: note = A/Darwin/9/2021 (H3N2) NA
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 211 - protein, 469 aa, unidentified
  source 1..469: note = Influenza A virus
  REGION 1..469: note = A/Darwin/6/2021 (H3N2) NA
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 212 - protein, 466 aa, unidentified
  source 1..466: note = Influenza B virus
  REGION 1..466: note = B/Shangdong/7/1997 NA
MLPSTIQTLTLFLTSGGVLLSLYVSALLSYLLYSDILLKFSPTKVIAPTMSLDCANASNVQAVNHSATEEMTFLLPEPEWTYPRLSCQGSTFQKALLISPHRFGEAKGNSAPLIIREPFIACGPKECKHFALTHYAAQPGGYYNGTREDRNKLRHLISVNLGKIPTVENSIFHMAAWSGSACHDGREWTYIGVDGPDSNALIKIKYGEAYTDTYHSYANNILRTQESACNCIGGDCYLMITDGSASGISRCRFLKIREGRIIKEIFPTGRVEHTEECTCGFASNKTIECACRDNSYTAKRPFVKLNVETDTAEIRLMCTETYLDTPRPDDGSITGPCESNGDKGNGGIKGGFVHQRMASRIGRWYSRTMSKTKRMGMELYVKYDGDPWTDSDALAPSGVMVSIEEPGWYSFGFEIKDKKCDVPCIGIEMVHDGGKKTWHSAATAIYCLMGSGQLLWDTVTGVDMAL

SEQ ID NO: 213 - protein, 466 aa, unidentified
  source 1..466: note = Influenza B virus
  REGION 1..466: note = B/Hong Kong/330/2001 NA
MLPSTIQTLTLFLTSGGVLLSLYVSALLSYLLYSDILLKFSPTKIIAPTTSLDSANASNFQAVNHSATKEMTFLLPEPEWTYPRLSCQGSTFQKALLISPHRFGEAKGNSAPLIIREPFIACGPKECKHFALTHYAAQPGGYYNGTREDRNKLRHLISVNLGKIPTVENSIFHMAAWSGSACHDGREWTYIGVDGPDSNALIKIKYGEAYTDTYHSYANNILRTQESACNCIGGDCYLMITDGSASGISKCRFLKIREGRIVKEIFPTGRVEHTEECTCGFASNKTIECACRDNSYTAKRPFVKLNVETDTAEIRLMCTETYLDTPRPDDGSITGPCESNGDKGSGGIKGGFVHQRMASKIGRWYSRTMSKTKRMGMELYVKYDGDPWTDSDALAPSGVMVSIEEPGWYSFGFEIKDKKCDVPCIGIEMVHDGGKTTWHSAATAIYCLMGSGQLLWDTITGVDMAL

SEQ ID NO: 214 - protein, 466 aa, unidentified
  source 1..466: note = Influenza B virus
  REGION 1..466: note = B/Malaysia/2506/2004 NA
MLPSTIQTLTLFLTSGGVLLSLYVSASLSYLPYSDILLKFPSTEITAPTMPLDCANASNVQAVNRSATKGVTLLLPEPEWTYPRLSCPGSTFQKALLISPHRFGETKGNSAPLIIREPFIACGPKECKHFALTHYAAQPGGYYNGTRGDRNKLRHLISVKLGKIPTVENSIFHMAAWSGSACHDGKEWTYIGVDGPDNNALLKIKYGEAYTDTYHSYANNILRTQESACNCIGGNCYLMITDGSASGVSECRFLKIREGRIIKEIFPTGRIKHTEECTCGFASNKTIECACRDNSYTAKRPFVKLNVETDTAEIRLMCTETYLDTPRPDDGSITGPCESNGDKGSGGIKGGFVHQRMASKIGRWYSRTMSKTKRMGMGLYVKYDGDPWADSDALAFSGVMVSMEEPGWYSFGFEIKDKKCDVPCIGIEMVHDGGKETWHSAATAIYCLMGSGQLLWDTVTGVNMAL

SEQ ID NO: 215 - protein, 466 aa, unidentified
  source 1..466: note = Influenza B virus
  REGION 1..466: note = B/Brisbane/60/2008 NA
MLPSTIQTLTLFLTSGGVLLSLYVSASLSYLLYSDILLKFSPTEITAPTMPLDCANASNVQAVNRSATKGVTLLLPEPEWTYPRLSCPGSTFQKALLISPHRFGETKGNSAPLIIREPFIACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANKILRTQESACNCIGGNCYLMITDGSASGVSECRFLKIREGRIIKEIFPTGRVKHTEECTCGFASNKTIECACRDNSYTAKRPFVKLNVETDTAEIRLMCTDTYLDTPRPNDGSITGPCESNGDKGSGGIKGGFVHQRMESKIGRWYSRTMSKTERMGMGLYVKYDGDPWADSDALAFSGVMVSMKEPGWYSFGFEIKDKKCDVPCIGIEMVHDGGKETWHSAATAIYCLMGSGQLLWDTVTGVDMAL

SEQ ID NO: 216 - protein, 466 aa, unidentified
  source 1..466: note = Influenza B virus
  REGION 1..466: note = B/Colorado/06/2017 NA
MLPSTIQTLTLFLTSGGVLLSLYVSASLSYLLYSDILLKFSPTEITAPTMPLDCANASNVQAVNRSATKGVTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 217 - protein, 466 aa, unidentified
  source 1..466: note = Influenza B virus
  REGION 1..466: note = B/Washington/02/2019 NA
MLPSTIQTLTLFLTSGGVLLSLYVSASLSYLLYSDILLKFSPTEITAPTMPLDCANASNVQAVNRSATKGVTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSKTERMGMGLYVKYGGDPWADSDALTFSGVMVSMKEPGWYSFGFEIKDKKCDVPCIGIEMVHDGGKETWHSAATAIYCLMGSGQLLWDTVTGVDMAL

SEQ ID NO: 218 - protein, 466 aa, unidentified
  source 1..466: note = Influenza B virus
  REGION 1..466: note = B/Austria/1359417/2021 NA
MLPSTIQTLTLFLTSGGVLLSLYVSASLSYLLYSDILLKFSQTEITAPTMPLDCANASNVQAVNRSATKGATLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 219 - protein, 466 aa, unidentified
  source 1..466: note = Influenza B virus
  REGION 1..466: note = B/Beijing/184/1993 NA
MLPSTIQTLTLFLTSGGVLLSLYVSASLSYLLYSDILLKFSPTEITAPTMPLDCANASNVQAVNRSATKGVTLLLPEPEWTYPRLSCPGSTFQKALLISPHRFGETRGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYANNILRTQESACNCIGGDCYLMITDGSASGTSKCRFLKIREGRIIKEIFPTGRVEHTEECTCGFASNKTIECACRDNNYTAKRPFVKLNVETDAAEIRLMCTETYLDTPRPDDGSITGPCESNGDKGRGGIKGGFVHQRMASKIGRWYSRTMSKTERMGMELYVKYDGDPWTDSDALALSGVMVSMEEPGWYSFGFEIKDKKCDVPCIGIEMVHDGGKKTWHSAATAIYCLMGSGQLLWDTVTGVDMAL

SEQ ID NO: 220 - protein, 466 aa, unidentified
  source 1..466: note = Influenza B virus
  REGION 1..466: note = B/Sichuan/379/1999 NA
MLPSTIQTLTLFLTSGGVLLSLYVSASLSYLLYSDILLKFSPTEITAPTMPLDCANASNVQAVNRSATKGVTFLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGKEWTYIGVDGPDSNALLKIKYGEAYTDTYHSYANNILRTQESACNCIGGNCYLMITDGSASGISECRFLKIREGRIIKEIFPTGRVKHTEECTCGFASNKTIECACRDNSYTAKRPFVKLNVETDTAEIRLMCTETYLDTPRPDDGSITGPCESNGDKGSGGIKGGFVHQRMASKIGRWYSRTMSKTKRMGMGLYVKYDGDPWTDSDALALSGVMVSMEEPGWYSFGFEIKDKKCDVPCIGIEMVHDGGKETWHSAATAIYCLMGSGQLLWDTVTGVDMAL

SEQ ID NO: 221 - protein, 466 aa, unidentified
  source 1..466: note = Influenza B virus
  REGION 1..466: note = B/Shanghai/361/2002 NA
MLPSTIQTLTLFLTSGGVLLSLYVSASLSYLLYSDILLKFSTTEITAPTMPLDCANASNVQAVNRSATKGVTLLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGKEWTYIGVDGPDSNALLKIKYGEAYTDTYHSYANNILRTQESACNCIGGNCYLMITDGSASGISECRFLKIREGRIIKEIFPTGRVKHTEECTCGFASNKTIECACRDNSYTAKRPFVKLNVETDTAEIRLMCTETYLDTPRPDDGSITGPCESNGNKGSGGIKGGFVHQRMASKIGRWYSRTMSKTKRMGMGLYVKYDGDPWIDSDALALSGVMVSMEEPGWYSFGFEIKDKKCDVPCIGIEMVHDGGKETWHSAATAIYCLMGSGQLLWDTVTGVDMAL

SEQ ID NO: 222 - protein, 466 aa, unidentified
  source 1..466: note = Influenza B virus
  REGION 1..466: note = B/Florida/4/2006 NA
MLPSTIQTLTLFLTSGGVLLSLYVSASLSYLLYSDILLKFSQTEITAPIMPLDCANASNVQAVNRSAAKGVTLLLPEPEWTYPRLSCPGSTFQKALLISPHRFGETKGNSAPLIIREPFIACGPTECKHFALTHYAAQPGGYYNGTREDRNKLRHLISVKLGKIPTVENSIFHMAAWSGSACHDGKEWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTETYLDTPRPNDGSITGPCESDGDKGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVDMAL

SEQ ID NO: 223 - protein, 466 aa, unidentified
  source 1..466: note = Influenza B virus
  REGION 1..466: note = B/Wisconsin/1/2010 NA
MLPSTIQTLTLFLTSGGVLLSLYVSASLSYLLYSDILLKFSRTEITAPIMPLDCANASNVQAVNRSATKGVTLLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTETYLDTPRPNDGSITGPCESNGDKGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 224 - protein, 466 aa, unidentified
  source 1..466: note = Influenza B virus
  REGION 1..466: note = B/Massachusetts/2/2012 NA
MLPSTIQTLTLFLTSGGVLLSLYVSASLSYLLYSDILLKFSQTEITAPIMPLDCANASNVQAVNHSAAKGVTLLLPEPEWTYPRLSCPGSTFQKALLISPHRFGEIKGNSAPLIIREPFIACGPTECKHFALTHYAAQPGGYYNGTREDRNKLRHLISVKLGKIPTVENSIFHMAAWSGSACHDGKEWTYIGVDGPDSNALLKIKYGEAYTDTYHSYAKNILRTQESACNCIGGDCYLMITDGPASGVSECRFLKIREGRIIKEIFPTGRVKHTEECTCGFASNKTIECACRDNRYTAKRPFVKLNVETDTAEIRLMCTETYLDTPRPNDGSITGPCESDGDKGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVDMAL

SEQ ID NO: 225 - protein, 466 aa, unidentified
  source 1..466: note = Influenza B virus
  REGION 1..466: note = B/Phuket/3073/2013 NA
MLPSTIQTLTLFLTSGGVLLSLYVSASLSYLLYSDILLKFSRTEVTAPIMPLDCANASNVQAVNRSATKGVTPLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 226 - protein, 29 aa, unidentified
  source 1..29: note = Influenza A virus
  REGION 1..29: note = A/Wisconsin/67/2022 (H1N1)pdm09 N1 NA Signal Peptide
MNPNQKIITIGSVCMTIGTANLILQIGNI

SEQ ID NO: 227 - protein, 29 aa, unidentified
  source 1..29: note = Influenza A virus
  REGION 1..29: note = A/Darwin/6/2021 (H3N2) N2 NA Signal Peptide
MNPNQKIITIGSVSLTISTICFFMQIAIL

SEQ ID NO: 228 - protein, 31 aa, unidentified
  source 1..31: note = Influenza B virus
  REGION 1..31: note = B/Austria/1359417/2021 B/Victoria lineage NA Signal Peptide
MLPSTIQTLTLFLTSGGVLLSLYVSASLSYL

SEQ ID NO: 229 - protein, 31 aa, unidentified; identical to SEQ ID NO: 228
  source 1..31: note = Influenza B virus
  REGION 1..31: note = B/Phuket/3073/2013 B/Yamagata lineage NA Signal Peptide

SEQ ID NO: 230 - protein, 440 aa, unidentified
  source 1..440: note = Influenza A virus
  REGION 1..440: note = A/Wisconsin/67/2022 (H1N1)pdm09 N1 NA without SP
ISIWVSHSIQIGNQSQIETCNKSVITYENNTWVNQTFVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDN

SEQ ID NO: 231 - protein, 440 aa, unidentified
  source 1..440: note = Influenza A virus
  REGION 1..440: note = A/Darwin/6/2021 (H3N2) N2 NA without SP
ITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 232 - protein, 435 aa, unidentified
  source 1..435: note = Influenza B virus
  REGION 1..435: note = B/Austria/1359417/2021 B/Victoria lineage NA without SP
LYSDILLKFSQTEITAPTMPLDCANASNVQAVNRSATKGATLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 233 - protein, 435 aa, unidentified
  source 1..435: note = Influenza B virus
  REGION 1..435: note = B/Phuket/3073/2013 B/Yamagata lineage NA without SP
LYSDILLKFSRTEVTAPIMPLDCANASNVQAVNRSATKGVTPLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL